(19) United States
(12) Patent Application Publication
(10) Pub. No.: US 2024/0252475 A1
(43) Pub. Date: Aug. 1, 2024

(54) TIOTROPIUM COMBINATION PRODUCT COMPOSITIONS AND RELATED METHODS

(71) Applicant: Somerset Therapeutics, LLC, Hollywood, FL (US)

(72) Inventors: Lin Yang, Metuchen, NJ (US); Julianne Berry, Westfield, NJ (US); Ilango Subramanian, Warren, NJ (US); Veerappan Subramanian, Warren, NJ (US); Aman Trehan, Hillsborough, NJ (US)

(21) Appl. No.: 18/423,281

(22) Filed: Jan. 25, 2024

Related U.S. Application Data
(60) Provisional application No. 63/481,793, filed on Jan. 26, 2023.
(60) Provisional application No. 63/481,794, filed on Jan. 26, 2023.
(60) Provisional application No. 63/481,796, filed on Jan. 26, 2023.

(51) Int. Cl. A61K 31/4436; A61K 31/166; A61K 31/56; A61K 31/58

(52) U.S. Cl. CPC A61K 31/4436; A61K 31/166; A61K 31/56; A61K 31/58

(57) ABSTRACT

The present invention is directed to pharmaceutical composition(s) comprising a tiotropium compound, a formoterol compound, and a propellant component. The present invention is further directed to the process of preparing such composition(s) and method(s) of their use for the treatment of asthma, COPD, and related respiratory disorders.

### RELATED APPLICATIONS/PRIORITY

This patent application claims priority to U.S. Provisional Patent Application Nos. 63/481,796, 63/481,794, and 63/481,793, which each were filed on Jan. 26, 2023. This disclosure incorporates by reference the entirety of each of these referenced priority applications.

### FIELD OF THE INVENTION

The invention primarily relates to the field of pharmaceutical compositions for the treatment of conditions, including asthma, chronic obstructive pulmonary disorder (COPD), and related conditions.

### BACKGROUND OF THE INVENTION

Asthma is a long-term condition affecting both children and adults. According to the World Health Organization (WHO), asthma, the cough, wheeze, shortness of breath, and chest tightness symptoms of which are caused by the inflammation and narrowing of the small airways in the lungs, affected an estimated 262 million people worldwide in 2019, causing 455,000 deaths. Similarly, chronic obstructive pulmonary disease (COPD), referring to a group of diseases causing airflow blockage and breathing-related difficulties, is associated with challenges such as mucus hyper secretion, emphysema, and bronchiolitis. According to the Centers for Disease Control (CDC), COPD affects more than 15 million Americans alone, killing more than 150,000 Americans per year. The WHO reports COPD as the third leading cause of death globally, accounting for 3.23 million deaths in 2019.

Common treatments for conditions such as asthma and COPD include inhaled medication(s) which are typically delivered via an inhalation device (e.g., an “inhaler”). An inhaler can be, more specifically, an inhaler capable of delivering a controlled and consistent/measured dose of medication, referred to as a “metered dose inhaler” (MDI) or a “pressurized metered dose inhaler” (pMDI)). Medication(s) delivered by such devices most commonly include, e.g., bronchodilator(s) for dilating air passages and steroid(s) to reduce inflammation in the air passages. Drug delivery by inhalation for the treatment of breathing-related diseases has proven to be very effective. Unfortunately, however, successful formulation of compositions for delivery by MDIs has proven very challenging.

While directly targeting the lung, the end organ for the treatment of lung-related disease(s) and condition(s), is attractive due to the ability to directly access the lung via inhaled treatment(s), multiple factors contribute to making such delivery safe and effective. See, e.g., Labiris, et. al., in, “Pulmonary drug delivery. Part I: Physiological factors affecting therapeutic effectiveness of aerosolized medications,” Br J Clin Pharmacol. 2003 December; 56(6): 588-599. As summarized therein, as a major port of entry to the body, the lung has evolved numerous defense mechanisms to prevent the invasion of the body by undesirable airborne particles. Abundant and complicated air passageways, including significant branching and narrowing of the same, and environmental control systems such as, e.g., temperature and humidity control, make successful drug delivery to the lung difficult. Further, mucociliary clearance mechanism(s) and cellular clearance mechanism(s), e.g., as represented by the abundant presence of alveolar macrophages, are present specifically to maintain the sterility of the lung. Such defense mechanisms establish barriers to the effective delivery of inhaled therapeutics. Controlling where in the respiratory tract an inhaled therapeutic is deposited presents its own challenges. A drug may be more or less efficacious depending on how close to a target treatment site it is able to be delivered; yet directing precisely where an inhaled droplet or particle, e.g., comprising the therapeutic agent to be delivered, lands within the respiratory tract upon inhalation is impacted by a number of factors (such as, e.g., size, weight, force of inhalation, etc.) and is, again, quite challenging to control. (Labiris, et. al., Supra.)

The challenge of successfully formulating compositions capable of delivery by MDI is clearly demonstrated in the relevant art. Researchers working to meet the myriad of formulation challenges present in inhaled drug development repeatedly find that even slight differences in compositions, e.g., propellants and/or excipients of compositions, lead to quite different results. For example, leading pharmaceutical company Pfizer, failed to successfully develop inhaled insulin because the product actually decreased lung function, resulting in a more than 2.8-billion-dollar loss on the product (forbes.com/2007/10/18/pharmacuticals-pfizer-exubera-biz-sci-cx-mh-1018pfizer.html?sh=7b12ec471040). Several continued and significant difficulties with the development of inhaled pharmaceutical products continue to exist despite such failure. See, e.g., Anderson et al. Pharmacological Reviews January 2022, 74 (1) 48-118; doi.org/10.1124/pharmrev.120.000108.

One specific development challenge faced by formulators developing compositions to be delivered by inhalation, e.g., by MDI, is the need to use one or more propellant(s) to develop appropriate pressure within the delivery container to expel the composition in the form of a vapor, e.g., to aerosolize the components of the composition upon actuation of the delivery device. A propellant provides the force to expel the concentrated composition from the delivery container and establishes the form of composition upon delivery, e.g., as an aerosolized spray. Further, propellants can serve as a solvent or vehicle for a concentrated composition. (See, e.g., Kar, et. al, Current Developments in Excipient Science, Section 2.2.2.12 (Propellants), in Basic Fundamentals of Drug Delivery, 2019).

Identification of appropriate propellant(s) meeting a myriad of requirements requires significant experimentation. Suitable and effective propellants must comprise a boiling point and vapor pressure suitable for allowing liquification in a closed container at room temperature but capable of being maintained at a sufficiently high pressure such that when the MDI is activated, it is capable of delivering the composition (e.g., APIs) in atomized form. Further, suitable propellant(s) must have a sufficiently low acute and chronic toxicity and a sufficiently high cardiac sensitization threshold. Still further, a suitable and effective propellant must have a sufficiently high chemical stability when in contact with component(s) of the composition, such as, e.g., API(s), as well as components of the MDI itself, such as one or more metallic or non-metallic components. Still further, suitable propellant(s) must have a low propensity to extract low molecular weight substances from any elastomeric material(s) in the MDI; must comprise the ability to maintain API(s) of the composition in homogeneous solution, stable suspension, or stable dispersion to permit reproducible drug delivery when in use; and, e.g., must have a sufficiently low flammability so as to form a non-flammable or low-flammable mixture when mixed with air in the respiratory tract of the user. With regard to the ability to maintain API(s) of the composition in, e.g., stable suspension, the density of the propellant in liquid form must ideally be similar to that of the solid API(s) in order to avoid rapid sinking or floating of drug particles in the liquid; elements which may vary from API-to-API. Formulation of aerosols having, e.g., appropriate droplet size/volume is relevant to a composition's ability to deliver one or more pharmaceutical ingredients maintained therein to a target location within the lung, as is, e.g., the force at which such an aerosol is inhaled. Thus, not only is attaining a stable, homogenous composition challenging, but getting such a composition into a delivery form which can deposit a sufficient amount of API successfully to a target site within the lung(s) only exaggerates the formulation challenges.

Not only do chemical characteristics of propellant(s) require careful evaluation, but propellant(s) as a class of excipients face environmental regulations, or, at a minimum, environmental pressures, impacting their use. CFCs are used in small quantities in the medical field, such as, e.g., in MDIs, under an essential use exemption provided by the Montreal Protocol, a global agreement finalized in 1987 directed to protecting the stratospheric ozone layer. However, as a result of pressure to reduce CFC use, substances identifiable as hydrofluoroalkanes (HFAs) are now commonly used as propellants. HFAs are alkane compounds (aliphatic hydrocarbons, comprising chains of single-bonded carbon atoms). Such compounds are saturated compounds. Compounds such as, e.g., 1,1,1,2-tetrafluoroethane (HFA-134a) and 1,1,1,2,3,3,3-heptafluoropropane (HFA-227ea) are exemplary HFA compounds. United States patent publication number 2019/0374519 (to Corr; “the '519 publication”) alludes briefly to hydrofluoroolefin alternatives such as HFO-1234yl and HFO-1234ze. However, the '519 publication describes such alternatives as likely being toxic. The '519 publication discloses acid stabilizer-free pharmaceutical composition(s) comprising: (i) a drug component comprising tiotropium bromide monohydrate; and (ii) a propellant component, wherein at least 90 wt. % of the propellant component is 1,1-difluoroethane (HFA-152a).

Addressing environmental safety concerns by way of formulation adjuvants has been proposed. See, e.g., the disclosure of PCT publication WO2010/052466 (to Lulla, et. al.), the “466 publication.” The '466 publication discloses pharmaceutical aerosol compositions of tiotropium complexed with an adjuvant (PVP) for the treatment of respiratory disorders. The '466 publication further cites EP0372777, WO91/11173, and WO91/14422 as proposing, “ . . . the addition of one or more adjuvants such as alcohols, alkanes, dimethyl ether, surfactants (including fluorinated and non-fluorinated surfactants, carboxylic acids, polyethoxylates etc.) and even conventional chlorofluorocarbon propellants in small amounts intended to minimize potential ozone damage, but have not been able to establish any unexpected advantage.”

Still further challenges related to the use of new propellant(s) or propellant system(s) are described in, e.g., EP1870090 (to Calzada Pratarso; “the '090 patent”). The '090 patent teaches that, “ . . . new propellants require changes in the additives or coadjuvants necessary so that the preparation provides a medicament with pharmaceutically suitable characteristics.” This leads to a second specific challenge faced by formulators developing compositions to be delivered by MDI: compositional stability.

Achieving stability of compositions designed for delivery by inhalation, e.g., via an MDI, has proven to be nuanced and fraught with failure. In delivering compositions to the lung by inhalation, solutions are preferable to suspensions, as consistency in dosing is critical. However, is it difficult to solubilize an effective amount of drug for aerosolized delivery without requiring complicated manufacturing processes or, e.g., without requiring special care in design of the MDI to be used to deliver the composition. Excipient and propellant selection are critical. Exemplary challenges are disclosed in, e.g., the above-cited '466 publication, wherein the propellant HFA-134a is described as a poor solvent and propellant HFA-227 is described as poorly solubilizing or dispersing common surfactants. US patent publication number 2019/0054010 (to Slowey, et. al.) (the “'010 publication”) discloses a composition comprising tiotropium or a pharmaceutically acceptable salt or solvate thereof in a concentration of 0.0075 wt. % to 0.015 wt. % based on the weight of tiotropium bromide; citric acid or a salt thereof; glycerol; ethanol; and a propellant consisting essentially of HFC 134a; wherein the formulation is a solution. The '010 publication teaches that simple swapping out of propellants to address such issue(s) described above is not an option. The '010 publication exemplifies that in the compositions proposed therein, even though in the art HFA-134a and HFA-227 are often deemed to be equivalent, solution compositions comprising HFA-134a cannot be formed if HFA-134a is replaced with HFA-227. That is, with all other constituents/excipients remaining constant, the same composition goes from soluble (solution) to insoluble upon swapping propellants.

Returning to the aforementioned disclosure of the '466 publication, the '466 publication cites WO91/04011 and PCT/GB2008/002029 as attempting to address solubility problems by coating drug(s) with only surfactants, however, cites such approaches as yielding unsatisfactory results. The '466 publication presents the issue experienced by formulators related to the polar, hygroscopic nature of HFAs, whereby the ingress of water into MDIs is problematic; a problem worsened by use of polar, water miscible cosolvents such as alcohol. Such a scenario presents a catch-22. Use of polar, water miscible cosolvents aids in the solubilization of drug(s), however moisture in MDIs is problematic, as it adversely affects the physical stability of the composition maintained therein, e.g., by promoting the swelling of drug particles (e.g., increase in particle size) and the aggregation of particles, decreasing the likelihood of such particles being deposited in the far-reaches of the respiratory tract and, further, potentially detrimentally impacting performance of the MDI itself.

As if such challenges were not enough, even if one is able to successfully achieve a suitably consistent and efficacious composition, such compositions are often incompatible with typical, e.g., standard, metered dose inhalers or parts thereof, made by traditional manufacturing techniques and using typical/commonly used materials. Such compositions often require special manufacturing efforts, increasing manufacturing time and cost. For example, US patent publication number 2021/0260310 (to Slowey, et. al.) discloses composition(s) comprising a propellant such as HFC 134a; ethanol; citric acid; and tiotropium or a pharmaceutically acceptable salt or solvate thereof dissolved in the composition and proposes use of an aerosol canister having an internal surface made of a non-metal material, the non-metal material (such as, e.g., a silane primer composition) contacting the composition as opposed to the metal body of the canister maintaining the composition therein. Further, disclosure is directed to one or more valves, gaskets, seals, O-rings, etc. of the MDI being coated with one or more coatings to prevent incompatibility between the composition and any component of the MDI with which the composition may make contact. Similarly, US patent publication 2015/0250713 (to Malhotra et. al., the “'713 publication”) suggests that compositions provided therein can be dispensed from anodized containers or containers coated with a suitable polymer, such as, e.g., fluorocarbon copolymers such as FEP-PES (fluorinated ethylene propylene and polyethersulphone), PFA-PES (perfluoroalkoxyalkane and polyethersulphone), epoxy and ethylene or combinations thereof. Still further, EP2201934 (to CHIESI Farmaceutici S.p.A.) discloses aerosolized compositions which address stability concerns by use of mineral acid such as, e.g., hydrochloric, phosphoric, nitric, and sulfuric acid, but which are delivered from devices wherein canisters, valves, seals, etc. are selected so as to prevent corrosion, e.g., use of valves, rings, and the like made of a butyl or halo-butyl rubber to address compatibility concerns.

Additional art relevant to the development of inhaled compositions for the treatment of respiratory-related disorder(s) include, e.g., U.S. Pat. No. 6,537,524, describing the administration of, either separately or together (e.g., administered simultaneously, sequentially, or separately), formoterol or a pharmaceutically acceptable salt thereof or a solvate of formoterol or such a salt, and a tiotropium compound, in organic-acid free compositions provided in effective amount(s) for the treatment of an inflammatory or obstructive airway disease. Such medicament(s) are provided via a metered dose inhaler comprising HFA-227 and HFA-134a as propellant(s). US 2003/171586 (the “586 publication”) discloses crystalline tiotropium bromide and a process of manufacturing the same for dry powder inhalation. The '586 publication further discloses inhalable aerosols which may contain HFA-134a, HFA-227 or mixtures thereof as propellant(s). US 2005/0058606 discloses aerosol formulations comprising the single active pharmaceutical ingredient, tiotropium, or a pharmaceutically acceptable salt or hydrate thereof, an HFC propellant, a solvent, and an acid selected from the group consisting of one or more inorganic and organic acids. The propellant(s) used in examples therein are HFA-134a or HFC-227. The '713 publication referenced above discloses tiotropium compound and formoterol compound MDI formulation(s) comprising HFA propellant(s), e.g., at least two propellants for compositions provided as solutions, and optionally, one or more pharmaceutically acceptable excipients. Examples provided therein of suitable HFA propellants are HFA-32, HFA-143(a), HFA-134, HFA-152a, HFA-134(a) and HFA-227, and any mixture thereof, with the stated preference being HFA-134(a) and HFA-227 or a mixture thereof. The '713 publication teaches that a tiotropium HFA-227 composition is more stable than a tiotropium HFA-134(a) composition. The '090 patent referenced above discloses a formulation of suspended aerosols using hydrofluorocarbons as propellants, wherein stability is achieved by adding a dispersion coadjutant such as ethanol in very small quantities, e.g., in amounts of less than 0.30% w/w, and the use of valves provided with seals compatible with the formulation. Chlorofluorocarbonated (CFC) propellant alternatives were researched, and 1,1,1,2-tetrafluoroethane (HFA 134a) and 1,1,1,2,3,3,3-heptafluoroethane (HFA 227) were identified as suitable propellants. WO 2004/054580 discloses single active (tiotropium) solution formulation(s) comprising inorganic acid or organic acid, characterized in that the concentration of the acid is in a range that corresponds with a pH range of 2.5-4.5 in aqueous solution. Finally, combination compositions in the form of suspension(s) comprising a tiotropium compound and a formoterol compound provided by MDI is available in India marketed by Cipla and Precept Pharm under the brand name Duova and OVA® F-9 Lite inhaler. Therein, HFA-227 and HFA-134a propellant(s) (respectively) are used.

Clearly, the combination of challenges presented from, at a minimum, (a) lung anatomy and physiology, (b) propellant nuances, (c) compositional solubility & stability, and (d) device compatibility, make successful formulation of efficacious compositions for delivery by inhalation, e.g., for delivery via a metered dose inhaler, a formidable task. Obtaining stable, easy to manufacture composition(s) requires diligent formulation research and development effort, significant monetary investment, and creative ingenuity.

### Construction, Terms, and Acronyms

This section offers guidelines for reading this disclosure. The intended audience for this disclosure (“readers”) are persons having ordinary skill in the practice of technologies discussed or used herein. Readers may also be called “skilled persons,” and such technologies called “the art.” Terms such as “understood,” “known,” and “ordinary meaning,” refer to the general knowledge of skilled persons.

The term “uncontradicted” means not contradicted by this disclosure, logic, or plausibility based on knowledge of skilled persons.

Disclosed here are several different but related exemplary aspects of the invention (referred also to as, e.g., “cases,” “facets,” or “embodiments”). The invention encompasses all aspects as described individually and as can be arrived at by any combination of such individual aspects. The breadth and scope of the invention should not be limited by any exemplary embodiment(s). No language in this disclosure should be construed as indicating any element/step is essential to the practice of the invention unless such a requirement is explicitly stated. Uncontradicted, any aspect(s) can be combined with any other aspect(s).

Uncontradicted, all technical/scientific terms used here generally have the same meanings as commonly understood by skilled persons, regardless of any narrower examples or descriptions provided here (including any term introduced initially in quotations). However, aspects characterized by the inclusion of elements, steps, etc., associated with specific descriptions provided here are distinct embodiments of the invention. Uncontradicted, disclosure of any aspect using known terms, which terms are narrowed by example or otherwise in this disclosure, implicitly discloses related aspects in which such terms are alternatively interpreted using the broadest reasonable interpretation of skilled persons.

Uncontradicted, “or” means “and/or” here, regardless of any occasional inclusion of “and/or” (e.g., phrases such as “A, B, or C” and “A, B, and/or C” simultaneously disclose aspects including (1) all of A, B, and C; (2) A and C; (3) A and B; (4) B and C; (5) only A; (6) only B; and (7) only C (and also support sub-groupings, such as “A or B,” “A or C,” etc.)).

Uncontradicted, “also” means “also or alternatively.” Uncontradicted, “here” & “herein” mean “in this disclosure.” The term “i.a.” means “inter alia” or “among other things.” “Also known as” is abbreviated “aka” or “AKA.” “Elsewhere” means “elsewhere herein.”

For conciseness, symbols are used where appropriate. E.g., “&” is used for “and,” & “˜” for “about.” Symbols such as < and > are given their ordinary meaning (e.g., “≤” means “less than or equal to” & “≥” means “greater than or equal to”). A slash “/” can represent “or” (“A/B” means “A or B”) or identify synonyms of an element, as will be clear from context.

The inclusion of “(s)” after an element or a step indicates that ≥1 of such an element is present, step performed, and the like. E.g., “element(s)” means both 1 element or ≥2 elements, with the understanding that each thereof is an independent aspect of the invention.

Use of the abbreviation “etc.” (or “et cetera”) in association with a list of elements/steps means any or all suitable combinations of the recited elements/steps or any known equivalents of such recited elements/steps for achieving the function(s) of such elements/steps that are known in the art. Terms such as “and combinations,” or “or combinations” regarding listed elements/steps means any or all possible/suitable combinations of such elements/steps.

Aspects may be described as suitable for use(s) disclosed herein. Uncontradicted, terms such as “suitability” means acceptable or appropriate for performing a particular function/achieving particular state(s)/outcome(s), and typically means effective, practical, and non-deleterious/harmful in the context the term is used. E.g., uncontradicted, the term “suitable” means appropriate, acceptable, or in contexts sufficient, or providing at least generally or substantially all of an intended function, without causing or imparting significant negative/detrimental impact.

Uncontradicted, heading(s) (e.g., “Construction, Terms . . . ”) and subheadings are included for convenience and do not limit the scope of any aspect(s). Uncontradicted, aspect(s), step(s), or element(s) described under one heading can apply to other aspect(s) or step(s)/element(s) here.

Ranges of values are used to represent each value falling within such range that are within an order of magnitude of the smallest endpoint of the range without having to explicitly write each value of the range. E.g., a recited range of 1-2 implicitly discloses each of 1.0, 1.1, 1.2, . . . 1.9, and 2.0 and 10-100 implicitly discloses each of 10, 11, 12, . . . 98, 99, and 100). Uncontradicted, all ranges include the range's endpoints, regardless of how a range is described. E.g., “between 1-5” includes 1 and 5 in addition to 2, 3, and 4 (and all numbers between such numbers within an order of magnitude of such endpoints, e.g., 1.0, 1.1, . . . 4.9, and 5.0). For the avoidance of doubt, any number within a range, regardless of the order of magnitude of the number, is covered by the range (e.g., a range of 2-20 covers 18.593).

Terms of approximation (e.g., “about,” “˜,” or “approximately”) are used (1) to refer to a set of related values or (2) where a precise value is difficult to define (e.g., due to limits of measurement). Uncontradicted, all exact values provided here simultaneously/implicitly disclose corresponding approximate values and vice versa (e.g., disclosure of “about 10” provides explicit support for the use of 10 exactly in such aspect/description). Ranges described with approximate value(s) include all values encompassed by each approximate endpoint, regardless of presentation (e.g., “about 10-20” has the same meaning as “about 10-about 20”). The scope of value(s) encompassed by an approximate term typically depends on the context of the disclosure, criticality or operability, statistical significance, understanding in the art, etc. In the absence of guidance here or in the art for an element, terms such as “about” when used in connection with an element should be interpreted as ±10% of the indicated value(s) and implicitly disclosing ±5%, ±2%, ±1%, and ±0.5%.

Lists of aspects, elements, steps, and features are sometimes employed for conciseness. Unless indicated, each member of each list should be viewed as an independent aspect. Each aspect defined by any individual member of a list can have, and often will have, nonobvious properties vis-a-vis aspects characterized by other members of the list.

Uncontradicted, the terms “a” and “an” and “the” and similar referents encompass both the singular and the plural form of the referenced element, step, or aspect. Uncontradicted, terms in the singular implicitly convey the plural and vice versa herein (in other words, disclosure of an element/step implicitly discloses corresponding use of such/similar elements/steps and vice versa). Hence, e.g., a passage regarding an aspect including X step supports a corresponding aspect including several X steps. Uncontradicted, any mixed use of a referent such as “a” in respect of one element/step or characteristic and “one or more of” with respect to another element/step or characteristic in a paragraph, sentence, aspect, or claim, does not change the meaning of such referents. Thus, for example, if a paragraph describes a composition comprising “an X” and “one or more Ys,” the paragraph should be understood as providing disclosure of “one or more Xs” and “one or more Ys.”

“Significant” and “significantly” mean results/characteristics that are statistically significant using ≥1 appropriate test(s)/trial(s) in the given context (e.g., p≤0.05/0.01). “Detectable” means measurably present/different using known detection tools/techniques. The acronym “DOS” (or “DoS”) means “detectable(ly) or significant(ly).”

Uncontradicted, any value here that is not accompanied by a unit of measurement (e.g., a weight of 50 or a length of 20), any previously provided unit for the same element/step or the same type of element/step will apply, or, in cases where no such disclosure exists, the unit most commonly used in association with such an element/step in the art will apply.

Uncontradicted, the terms “including,” “containing,” “comprising,” and “having” mean “including, but not limited to” or “including, without limitation.” Uncontradicted, use of terms such as comprising and including regarding elements/steps means including any detectable number or amount of an element or including any detectable performance of a step/number of steps (with or without other elements/steps).

For conciseness, description of an aspect “comprising” or “including” an element, with respect to a collection/whole (e.g., a system, device, or composition), implicitly provides support for any detectable amount/number or ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the whole/collection being made up of the element, or essentially all of the whole/collection being made up of the element (i.e., that the collection consists essentially of the referenced element). Similarly, a method described as including a step with respect to an effect/outcome implicitly provides support for the referenced step providing ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the effect/outcome, representing ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the steps/effort performed, or both. Explicit listing of percentages of elements/steps in connection with aspects does not limit or contradict such implicit disclosure.

Uncontradicted, terms such as “comprising” when used in connection with a step of a method provide implicit support for performing the step once, ≥2 times, or until an associated function/effect is achieved.

Uncontradicted, the term “one” means a single type, single iteration/copy/thing, of a recited element or step, or both, which will be clear from context. For example, the referent “one” used with a component of a composition can refer to one type of element (which may be present in numerous copies, as in the case of an ingredient in a composition), one unit of the element, or both. Similarly, “one” component, a “single” component, or the “only component” of a system typically means 1 type of element (which may be present in numerous copies), 1 instance/unit of the element, or both. Further, “one” step of a method typically means performing one type of action (step), one iteration of a step, or both. Uncontradicted, a disclosure of “one” element provides support for both, but uncontradicted, any claim to any “one” element means one type of such an element (e.g., a component of a composition/system).

The term “some” means ≥2 copies/instances or ≥5% of a listed collection/whole is, or is made up of, an element. Regarding method(s), some means ≥5% of an effect, effort, or both, is made up of or is attributable to a step (e.g., as in “some of the method is performed by step Y”) or indicates a step is performed ≥2 times (e.g., as in “step X is repeated some number of times”). “Predominately,” “most,” or “mostly,” means detectably ≥50% (e.g., mostly comprises, predominately includes, etc., mean ≥50%) (e.g., a system that mostly includes element X is composed of ≥50% of element X). The term “generally” means ≥75% (e.g., generally consists of, generally associated with, generally comprises, etc., means ≥75%) (e.g., a method that generally consists of step X means that 75% of the effort or effect of the method is attributable to step X). “Substantially” or “nearly” means ≥95% (e.g., nearly all, substantially consists of, etc., mean ≥95%) (e.g., a collection that nearly entirely is made up of element X means that at least 95% of the elements in the collection are element X). Terms such as “generally free” of an element or “generally lacking” an element mean comprising ≤˜25% of an element and terms such as “substantially free” of an element mean comprising ≤˜5% of an element.

In certain embodiments describing API(s), excipient(s), or both present in amounts of “at least” or “greater than” a given amount or, e.g., present in amounts of “no more than” or “no greater than” or “less than” a given amount, the reader should interpret such disclosure as disclosing, e.g., encompassing and explicitly including, such undefined low or high amount(s) ranging to the opposite amount (high or low) that is maximally/minimally therapeutically effective, typically suitable, or both. For example, use of the phrase “at least” (and similar descriptors) in connection with an amount of a component of a formulation or of an entire formulation/composition can be interpreted as at least the amount described but that is no more than a maximally suitable or therapeutically effective amount (in the individual or in a population, such as determined in a clinical study). Similarly, phrases such as “less than” (and similar descriptors) an indicated amount can be interpreted referring to an amount that is still suitable (including, where appropriate, no amount, e.g., 0 units of the indicated component) or therapeutically effective (e.g., an amount that results in a DOS result in a significant number of individuals in a well-controlled and adequate study), but is less than the indicated amount.

The phrase “substantially identical” may be used in certain contexts to reflect that tests that would be considered substantially identical by those of skill in the art (not differing meaningfully in terms of outcome) or that component(s) or step(s) can achieve the same result in a similar way as a referenced set of component(s)/step(s) so as to not meaningfully differ in intended result and manner of achieving such a result. It will be appreciated that the phrase “substantially identical” in such contexts comprises the use of identical amounts, identical formulations, and identical conditions, or, e.g., in other respects, composition(s) demonstrate an identical performance as a comparator.

Uncontradicted, any aspect described with respect to an optionally present element(s)/step(s) also provides implicit support for corresponding aspect(s) in which one, some, most, generally all, nearly all, essentially all, or all of such element(s) are lacking/step(s) not performed, in respect of the relevant aspect. E.g., disclosure of a system comprising element X implicitly also supports a system lacking element X.

Uncontradicted, changes to tense or presentation of terms (e.g., using “comprises predominately” in place of “predominately comprises”) do not change the meaning of the corresponding term/phrase.

Uncontradicted, all methods provided here can be performed in any suitable order regardless of presentation (e.g., a method comprising steps A, B, and C, can be performed in the order C, B, and A; B and A and C simultaneously, etc.). Uncontradicted, elements of a composition can be assembled in any suitable manner by any suitable method. In general, any methods and materials similar or equivalent to those described here can be used in the practice of embodiments. Uncontradicted, the use of ordinal numbers such as “first,” “second,” “third,” etc. is to distinguish respective elements rather than to denote a particular order of those elements.

Uncontradicted, any elements, steps, components, or features of aspects and all variations thereof, etc., described in any part(s) of this disclosure, are within the scope of the invention.

Elements associated with a function can be described as “means for” performing a function in a composition/device/system or a “step for” performing a part of a method, and parts of this disclosure refer to “equivalents,” which means equivalents known in the art for achieving a referenced function associated with disclosed mean(s)/step(s). However, no element of this disclosure or claim should be interpreted as limited to a “means-plus-function” construction unless such intent is clearly indicated by the use of the terms “means for” or “step for.” Terms such as “configured to” or “adapted to” do not indicate “means-plus-function” interpretation, but, rather, describe element(s)/step(s) configured to, designed to, selected to, or adapted to achieve a certain performance, characteristic, property, etc. using teachings provided here or in the art.

All references (e.g., publications, patent applications, and patents) cited herein are hereby incorporated by reference as if each reference were individually and specifically indicated to be incorporated by reference and set forth in its entirety herein. Uncontradicted, any suitable principles, methods, or elements of such references (collectively “teachings”) can be combined with or adapted to aspects. However, citation/incorporation of patent documents is limited to the technical disclosure thereof and does not reflect any view regarding the validity, patentability, etc., thereof. In the event of any conflict between this disclosure and the teachings of such documents, the content of this disclosure will take precedence regarding interpreting aspects of the invention. Numerous references are cited here to concisely incorporate known information and aid skilled persons in putting aspects into practice. While efforts have been made to include the most relevant references for such purposes, readers will understand that not every aspect of every cited reference will apply to every aspect of the invention. In aspects, the inventions described herein differ from the prior art in one or more respects. Accordingly, compositions and methods described herein can be characterized by the lack of any one or more of the various components, characteristics, and steps described in any of the prior art references cited herein.

### Additional Terms, Concepts, and Acronyms

The following description of certain terms and acronyms is provided to assist readers in understanding the invention. Additional acronyms may be only provided in other parts of this disclosure and acronyms that are well known in the art may not be provided here.

Uncontradicted, any description of weight is weight percent (“wt. %”).

Except where explicitly indicated or clearly indicated by context, “improved” herein means “increased.” In aspects, “improved” means “reduced,” such as with respect to the toxicity of a composition. Those skilled in the art should interpret use of such language in context. Uncontradicted, terms such as “enhanced,” “improved,” and the like are used synonymously.

“Pharmaceutical suitability”, “pharmaceutically suitable”, or, e.g., “pharmaceutically suitability” are phrases typically used to refer to composition(s) that are safe and effective for pharmaceutical administration and application including administration by inhalation, having sufficient potency, purity, strength, quality, and safety for pharmaceutical application, in cases specifically to the lung/airways, as may be judged by regulatory authority review, and as established by, e.g., one or more well controlled and adequate clinical studies performed in compliance with generally prevailing regulatory authority standards. Composition(s) described as “suitable for inhalation” should be interpreted to mean suitable for delivery by inhalation, such as, e.g., delivery as an aerosolized composition, when provided in a potency, purity, strength, or quality making it safe for inhalation. Component(s) or constituent(s)/agent(s) described as “suitable for inhalation” should be interpreted in a similar manner. Uncontradicted, a description of “suitability” implicitly means that the referenced element, step, etc., is pharmaceutically suitable/suitable for inhalation or otherwise medically suitable (e.g., safe and effective as determined by proper nonclinical/clinical testing).

Agent(s)/constituent(s), including, e.g., excipient(s), API(s), or both, herein are typically present in composition(s)/formulation(s) in “effective amounts,” and, uncontradicted, any described class of API or component, agent, constituent, e.g., specific constituent, is understood to be present in the associated composition/formulation in an effective amount, which generally means, in this context, an amount that is effective for the described function(s) associated with the agent (it being understood that some agent/API compound(s)/ingredient(s) exhibit more than one effect). E.g., a suspension agent will be understood to be present in a composition/formulation in an amount that is effective to impart an indicated suspension-related effect, a suspension effect that is required for suitability (e.g., suitable delivery) of the composition, or an effect that imparts a significant suspension effect (e.g., suspension of one or more API(s)) on a composition (with respect to a comparator composition lacking the compound(s)/ingredient(s)).

Herein, the term “compositionally compatible” is used to describe composition component(s) which, when present in composition(s) or, e.g., when present in given amount(s), do not detectably or significantly, e.g., measurably detrimentally, decrease the chemical stability of one or more compounds of the composition, decrease the physical stability of the composition, or both. In aspects, a compositionally compatible component or compound/agent is a component/compound/agent which does not detectably or significantly increase one or more API-related impurities over a given storage period under given storage condition(s). In aspects, a compositionally compatible component or compound/agent is a component/compound/agent which does not detectably or significantly impact the formulation status, such as, e.g., the composition maintains status as a solution (that is, e.g., when present or when present in given amounts, does not cause one or more APIs to come out of solution (does not detectably or significantly decrease the solubility of one or more APIs.) In aspects, a compositionally incompatible component/compound/agent is, e.g., a compound or agent which, when present in the composition, either in any amount or above a provided threshold or under certain conditions, causes the composition to demonstrate a detectably or significantly reduced chemical stability, physical stability, or both.

Elements in this specification that are referred to as “common,” “commonly used,” and the like, refer to elements common within the context of the compositions, articles, such as inhalers and metered dose inhalers, and methods of this disclosure; this terminology is not used to mean that these features are present, much less common, in the prior art.

In some cases, descriptions of terms and/or acronyms are repeated one or more times in the following portions of the disclosure to aid readability.

### SUMMARY OF THE INVENTION

The inventions described and claimed herein have many attributes and aspects including, but not limited to, those set forth in, e.g., described or referenced in, this Summary. This Summary of the Invention (“Summary”) is not intended to be all-inclusive, and the scope of the invention is not limited to or by the aspects, features, elements, or embodiments provided in this Summary, which is included for illustrative purposes only and not restriction. Any of the aspects described under this section can be combined with any other aspect described in this section or with any other aspect of this disclosure.

In aspects, the invention provides pharmaceutically acceptable composition(s) comprising an active pharmaceutical ingredient (API) component comprising one or more API compound(s)/constituent(s) and a propellant component. In aspects, each constituent of the API component is at least substantially dissolved. In aspects, each constituent of the API component is at least substantially dissolved such that a sufficiently homogeneous mixture is formed so as to form a solution. In aspects, composition(s), e.g., solution(s), allow light to at least substantially uniformly pass through the composition(s). In aspects, the API component of composition(s) comprises a tiotropium compound and a formoterol compound. In aspects the tiotropium compound is a salt of tiotropium, such as, e.g., tiotropium bromide. In aspects, the tiotropium compound is present in an amount of between about 0.005 wt. % and about 0.01 wt. % of composition(s). In aspects, the formoterol compound is a salt of formoterol, such as, e.g., formoterol fumarate, e.g., formoterol fumarate dihydrate. In aspects, the formoterol compound is present in an amount of between about 0.005 wt. % and about 0.01% of composition(s). In aspects, the propellant component of composition(s) comprises one or more propellant compound(s). In aspects, the propellant compound(s) is/are selected from HFA-134a, HFA-152a, and HFO-1234ze.

In aspects, composition(s) comprise a solvent component. In aspects, a solvent component comprises an effective amount of at least one C1-C6 alcohol characterizable as an alkyl alcohol, an aliphatic alcohol, or both. In aspects, a C1-C6 alcohol is present in an amount of at least about 6 wt. % of composition(s), such as, e.g., at least about 11.25 wt. %, at least about 12 wt. %, or, e.g., in an amount of between about 12 wt. % and about 18 wt. % of composition(s). In aspects, the C1-C6 alcohol is ethanol.

In aspects, composition(s) comprise a stabilizing component. In aspects, composition(s) comprise an effective amount of pharmaceutically acceptable and compositionally compatible small compound stabilizing agent(s). In aspects, such agent(s) have a molecular weight of less than about 200 g/mol, provide detectable or significant chelating activity, or both. In aspects, small compound stabilizing agent(s) detectably or significantly prevent or delay any change in the homogeneity of composition(s). In aspects, small compound stabilizing agent(s) detectably or significantly prevent or delay any change in the homogeneity of composition(s) resulting in (a) the amount of light capable of passing through composition(s) to be reduced or (b) composition(s) no longer being characterizable as a solution. In aspects, composition(s) comprise one or more acid(s). In aspects, such acid(s) are pharmaceutically acceptable and compositionally compatible small compound stabilizing agent(s). In aspects, small compound stabilizing agent(s) is/are an acid such as an organic acid, e.g., citric acid or, e.g., maleic acid.

In aspects, composition(s) comprise a ratio of pharmaceutically compatible small compound stabilizing agent(s) to an at least one C1-C6 alcohol characterizable as an alkyl alcohol, an aliphatic alcohol, or both, which is not greater than about 1:300, such as not greater than about 1:350 or, e.g., not greater than about 1:400.

In aspects, composition(s) further comprise a sugar alcohol, such as, e.g., glycerol.

In aspects, composition(s) do not comprise any compound comprising at least 12 contiguous carbons which demonstrates detectable or significant surfactant activity. In aspects, composition(s) do not comprise a compound comprising a ratio of carbon atoms to any non-hydrogen atom in the compound of at least about 1.5:1, e.g., at least about 4:1, which demonstrates detectable or significant surfactant activity. In aspects, composition(s) are free of any compound providing a significant level of surfactant activity.

In aspects, the invention provides composition(s) such as those described in this Summary which comprise less than about 0.2 wt. % total impurities, e.g., less than about 0.1 wt. % total impurities, generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 1 month, such as, e.g., at least about 3 months, at least about 6 months, at least about 9 months, or, e.g., at least about 12 months.

In aspects, the invention provides method(s) of manufacturing composition(s) such as those described in this Summary. In aspects, the invention provides methods of using composition(s) such as those described in this summary in the direct or prophylactic treatment of chronic obstructive pulmonary disease (COPD), asthma, one or more similar or related disorders (e.g., disorders related to the obstruction of airflow to or from the lung(s), any one or more symptoms related to any one or more of such condition, or any combination of any or all thereof. In aspects, such method(s) comprise administering a therapeutically effective amount of composition(s) such as those described in this Summary. In further aspects, the invention provides kit(s) comprising one or more composition(s) such as composition(s) described in this Summary. In aspect(s), kits comprise one or more composition(s) and one or more device(s) for delivering such composition(s), such as, e.g., one or more devices suitable for delivering composition(s) by inhalation. In aspects such device(s) comprise an inhaler device, such as, e.g., a metered dose inhaler.

In aspects, the invention provides pharmaceutically acceptable composition(s) comprising an active pharmaceutical ingredient (API) component and a propellant component. In aspects, each constituent, e.g., compound, of the API component is at least substantially dissolved. In aspects, each constituent of the API component is at least substantially dissolved such that a sufficiently homogeneous mixture is formed so as to form a solution. In aspects, composition(s), e.g., solution(s), allow light to at least substantially uniformly pass through them. In aspects, the API component comprises a tiotropium compound and a formoterol compound. In aspects the tiotropium compound is a salt of tiotropium, such as, e.g., tiotropium bromide. In aspects, the tiotropium compound is present in an amount of between about 0.005 wt. % and about 0.01 wt. % of the composition(s). In aspects, the formoterol compound is a salt of formoterol, such as, e.g., formoterol fumarate, e.g., formoterol fumarate dihydrate. In aspects, the formoterol compound is present in an amount of between about 0.005 wt. % and about 0.01% of the composition(s). In aspects, the propellant component comprises one or more propellant compounds. In aspects, the propellant component of composition(s) is at least substantially uniform, e.g., the propellant component is composed of at least about 95% of the same propellant compound. In certain aspects, a propellant component of composition(s) is at least about 96% composed of the same propellant compound wherein such compound establishes a dominant propellant. In aspects, the propellant component comprises a single propellant. In aspects, the propellant component comprises HFA-134a, HFA-152a, HFO-1234ze, or a combination thereof.

In aspects, composition(s) comprise a solvent component. In aspects, a solvent component comprises an effective amount of at least one C1-C6 alcohol characterizable as an alkyl alcohol, an aliphatic alcohol, or both. In aspects, the C1-C6 alcohol is present in an amount of at least about 6 wt. % of composition(s), such as, e.g., at least about 11.25 wt. %, at least about 12 wt. %, or, e.g., in an amount of between about 12 wt. % and about 18 wt. %. In aspects, the C1-C6 alcohol is ethanol.

In aspects, composition(s) comprise an effective amount of pharmaceutically acceptable and compositionally compatible small compound stabilizing agent(s). In aspects, such agent(s) have a molecular weight of less than about 200 g/mol, provide(s) detectable or significant chelating activity, or both. In aspects, small compound stabilizing agent(s) detectably or significantly prevent(s) or delay(s) any change in the homogeneity of composition(s). In aspects, small compound stabilizing agent(s) detectably or significantly prevent(s) or delay(s) any change in the homogeneity of composition(s) resulting in (a) the amount of light capable of passing through composition(s) to be reduced or (b) composition(s) no longer being characterizable as solution(s). In aspects, composition(s) comprise one or more acid(s). In aspects, such acid(s) are pharmaceutically acceptable and compositionally compatible small compound stabilizing agent(s). In aspects, such small compound stabilizing agent(s) is/are acid(s) such as organic acid(s), e.g., citric acid or, e.g., maleic acid. In aspects, composition(s) further comprise a sugar alcohol, such as, e.g., glycerol. In aspects, composition(s) are solution(s) wherein the (a) pharmaceutically acceptable and compositionally compatible small compound stabilizing agent comprises an organic acid, (b) composition(s) comprise a small compound stabilizing agent and a sugar alcohol, or (c) both (a) and (c) are true.

In aspects, composition(s) comprise a ratio of the percent of the propellant component represented by a dominant propellant to pharmaceutically acceptable and compositionally compatible small compound stabilizing agent(s) is at least about 320:1, such as, e.g., between about 320:1 and about 333:1.

In aspects, composition(s) comprise a ratio of pharmaceutically compatible small compound stabilizing agent(s) to an at least one C1-C6 alcohol characterizable as an alkyl alcohol, an aliphatic alcohol, or both which is not greater than about 1:300, such as not greater than about 1:350 or, e.g., not greater than about 1:400.

In aspects, composition(s) do not comprise any compound comprising at least 12 contiguous carbons which demonstrates detectable or significant surfactant activity. In aspects, composition(s) do not comprise a compound comprising a ratio of carbon atoms to any non-hydrogen atom in the compound of at least about 1.5:1, e.g., at least about 4:1, which demonstrates detectable or significant surfactant activity. In aspects, composition(s) are free of any compound providing a significant level of surfactant activity.

In aspects, the invention provides composition(s) such as those described in this Summary which comprise less than about 0.2 wt. % total impurities, e.g., less than about 0.1 wt. % total impurities, generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 1 month, such as, e.g., at least about 3 months, at least about 6 months, at least about 9 months, or, e.g., at least about 12 months.

In aspects, the invention provides method(s) of manufacturing composition(s) such as those described in this Summary. In aspects, the invention provides methods of using composition(s) such as those described in this summary in the direct or prophylactic treatment of chronic obstructive pulmonary disease (COPD), asthma, one or more similar or related disorders (e.g., disorders related to the obstruction of airflow to or from the lung(s), any one or more symptoms related to any one or more of such condition, or any combination of any or all thereof. In aspects, such method(s) comprise administering a therapeutically effective amount of composition(s) such as those described in this Summary. In further aspects, the invention provides kit(s) comprising one or more composition(s) such as composition(s) described in this Summary. In aspect(s), kits comprise one or more composition(s) and one or more device(s) for delivering such composition(s), such as, e.g., one or more devices suitable for delivering composition(s) by inhalation. In aspects such device(s) comprise an inhaler device, such as, e.g., a metered dose inhaler.

In aspects, the invention provides pharmaceutically acceptable composition(s) suitable for delivery by inhalation. In aspects, composition(s) comprise an active pharmaceutical ingredient (API) component and a propellant component. In aspects, the API component of composition(s) comprises at least two API compounds. In aspects, the API component comprises (a) an anticholinergic compound fraction comprising one or more anticholinergic agent(s), and (b) a non-anticholinergic compound fraction comprising one or more non-anticholinergic compound agent(s). In aspects, composition(s) comprise a propellant component comprising one or more propellant compound(s) each having a global warming potential of less than about 1500, such as less than about 1000, less than about 100, or, e.g., less than about 10. In aspects, the propellant component comprises HFA-134a, HFA-152a, HFO-1234ze, or a combination thereof. According to certain aspects, the propellant component is at least substantially uniform. In aspects, the propellant component is at least about 95% composed of the same propellant compound, establishing a dominant propellant compound. In aspects, the propellant component comprises a single propellant compound.

In aspects, composition(s) comprise one or more pharmaceutically acceptable and compositionally compatible small acid compound stabilizing agent(s). In aspects, such agent(s) have a molecular weight of less than about 200 g/mol, provide detectable or significant chelating activity, or both. In aspects, such agent(s) detectably or significantly prevent or delay any detectable or significant change in the homogeneity of composition(s). In aspects, the small compound stabilizing agent(s) detectably or significantly prevent(s) or delay(s) any change in the homogeneity of composition(s) resulting in (a) the amount of light capable of passing through composition(s) to be reduced or (b) composition(s) no longer being characterizable as solution(s). In aspects, composition(s) comprise one or more acid(s). In aspects, such acid(s) are pharmaceutically acceptable and compositionally compatible small compound stabilizing agent(s). In aspects, such a small compound stabilizing agent(s) is/are an acid such as an organic acid, e.g., citric acid or, e.g., maleic acid.

In aspects, the invention provides composition(s) wherein the anticholinergic compound fraction of the API component comprises a tiotropium compound, e.g., a salt of tiotropium, such as, e.g., tiotropium bromide. In aspects, a tiotropium compound is present in composition(s) in an amount of between about 0.005 wt. % and about 0.01 wt. % of the composition(s). In aspects, composition(s) comprise a non-anticholinergic compound fraction of an API component comprising at least one beta adrenergic agonist compound, such as, e.g., a formoterol compound, e.g., a salt of formoterol, such as, for example formoterol fumarate, e.g., formoterol fumarate dihydrate. In aspects, a formoterol compound is present in composition(s) in an amount of between about 0.005 wt. % and about 0.01 wt. % of the composition(s). In aspects, a non-anticholinergic compound fraction of an API component of composition(s) comprises one or more anti-inflammatory compounds, e.g., one or more steroid anti-inflammatory compound(s). In aspects, a non-anticholinergic compound fraction of an API component of composition(s) comprises one or more corticosteroid compound(s). In aspects, a non-anticholinergic compound fraction of an API component of composition(s) comprises one or more corticosteroid compound(s) selected from beclomethasone, ciclesonide, and a fluticasone compound. In aspects, the invention provides composition(s) wherein a non-anticholinergic compound fraction of an API component comprises an inhalable corticosteroid compound present in the composition in an amount of between about 0.18 wt. % and about 0.36 wt. %. In aspects, composition(s) provided by the invention comprise a non-anticholinergic compound fraction of the API component comprising at least two non-anticholinergic compounds. In aspects, the at least two anticholinergic compounds comprise at least one beta-adrenergic agonist, such as, e.g., a formoterol compound, e.g., a salt of formoterol, e.g., formoterol fumarate and, e.g., one or more anti-inflammatory compound(s), such as one or more steroid anti-inflammatory compound(s), e.g., one or more corticosteroid compound(s) such as an inhaled corticosteroid selected from beclomethasone, ciclesonide, and a fluticasone compound.

In aspects, composition(s) provided by the invention comprise a ratio of the percent of the propellant component represented by a dominant propellant (dominant propellant compound) to pharmaceutically acceptable and compositionally compatible small compound stabilizing agent(s) is at least about 320:1, such as, e.g., between about 320:1 and about 333:1.

In aspects, compositions comprise a ratio of the one or more pharmaceutically acceptable and compositionally compatible small compound stabilizing agent(s) to the non-anticholinergic compound fraction of the active pharmaceutical ingredient component of between about 1:370 and about 20:1, such as, e.g., between about 1:300 and about 20:1, between about 1:200 and about 20:1, between about 1:100 and about 20:1, between about 1:50 and about 20:1, between about 1:20 and about 20:1, between about 1:10 and about 20:1, between about 1:5 and about 20:1, between about 1:5 and about 10:1, between about 1:5 and about 5:1, or, e.g., between about 1:5 and about 1:1.

In aspects, composition(s) do not comprise any compound comprising at least 12 contiguous carbons which demonstrates detectable or significant surfactant activity. In aspects, composition(s) do not comprise a compound comprising a ratio of carbon atoms to any non-hydrogen atom in the compound of at least about 1.5:1, e.g., at least about 4:1, which demonstrates detectable or significant surfactant activity. In aspects, composition(s) are free of any compound providing a significant level of surfactant activity. In alternative aspects, composition(s) herein comprise a surfactant component. In aspects, composition(s) comprise a surfactant component comprising one or more surfactant compound(s). In aspects, surfactant compound(s) comprise at least 12 contiguous carbons and demonstrate detectable or significant surfactant activity. In aspects, surfactant compound(s) comprise a ratio of carbon atoms to any non-hydrogen atom in the compound of at least about 1.5:1, such as, e.g., at least about 4:1.

According to aspects, compositions(s) comprise one or more excipient component(s). In aspects, composition(s) comprise a solvent component, a suspension component, a bulking component, an absorbent component, or a combination of any or all thereof. In aspects, composition(s) comprise a solvent component. In aspects, composition(s) comprise a solvent component that detectably or significantly prevents or delays any change in the homogeneity of the composition. In aspects, the solvent component comprises one or more solvent compound(s). In aspects, a solvent compound is present in an amount representing at least about 5 wt. % of composition(s), such as, e.g., at least about 6 wt. %, at least about 8 wt. %, at least about 10 wt. %, at least about 11 wt. %, or at least about 12 wt. %, such as, e.g., between about 12 wt. % and about 18 wt. % of composition(s). In aspects, a solvent component of composition(s) comprises, e.g., one or more C1-C6 alcohol(s), one or more sugar alcohol(s), or both. In aspects, composition(s) comprise a solvent component comprising ethanol, glycerol, or both. In aspects, composition(s) comprise a suspension component. In aspects, a suspension component of composition(s) comprises, e.g., one or more of PEG 600, PEG 1000, and glycine. In aspects, composition(s) comprise a bulking component. In aspects, a bulking component of composition(s) comprises, e.g., one or more of lactose, magnesium stearate, and mannitol. In aspects, composition(s) comprise an absorbent component. In aspects, an absorbent component of composition(s) comprises, e.g., sodium cromoglycate.

In aspects, the invention provides composition(s) such as those described in this Summary wherein the active pharmaceutical ingredient component is at least substantially dissolved such that a sufficiently homogeneous mixture is formed so as to form a solution which allows light to at least substantially uniformly pass through the composition(s). In alternative aspects, at least a significant portion of one or more compound(s)/constituent(s) of the active pharmaceutical ingredient component remains undissolved such that light cannot pass uniformly through the composition and the composition is characterizable as a suspension or a hybrid solution-suspension.

In aspects, the invention provides composition(s) such as those described in this Summary which comprise less than about 0.2 wt. % total impurities, e.g., less than about 0.1 wt. % total impurities, generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 1 month, such as, e.g., at least about 3 months, at least about 6 months, at least about 9 months, or, e.g., at least about 12 months.

In aspects, the invention provides method(s) of manufacturing composition(s) such as those described in this Summary. In aspects, the invention provides methods of using composition(s) such as those described in this summary in the direct or prophylactic treatment of chronic obstructive pulmonary disease (COPD), asthma, one or more similar or related disorders (e.g., disorders related to the obstruction of airflow to or from the lung(s), any one or more symptoms related to any one or more of such condition, or any combination of any or all thereof. In aspects, such method(s) comprise administering a therapeutically effective amount/number or concentration of composition(s) such as those described in this Summary. In further aspects, the invention provides kit(s) comprising one or more composition(s) such as composition(s) described in this Summary. In aspect(s), kits comprise one or more composition(s) and one or more device(s) for delivering such composition(s), such as, e.g., one or more devices suitable for delivering composition(s) by inhalation. In aspects such device(s) comprise an inhaler device, such as, e.g., a metered dose inhaler.

### EXEMPLARY ASPECTS OF THE INVENTION

The following is a non-limiting list of exemplary aspects of the invention, which illustrates embodiments of the invention in a summary form to aid readers in quickly understanding the overall scope of the invention.

It is intended that these listed exemplary aspects begin with the first listed aspect (ASPECT 1) and thereafter be numbered sequentially and incrementally by the inclusion of a reference placed near or at the end of the listed aspect (ASPECT 2, ASPECT, 3, etc.). Like patent claims, these aspects of the invention listed in the paragraphs of this section may refer to (depend on/from) one or more other aspects referenced in other paragraphs. Readers will understand that such references mean that the features/characteristics or steps of such referenced aspects are incorporated into/combined with the referring aspect. For example, if an aspect in a paragraph (e.g., a paragraph indicated by text at the end of the paragraph as aspect 2) refers to another aspect by one or more aspect numbers (e.g., aspect 1 or “any one of aspects 1-3”), it will be understood to include the elements, steps, or characteristics of such referenced aspects (e.g., aspect 1) in addition to those of the aspect in which the reference is made (e.g., if aspect 2 refers to aspect 1, it provides a description of an object or method including the features of both aspect 1 and aspect 2). Reference to ranges of aspects should be interpreted as referencing all such aspects individually, each as unique embodiments of the invention, and in combination with one another as unique embodiment(s) of the invention, according to the presentation provided of such aspects unless such an aspect within such a referenced range is either contradictory or non-sensical. If contradicted, reference to the contradictory aspect should be excluded. In case of a missing aspect reference or repeated aspect reference, the order of placement of the actual recited aspect in the list that is associated with the repeated aspect reference or missing aspect reference will control (e.g., if there is an unlabeled aspect located between a first aspect labeled ASPECT 1 and a third aspect labeled aspect ASPECT 2, the unlabeled aspect should be treated as ASPECT 2, and the aspect labeled as ASPECT 2 treated as ASPECT 3, etc.), and all numbering in the list (including all references to aspects in the list) be interpreted as accordingly modified (e.g., if the fourth aspect in such list was labeled as ASPECT 3, it should be interpreted as being labeled as ASPECT 4, and if such aspect referred to “any one or both of aspect 1 or aspect 2,” it should be read as referring to “any one or more of aspects 1-3”). Similarly, if an aspect is misnumbered (e.g., by a number in the sequence being skipped or otherwise missing), readers will similarly construe this list of aspects according to the order of placement of the recited aspects, over the numerical references. Further, if one or more of the listed exemplary aspects of the invention in this section fails to reference any other aspects of the invention, such aspect, uncontradicted, should be interpreted as applying to or as capable of being incorporated into, any one or more other exemplary aspect(s) provided in this section.

### Solution Composition Embodiments

The following is a first listed set of representative aspects of the invention according to a first exemplary embodiment directed to pharmaceutical compositions of the invention in the form of a solution and related methods of manufacture and use. As noted elsewhere, uncontradicted any aspect in any section under any heading of this disclosure can be combined with any aspect from any other section.

In a first aspect according to this first embodiment, the invention provides a pharmaceutically acceptable composition comprising an active pharmaceutical ingredient component in solution, wherein each constituent of the active pharmaceutical ingredient component is at least substantially dissolved such that the solution is sufficiently homogeneous so as to allow light to at least substantially uniformly pass through the composition, wherein the active pharmaceutical ingredient component comprises an effective amount of at least one tiotropium compound (a tiotropium compound); at least one formoterol compound (a formoterol compound); and at least one pharmaceutically acceptable inhalable corticosteroid compound, and wherein the composition further comprises (1) an effective amount of at least one C1-C6 alcohol characterizable as an alkyl alcohol, an aliphatic alcohol, or both; (2) an effective amount of one or more pharmaceutically acceptable and compositionally compatible small compound stabilizing agent(s) each having a molecular weight of less than about 200 g/mol, providing detectable or significant chelating activity, or both, wherein the small compound stabilizing agent(s) is/are capable of detectably or significantly preventing or delaying any change in the homogeneity of the composition resulting in (a) the amount of light capable of passing through the composition to be reduced or (b) the composition no longer being characterizable as a solution; and (3) a propellant component comprising one or more pharmaceutically acceptable propellant compounds, wherein the ratio of the pharmaceutically compatible small compound stabilizing agent to the at least one C1-C6 alcohol characterizable as an alkyl alcohol, an aliphatic alcohol, or both is not greater than about 1:300. ASPECT 1.

In aspects, the invention provides the composition of aspect 1, wherein the tiotropium compound is a salt of tiotropium. ASPECT 2.

In aspects, the invention provides the composition of any one or both of aspect 1 and aspect 2, wherein the tiotropium compound is tiotropium bromide. ASPECT 3.

In aspects, the invention provides the composition of any one or more of aspects 1-3, wherein the tiotropium compound is present in an amount of between about 0.005 wt. % and about 0.01 wt. % of the composition. ASPECT 4.

In aspects, the invention provides the composition of any one or more of aspects 1-4, wherein the formoterol compound is a salt of formoterol. ASPECT 5.

In aspects, the invention provides the composition of any one or more of aspects 1-5, wherein the formoterol compound is formoterol fumarate. ASPECT 6.

In aspects, the invention provides the composition of any one or more of aspects 1-6, wherein the formoterol compound is formoterol fumarate dihydrate. ASPECT 7.

In aspects, the invention provides the composition of any one or more of aspects 1-7, wherein the formoterol compound is present in an amount of between about 0.005 wt. % and about 0.01 wt. % of the composition. ASPECT 8.

In aspects, the invention provides the composition of any one or more of aspects 1-8, wherein the formoterol compound is present in an amount of about 0.008 wt. % of the composition. ASPECT 9.

In aspects, the invention provides the composition of any one or more of aspects 1-9, wherein the C1-C6 alcohol is present in an amount of at least about 6 wt. % of the composition. ASPECT 10.

In aspects, the invention provides the composition of any one or more of aspects 1-10, wherein the C1-C6 alcohol is present in an amount of at least about 11.25 wt. % of the composition. ASPECT 11.

In aspects, the invention provides the composition of any one or more of aspects 1-11, wherein the C1-C6 alcohol is present in an amount of between about 12 wt. % and about 18 wt. % of the composition. ASPECT 12.

In aspects, the invention provides the composition of any one or more of aspects 1-12, wherein the C1-C6 alcohol is ethanol. ASPECT 13.

In aspects, the invention provides the composition of any one or more of aspects 1-13, wherein one or more of the propellant compounds of the propellant component is selected from HFA-134a, HFA-152a, HFO-1234ze, or a combination thereof. ASPECT 14.

In aspects, the invention provides the composition of any one or more of aspects 1-14, wherein one or more of the propellant compounds of the propellant component is HFA-134a. ASPECT 15.

In aspects, the invention provides the composition of any one or more of aspects 1-14, wherein one or more of the propellant compounds of the propellant component is HFA-152a. ASPECT 16.

In aspects, the invention provides the composition of any one or more of aspects 1-14, wherein one or more of the propellant compounds of the propellant component is HFO-1234ze. ASPECT 17.

In aspects, the invention provides the composition of any one or more of aspects 1-17, wherein the propellant component is at least substantially uniform and wherein the propellant component is at least about 95% composed of the same propellant compound. ASPECT 18.

In aspects, the invention provides the composition of any one or more of aspects 1-18, wherein the propellant component is at least substantially uniform and wherein the propellant component is at least about 96% composed of the same propellant compound. ASPECT 19.

In aspects, the invention provides the composition of any one or more of aspects 1-19, wherein the propellant component is at least about 96% composed of the same propellant compound wherein such compound establishes a dominant propellant, and wherein the ratio of the percent of the propellant component represented by the dominant propellant to at least one of the one or more pharmaceutically acceptable and compositionally compatible small compound stabilizing agent(s) is at least about 320:1. ASPECT 20.

In aspects, the invention provides the composition of any one or more of aspects 1-20, wherein the propellant component is at least about 96% composed of the same propellant compound wherein such compound establishes a dominant propellant, and wherein the ratio of the percent of the propellant component represented by the dominant propellant to at least one of the one or more pharmaceutically acceptable and compositionally compatible small compound stabilizing agent(s) is at least about 323:1. ASPECT 21.

In aspects, the invention provides the composition of any one or more of aspects 1-21, wherein the propellant component is at least about 96% composed of the same propellant compound wherein such compound establishes a dominant propellant, and wherein the ratio of the percent of the propellant component represented by the dominant propellant to at least one of the one or more pharmaceutically acceptable and compositionally compatible small compound stabilizing agent(s) is at least about 326:1. ASPECT 22.

In aspects, the invention provides the composition of any one or more of aspects 1-22, wherein the propellant component is at least about 96% composed of the same propellant compound wherein such compound establishes a dominant propellant, and wherein the ratio of the percent of the propellant component represented by the dominant propellant to at least one of the one or more pharmaceutically acceptable and compositionally compatible small compound stabilizing agent(s) is at least about 330:1. ASPECT 23.

In aspects, the invention provides the composition of any one or more of aspects 1-23, wherein the propellant component is at least about 96% composed of the same propellant compound wherein such compound establishes a dominant propellant, and wherein the ratio of the percent of the propellant component represented by the dominant propellant to at least one of the one or more pharmaceutically acceptable and compositionally compatible small compound stabilizing agent(s) is at least about 333:1. ASPECT 24.

In aspects, the invention provides the composition of any one or more of aspects 1-24, wherein the pharmaceutically acceptable and compositionally compatible small compound stabilizing agent(s) comprise an acid. ASPECT 25.

In aspects, the invention provides the composition of any one or more of aspects 1-25, wherein the pharmaceutically acceptable and compositionally compatible small compound stabilizing agent(s) comprise an organic acid. ASPECT 26.

In aspects, the invention provides the composition of any one or more of aspects 1-26, wherein the pharmaceutically acceptable and compositionally compatible small compound stabilizing agent(s) is/are selected from citric acid and maleic acid. ASPECT 27.

In aspects, the invention provides the composition of any one or more of aspects 1-27, wherein the pharmaceutically acceptable and compositionally compatible small compound stabilizing agent(s) comprise citric acid. ASPECT 28.

In aspects, the invention provides the composition of any one or more of aspects 1-27, wherein the pharmaceutically acceptable and compositionally compatible small compound stabilizing agent(s) comprise maleic acid. ASPECT 29.

In aspects, the invention provides the composition of aspect 28, wherein citric acid is present in an amount of between about 0.01 wt. % and about 0.1 wt. % of the composition. ASPECT 30.

In aspects, the invention provides the composition of aspect 30, wherein the citric acid is present in an amount of about 0.04 wt. % of the composition. ASPECT 31.

In aspects, the invention provides the composition of aspect 29, wherein the maleic acid is present in an amount of between about 0.001 wt. % and about 0.1 wt. % of the composition. ASPECT 32.

In aspects, the invention provides the composition of aspect 32, wherein the maleic acid is present in an amount of about 0.002 wt. %-about 0.05 wt. % of the composition. ASPECT 33.

In aspects, the invention provides the composition of any one or more of aspects 1-33, wherein the composition further comprises a sugar alcohol. ASPECT 34.

In aspects, the invention provides the composition of aspect 34, wherein the sugar alcohol is glycerol. ASPECT 35.

In aspects, the invention provides the composition of any one or both of aspects 34 and 35, wherein the composition comprises a sugar alcohol, e.g., glycerol, in an amount of between about 0.00001 wt. % and about 1 wt. % of the composition. ASPECT 36.

In aspects, the invention provides the composition of any one or more of aspects 1-36, wherein the composition comprises a detectable or significant amount of water. ASPECT 37.

In aspects, the invention provides the composition of aspect 37, wherein the composition comprises water in an amount of between about 0.02 wt. % and about 0.1 wt. % of the composition. ASPECT 38.

In aspects, the invention provides the composition of any one or more of aspects 1-38, wherein the ratio of the pharmaceutically acceptable and compositionally compatible small compound stabilizing agent to the at least one C1-C6 alcohol characterizable as an alkyl alcohol, and aliphatic alcohol, or both, is not greater than about 1:225, not greater than about 1:250, not greater than about 1:275, not greater than about 1:300, not greater than about 1:325, or not greater than about 1:350. ASPECT 39.

In aspects, the invention provides the composition of any one or more of aspects 1-39, wherein the ratio of the pharmaceutically acceptable and compositionally compatible small compound stabilizing agent to the at least one C1-C6 alcohol characterizable as an alkyl alcohol, and aliphatic alcohol, or both, is not greater than about 1:400. ASPECT 40.

In aspects, the invention provides the composition of any one or more of aspects 1-40, wherein the composition does not comprise (e.g., is free of) any compound comprising at least about 12 contiguous carbons which demonstrates detectable or significant surfactant activity. ASPECT 41.

In aspects, the invention provides the composition of any one or more of aspects 1-41, wherein the composition does not comprise (e.g., is free of) a compound comprising a ratio of carbon atoms to any non-hydrogen atom in the compound of at least about 1.5:1 which demonstrates detectable or significant surfactant activity. ASPECT 42.

In aspects, the invention provides the composition of any one or more of aspects 1-42, wherein the composition does not comprise (e.g., is free of) a compound comprising a ratio of carbon atoms to any non-hydrogen atom in the compound of at least about 4:1 which demonstrates detectable or significant surfactant activity. ASPECT 43.

In aspects, the invention provides the composition of any one or more of aspects 1-43, wherein the composition does not comprise (e.g., is free of) any compound providing a significant level of surfactant activity. ASPECT 44.

In aspects, the invention provides a pharmaceutically acceptable composition comprising an active pharmaceutical ingredient component wherein each constituent of the active pharmaceutical ingredient component is at least substantially dissolved and the solution sufficiently homogeneous mixed to allow light to at least substantially uniformly pass through the composition, wherein the an active pharmaceutical ingredient component comprises an effective amount of a tiotropium compound, such as tiotropium or a pharmaceutically acceptable salt or solvate thereof, and an effective amount of a formoterol compound, such as formoterol or a pharmaceutically acceptable salt or solvate thereof, and the composition further comprises (1) at least one C1-C6 alcohol characterizable as an alkyl alcohol, an aliphatic alcohol, or both, present in an amount of at least about 11.25 wt. % of the composition; (2) a pharmaceutically acceptable and compositionally compatible acid compound capable of detectably or significantly preventing or delaying any change in the homogeneity of the composition resulting in (a) the amount of light capable of passing through the composition to be reduced or (b) the composition no longer being characterizable as a solution; and (3) a propellant component comprising one or more pharmaceutically acceptable propellant compounds. ASPECT 45.

In aspects, the invention provides the composition of aspect 45, wherein the tiotropium compound is a salt of tiotropium. ASPECT 46.

In aspects, the invention provides the composition of any one or both of aspect 45 and aspect 46, wherein the tiotropium compound is tiotropium bromide. ASPECT 47.

In aspects, the invention provides the composition of any one or more of aspects 45-47, wherein the tiotropium compound is present in an amount of between about 0.005 wt. % and about 0.01 wt. % of the composition. ASPECT 48.

In aspects, the invention provides the composition of any one or more of aspects 45-48, wherein the formoterol compound is a salt of formoterol. ASPECT 49.

In aspects, the invention provides the composition of any one or more of aspects 45-49, wherein the formoterol compound is formoterol fumarate. ASPECT 50.

In aspects, the invention provides the composition of any one or more of aspects 45-50, wherein the formoterol compound is formoterol fumarate dihydrate. ASPECT 51.

In aspects, the invention provides the composition of any one or more of aspects 45-51, wherein the formoterol compound is present in an amount of between about 0.005 wt. % and about 0.01 wt. % of the composition. ASPECT 52.

In aspects, the invention provides the composition of any one or more of aspects 45-52, wherein the formoterol compound is present in an amount of about 0.008 wt. % of the composition. ASPECT 53.

In aspects, the invention provides the composition of any one or more of aspects 45-53, wherein the C1-C6 alcohol is present in an amount of between about 12 wt. % and about 18 wt. % of the composition. ASPECT 54.

In aspects, the invention provides the composition of any one or more of aspects 45-54, wherein the C1-C6 alcohol is ethanol. ASPECT 55.

In aspects, the invention provides the composition of any one or more of aspects 45-55, wherein one or more of the propellant compounds of the propellant component is selected from HFA-134a, HFA-152a, HFO-1234ze, or a combination thereof. ASPECT 56.

In aspects, the invention provides the composition of any one or more of aspects 45-56, wherein one or more of the propellant compounds of the propellant component is HFA-134a. ASPECT 57.

In aspects, the invention provides the composition of any one or more of aspects 45-56, wherein one or more of the propellant compounds of the propellant component is HFA-152a. ASPECT 58.

In aspects, the invention provides the composition of any one or more of aspects 45-56, wherein one or more of the propellant compounds of the propellant component is HFO-1234ze. ASPECT 59.

In aspects, the invention provides the composition of any one or more of aspects 45-59, wherein the propellant component is at least substantially uniform and wherein the propellant component is at least about 95% composed of the same propellant compound. ASPECT 60.

In aspects, the invention provides the composition of any one or more of aspects 45-60, wherein the propellant component is at least substantially uniform and wherein the propellant component is at least about 96% composed of the same propellant compound. ASPECT 61.

In aspects, the invention provides the composition of any one or more of aspects 45-61, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible acid compound capable of detectably or significantly preventing or delaying any change in the homogeneity of the composition is at least about 320:1. ASPECT 62.

In aspects, the invention provides the composition of any one or more of aspects 45-62, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible acid compound capable of detectably or significantly preventing or delaying any change in the homogeneity of the composition is at least about 323:1. ASPECT 63.

In aspects, the invention provides the composition of any one or more of aspects 45-63, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible acid compound capable of detectably or significantly preventing or delaying any change in the homogeneity of the composition is at least about 326:1. ASPECT 64.

In aspects, the invention provides the composition of any one or more of aspects 45-64, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible acid compound capable of detectably or significantly preventing or delaying any change in the homogeneity of the composition is at least about 330:1. ASPECT 65.

In aspects, the invention provides the composition of any one or more of aspects 45-65, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible acid compound capable of detectably or significantly preventing or delaying any change in the homogeneity of the composition is at least about 333:1. ASPECT 66.

In aspects, the invention provides the composition of any one or more of aspects 45-66, wherein the pharmaceutically acceptable and compositionally compatible acid compound capable of detectably or significantly preventing or delaying any change in the homogeneity of the composition is a small compound stabilizing agent having a molecular weight of less than about 200 g/mol, provides detectable or significant chelating activity, or both. ASPECT 66.

In aspects, the invention provides the composition of any one or more of aspects 45-67, wherein the pharmaceutically acceptable and compositionally compatible acid compound is a small compound organic acid stabilizing agent. ASPECT 68.

In aspects, the invention provides the composition of any one or more of aspects 45-68, wherein the pharmaceutically acceptable and compositionally compatible small compound stabilizing agent (e.g., pharmaceutically acceptable and compositionally compatible acid compound capable of detectably or significantly preventing or delaying any change in the homogeneity of the composition) is selected from citric acid and maleic acid. ASPECT 69.

In aspects, the invention provides the composition of any one or more of aspects 45-69, wherein the pharmaceutically acceptable and compositionally compatible small compound stabilizing agent (e.g., pharmaceutically acceptable and compositionally compatible acid compound capable of detectably or significantly preventing or delaying any change in the homogeneity of the composition) is citric acid. ASPECT 70.

In aspects, the invention provides the composition of any one or more of aspects 45-69, wherein the pharmaceutically acceptable and compositionally compatible small compound stabilizing agent (e.g., pharmaceutically acceptable and compositionally compatible acid compound capable of detectably or significantly preventing or delaying any change in the homogeneity of the composition) is maleic acid. ASPECT 71.

In aspects, the invention provides the composition of aspect 70, wherein the citric acid is present in an amount of between about 0.01 wt. % and about 0.1 wt. % of the composition. ASPECT 72.

In aspects, the invention provides the composition of aspect 72, wherein the citric acid is present in an amount of about 0.05 wt. % of the composition. ASPECT 73.

In aspects, the invention provides the composition of aspect 71, wherein the maleic acid is present in an amount of between about 0.01 wt. % and about 0.1 wt. % of the composition. ASPECT 74.

In aspects, the invention provides the composition of aspect 74, wherein the maleic acid is present in an amount of about 0.05 wt. % of the composition. ASPECT 75.

In aspects, the invention provides the composition of any one or more of aspects 45-33, wherein the composition further comprises a sugar alcohol. ASPECT 76.

In aspects, the invention provides the composition of aspect 76, wherein the sugar alcohol is glycerol. ASPECT 77.

In aspects, the invention provides the composition of any one or both of aspects 76 and 77, wherein the composition comprises a sugar alcohol, e.g., glycerol, in an amount of between about 0.00001 wt. % and about 1 wt. % of the composition. ASPECT 78.

In aspects, the invention provides the composition of any one or more of aspects 45-78, wherein the composition comprises a detectable or significant amount of water. ASPECT 79.

In aspects, the invention provides the composition of aspect 79, wherein the composition comprises water in an amount of between about 0.02 wt. % and about 0.1 wt. % of the composition. ASPECT 80.

In aspects, the invention provides the composition of any one or more of aspects 45-80, wherein the ratio of the pharmaceutically acceptable and compositionally compatible small compound stabilizing agent to the at least one C1-C6 alcohol characterizable as an alkyl alcohol, an aliphatic alcohol, or both, is not greater than about 1:300. ASPECT 81.

In aspects, the invention provides the composition of any one or more of aspects 45-81, wherein the ratio of the pharmaceutically acceptable and compositionally compatible small compound stabilizing agent to the at least one C1-C6 alcohol characterizable as an alkyl alcohol, an aliphatic alcohol, or both, is not greater than about 1:350. ASPECT 82.

In aspects, the invention provides the composition of any one or more of aspects 45-82, wherein the ratio of the pharmaceutically acceptable and compositionally compatible small compound stabilizing agent to the at least one C1-C6 alcohol characterizable as an alkyl alcohol, and aliphatic alcohol, or both, is not greater than about 1:400. ASPECT 83.

In aspects, the invention provides the composition of any one or more of aspects 45-83, wherein the composition does not comprise (e.g., is free of) any compound comprising at least about 12 contiguous carbons which demonstrates detectable or significant surfactant activity. ASPECT 84.

In aspects, the invention provides the composition of any one or more of aspects 45-84, wherein the composition is free of a compound comprising a ratio of carbon atoms to any non-hydrogen atom in the compound of at least about 1.5:1 which demonstrates detectable or significant surfactant activity. ASPECT 85.

In aspects, the invention provides the composition of any one or more of aspects 45-85, wherein the composition is free of a compound comprising a ratio of carbon atoms to any non-hydrogen atom in the compound of at least about 4:1 which demonstrates detectable or significant surfactant activity. ASPECT 86.

In aspects, the invention provides the composition of any one or more of aspects 45-86, wherein the composition does not comprise (e.g., is free of) any compound providing a significant level of surfactant activity. ASPECT 87.

In aspects, the invention provides a pharmaceutical composition comprising an active pharmaceutical ingredient component in solution wherein each constituent of the active pharmaceutical ingredient component is at least substantially dissolved and the solution sufficiently homogeneous mixed to allows light to at least substantially uniformly pass through the composition, wherein the active pharmaceutical ingredient component comprises a tiotropium compound (i.e., at least one tiotropium compound) and a formoterol compound (at least one formoterol compound), and wherein the composition further comprises (1) at least one C1-C6 alcohol characterizable as an alkyl alcohol, an aliphatic alcohol, or both in an amount of at least about 6 wt. % of the composition; (2) at least one pharmaceutically acceptable and compositionally compatible organic acid; and (3) a propellant component comprising one or more pharmaceutically acceptable propellant compound(s), wherein the composition is free of any compound that provides/exhibits significant surfactant activity. ASPECT 88.

In aspects, the invention provides the composition of aspect 88, wherein the tiotropium compound is a salt of tiotropium. ASPECT 89.

In aspects, the invention provides the composition of any one or both of aspect 88 and aspect 89, wherein the tiotropium compound is tiotropium bromide. ASPECT 90.

In aspects, the invention provides the composition of any one or more of aspects 88-90, wherein the tiotropium compound is present in an amount of between about 0.005 wt. % and about 0.01 wt. % of the composition. ASPECT 91.

In aspects, the invention provides the composition of any one or more of aspects 88-91, wherein the formoterol compound is a salt of formoterol. ASPECT 92.

In aspects, the invention provides the composition of any one or more of aspects 88-92, wherein the formoterol compound is formoterol fumarate. ASPECT 93.

In aspects, the invention provides the composition of any one or more of aspects 88-93, wherein the formoterol compound is formoterol fumarate dihydrate. ASPECT 94.

In aspects, the invention provides the composition of any one or more of aspects 88-94, wherein the formoterol compound is present in an amount of between about 0.005 wt. % and about 0.01 wt. % of the composition. ASPECT 95.

In aspects, the invention provides the composition of any one or more of aspects 88-95, wherein the formoterol compound is present in an amount of about 0.008 wt. % of the composition. ASPECT 96.

In aspects, the invention provides the composition of any one or more of aspects 88-96, wherein the C1-C6 alcohol is present in an amount of at least about 11.25 wt. % of the composition. ASPECT 97.

In aspects, the invention provides the composition of any one or more of aspects 88-97, wherein the C1-C6 alcohol is present in an amount of between about 12 wt. % and about 18 wt. % of the composition. ASPECT 98.

In aspects, the invention provides the composition of any one or more of aspects 88-98, wherein the C1-C6 alcohol is ethanol. ASPECT 99.

In aspects, the invention provides the composition of any one or more of aspects 88-99, wherein one or more of the one or more propellant compounds of the propellant component is selected from HFA-134a, HFA-152a, HFO-1234ze, or a combination thereof. ASPECT 100.

In aspects, the invention provides the composition of any one or more of aspects 88-100, wherein one or more of the propellant compounds of the propellant component is HFA-134a. ASPECT 101.

In aspects, the invention provides the composition of any one or more of aspects 88-100, wherein one or more of the propellant compounds of the propellant component is HFA-152a. ASPECT 102.

In aspects, the invention provides the composition of any one or more of aspects 88-100, wherein one or more of the propellant compounds of the propellant component is HFO-1234ze. ASPECT 103.

In aspects, the invention provides the composition of any one or more of aspects 88-103, wherein the propellant component is at least substantially uniform and wherein the propellant component is at least about 95% composed of the same propellant compound. ASPECT 104.

In aspects, the invention provides the composition of any one or more of aspects 88-104, wherein the propellant component is at least substantially uniform and wherein the propellant component is at least about 96% composed of the same propellant compound. ASPECT 105.

In aspects, the invention provides the composition of any one or more of aspects 88-105, wherein the propellant component is at least about 96% composed of the same propellant compound wherein such compound establishes a dominant propellant, and wherein the ratio of the percent of the propellant component represented by the dominant propellant to the at least one organic acid is at least about 320:1. ASPECT 106.

In aspects, the invention provides the composition of any one or more of aspects 88-106, wherein the propellant component is at least about 96% composed of the same propellant compound wherein such compound establishes a dominant propellant, and wherein the ratio of the percent of the propellant component represented by the dominant propellant to the at least one organic acid is at least about 323:1. ASPECT 107.

In aspects, the invention provides the composition of any one or more of aspects 88-107, wherein the propellant component is at least about 96% composed of the same propellant compound wherein such compound establishes a dominant propellant, and wherein the ratio of the percent of the propellant component represented by the dominant propellant to the at least one organic acid is at least about 326:1. ASPECT 108.

In aspects, the invention provides the composition of any one or more of aspects 88-108, wherein the propellant component is at least about 96% composed of the same propellant compound wherein such compound establishes a dominant propellant, and wherein the ratio of the percent of the propellant component represented by the dominant propellant to the at least one organic acid is at least about 330:1. ASPECT 109.

In aspects, the invention provides the composition of any one or more of aspects 88-109, wherein the propellant component is at least about 96% composed of the same propellant compound wherein such compound establishes a dominant propellant, is at least about 333:1. ASPECT 110.

In aspects, the invention provides the composition of any one or more of aspects 88-110, wherein the organic acid of the composition is a stabilizing agent having a molecular weight of less than about 200 g/mol, provides detectable or significant chelating activity, or both. ASPECT 111.

In aspects, the invention provides the composition of any one or more of aspects 88-111, wherein the organic acid is a small compound stabilizing agent capable of detectably of significantly preventing or delaying a change in the homogeneity of the composition. ASPECT 112.

In aspects, the invention provides the composition of any one or more of aspects 88-112, wherein the organic acid is selected from citric acid and maleic acid. ASPECT 113.

In aspects, the invention provides the composition of any one or more of aspects 88-113, wherein the organic acid is citric acid. ASPECT 114.

In aspects, the invention provides the composition of any one or more of aspects 88-113, wherein the organic acid is maleic acid. ASPECT 115.

In aspects, the invention provides the composition of aspect 114, wherein the citric acid is present in an amount of between about 0.01 wt. % and about 0.1 wt. % of the composition. ASPECT 116.

In aspects, the invention provides the composition of aspect 116, wherein the citric acid is present in an amount of about 0.05 wt. % of the composition. ASPECT 117.

In aspects, the invention provides the composition of aspect 115, wherein the maleic acid is present in an amount of between about 0.01 wt. % and about 0.1 wt. % of the composition. ASPECT 118.

In aspects, the invention provides the composition of aspect 118, wherein the maleic acid is present in an amount of about 0.05 wt. % of the composition. ASPECT 119.

In aspects, the invention provides the composition of any one or more of aspects 88-119, wherein the composition further comprises a sugar alcohol. ASPECT 120.

In aspects, the invention provides the composition of aspect 120, wherein the sugar alcohol is glycerol. ASPECT 121.

In aspects, the invention provides the composition of any one or both of aspects 120 and 121, wherein the composition comprises a sugar alcohol, e.g., glycerol, in an amount of between about 0.00001 wt. % and about 1 wt. % of the composition. ASPECT 122.

In aspects, the invention provides the composition of any one or more of aspects 88-122, wherein the composition comprises a detectable or significant amount of water. ASPECT 123.

In aspects, the invention provides the composition of aspect 123, wherein the composition comprises water in an amount of between about 0.02 wt. % and about 0.1 wt. % of the composition. ASPECT 124.

In aspects, the invention provides the composition of any one or more of aspects 88-124, wherein the ratio of the organic acid stabilizing agent to the at least one C1-C6 alcohol characterizable as an alkyl alcohol, an aliphatic alcohol, or both, is not greater than about 1:300. ASPECT 125.

In aspects, the invention provides the composition of any one or more of aspects 88-125, wherein the ratio of the organic acid stabilizing agent to the at least one C1-C6 alcohol characterizable as an alkyl alcohol, an aliphatic alcohol, or both, is not greater than about 1:350. ASPECT 126.

In aspects, the invention provides the composition of any one or more of aspects 88-126, wherein the ratio of the organic acid stabilizing agent to the at least one C1-C6 alcohol characterizable as an alkyl alcohol, and aliphatic alcohol, or both, is not greater than about 1:400. ASPECT 127.

In aspects, the invention provides the composition of any one or more of aspects 88-127, wherein the composition does not comprise (e.g., is free of) any compound comprising at least about 12 contiguous carbons which demonstrates detectable or significant surfactant activity. ASPECT 128.

In aspects, the invention provides the composition of any one or more of aspects 88-128, wherein the composition is free of a compound comprising a ratio of carbon atoms to any non-hydrogen atom in the compound of at least about 1.5:1 which demonstrates detectable or significant surfactant activity. ASPECT 129.

In aspects, the invention provides the composition of any one or more of aspects 88-129, wherein the composition is free of a compound comprising a ratio of carbon atoms to any non-hydrogen atom in the compound of at least about 4:1 which demonstrates detectable or significant surfactant activity. ASPECT 130.

In aspects, the invention provides the composition of any one or more of aspects 88-130, wherein the composition does not comprise (e.g., is free of) any compound providing a significant level of surfactant activity. ASPECT 131.

In aspects, the invention provides a pharmaceutically acceptable composition comprising an active pharmaceutical ingredient component in solution, wherein each constituent of the active pharmaceutical ingredient component is at least substantially dissolved such that the solution is sufficiently homogeneous so as to allow light to at least substantially uniformly pass through the composition, wherein the active pharmaceutical ingredient component comprises an effective amount of at least one tiotropium compound; at least one formoterol compound; and at least one pharmaceutically acceptable inhalable corticosteroid compound, and wherein the composition further comprises (1) at least one C1-C6 alcohol characterizable as an alkyl alcohol, an aliphatic alcohol, or both; (2) a pharmaceutically acceptable and compositionally compatible acid compound capable of detectably or significantly preventing or delaying any change in the homogeneity of the composition resulting in (a) the amount of light capable of passing through the composition to be reduced or (b) the composition no longer being characterizable as a solution; and (3) an at least substantially uniform propellant component, wherein the propellant component is at least about 95% composed of the same propellant compound. ASPECT 132.

In aspects, the invention provides the composition of aspect 132, wherein the tiotropium compound is a salt of tiotropium. ASPECT 133.

In aspects, the invention provides the composition of any one or both of aspect 132 and aspect 133, wherein the tiotropium compound is tiotropium bromide. ASPECT 134.

In aspects, the invention provides the composition of any one or more of aspects 132-134, wherein the tiotropium compound is present in an amount of between about 0.005 wt. % and about 0.01 wt. % of the composition. ASPECT 135.

In aspects, the invention provides the composition of any one or more of aspects 132-135, wherein the formoterol compound is a salt of formoterol. ASPECT 136.

In aspects, the invention provides the composition of any one or more of aspects 132-136, wherein the formoterol compound is formoterol fumarate. ASPECT 137.

In aspects, the invention provides the composition of any one or more of aspects 132-137, wherein the formoterol compound is formoterol fumarate dihydrate. ASPECT 138.

In aspects, the invention provides the composition of any one or more of aspects 132-138, wherein the formoterol compound is present in an amount of between about 0.005 wt. % and about 0.01 wt. % of the composition. ASPECT 139.

In aspects, the invention provides the composition of any one or more of aspects 132-139, wherein the formoterol compound is present in an amount of about 0.008 wt. % of the composition. ASPECT 140.

In aspects, the invention provides the composition of any one or more of aspects 132-140, wherein the C1-C6 alcohol is present in an amount of at least about 6 wt. % of the composition. ASPECT 141.

In aspects, the invention provides the composition of any one or more of aspects 132-141, wherein the C1-C6 alcohol is present in an amount of at least about 11.25 wt. % of the composition. ASPECT 142.

In aspects, the invention provides the composition of any one or more of aspects 132-142, wherein the C1-C6 alcohol is present in an amount of between about 12 wt. % and about 18 wt. % of the composition. ASPECT 143.

In aspects, the invention provides the composition of any one or more of aspects 132-143, wherein the C1-C6 alcohol is ethanol. ASPECT 144.

In aspects, the invention provides the composition of any one or more of aspects 132-144, wherein at least one of the one or more of the propellant compounds of the propellant component is selected from HFA-134a, HFA-152a, HFO-1234ze, or a combination thereof. ASPECT 145.

In aspects, the invention provides the composition of any one or more of aspects 132-145, wherein one or more of the propellant compounds of the propellant component is HFA-134a. ASPECT 146.

In aspects, the invention provides the composition of any one or more of aspects 132-145, wherein one or more of the propellant compounds of the propellant component is HFA-152a. ASPECT 147.

In aspects, the invention provides the composition of any one or more of aspects 132-145, wherein one or more of the propellant compounds of the propellant component is HFO-1234ze. ASPECT 148.

In aspects, the invention provides the composition of any one or more of aspects 132-148, wherein the propellant component is at least substantially uniform and wherein the propellant component is at least about 96% composed of the same propellant compound. ASPECT 149.

In aspects, the invention provides the composition of any one or more of aspects 132-149, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible acid compound capable of detectably or significantly preventing or delaying any change in the homogeneity of the composition is at least about 320:1. ASPECT 150.

In aspects, the invention provides the composition of any one or more of aspects 132-150, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible acid compound capable of detectably or significantly preventing or delaying any change in the homogeneity of the composition is at least about 323:1. ASPECT 151.

In aspects, the invention provides the composition of any one or more of aspects 132-151, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible acid compound capable of detectably or significantly preventing or delaying any change in the homogeneity of the composition is at least about 326:1. ASPECT 152.

In aspects, the invention provides the composition of any one or more of aspects 132-152, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible acid compound capable of detectably or significantly preventing or delaying any change in the homogeneity of the composition is at least about 330:1. ASPECT 153.

In aspects, the invention provides the composition of any one or more of aspects 132-153, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible acid compound capable of detectably or significantly preventing or delaying any change in the homogeneity of the composition is at least about 333:1. ASPECT 154.

In aspects, the invention provides the composition of any one or more of aspects 132-154, wherein the pharmaceutically acceptable and compositionally compatible acid compound capable of detectably or significantly preventing or delaying any change in the homogeneity of the composition is a small compound stabilizing agent having a molecular weight of less than about 200 g/mol, provides detectable or significant chelating activity, or both. ASPECT 155.

In aspects, the invention provides the composition of any one or more of aspects 132-155, wherein the pharmaceutically acceptable and compositionally compatible acid compound is a small compound organic acid stabilizing agent. ASPECT 156.

In aspects, the invention provides the composition of any one or more of aspects 132-156, wherein the pharmaceutically acceptable and compositionally compatible small compound stabilizing agent (e.g., pharmaceutically acceptable and compositionally compatible acid compound capable of detectably or significantly preventing or delaying any change in the homogeneity of the composition) is selected from citric acid and maleic acid. ASPECT 157.

In aspects, the invention provides the composition of any one or more of aspects 132-157, wherein the pharmaceutically acceptable and compositionally compatible small compound stabilizing agent (e.g., pharmaceutically acceptable and compositionally compatible acid compound capable of detectably or significantly preventing or delaying any change in the homogeneity of the composition) is citric acid. ASPECT 158.

In aspects, the invention provides the composition of any one or more of aspects 132-157, wherein the pharmaceutically acceptable and compositionally compatible small compound stabilizing agent (e.g., pharmaceutically acceptable and compositionally compatible acid compound capable of detectably or significantly preventing or delaying any change in the homogeneity of the composition) is maleic acid. ASPECT 159.

In aspects, the invention provides the composition of aspect 158, wherein the citric acid is present in an amount of between about 0.01 wt. % and about 0.1 wt. % of the composition. ASPECT 160.

In aspects, the invention provides the composition of aspect 160, wherein the citric acid is present in an amount of about 0.05 wt. % of the composition. ASPECT 161.

In aspects, the invention provides the composition of aspect 159, wherein the maleic acid is present in an amount of between about 0.01 wt. % and about 0.1 wt. % of the composition. ASPECT 162.

In aspects, the invention provides the composition of aspect 32, wherein the maleic acid is present in an amount of about 0.05 wt. % of the composition. ASPECT 163.

In aspects, the invention provides the composition of any one or more of aspects 132-163, wherein the composition further comprises a sugar alcohol. ASPECT 164.

In aspects, the invention provides the composition of aspect 164, wherein the sugar alcohol is glycerol. ASPECT 165.

In aspects, the invention provides the composition of any one or both of aspects 164 and 165, wherein the composition comprises a sugar alcohol, e.g., glycerol, in an amount of between about 0.00001 wt. % and about 1 wt. % of the composition. ASPECT 166.

In aspects, the invention provides the composition of any one or more of aspects 132-166, wherein the composition comprises a detectable or significant amount of water. ASPECT 167.

In aspects, the invention provides the composition of aspect 167, wherein the composition comprises water in an amount of between about 0.02 wt. % and about 0.1 wt. % of the composition. ASPECT 168.

In aspects, the invention provides the composition of any one or more of aspects 132-168, wherein the ratio of the pharmaceutically acceptable and compositionally compatible acid compound to the at least one C1-C6 alcohol characterizable as an alkyl alcohol, an aliphatic alcohol, or both, is not greater than about 1:300. ASPECT 169.

In aspects, the invention provides the composition of any one or more of aspects 132-169, wherein the ratio of the pharmaceutically acceptable and compositionally compatible acid compound to the at least one C1-C6 alcohol characterizable as an alkyl alcohol, an aliphatic alcohol, or both, is not greater than about 1:350. ASPECT 170.

In aspects, the invention provides the composition of any one or more of aspects 132-170, wherein the ratio of the pharmaceutically acceptable and compositionally compatible acid compound to the at least one C1-C6 alcohol characterizable as an alkyl alcohol, an aliphatic alcohol, or both, is not greater than about 1:400. ASPECT 171.

In aspects, the invention provides the composition of any one or more of aspects 132-171, wherein the composition does not comprise (e.g., is free of) any compound comprising at least about 12 contiguous carbons which demonstrates detectable or significant surfactant activity. ASPECT 172.

In aspects, the invention provides the composition of any one or more of aspects 132-172, wherein the composition is free of a compound comprising a ratio of carbon atoms to any non-hydrogen atom in the compound of at least about 1.5:1 which demonstrates detectable or significant surfactant activity. ASPECT 173.

In aspects, the invention provides the composition of any one or more of aspects 132-173, wherein the composition is free of a compound comprising a ratio of carbon atoms to any non-hydrogen atom in the compound of at least about 4:1 which demonstrates detectable or significant surfactant activity. ASPECT 174.

In aspects, the invention provides the composition of any one or more of aspects 132-174, wherein the composition does not comprise (e.g., is free of) any compound providing a significant level of surfactant activity. ASPECT 175.

In aspects, the invention provides a pharmaceutically acceptable composition comprising an active pharmaceutical ingredient component in solution, wherein each constituent of the active pharmaceutical ingredient component is at least substantially dissolved such that the solution is sufficiently homogeneous so as to allow light to at least substantially uniformly pass through the composition, wherein the active pharmaceutical ingredient component comprises an effective amount of at least one tiotropium compound; at least one formoterol compound; and at least one pharmaceutically acceptable inhalable corticosteroid compound, and wherein the composition further comprises (1) at least one C1-C6 alcohol characterizable as an alkyl alcohol, an aliphatic alcohol, or both; (2) a pharmaceutically acceptable and compositionally compatible small compound stabilizing agent having a molecular weight of less than about 200 g/mol, providing detectable or significant chelating activity, or both, wherein the small compound stabilizing agent is capable of detectably or significantly preventing or delaying any change in the homogeneity of the composition resulting in (a) the amount of light capable of passing through the composition to be reduced or (b) the composition no longer being characterizable as a solution, wherein (I) the pharmaceutically acceptable and compositionally compatible small compound stabilizing agent comprises an organic acid, (II) the composition further comprises a sugar alcohol, or both (I) and (II) are true; and (3) an at least substantially uniform propellant component, wherein the propellant component is at least about 95% composed of the same propellant compound. ASPECT 176.

In aspects, the invention provides the composition of aspect 176, wherein the tiotropium compound is a salt of tiotropium. ASPECT 177.

In aspects, the invention provides the composition of any one or both of aspect 176 and aspect 177, wherein the tiotropium compound is tiotropium bromide. ASPECT 178.

In aspects, the invention provides the composition of any one or more of aspects 176-178, wherein the tiotropium compound is present in an amount of between about 0.005 wt. % and about 0.01 wt. % of the composition. ASPECT 179.

In aspects, the invention provides the composition of any one or more of aspects 176-179, wherein the formoterol compound is a salt of formoterol. ASPECT 180.

In aspects, the invention provides the composition of any one or more of aspects 176-180, wherein the formoterol compound is formoterol fumarate. ASPECT 181.

In aspects, the invention provides the composition of any one or more of aspects 176-181, wherein the formoterol compound is formoterol fumarate dihydrate. ASPECT 182.

In aspects, the invention provides the composition of any one or more of aspects 176-182, wherein the formoterol compound is present in an amount of between about 0.005 wt. % and about 0.01 wt. % of the composition. ASPECT 183.

In aspects, the invention provides the composition of any one or more of aspects 176-183, wherein the formoterol compound is present in an amount of about 0.008 wt. % of the composition. ASPECT 184.

In aspects, the invention provides the composition of any one or more of aspects 176-184, wherein the C1-C6 alcohol is present in an amount of at least about 6 wt. % of the composition. ASPECT 185.

In aspects, the invention provides the composition of any one or more of aspects 176-185, wherein the C1-C6 alcohol is present in an amount of at least about 11.25 wt. % of the composition. ASPECT 186.

In aspects, the invention provides the composition of any one or more of aspects 176-186, wherein the C1-C6 alcohol is present in an amount of between about 12 wt. % and about 18 wt. % of the composition. ASPECT 187.

In aspects, the invention provides the composition of any one or more of aspects 176-187, wherein the C1-C6 alcohol is ethanol. ASPECT 188.

In aspects, the invention provides the composition of any one or more of aspects 176-188, wherein one or more of the propellant compounds of the propellant component is selected from HFA-134a, HFA-152a, HFO-1234ze, or a combination thereof. ASPECT 189.

In aspects, the invention provides the composition of any one or more of aspects 176-188, wherein one or more of the propellant compounds of the propellant component is HFA-134a. ASPECT 190.

In aspects, the invention provides the composition of any one or more of aspects 176-188, wherein one or more of the propellant compounds of the propellant component is HFA-152a. ASPECT 191.

In aspects, the invention provides the composition of any one or more of aspects 176-188, wherein one or more of the propellant compounds of the propellant component is HFO-1234ze. ASPECT 192.

In aspects, the invention provides the composition of any one or more of aspects 176-192, wherein the propellant component is at least substantially uniform and wherein the propellant component is at least about 96% composed of the same propellant compound. ASPECT 193.

In aspects, the invention provides the composition of any one or more of aspects 176-193, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible small compound stabilizing agent is at least about 320:1. ASPECT 194.

In aspects, the invention provides the composition of any one or more of aspects 176-194, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible small compound stabilizing agent is at least about 323:1. ASPECT 195.

In aspects, the invention provides the composition of any one or more of aspects 176-195, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible small compound stabilizing agent is at least about 326:1. ASPECT 196.

In aspects, the invention provides the composition of any one or more of aspects 176-196, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible small compound stabilizing agent is at least about 330:1. ASPECT 197.

In aspects, the invention provides the composition of any one or more of aspects 176-197, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible small compound stabilizing agent is at least about 333:1. ASPECT 198.

In aspects, the invention provides the composition of any one or more of aspects 176-198, wherein the pharmaceutically acceptable and compositionally compatible small compound stabilizing agent is an acid. ASPECT 199.

In aspects, the invention provides the composition of any one or more of aspects 176-199, wherein the pharmaceutically acceptable and compositionally compatible small compound stabilizing agent is an organic acid. ASPECT 200.

In aspects, the invention provides the composition of any one or more of aspects 176-200, wherein the pharmaceutically acceptable and compositionally compatible small compound stabilizing agent is selected from citric acid and maleic acid. ASPECT 201.

In aspects, the invention provides the composition of any one or more of aspects 176-201, wherein the pharmaceutically acceptable and compositionally compatible small compound stabilizing agent is citric acid. ASPECT 202.

In aspects, the invention provides the composition of any one or more of aspects 176-201, wherein the pharmaceutically acceptable and compositionally compatible small compound stabilizing agent is maleic acid. ASPECT 203.

In aspects, the invention provides the composition of aspect 202, wherein the citric acid is present in an amount of between about 0.01 wt. % and about 0.1 wt. % of the composition. ASPECT 204.

In aspects, the invention provides the composition of aspect 204, wherein the citric acid is present in an amount of about 0.05 wt. % of the composition. ASPECT 205.

In aspects, the invention provides the composition of aspect 203, wherein the maleic acid is present in an amount of between about 0.01 wt. % and about 0.1 wt. % of the composition. ASPECT 206.

In aspects, the invention provides the composition of aspect 206, wherein the maleic acid is present in an amount of about 0.05 wt. % of the composition. ASPECT 207.

In aspects, the invention provides the composition of any one or more of aspects 176-207, wherein the composition comprises a sugar alcohol. ASPECT 208.

In aspects, the invention provides the composition of aspect 208, wherein the sugar alcohol is glycerol. ASPECT 209.

In aspects, the invention provides the composition of any one or both of aspects 208 and 209, wherein the composition comprises a sugar alcohol, e.g., glycerol, in an amount of between about 0.00001 wt. % and about 1 wt. % of the composition. ASPECT 210.

In aspects, the invention provides the composition of any one or more of aspects 176-210, wherein the composition comprises a detectable or significant amount of water. ASPECT 211.

In aspects, the invention provides the composition of aspect 211, wherein the composition comprises water in an amount of between about 0.02 wt. % and about 0.1 wt. % of the composition. ASPECT 212.

In aspects, the invention provides the composition of any one or more of aspects 176-212, wherein the ratio of the pharmaceutically acceptable and compositionally compatible small compound stabilizing agent to the at least one C1-C6 alcohol characterizable as an alkyl alcohol, an aliphatic alcohol, or both, is not greater than about 1:300. ASPECT 213.

In aspects, the invention provides the composition of any one or more of aspects 176-213, wherein the ratio of the pharmaceutically acceptable and compositionally compatible small compound stabilizing agent to the at least one C1-C6 alcohol characterizable as an alkyl alcohol, an aliphatic alcohol, or both, is not greater than about 1:350. ASPECT 214.

In aspects, the invention provides the composition of any one or more of aspects 176-214, wherein the ratio of the pharmaceutically acceptable and compositionally compatible small compound stabilizing agent to the at least one C1-C6 alcohol characterizable as an alkyl alcohol, and aliphatic alcohol, or both, is not greater than about 1:400. ASPECT 215.

In aspects, the invention provides the composition of any one or more of aspects 176-215, wherein the composition does not comprise (e.g., is free of) any compound comprising at least about 12 contiguous carbons which demonstrates detectable or significant surfactant activity. ASPECT 216.

In aspects, the invention provides the composition of any one or more of aspects 176-216, wherein the composition is free of a compound comprising a ratio of carbon atoms to any non-hydrogen atom in the compound of at least about 1.5:1 which demonstrates detectable or significant surfactant activity. ASPECT 217.

In aspects, the invention provides the composition of any one or more of aspects 176-217, wherein the composition is free of a compound comprising a ratio of carbon atoms to any non-hydrogen atom in the compound of at least about 4:1 which demonstrates detectable or significant surfactant activity. ASPECT 218.

In aspects, the invention provides the composition of any one or more of aspects 176-218, wherein the composition does not comprise (e.g., is free of) any compound providing a significant level of surfactant activity. ASPECT 219.

In aspects, the invention provides a pharmaceutically acceptable composition comprising an active pharmaceutical ingredient component in solution, wherein each constituent of the active pharmaceutical ingredient component is at least substantially dissolved such that the solution is sufficiently homogeneous so as to allow light to at least substantially uniformly pass through the composition, wherein the active pharmaceutical ingredient component comprises an effective amount of at least one tiotropium compound; at least one formoterol compound; and at least one pharmaceutically acceptable inhalable corticosteroid compound, and wherein the composition further comprises (1) at least one C1-C6 alcohol characterizable as an alkyl alcohol, an aliphatic alcohol, or both; (2) a pharmaceutically acceptable and compositionally compatible organic acid; and (3) an at least substantially uniform and pharmaceutically acceptable propellant component, wherein the propellant component is at least about 96% composed of the same propellant compound establishing the percent of the propellant component represented by a dominant propellant, and wherein ratio of the percent of the propellant component represented by the dominant propellant to the pharmaceutically acceptable and compositionally compatible organic acid is at least about 320:1. ASPECT 220.

In aspects, the invention provides the composition of aspect 220, wherein the tiotropium compound is a salt of tiotropium. ASPECT 221.

In aspects, the invention provides the composition of any one or both of aspect 220 and aspect 221, wherein the tiotropium compound is tiotropium bromide. ASPECT 222.

In aspects, the invention provides the composition of any one or more of aspects 220-222, wherein the tiotropium compound is present in an amount of between about 0.005 wt. % and about 0.01 wt. % of the composition. ASPECT 223.

In aspects, the invention provides the composition of any one or more of aspects 220-223, wherein the formoterol compound is a salt of formoterol. ASPECT 224.

In aspects, the invention provides the composition of any one or more of aspects 220-224, wherein the formoterol compound is formoterol fumarate. ASPECT 225.

In aspects, the invention provides the composition of any one or more of aspects 220-225, wherein the formoterol compound is formoterol fumarate dihydrate. ASPECT 226.

In aspects, the invention provides the composition of any one or more of aspects 220-226, wherein the formoterol compound is present in an amount of between about 0.005 wt. % and about 0.01 wt. % of the composition. ASPECT 227.

In aspects, the invention provides the composition of any one or more of aspects 220-227, wherein the formoterol compound is present in an amount of about 0.008 wt. % of the composition. ASPECT 228.

In aspects, the invention provides the composition of any one or more of aspects 220-228, wherein the C1-C6 alcohol is present in an amount of at least about 6 wt. % of the composition. ASPECT 229.

In aspects, the invention provides the composition of any one or more of aspects 220-229, wherein the C1-C6 alcohol is present in an amount of at least about 11.25 wt. % of the composition. ASPECT 230.

In aspects, the invention provides the composition of any one or more of aspects 220-230, wherein the C1-C6 alcohol is present in an amount of between about 12 wt. % and about 18 wt. % of the composition. ASPECT 231.

In aspects, the invention provides the composition of any one or more of aspects 220-231, wherein the C1-C6 alcohol is ethanol. ASPECT 232.

In aspects, the invention provides the composition of any one or more of aspects 220-232, wherein one or more of the propellant compounds of the propellant component is selected from HFA-134a, HFA-152a, HFO-1234ze, or a combination thereof. ASPECT 233.

In aspects, the invention provides the composition of any one or more of aspects 220-233, wherein one or more of the propellant compounds of the propellant component is HFA-134a. ASPECT 234.

In aspects, the invention provides the composition of any one or more of aspects 220-233, wherein one or more of the propellant compounds of the propellant component is HFA-152a. ASPECT 235.

In aspects, the invention provides the composition of any one or more of aspects 220-233, wherein one or more of the propellant compounds of the propellant component is HFO-1234ze. ASPECT 236.

In aspects, the invention provides the composition of any one or more of aspects 220-236, wherein the ratio of the percent of the propellant component represented by the dominant propellant to the pharmaceutically acceptable and compositionally compatible organic acid is at least about 323:1. ASPECT 237.

In aspects, the invention provides the composition of any one or more of aspects 220-237, wherein the ratio of the percent of the propellant component represented by the dominant propellant to the pharmaceutically acceptable and compositionally compatible small compound stabilizing agent is at least about 326:1. ASPECT 238.

In aspects, the invention provides the composition of any one or more of aspects 220-238, wherein the ratio of the percent of the propellant component represented by the dominant propellant to the pharmaceutically acceptable and compositionally compatible small compound stabilizing agent is at least about 330:1. ASPECT 239.

In aspects, the invention provides the composition of any one or more of aspects 220-23, wherein the ratio of the percent of the propellant component represented by the dominant propellant to the pharmaceutically acceptable and compositionally compatible small compound stabilizing agent is at least about 333:1. ASPECT 240.

In aspects, the invention provides the composition of any one or more of aspects 220-240, wherein the pharmaceutically acceptable and compositionally compatible organic acid is a small compound stabilizing agent capable of detectably or significantly preventing or delaying any change in the homogeneity of the composition. ASPECT 241.

In aspects, the invention provides the composition of any one or more of aspects 220-241, wherein the pharmaceutically acceptable and compositionally compatible organic acid is an organic acid having a molecular weight of less than about 200 g/mol, providing detectable or significant chelating activity, or both. ASPECT 242.

In aspects, the invention provides the composition of any one or more of aspects 220-242, wherein the pharmaceutically acceptable and compositionally compatible small compound stabilizing agent (e.g., pharmaceutically acceptable and compositionally compatible organic acid) is selected from citric acid and maleic acid. ASPECT 243.

In aspects, the invention provides the composition of any one or more of aspects 220-243, wherein the pharmaceutically acceptable and compositionally compatible small compound stabilizing agent (e.g., pharmaceutically acceptable and compositionally compatible organic acid) is citric acid. ASPECT 244.

In aspects, the invention provides the composition of any one or more of aspects 220-243, wherein the pharmaceutically acceptable and compositionally compatible small compound stabilizing agent (e.g., pharmaceutically acceptable and compositionally compatible organic acid) is maleic acid. ASPECT 245.

In aspects, the invention provides the composition of aspect 244, wherein the citric acid is present in an amount of between about 0.01 wt. % and about 0.1 wt. % of the composition. ASPECT 246.

In aspects, the invention provides the composition of aspect 246, wherein the citric acid is present in an amount of about 0.05 wt. % of the composition. ASPECT 247.

In aspects, the invention provides the composition of aspect 245, wherein the maleic acid is present in an amount of between about 0.01 wt. % and about 0.1 wt. % of the composition. ASPECT 248.

In aspects, the invention provides the composition of aspect 248, wherein the maleic acid is present in an amount of about 0.05 wt. % of the composition. ASPECT 249.

In aspects, the invention provides the composition of any one or more of aspects 220-249, wherein the composition further comprises a sugar alcohol. ASPECT 250.

In aspects, the invention provides the composition of aspect 250, wherein the sugar alcohol is glycerol. ASPECT 251.

In aspects, the invention provides the composition of any one or both of aspects 250 and 251, wherein the composition comprises a sugar alcohol, e.g., glycerol, in an amount of between about 0.00001 wt. % and about 1 wt. % of the composition. ASPECT 252.

In aspects, the invention provides the composition of any one or more of aspects 220-252, wherein the composition comprises a detectable or significant amount of water. ASPECT 253.

In aspects, the invention provides the composition of aspect 253, wherein the composition comprises water in an amount of between about 0.02 wt. % and about 0.1 wt. % of the composition. ASPECT 254.

In aspects, the invention provides the composition of any one or more of aspects 220-254, wherein the ratio of the pharmaceutically acceptable and compositionally compatible small compound stabilizing agent (e.g., pharmaceutically acceptable and compositionally compatible organic acid) to the at least one C1-C6 alcohol characterizable as an alkyl alcohol, an aliphatic alcohol, or both, is not greater than about 1:300. ASPECT 255.

In aspects, the invention provides the composition of any one or more of aspects 220-255, wherein the ratio of the pharmaceutically acceptable and compositionally compatible small compound stabilizing agent (e.g., pharmaceutically acceptable and compositionally compatible organic acid) to the at least one C1-C6 alcohol characterizable as an alkyl alcohol, an aliphatic alcohol, or both, is not greater than about 1:350. ASPECT 256.

In aspects, the invention provides the composition of any one or more of aspects 220-256, wherein the ratio of the pharmaceutically acceptable and compositionally compatible small compound stabilizing agent (e.g., pharmaceutically acceptable and compositionally compatible organic acid) to the at least one C1-C6 alcohol characterizable as an alkyl alcohol, and aliphatic alcohol, or both, is not greater than about 1:400. ASPECT 257.

In aspects, the invention provides the composition of any one or more of aspects 220-257, wherein the composition does not comprise (e.g., is free of) any compound comprising at least about 12 contiguous carbons which demonstrates detectable or significant surfactant activity. ASPECT 258.

In aspects, the invention provides the composition of any one or more of aspects 220-258, wherein the composition is free of a compound comprising a ratio of carbon atoms to any non-hydrogen atom in the compound of at least about 1.5:1. ASPECT 259.

In aspects, the invention provides the composition of any one or more of aspects 220-259, wherein the composition is free of a compound comprising a ratio of carbon atoms to any non-hydrogen atom in the compound of at least about 4:1. ASPECT 260.

In aspects, the invention provides the composition of any one or more of aspects 220-260, wherein the composition does not comprise (e.g., is free of) any compound providing a significant level of surfactant activity. ASPECT 261.

In aspects, the invention provides the composition described in any one or more of aspects 1-261, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 1 month. ASPECT 262.

In aspects, the invention provides the composition described in any one or more of aspects 1-262, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 3 months. ASPECT 263.

In aspects, the invention provides the composition described in any one or more of aspects 1-263, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 6 months. ASPECT 264.

In aspects, the invention provides the composition described in any one or more of aspects 1-264, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 9 months. ASPECT 265.

In aspects, the invention provides the composition described in any one or more of aspects 1-265, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 12 months. ASPECT 266.

In aspects, the invention provides the composition described in any one or more of aspects 262-266, wherein the composition comprises less than about 0.1 wt. % total impurities generated by the degradation of (a) any one API, (b) any combination of multiple API(s), or (c) both (a) and (b) based on the total weight of such one, more, or All API(s) in the composition and any associated impurity(ies), after storage at about 40° C. and about 75% relative humidity for a period of at least about 1 month, such as at least about 3 months, at least about 6 months, at least about 9 months, or, e.g., at least about 12 months. ASPECT 267

In aspects, the invention provides a method of manufacturing any one or more of the composition(s) (pharmaceutical composition(s)) described in any one or more of aspects 1-267, wherein the method comprises (1) collecting and combining all API(s) of a composition; (2) adding non-volatile composition component(s) to the API(s); (3) mixing the combination of components until sufficiently mixed to facilitate addition of a propellant; (4) filling container(s) with the desired quantity of composition by a cold filling method comprising (a) adding the required amount of propellant to the mixed composition; (b) mixing the resulting combination of non-propellant components with the propellant until the resulting composition is completely dissolved or until the composition is uniformly suspended; (c) chilling the resulting composition to a temperature of between about −50° C.-about −60° C.; (d) adding the composition to a final packaging container (e.g., a canister); and (e) allowing the container to warm to ambient temperature, resulting in an increase in pressure within the container. ASPECT 268.

In aspects, the invention provides a method of manufacturing any one or more of the composition(s) described in any one or more of aspects 1-267, wherein the method comprises (1) collecting and combining all API(s) of a composition; (2) adding non-volatile composition component(s) to the API(s); (3) mixing the combination of components until sufficiently mixed to facilitate addition of a propellant; (4) filling container(s) with the desired quantity of composition by a pressure filling method comprising (a) adding a required amount of propellant to the mixed composition; (b) adding the mixture of non-propellant components and propellant to a final packaging container (e.g., canister) by way of applied pressure. ASPECT 269.

In aspects, the invention provides a method of directly treating or prophylactically treating in a mammal chronic obstructive pulmonary disease (COPD), asthma, one or more similar or related disorders, such as, e.g., disorders related to the obstruction of airflow to or from the lung(s); any one or more symptoms related to any one or more of such conditions; or any combination of any or all thereof, the method comprising administering a therapeutically effective amount of a composition described in any one or more of aspects 1-267. ASPECT 270.

In aspects, the invention provides a method of using a therapeutically effective amount of any one or more composition(s) described in any one or more of aspects 1-267 in the direct treatment or prophylactic treatment of a mammal suffering from or at risk of suffering from chronic obstructive pulmonary disease (COPD), asthma, one or more similar or related respiratory disorders, such as, e.g., disorders related to the obstruction of airflow to or from the lung(s); any one or more symptoms related to any one or more of such conditions; or any combination of any or all thereof. ASPECT 271.

In aspects, the invention provides a method of detectably or significantly expanding, e.g., dilating, one or more airway(s), e.g., bronchioles, in a recipient suffering from restriction of such one or more airways comprising administration of a therapeutically effective amount of a composition described in any one or more of aspects 1-267. ASPECT 272.

In aspects, the invention provides the method of any one or more of aspects 270-272, wherein the application of the method results in a detectably or significantly greater efficiency of delivery of API compared to a reference product, wherein the reference product is one or more of the reference products described herein. ASPECT 273.

In aspects, the invention provides a kit comprising at least one container comprising any composition described in any one or more of aspects 1-267. ASPECT 274.

In aspects, the invention provides the kit of aspect 274, wherein the kit comprises at least two or more containers each comprising a composition described in any one or more of aspects 1-267. ASPECT 275.

In aspects, the invention provides the kit of any one or both of aspects 274-275, wherein the kit further comprises one or more delivery device(s) designed to effectively deliver the composition(s) provided by the kit. ASPECT 276.

In aspects, the invention provides the kit of aspect 276, wherein at least one of the one or more delivery device(s) is a device suitable for providing the composition(s) to a recipient by inhalation (e.g., an inhaler device). ASPECT 277.

In aspects, the invention provides the kit of aspect 277, wherein the inhaler device is a metered dose inhaler. ASPECT 278.

In aspects, the invention provides the kit of any one or more of aspects 274-278, wherein the kit is used in any one or more of the method(s) described in aspects 270-273. ASPECT 279.

In aspects, the invention provides any one or more of the methods described in this Exemplary Aspects of the Invention section, wherein the application of the method results in (1) a detectably or significantly greater efficiency of delivery of one or more API(s) compared to a reference product (wherein the reference product is one or more reference products such as those described herein); (2) a detectable or significant clinical improvement in one or more measures of chronic obstructive pulmonary disease, asthma, one or more similar or related disorders, such as, e.g., disorders related to the obstruction of airflow to or from the lung(s), any one or more symptoms related to any one or more of such condition(s), or any combination of any or all thereof; (3) a detectably or significantly greater clinical improvement in one or more measures of chronic obstructive pulmonary disease, asthma, one or more similar or related disorders, such as, e.g., disorders related to the obstruction of airflow to or from the lung(s), any one or more symptoms related to any one or more of such condition(s), or any combination of any or all thereof, compared to a reference product (wherein the reference product is one or more reference product(s) as described herein); or (4) any combination of any of (1)-(3), wherein efficacy, outcome(s), or clinical measure of the efficacy of such method(s) (such as, e.g., a performance characteristic, such as, e.g., a detectable or significant improvement in delivery efficiency; a detectable or significant reduction in frequency, severity, or frequency and severity of symptomatic attack (e.g., of acute asthmatic or bronchoconstrictor attack); detectable or significant improvement in lung function; a detectable or significant improvement in airway hyperreactivity; a detectable or significant reduction in the requirement for one or more other symptomatic therapy(ies), e.g., one or more therapy(ies) for or intended to restrict or abort symptomatic attack when it occurs; a detectable or significant reduction in morning dipping (treatment between about 4 AM and about 6 AM, or, e.g., other similar period of time generally most distant from any previous treatment/administration of composition(s)); or any combination thereof), is/are determined by an appropriately conducted and appropriately powered clinical study, such as an appropriately conducted clinical study recognized by an accepted regulatory authority such as, e.g., the U.S. FDA. or similar or related measure of composition efficacy/performance or other measure(s) of efficacy described herein or otherwise known in the art. ASPECT 280.

### Suspension Composition Embodiments

The following is a second listed set of representative aspects of the invention according to a second exemplary embodiment of the invention, which is directed to suspension compositions and related methods of making and using the same.

In a first aspect according to this second embodiment, the invention provides a pharmaceutical composition comprising an active pharmaceutical ingredient component wherein at least a significant portion of one or more constituents of the active pharmaceutical ingredient component remain undissolved such that light cannot pass uniformly through the composition and the composition is characterizable as a suspension or hybrid solution-suspension, wherein the active pharmaceutical ingredient component comprises a tiotropium compound (at least one tiotropium compound) and a formoterol compound (at least one formoterol compound), and wherein the composition further comprises (1) one or more pharmaceutically acceptable and compositionally compatible small compound stabilizing agent(s) having a molecular weight of less than about 200 g/mol, providing detectable or significant chelating activity, or both, wherein the small compound stabilizing agent is capable of detectably or significantly preventing or delaying any change in the homogeneity of the composition and the pharmaceutically acceptable and compositionally compatible small compound stabilizing agent(s) is present in an amount of at least about 0.04 wt. %; (2) a surfactant compound, wherein the surfactant compound does not meet the criteria of claim element (1) above; and (3) a propellant component comprising one or more pharmaceutically acceptable propellant compounds, wherein the propellant component does not comprise HFA-227. ASPECT 1.

In aspects, the invention provides the composition of aspect 1, wherein the tiotropium compound is a salt of tiotropium. ASPECT 2.

In aspects, the invention provides the composition of any one or both of aspect 1 and aspect 2, wherein the tiotropium compound is tiotropium bromide. ASPECT 3.

In aspects, the invention provides the composition of any one or more of aspects 1-3, wherein the tiotropium compound is present in an amount of between about 0.005 wt. % and about 0.01 wt. % of the composition. ASPECT 4.

In aspects, the invention provides the composition of any one or more of aspects 1-4, wherein the formoterol compound is a salt of formoterol. ASPECT 5.

In aspects, the invention provides the composition of any one or more of aspects 1-5, wherein the formoterol compound is formoterol fumarate. ASPECT 6.

In aspects, the invention provides the composition of any one or more of aspects 1-6, wherein the formoterol compound is formoterol fumarate dihydrate. ASPECT 7.

In aspects, the invention provides the composition of any one or more of aspects 1-7, wherein the formoterol compound is present in an amount of between about 0.005 wt. % and about 0.01 wt. % of the composition. ASPECT 8.

In aspects, the invention provides the composition of any one or more of aspects 1-8, wherein the formoterol compound is present in an amount of about 0.008 wt. % of the composition. ASPECT 9.

In aspects, the invention provides the composition of any one or more of aspects 1-9, wherein the active pharmaceutical ingredient component of the composition further comprises one or more additional active pharmaceutical ingredient(s) (API(s)). ASPECT 10.

In aspects, the invention provides the composition of aspect 10, wherein the one or more additional API(s) comprises one or more antiinflammatory compound(s). ASPECT 11.

In aspects, the invention provides the composition of any one or both of aspect 10 and aspect 11, wherein the antiinflammatory compound(s) comprise one or more inhaled corticosteroid(s). ASPECT 12.

In aspects, the invention provides the composition of any one or more of aspects 10-12, wherein the one or more inhaled corticosteroid(s) are selected from beclomethasone, ciclesonide, a fluticasone compound, or a combination of any or all thereof. ASPECT 13.

In aspects, the invention provides the composition of aspect 13, wherein the one or more inhaled corticosteroid(s) is a fluticasone compound. ASPECT 14.

In aspects, the invention provides the composition of aspect 14, wherein the fluticasone compound is fluticasone furoate. ASPECT 15.

In aspects, the invention provides the composition of any one or more of aspects 1-15, wherein the composition is provided as a hybrid solution-suspension. ASPECT 16.

In aspects, the invention provides the composition of any one or more of aspects 1-16, wherein the composition further comprises one or more excipient component(s) each comprising one or more excipient compound(s). ASPECT 17.

In aspects, the invention provides the composition of any one or more of aspects 1-17, wherein the composition comprises one or more of a solvent component, a suspension component, a bulking component, and an absorbent component. ASPECT 18.

In aspects, the invention provides the composition of any one or more of aspects 1-18, wherein the composition comprises a solvent component comprising one or more solvent compound(s). ASPECT 19.

In aspects, the invention provides the composition of any one or more of aspects 1-19, wherein the composition comprises a solvent component comprising one or more C1-C6 alcohols, one or more sugar alcohols, or both. ASPECT 20.

In aspects, the invention provides the composition of any one or more of aspects 1-20, wherein the composition comprises a solvent component comprising a C1-C6 alcohol characterizable as an alkyl alcohol, an aliphatic alcohol, or both. ASPECT 21.

In aspects, the invention provides the composition of any one or more of aspects 1-21, wherein the composition comprises a solvent component comprising a C1-C6 alcohol present in an amount of between about 0.1 wt. % and about 18 wt. % of the composition. ASPECT 22.

In aspects, the invention provides the composition of aspect 22, wherein the C1-C6 alcohol is present in an amount of between about 0.1 wt. % and about 2.5 wt. %. ASPECT 23.

In aspects, the invention provides the composition of aspect 22, wherein the C1-C6 alcohol is present in an amount of at least about 6 wt. % of the composition. ASPECT 24.

In aspects, the invention provides the composition of aspect 24, wherein the C1-C6 alcohol is present in an amount of at least about 11.25 wt. % of the composition. ASPECT 25.

In aspects, the invention provides the composition of aspect 25, wherein the C1-C6 alcohol is present in an amount of between about 12 wt. % and about 18 wt. % of the composition. ASPECT 26.

In aspects, the invention provides the composition of any one or more of aspects 20-26, wherein the C1-C6 alcohol is ethanol. ASPECT 27.

In aspects, the invention provides the composition of any one or more of aspects 1-27, wherein the composition comprises a solvent component comprising a sugar alcohol. ASPECT 28.

In aspects, the invention provides the composition of aspect 28, wherein the sugar alcohol is glycerol. ASPECT 29.

In aspects, the invention provides the composition of any one or both of aspects 28 and 29, wherein the composition comprises a sugar alcohol, e.g., glycerol, in an amount of between about 0.00001 wt. % and about 1 wt. % of the composition. ASPECT 30.

In aspects, the invention provides the composition of any one or more of aspects 1-30, wherein the propellant component comprises one or more propellant compounds, and one or more of the propellant compounds of the propellant component is selected from HFA-134a, HFA-152a, HFO-1234ze, or a combination thereof. ASPECT 31.

In aspects, the invention provides the composition of any one or more of aspects 1-31, wherein the propellant component comprises one or more propellant compounds, and one or more of the propellant compounds of the propellant component is HFA-134a. ASPECT 32.

In aspects, the invention provides the composition of any one or more of aspects 1-31, wherein the propellant component comprises one or more propellant compounds, and one or more of the propellant compounds of the propellant component is HFA-152a. ASPECT 33.

In aspects, the invention provides the composition of any one or more of aspects 1-31, wherein the propellant component comprises one or more propellant compounds, and one or more of the propellant compounds of the propellant component is HFO-1234ze. ASPECT 34.

In aspects, the invention provides the composition of any one or more of aspects 1-34, wherein the propellant component is at least substantially uniform and wherein the propellant component is at least about 95% composed of the same propellant compound. ASPECT 35.

In aspects, the invention provides the composition of any one or more of aspects 1-35, wherein the propellant component is at least substantially uniform and wherein the propellant component is at least about 96% composed of the same propellant compound. ASPECT 36.

In aspects, the invention provides the composition of any one or more of aspects 1-36, wherein the pharmaceutically acceptable and compositionally compatible small compound stabilizing agent(s) comprise an acid. ASPECT 37.

In aspects, the invention provides the composition of any one or more of aspects 1-37, wherein the pharmaceutically acceptable and compositionally compatible small compound stabilizing agent(s) comprise an organic acid. ASPECT 38. 1-38, wherein the pharmaceutically acceptable and compositionally compatible small compound stabilizing agent(s) is/are selected from citric acid and maleic acid. ASPECT 39.

In aspects, the invention provides the composition of any one or more of aspects 1-39, wherein the pharmaceutically acceptable and compositionally compatible small compound stabilizing agent is citric acid. ASPECT 40.

In aspects, the invention provides the composition of any one or more of aspects 1-39, wherein the pharmaceutically acceptable and compositionally compatible small compound stabilizing agent is maleic acid. ASPECT 41.

In aspects, the invention provides the composition of any one or more of aspects 1-41, wherein the stabilizing agent(s), e.g., citric acid or maleic acid, is present in an amount of at least about 0.045 wt. % of the composition. ASPECT 42.

In aspects, the invention provides the composition of any one or more of aspects 1-42, wherein the stabilizing agent(s), e.g., citric acid or maleic acid, is present in an amount of at least about 0.05 wt. % of the composition. ASPECT 43.

In aspects, the invention provides the composition of any one or more of aspects 1-43, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 320:1. ASPECT 44.

In aspects, the invention provides the composition of any one or more of aspects 1-44, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 323:1. ASPECT 45.

In aspects, the invention provides the composition of any one or more of aspects 1-45, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 326:1. ASPECT 46.

In aspects, the invention provides the composition of any one or more of aspects 1-46, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 330:1. ASPECT 47.

In aspects, the invention provides the composition of any one or more of aspects 17-47, wherein the ratio of the pharmaceutically acceptable and compositionally compatible small compound stabilizing agent(s) to the solvent component, e.g., a solvent compound, e.g., to at least one C1-C6 alcohol characterizable as an alkyl alcohol, an aliphatic alcohol, or both, is not greater than about 1:400. ASPECT 48.

In aspects, the invention provides the composition of any one or more of aspects 17-48, wherein the ratio of the pharmaceutically acceptable and compositionally compatible small compound stabilizing agent(s) to the solvent component, e.g., a solvent compound, e.g., to at least one C1-C6 alcohol characterizable as an alkyl alcohol, an aliphatic alcohol, or both, is not greater than about 1:350. ASPECT 49.

In aspects, the invention provides the composition of any one or more of aspects 17-49, wherein the ratio of the pharmaceutically acceptable and compositionally compatible small compound stabilizing agent(s) to the solvent component, e.g., a solvent compound, e.g., to at least one C1-C6 alcohol characterizable as an alkyl alcohol, and aliphatic alcohol, or both, is not greater than about 1:300, is not greater than about 275, is not greater than about 250, or is not greater than about 225. ASPECT 50.

In aspects, the invention provides the composition of any one or more of aspects 1-50, wherein the surfactant compound is a compound comprising at least about 12 contiguous carbons and demonstrates detectable or significant surfactant activity. ASPECT 51.

In aspects, the invention provides the composition of any one or more of aspects 1-51, wherein the surfactant compound is a compound comprising a ratio of carbon atoms to any non-hydrogen atom in the compound of at least about 1.5:1 and demonstrates detectable or significant surfactant activity. ASPECT 52.

In aspects, the invention provides the composition of any one or more of aspects 1-52, wherein the composition is free of a compound comprising a ratio of carbon atoms to any non-hydrogen atom in the compound of at least about 4:1 and demonstrates detectable or significant surfactant activity. ASPECT 53.

In aspects, the invention provides the composition of any one or more of aspects 1-53, wherein the composition comprises a detectable or significant amount of water. ASPECT 54.

In aspects, the invention provides the composition of aspect 54, wherein the composition comprises water in an amount of between about 0.02 wt. % and about 0.1 wt. % of the composition. ASPECT 55.

In aspects, the invention provides the composition of any one or more of aspects 17-55, wherein the composition comprises a suspension component comprising one or more suspension agent(s). ASPECT 56.

In aspects, the invention provides the composition of aspect 56, wherein the suspension component comprises PEG 400, PEG 600, PEG 1000, PVP K25, PVP K30, PVP VA64, lysine, glycine, leucine, phospholipid, cyclodextrin, a cremophor compound, or a combination thereof. ASPECT 57.

In aspects, the invention provides the composition of any one or both of aspect 56 and aspect 57, wherein the suspension component comprises PEG 600, PEG 1000, glycine, or a combination thereof. ASPECT 58.

In aspects, the invention provides the composition of any one or more of aspects 17-58, wherein the composition comprises a suspension component in an amount of between about 0.001 wt. % and about 10 wt. % of the composition. ASPECT 59.

In aspects, the invention provides the composition of any one or more of aspects 17-58, wherein the composition comprises a bulking component comprising one or more bulking agent(s). ASPECT 60.

In aspects, the invention provides the composition of aspect 56, wherein the bulking component comprises monosaccharide(s), disaccharide(s), oligosaccharide(s), and polysaccharide(s) for example lactose, maltose, glucose, fructose, galactose arabinose, dextrose, ribose, sucrose, sorbitol, mannitol, xylose, trehalose, raffinose, melezitose, glycerol, erythritol, xylitol, maltitol, lactitol, and D & L series of rare sugars, magnesium stearate, or a combination thereof. ASPECT 61.

In aspects, the invention provides the composition of any one or both of aspect 60 and aspect 61, wherein the bulking component comprises lactose, magnesium stearate, mannitol, or a combination of any or all thereof. ASPECT 62.

In aspects, the invention provides the composition of any one or more of aspects 17-62, wherein the composition comprises a bulking component in an amount of between about 0.001 wt. % and about 10 wt. % of the composition. ASPECT 59.

In aspects, the invention provides the composition of any one or more of aspects 17-63, wherein the composition comprises an absorbent component comprising one or more absorbing agent(s). ASPECT 64.

In aspects, the invention provides the composition of aspect 64, wherein the absorbent component comprises sodium cromoglycate. ASPECT 65.

In aspects, the invention provides the composition of any one or both of aspect 64 and aspect 65, wherein the composition comprises an absorbent component in an amount of between about 0.001 wt. % and about 0.1 wt. % of the composition. ASPECT 66.

In aspects, the invention provides the composition described in anyone or more of aspects 1-66, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 1 month. ASPECT 67.

In aspects, the invention provides the composition described in anyone or more of aspects 1-66, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 3 months. ASPECT 68.

In aspects, the invention provides the composition described in anyone or more of aspects 1-68, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 6 months. ASPECT 69.

In aspects, the invention provides the composition described in anyone or more of aspects 1-69, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 9 months. ASPECT 70.

In aspects, the invention provides the composition described in anyone or more of aspects 1-70, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 12 months. ASPECT 71.

In aspects, the invention provides the composition described in anyone or more of aspects 67-71, wherein the composition comprise less than about 0.1 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 1 month, such as, e.g., a period of at least about 3 months, at least about 6 months, at least about 9 months, or, e.g., at least about 12 months. ASPECT 72.

In aspects, the invention provides a method of manufacturing any one or more of the composition(s) described in any one or more of aspects 1-72, wherein the method comprises (1) collecting and combining all API(s) of a composition; (2) adding non-volatile composition component(s) to the API(s); (3) mixing the combination of components until sufficiently mixed to facilitate addition of a propellant; (4) filling container(s) with the desired quantity of composition by a cold filling method comprising (a) adding the required amount of propellant to the mixed composition; (b) mixing the resulting combination of non-propellant components with the propellant until the resulting composition is completely dissolved or until the composition is uniformly suspended; (c) chilling the resulting composition to a temperature of between about −50° C.-about −60° C.; (d) adding the composition to a final packaging container (e.g., a canister); and (e) allowing the container to warm to ambient temperature, resulting in an increase in pressure within the container. ASPECT 73.

In aspects, the invention provides a method of manufacturing any one or more of the composition(s) described in any one or more of aspects 1-72, wherein the method comprises (1) collecting and combining all API(s) of a composition; (2) adding non-volatile composition component(s) to the API(s); (3) mixing the combination of components until sufficiently mixed to facilitate addition of a propellant; (4) filling container(s) with the desired quantity of composition by a pressure filling method comprising (a) adding a required amount of propellant to the mixed composition; (b) adding the mixture of non-propellant components and propellant to a final packaging container (e.g., canister) by way of applied pressure. ASPECT 74.

In aspects, the invention provides a method of directly treating or prophylactically treating in a mammal chronic obstructive pulmonary disease (COPD), asthma, one or more similar or related disorders, such as, e.g., disorders related to the obstruction of airflow to or from the lung(s); any one or more symptoms related to any one or more of such conditions; or any combination of any or all thereof, the method comprising administering a therapeutically effective amount of a composition described in any one or more of aspects 1-72. ASPECT 75.

In aspects, the invention provides a method of using a therapeutically effective amount of any one or more composition(s) described in any one or more of aspects 1-72 in the direct treatment or prophylactic treatment of a mammal suffering from or at risk of suffering from chronic obstructive pulmonary disease (COPD), asthma, one or more similar or related respiratory disorders, such as, e.g., disorders related to the obstruction of airflow to or from the lung(s); any one or more symptoms related to any one or more of such conditions; or any combination of any or all thereof. ASPECT 76.

In aspects, the invention provides a method of detectably or significantly expanding, e.g., dilating, one or more airway(s), e.g., bronchioles, in a recipient suffering from restriction of such one or more airways comprising administration of a therapeutically effective amount of a composition described in any one or more of aspects 1-72. ASPECT 77.

In aspects, the invention provides the method of any one or more of aspects 75-77, wherein the application of the method results in a detectably or significantly greater efficiency of delivery of API compared to a reference product, wherein the reference product is one or more of the reference products described herein. ASPECT 78.

In aspects, the invention provides a kit comprising at least one container comprising any composition described in any one or more of aspects 1-72. ASPECT 79.

In aspects, the invention provides the kit of aspect 79, wherein the kit comprises at least two or more containers each comprising a composition described in any one or more of aspects 1-71. ASPECT 80.

In aspects, the invention provides the kit of any one or both of aspects 79-80, wherein the kit further comprises one or more delivery device(s) designed to effectively deliver the composition(s) provided by the kit. ASPECT 81.

In aspects, the invention provides the kit of aspect 81, wherein at least one of the one or more delivery device(s) is a device suitable for providing the composition(s) to a recipient by inhalation (e.g., an inhaler device). ASPECT 82.

In aspects, the invention provides the kit of aspect 82, wherein the inhaler device is a metered dose inhaler. ASPECT 83.

In aspects, the invention provides the kit of any one or more of aspects 79-83, wherein the kit is used in any one or more of the method(s) described in aspects 75-78. ASPECT 84.

In aspects, the invention provides any one or more of the methods described in this Exemplary Aspects of the Invention section, wherein the application of the method results in (1) a detectably or significantly greater efficiency of delivery of one or more API(s) compared to a reference product (wherein the reference product is one or more reference products such as those described herein); (2) a detectable or significant clinical improvement in one or more measures of chronic obstructive pulmonary disease, asthma, one or more similar or related disorders, such as, e.g., disorders related to the obstruction of airflow to or from the lung(s), any one or more symptoms related to any one or more of such condition(s), or any combination of any or all thereof; (3) a detectably or significantly greater clinical improvement in one or more measures of chronic obstructive pulmonary disease, asthma, one or more similar or related disorders, such as, e.g., disorders related to the obstruction of airflow to or from the lung(s), any one or more symptoms related to any one or more of such condition(s), or any combination of any or all thereof, compared to a reference product (wherein the reference product is one or more reference product(s) as described herein); or (4) any combination of any of (1)-(3), wherein efficacy, outcome(s), or clinical measure of the efficacy of such method(s) (such as, e.g., a performance characteristic, such as, e.g., a detectable or significant improvement in delivery efficiency; a detectable or significant reduction in frequency, severity, or frequency and severity of symptomatic attack (e.g., of acute asthmatic or bronchoconstrictor attack); detectable or significant improvement in lung function; a detectable or significant improvement in airway hyperreactivity; a detectable or significant reduction in the requirement for one or more other symptomatic therapy(ies), e.g., one or more therapy(ies) for or intended to restrict or abort symptomatic attack when it occurs; a detectable or significant reduction in morning dipping (treatment between about 4 AM and about 6 AM, or, e.g., other similar period of time generally most distant from any previous treatment/administration of composition(s)); or any combination thereof), is/are determined by an appropriately conducted and appropriately powered clinical study, such as an appropriately conducted clinical study recognized by an accepted regulatory authority such as, e.g., the U.S. FDA. or similar or related measure of composition efficacy/performance or other measure(s) of efficacy described herein or otherwise known in the art. ASPECT 85.

### Corticosteroid-Containing Composition Embodiments

In a first aspect according to this third embodiment, the invention provides a composition comprising an active pharmaceutical ingredient component wherein each constituent of the active pharmaceutical ingredient component is at least substantially dissolved such that the solution is sufficiently homogeneous so as to allow light to at least substantially uniformly pass through the composition, wherein the an active pharmaceutical ingredient component comprises an effective amount of at least one tiotropium compound (a tiotropium compound); an effective amount of at least one formoterol compound (a formoterol compound); and an effective amount of at least one pharmaceutically acceptable inhalable corticosteroid compound (a corticosteroid compound), wherein the composition further comprises (1) a solvent component present in an amount representing at least about 11 wt. % of the composition; (2) pharmaceutically acceptable and compositionally compatible small compound stabilizing agent(s) having a molecular weight of less than about 200 g/mol, providing detectable or significant chelating activity, or both, wherein the small compound stabilizing agent is capable of detectably or significantly preventing or delaying any change in the homogeneity of the composition resulting in (a) the amount of light capable of passing through the composition to be reduced or (b) the composition no longer being characterizable as a solution; and (3) a propellant component comprising one or more pharmaceutically acceptable propellant compounds. ASPECT 1.

In aspects, the invention provides the composition of aspect 1, wherein the tiotropium compound is a salt of tiotropium. ASPECT 2.

In aspects, the invention provides the composition of any one or both of aspect 1 and aspect 2, wherein the tiotropium compound is tiotropium bromide. ASPECT 3.

In aspects, the invention provides the composition of any one or more of aspects 1-3, wherein the tiotropium compound is present in an amount of between about 0.005 wt. % and about 0.01 wt. % of the composition. ASPECT 4.

In aspects, the invention provides the composition of any one or more of aspects 1-4, wherein the formoterol compound is a salt of formoterol. ASPECT 5.

In aspects, the invention provides the composition of any one or more of aspects 1-5, wherein the formoterol compound is formoterol fumarate. ASPECT 6.

In aspects, the invention provides the composition of any one or more of aspects 1-6, wherein the formoterol compound is formoterol fumarate dihydrate. ASPECT 7.

In aspects, the invention provides the composition of any one or more of aspects 1-7, wherein the formoterol compound is present in an amount of between about 0.005 wt. % and about 0.01 wt. % of the composition. ASPECT 8.

In aspects, the invention provides the composition of any one or more of aspects 1-8, wherein the formoterol compound is present in an amount of about 0.008 wt. % of the composition. ASPECT 9.

In aspects, the invention provides the composition of any one or more of aspects 1-9, wherein at least one of the one or more inhaled corticosteroid compound(s) is selected from beclomethasone, ciclesonide, a fluticasone compound, or a combination of any or all thereof. ASPECT 10.

In aspects, the invention provides the composition of any one or more of aspects 1-10, wherein the one or more inhaled corticosteroid(s) is beclomethasone. ASPECT 11.

In aspects, the invention provides the composition of any one or more of aspects 1-10, wherein the one or more inhaled corticosteroid(s) is ciclesonide. ASPECT 12.

In aspects, the invention provides the composition of any one or more of aspects 1-10, wherein the one or more inhaled corticosteroid(s) is a fluticasone compound. ASPECT 13.

In aspects, the invention provides the composition of aspect 13, wherein the fluticasone compound is fluticasone furoate. ASPECT 14.

In aspects, the invention provides the composition of any one or more of aspects 1-14, wherein the at least one pharmaceutically acceptable inhalable corticosteroid or a pharmaceutically acceptable salt or solvate thereof is present in the composition in an amount of between about 0.18 wt. % and about 0.36 wt. %. ASPECT 15.

In aspects, the invention provides the composition of any one or more of aspects 1-15, wherein the solvent component is capable of detectably or significantly preventing or delaying any change in the homogeneity of the composition. ASPECT 16.

In aspects, the invention provides the composition of any one or more of aspects 1-16, wherein the solvent component is present in an amount of at least about 11.25 wt. % of the composition. ASPECT 17.

In aspects, the invention provides the composition of any one or more of aspects 1-16, wherein the solvent component is present in an amount of at least about 12 wt. % of the composition. ASPECT 18.

In aspects, the invention provides the composition of any one or more of aspects 1-18, wherein the solvent component is present in an amount of between about 12 wt. % and about 18 wt. % of the composition. ASPECT 19.

In aspects, the invention provides the composition of any one or more of aspects 1-19, wherein the solvent component comprises a C1-C6 alcohol. ASPECT 20.

In aspects, the invention provides the composition of any one or more of aspects 1-20, wherein the solvent component comprises a C1-C6 alcohol characterizable as an alkyl alcohol, an aliphatic alcohol, or both. ASPECT 21.

In aspects, the invention provides the composition of any one or more of aspects 1-21, wherein the solvent component comprises ethanol. ASPECT 22.

In aspects, the invention provides the composition of any one or more of aspects 1-22, wherein one or more of the propellant compounds of the propellant component is selected from HFA-134a, HFA-152a, HFO-1234ze, or a combination thereof. ASPECT 23.

In aspects, the invention provides the composition of any one or more of aspects 1-23, wherein one or more of the propellant compounds of the propellant component is HFA-134a. ASPECT 24.

In aspects, the invention provides the composition of any one or more of aspects 1-23, wherein one or more of the propellant compounds of the propellant component is HFA-152a. ASPECT 25.

In aspects, the invention provides the composition of any one or more of aspects 1-23, wherein one or more of the propellant compounds of the propellant component is HFO-1234ze. ASPECT 26.

In aspects, the invention provides the composition of any one or more of aspects 1-26, wherein the propellant component does not comprise HFA-227. ASPECT 27.

In aspects, the invention provides the composition of any one or more of aspects 1-27, wherein the propellant component is at least substantially uniform and wherein the propellant component is at least about 95% composed of the same propellant compound. ASPECT 28.

In aspects, the invention provides the composition of any one or more of aspects 1-28, wherein the propellant component is at least substantially uniform and wherein the propellant component is at least about 96% composed of the same propellant compound. ASPECT 29.

In aspects, the invention provides the composition of any one or more of aspects 1-29, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 320:1. ASPECT 30.

In aspects, the invention provides the composition of any one or more of aspects 1-30, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 323:1. ASPECT 31.

In aspects, the invention provides the composition of any one or more of aspects 1-31, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 326:1. ASPECT 32.

In aspects, the invention provides the composition of any one or more of aspects 1-32, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 330:1. ASPECT 33.

In aspects, the invention provides the composition of any one or more of aspects 1-33, wherein the propellant component is at least about 96% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 333:1. ASPECT 34.

In aspects, the invention provides the composition of any one or more of aspects 1-34, wherein the pharmaceutically acceptable and compositionally compatible small compound stabilizing agent(s) comprise an acid. ASPECT 35.

In aspects, the invention provides the composition of any one or more of aspects 1-35, wherein the pharmaceutically acceptable and compositionally compatible small compound stabilizing agent(s) comprise an organic acid. ASPECT 36.

In aspects, the invention provides the composition of any one or more of aspects 1-36, wherein the pharmaceutically acceptable and compositionally compatible small compound stabilizing agent(s) is/are selected from citric acid and maleic acid. ASPECT 37.

In aspects, the invention provides the composition of any one or more of aspects 1-37, wherein the pharmaceutically acceptable and compositionally compatible small compound stabilizing agent(s) comprise citric acid. ASPECT 38.

In aspects, the invention provides the composition of any one or more of aspects 1-37, wherein the pharmaceutically acceptable and compositionally compatible small compound stabilizing agent(s) comprise maleic acid. ASPECT 39.

In aspects, the invention provides the composition of aspect 38, wherein the citric acid is present in an amount of between about 0.01 wt. % and about 0.1 wt. % of the composition. ASPECT 40.

In aspects, the invention provides the composition of aspect 40, wherein the citric acid is present in an amount of about 0.04 wt. % of the composition. ASPECT 41.

In aspects, the invention provides the composition of aspect 39, wherein the maleic acid is present in an amount of between about 0.001 wt. % and about 0.1 wt. % of the composition. ASPECT 42.

In aspects, the invention provides the composition of aspect 42, wherein the maleic acid is present in an amount of about 0.002 wt. % and about 0.05 wt. % of the composition. ASPECT 43.

In aspects, the invention provides the composition of any one or more of aspects 1-43, wherein the pharmaceutically acceptable and compositionally compatible small compound stabilizing agent(s) is/are present in an amount of at least about 0.04 wt. % of the composition. ASPECT 44.

In aspects, the invention provides the composition of any one or more of aspects 1-44, wherein the composition further comprises a sugar alcohol. ASPECT 45.

In aspects, the invention provides the composition of aspect 45, wherein the sugar alcohol is glycerol. ASPECT 46.

In aspects, the invention provides the composition of any one or both of aspects 45 and 46, wherein the composition comprises a sugar alcohol, e.g., glycerol, in an amount of between about 0.00001 wt. % and about 1 wt. % of the composition. ASPECT 47.

In aspects, the invention provides the composition of any one or more of aspects 1-47, wherein the composition comprises a detectable or significant amount of water. ASPECT 48.

In aspects, the invention provides the composition of aspect 48, wherein the composition comprises water in an amount of between about 0.02 wt. % and about 0.1 wt. % of the composition. ASPECT 49.

In aspects, the invention provides the composition of any one or more of aspects 1-49, wherein the ratio of the pharmaceutically acceptable and compositionally compatible small compound stabilizing agent to the solvent component, e.g., at least one C1-C6 alcohol characterizable as an alkyl alcohol, an aliphatic alcohol, or both, is not greater than about 1:400. ASPECT 50.

In aspects, the invention provides the composition of any one or more of aspects 1-50, wherein the ratio of the pharmaceutically acceptable and compositionally compatible small compound stabilizing agent to the solvent component, e.g., at least one C1-C6 alcohol characterizable as an alkyl alcohol, an aliphatic alcohol, or both, is not greater than about 1:350. ASPECT 51.

In aspects, the invention provides the composition of any one or more of aspects 1-51, wherein the ratio of the pharmaceutically acceptable and compositionally compatible small compound stabilizing agent to the solvent component, e.g., at least one C1-C6 alcohol characterizable as an alkyl alcohol, and aliphatic alcohol, or both, is not greater than about 1:300, not greater than about 1:275, not greater than about 1:250, or is not greater than about 1:225. ASPECT 52.

In aspects, the invention provides the composition of any one or more of aspects 1-52, wherein the composition does not comprise (e.g., is free of) any compound comprising at least about 12 contiguous carbons which demonstrates detectable or significant surfactant activity. ASPECT 53.

In aspects, the invention provides the composition of any one or more of aspects 1-53, wherein the composition is free of a compound comprising a ratio of carbon atoms to any non-hydrogen atom in the compound of at least about 1.5:1 which demonstrates detectable or significant surfactant activity. ASPECT 54.

In aspects, the invention provides the composition of any one or more of aspects 1-54, wherein the composition is free of a compound comprising a ratio of carbon atoms to any non-hydrogen atom in the compound of at least about 4:1 which demonstrates detectable or significant surfactant activity. ASPECT 55.

In aspects, the invention provides the composition of any one or more of aspects 1-55, wherein the composition does not comprise (e.g., is free of) any compound providing a significant level of surfactant activity. ASPECT 56.

In aspects, the invention provides a composition comprising an effective amount of an active pharmaceutical ingredient component wherein each constituent of the active pharmaceutical ingredient component is at least substantially dissolved such that the solution is sufficiently homogeneous so as to allow light to at least substantially uniformly pass through the composition, wherein the an active pharmaceutical ingredient component comprises an effective amount of a tiotropium compound (a tiotropium compound); an effective amount of a formoterol compound (a formoterol compound); and at least one pharmaceutically acceptable inhalable corticosteroid or a pharmaceutically acceptable salt or solvate thereof, wherein the composition further comprises (1) a solvent component capable of detectably or significantly preventing or delaying any change in the homogeneity of the composition; (2) pharmaceutically acceptable and compositionally compatible small compound stabilizing agent(s) having a molecular weight of less than about 200 g/mol, providing detectable or significant chelating activity, or both, wherein the small compound stabilizing agent is capable of detectably or significantly preventing or delaying any change in the homogeneity of the composition resulting in (a) the amount of light capable of passing through the composition to be reduced or (b) the composition no longer being characterizable as a solution; and (3) an at least substantially homogeneous propellant component wherein the propellant component is at least about 98% composed of the same propellant compound. ASPECT 57.

In aspects, the invention provides the composition of aspect 57, wherein the tiotropium compound is a salt of tiotropium. ASPECT 58.

In aspects, the invention provides the composition of any one or both of aspect 57 and aspect 58, wherein the tiotropium compound is tiotropium bromide. ASPECT 59.

In aspects, the invention provides the composition of any one or more of aspects 57-59, wherein the tiotropium compound is present in an amount of between about 0.005 wt. % and about 0.01 wt. % of the composition. ASPECT 60.

In aspects, the invention provides the composition of any one or more of aspects 57-60, wherein the formoterol compound is a salt of formoterol. ASPECT 61.

In aspects, the invention provides the composition of any one or more of aspects 57-61, wherein the formoterol compound is formoterol fumarate. ASPECT 62.

In aspects, the invention provides the composition of any one or more of aspects 57-62, wherein the formoterol compound is formoterol fumarate dihydrate. ASPECT 63.

In aspects, the invention provides the composition of any one or more of aspects 57-63, wherein the formoterol compound is present in an amount of between about 0.005 wt. % and about 0.01 wt. % of the composition. ASPECT 64.

In aspects, the invention provides the composition of any one or more of aspects 57-64, wherein the formoterol compound is present in an amount of about 0.008 wt. % of the composition. ASPECT 65.

In aspects, the invention provides the composition of any one or more of aspects 57-65, wherein at least one of the one or more inhaled corticosteroid(s) is selected from beclomethasone, ciclesonide, a fluticasone compound, or a combination of any or all thereof. ASPECT 66.

In aspects, the invention provides the composition of any one or more of aspects 57-66, wherein the one or more inhaled corticosteroid(s) is beclomethasone. ASPECT 67.

In aspects, the invention provides the composition of any one or more of aspects 57-66, wherein the one or more inhaled corticosteroid(s) is ciclesonide. ASPECT 68.

In aspects, the invention provides the composition of any one or more of aspects 57-66, wherein the one or more inhaled corticosteroid(s) is a fluticasone compound. ASPECT 69.

In aspects, the invention provides the composition of aspect 69, wherein the fluticasone compound is fluticasone furoate. ASPECT 70.

In aspects, the invention provides the composition of any one or more of aspects 57-70, wherein the at least one pharmaceutically acceptable inhalable corticosteroid or a pharmaceutically acceptable salt or solvate thereof is present in the composition in an amount of between about 0.18 wt. % and about 0.36 wt. %. ASPECT 71.

In aspects, the invention provides the composition of any one or more of aspects 57-71, wherein the solvent component is present in an amount of at least about 5 wt. % of the composition. ASPECT 72.

In aspects, the invention provides the composition of any one or more of aspects 57-72, wherein the solvent component is present in an amount of at least about 6 wt. % of the composition. ASPECT 73.

In aspects, the invention provides the composition of any one or more of aspects 57-73, wherein the solvent component is present in an amount of at least about 8 wt. % of the composition. ASPECT 74.

In aspects, the invention provides the composition of any one or more of aspects 57-74, wherein the solvent component is present in an amount of at least about 10 wt. % of the composition. ASPECT 75.

In aspects, the invention provides the composition of any one or more of aspects 57-75, wherein the solvent component is present in an amount of at least about 11 wt. % of the composition. ASPECT 76.

In aspects, the invention provides the composition of any one or more of aspects 57-76, wherein the solvent component is present in an amount of at least about 12 wt. % of the composition. ASPECT 77.

In aspects, the invention provides the composition of any one or more of aspects 57-77, wherein the solvent component is present in an amount of at least about 15 wt. % of the composition. ASPECT 78.

In aspects, the invention provides the composition of any one or more of aspects 57-78, wherein the solvent component is present in an amount of between about 15 wt. % and about 18 wt. % of the composition. ASPECT 79.

In aspects, the invention provides the composition of any one or more of aspects 57-79, wherein the solvent component comprises a C1-C6 alcohol. ASPECT 80.

In aspects, the invention provides the composition of any one or more of aspects 57-80, wherein the solvent component comprises a C1-C6 alcohol characterizable as an alkyl alcohol, an aliphatic alcohol, or both. ASPECT 81.

In aspects, the invention provides the composition of any one or more of aspects 57-81, wherein the solvent component comprises ethanol. ASPECT 82.

In aspects, the invention provides the composition of any one or more of aspects 57-82, wherein the propellant component comprises HFA-134a, HFA-152a, HFO-1234ze, or a combination thereof. ASPECT 83.

In aspects, the invention provides the composition of any one or more of aspects 57-83, wherein the propellant component comprises HFA-134a. ASPECT 84.

In aspects, the invention provides the composition of any one or more of aspects 57-83, wherein the propellant component comprises HFA-152a. ASPECT 85.

In aspects, the invention provides the composition of any one or more of aspects 57-83, wherein the propellant component comprises HFO-1234ze. ASPECT 86.

In aspects, the invention provides the composition of any one or more of aspects 57-86, wherein the propellant component does not comprise HFA-227. ASPECT 87.

In aspects, the invention provides the composition of any one or more of aspects 57-87, wherein the propellant component is at least about 98% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 320:1. ASPECT 88.

In aspects, the invention provides the composition of any one or more of aspects 57-88, wherein the propellant component is at least about 98% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 323:1. ASPECT 89.

In aspects, the invention provides the composition of any one or more of aspects 57-89, wherein the propellant component is at least about 98% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 326:1. ASPECT 90.

In aspects, the invention provides the composition of any one or more of aspects 57-90, wherein the propellant component is at least about 98% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 330:1. ASPECT 91.

In aspects, the invention provides the composition of any one or more of aspects 57-91, wherein the propellant component is at least about 98% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 333:1. ASPECT 92.

In aspects, the invention provides the composition of any one or more of aspects 57-92, wherein the pharmaceutically acceptable and compositionally compatible small compound stabilizing agent(s) comprise an acid. ASPECT 93.

In aspects, the invention provides the composition of any one or more of aspects 57-93, wherein the pharmaceutically acceptable and compositionally compatible small compound stabilizing agent(s) comprise an organic acid. ASPECT 94.

In aspects, the invention provides the composition of any one or more of aspects 57-94, wherein the pharmaceutically acceptable and compositionally compatible small compound stabilizing agent(s) is/are selected from citric acid and maleic acid. ASPECT 95.

In aspects, the invention provides the composition of any one or more of aspects 57-95, wherein the pharmaceutically acceptable and compositionally compatible small compound stabilizing agent(s) comprise citric acid. ASPECT 96.

In aspects, the invention provides the composition of any one or more of aspects 57-95, wherein the pharmaceutically acceptable and compositionally compatible small compound stabilizing agent(s) comprise maleic acid. ASPECT 97.

In aspects, the invention provides the composition of aspect 96, wherein the citric acid is present in an amount of between about 0.01 wt. % and about 0.1 wt. % of the composition. ASPECT 98.

In aspects, the invention provides the composition of aspect 98, wherein the citric acid is present in an amount of about 0.04 wt. % of the composition. ASPECT 99.

In aspects, the invention provides the composition of aspect 97, wherein the maleic acid is present in an amount of between about 0.001 wt. % and about 0.1 wt. % of the composition. ASPECT 100.

In aspects, the invention provides the composition of aspect 100, wherein the maleic acid is present in an amount of about 0.002 wt. %-about 0.05 wt. % of the composition. ASPECT 101.

In aspects, the invention provides the composition of any one or more of aspects 1-101, wherein the pharmaceutically acceptable and compositionally compatible small compound stabilizing agent(s) is/are present in an amount of at least about 0.04 wt. % of the composition. ASPECT 102.

In aspects, the invention provides the composition of any one or more of aspects 57-102, wherein the composition further comprises a sugar alcohol. ASPECT 103.

In aspects, the invention provides the composition of aspect 103, wherein the sugar alcohol is glycerol. ASPECT 104.

In aspects, the invention provides the composition of any one or both of aspects 103 and 104, wherein the composition comprises a sugar alcohol, e.g., glycerol, in an amount of between about 0.00001 wt. % and about 1 wt. % of the composition. ASPECT 105.

In aspects, the invention provides the composition of any one or more of aspects 57-105, wherein the composition comprises a detectable or significant amount of water. ASPECT 106.

In aspects, the invention provides the composition of aspect 106, wherein the composition comprises water in an amount of between about 0.02 wt. % and about 0.1 wt. % of the composition. ASPECT 107.

In aspects, the invention provides the composition of any one or more of aspects 57-107, wherein the ratio of the pharmaceutically acceptable and compositionally compatible small compound stabilizing agent to the solvent component, e.g., at least one C1-C6 alcohol characterizable as an alkyl alcohol, an aliphatic alcohol, or both, is not greater than about 1:300. ASPECT 108.

In aspects, the invention provides the composition of any one or more of aspects 57-108, wherein the ratio of the pharmaceutically acceptable and compositionally compatible small compound stabilizing agent to the solvent component, e.g., at least one C1-C6 alcohol characterizable as an alkyl alcohol, an aliphatic alcohol, or both, is not greater than about 1:350. ASPECT 109.

In aspects, the invention provides the composition of any one or more of aspects 57-109, wherein the ratio of the pharmaceutically acceptable and compositionally compatible small compound stabilizing agent to the solvent component, e.g., at least one C1-C6 alcohol characterizable as an alkyl alcohol, and aliphatic alcohol, or both, is not greater than about 1:400. ASPECT 110.

In aspects, the invention provides the composition of any one or more of aspects 57-110, wherein the composition does not comprise (e.g., is free of) any compound comprising at least about 12 contiguous carbons which demonstrates detectable or significant surfactant activity. ASPECT 111.

In aspects, the invention provides the composition of any one or more of aspects 57-111, wherein the composition is free of a compound comprising a ratio of carbon atoms to any non-hydrogen atom in the compound of at least about 1.5:1 which demonstrates detectable or significant surfactant activity. ASPECT 112.

In aspects, the invention provides the composition of any one or more of aspects 57-112, wherein the composition is free of a compound comprising a ratio of carbon atoms to any non-hydrogen atom in the compound of at least about 4:1 which demonstrates detectable or significant surfactant activity. ASPECT 113.

In aspects, the invention provides the composition of any one or more of aspects 57-113, wherein the composition does not comprise (e.g., is free of) any compound providing a significant level of surfactant activity. ASPECT 114.

In aspects, the invention provides composition comprising an effective amount of an active pharmaceutical ingredient component wherein each constituent of the active pharmaceutical ingredient component is at least substantially dissolved such that the solution is sufficiently homogeneous so as to allow light to at least substantially uniformly pass through the composition, wherein the an active pharmaceutical ingredient component comprises an effective amount of a tiotropium compound (a tiotropium compound); an effective amount of a formoterol compound (a formoterol compound); and an effective amount of at least one pharmaceutically acceptable inhalable corticosteroid or a pharmaceutically acceptable salt or solvate thereof, wherein the composition further comprises pharmaceutically acceptable and compositionally compatible small compound acid stabilizing agent(s) having a molecular weight of less than about 200 g/mol, providing detectable or significant chelating activity, or both, and a propellant component, wherein the propellant component does not comprise HFA-227. ASPECT 115.

In aspects, the invention provides the composition of aspect 115, wherein the tiotropium compound is a salt of tiotropium. ASPECT 116.

In aspects, the invention provides the composition of any one or both of aspect 115 and aspect 116, wherein the tiotropium compound is tiotropium bromide. ASPECT 117.

In aspects, the invention provides the composition of any one or more of aspects 115-117, wherein the tiotropium compound is present in an amount of between about 0.005 wt. % and about 0.01 wt. % of the composition. ASPECT 118.

In aspects, the invention provides the composition of any one or more of aspects 115-118, wherein the formoterol compound is a salt of formoterol. ASPECT 119.

In aspects, the invention provides the composition of any one or more of aspects 115-119, wherein the formoterol compound is formoterol fumarate. ASPECT 120.

In aspects, the invention provides the composition of any one or more of aspects 115-120, wherein the formoterol compound is formoterol fumarate dihydrate. ASPECT 121.

In aspects, the invention provides the composition of any one or more of aspects 115-121, wherein the formoterol compound is present in an amount of between about 0.005 wt. % and about 0.01 wt. % of the composition. ASPECT 122.

In aspects, the invention provides the composition of any one or more of aspects 115-122, wherein the formoterol compound is present in an amount of about 0.008 wt. % of the composition. ASPECT 123.

In aspects, the invention provides the composition of any one or more of aspects 115-123, wherein at least one of the one or more inhaled corticosteroid(s) is selected from beclomethasone, ciclesonide, a fluticasone compound, or a combination of any or all thereof. ASPECT 124.

In aspects, the invention provides the composition of any one or more of aspects 115-124, wherein the one or more inhaled corticosteroid(s) is beclomethasone. ASPECT 125.

In aspects, the invention provides the composition of any one or more of aspects 115-125, wherein the one or more inhaled corticosteroid(s) is ciclesonide. ASPECT 126.

In aspects, the invention provides the composition of any one or more of aspects 115-126, wherein the one or more inhaled corticosteroid(s) is a fluticasone compound. ASPECT 127.

In aspects, the invention provides the composition of aspect 127, wherein the fluticasone compound is fluticasone furoate. ASPECT 128.

In aspects, the invention provides the composition of any one or more of aspects 115-128, wherein the at least one pharmaceutically acceptable inhalable corticosteroid or a pharmaceutically acceptable salt or solvate thereof is present in the composition in an amount of between about 0.18 wt. % and about 0.36 wt. %. ASPECT 129.

In aspects, the invention provides the composition of any one or more of aspects 115-129, wherein the composition comprises a solvent component capable of detectably or significantly preventing or delaying any change in the homogeneity of the composition. ASPECT 130.

In aspects, the invention provides the composition of any one or more of aspects 115-130, wherein the solvent component is present in an amount of at least about 5 wt. % of the composition. ASPECT 131.

In aspects, the invention provides the composition of any one or more of aspects 115-131, wherein the solvent component is present in an amount of at least about 6 wt. % of the composition. ASPECT 132.

In aspects, the invention provides the composition of any one or more of aspects 115-132, wherein the solvent component is present in an amount of at least about 8 wt. % of the composition. ASPECT 133.

In aspects, the invention provides the composition of any one or more of aspects 115-133, wherein the solvent component is present in an amount of at least about 10 wt. % of the composition. ASPECT 134.

In aspects, the invention provides the composition of any one or more of aspects 115-134, wherein the solvent component is present in an amount of at least about 11 wt. % of the composition. ASPECT 135.

In aspects, the invention provides the composition of any one or more of aspects 115-135, wherein the solvent component is present in an amount of at least about 12 wt. % of the composition. ASPECT 136.

In aspects, the invention provides the composition of any one or more of aspects 115-136, wherein the solvent component is present in an amount of at least about 15 wt. % of the composition. ASPECT 137.

In aspects, the invention provides the composition of any one or more of aspects 115-137, wherein the solvent component is present in an amount of between about 15 wt. % and about 18 wt. % of the composition. ASPECT 138.

In aspects, the invention provides the composition of any one or more of aspects 115-138, wherein the solvent component comprises a C1-C6 alcohol. ASPECT 139.

In aspects, the invention provides the composition of any one or more of aspects 115-139, wherein the solvent component comprises a C1-C6 alcohol characterizable as an alkyl alcohol, an aliphatic alcohol, or both. ASPECT 140.

In aspects, the invention provides the composition of any one or more of aspects 115-81, wherein the solvent component comprises ethanol. ASPECT 141.

In aspects, the invention provides the composition of any one or more of aspects 115-141, wherein the propellant component comprises HFA-134a, HFA-152a, HFO-1234ze, or a combination thereof. ASPECT 142.

In aspects, the invention provides the composition of any one or more of aspects 115-142, wherein the propellant component comprises HFA-134a. ASPECT 143.

In aspects, the invention provides the composition of any one or more of aspects 115-142, wherein the propellant component comprises HFA-152a. ASPECT 85.

In aspects, the invention provides the composition of any one or more of aspects 115-142, wherein the propellant component comprises HFO-1234ze. ASPECT 145.

In aspects, the invention provides the composition of any one or more of aspects of 115-145, wherein the propellant component is at least substantially uniform and wherein the propellant component is at least about 95% composed of the same propellant compound. ASPECT 146.

In aspects, the invention provides the composition of any one or more o aspects 115-146, wherein the propellant component is at least substantially uniform and wherein the propellant component is at least about 96% composed of the same propellant compound. ASPECT 147.

In aspects, the invention provides the composition of any one or more of aspects 115-147, wherein the propellant component is at least about 98% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 320:1. ASPECT 148.

In aspects, the invention provides the composition of any one or more of aspects 115-148, wherein the propellant component is at least about 98% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 323:1. ASPECT 149.

In aspects, the invention provides the composition of any one or more of aspects 115-149, wherein the propellant component is at least about 98% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 326:1. ASPECT 150.

In aspects, the invention provides the composition of any one or more of aspects 115-150, wherein the propellant component is at least about 98% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 330:1. ASPECT 151.

In aspects, the invention provides the composition of any one or more of aspects 115-151, wherein the propellant component is at least about 98% composed of the same propellant compound wherein such compound establishes a dominant propellant, and wherein the ratio of the percent of the propellant component represented by the dominant propellant to the pharmaceutically acceptable and compositionally compatible small compound stabilizing agent(s) is at least about 333:1. ASPECT 152.

In aspects, the invention provides the composition of any one or more of aspects 115-152, wherein the small compound stabilizing agent(s) is/are capable of detectably or significantly preventing or delaying any change in the homogeneity of the composition resulting in (a) the amount of light capable of passing through the composition to be reduced or (b) the composition no longer being characterizable as a solution. ASPECT 153.

In aspects, the invention provides the composition of any one or more of aspects 115-153, wherein the pharmaceutically acceptable and compositionally compatible small compound stabilizing agent(s) comprise an acid. ASPECT 154.

In aspects, the invention provides the composition of any one or more of aspects 115-154, wherein the pharmaceutically acceptable and compositionally compatible small compound stabilizing agent(s) comprise an organic acid. ASPECT 155.

In aspects, the invention provides the composition of any one or more of aspects 115-155, wherein the pharmaceutically acceptable and compositionally compatible small compound stabilizing agent(s) is/are selected from citric acid and maleic acid. ASPECT 156.

In aspects, the invention provides the composition of any one or more of aspects 115-156, wherein the pharmaceutically acceptable and compositionally compatible small compound stabilizing agent(s) comprise citric acid. ASPECT 157.

In aspects, the invention provides the composition of any one or more of aspects 115-157, wherein the pharmaceutically acceptable and compositionally compatible small compound stabilizing agent(s) comprise maleic acid. ASPECT 158.

In aspects, the invention provides the composition of aspect 157, wherein the citric acid is present in an amount of between about 0.01 wt. % and about 0.1 wt. % of the composition. ASPECT 159.

In aspects, the invention provides the composition of aspect 159, wherein the citric acid is present in an amount of about 0.04 wt. % of the composition. ASPECT 160.

In aspects, the invention provides the composition of aspect 158, wherein the maleic acid is present in an amount of between about 0.001 wt. % and about 0.1 wt. % of the composition. ASPECT 161.

In aspects, the invention provides the composition of aspect 161, wherein the maleic acid is present in an amount of about 0.002 wt. %-about 0.05 wt. % of the composition. ASPECT 162.

In aspects, the invention provides the composition of any one or more of aspects 115-162, wherein the pharmaceutically acceptable and compositionally compatible small compound stabilizing agent(s) is/are present in an amount of at least about 0.04 wt. % of the composition. ASPECT 163.

In aspects, the invention provides the composition of any one or more of aspects 115-163, wherein the composition further comprises a sugar alcohol. ASPECT 164.

In aspects, the invention provides the composition of aspect 164, wherein the sugar alcohol is glycerol. ASPECT 165.

In aspects, the invention provides the composition of any one or both of aspects 164 and 165, wherein the composition comprises a sugar alcohol, e.g., glycerol, in an amount of between about 0.00001 wt. % and about 1 wt. % of the composition. ASPECT 166.

In aspects, the invention provides the composition of any one or more of aspects 115-166, wherein the composition comprises a detectable or significant amount of water. ASPECT 167.

In aspects, the invention provides the composition of aspect 167, wherein the composition comprises water in an amount of between about 0.02 wt. % and about 0.1 wt. % of the composition. ASPECT 168.

In aspects, the invention provides the composition of any one or more of aspects 115-168, wherein the ratio of the pharmaceutically acceptable and compositionally compatible small compound stabilizing agent(s) to the solvent component, e.g., at least one C1-C6 alcohol characterizable as an alkyl alcohol, an aliphatic alcohol, or both, is not greater than about 1:400. ASPECT 169.

In aspects, the invention provides the composition of any one or more of aspects 115-169, wherein the ratio of the pharmaceutically acceptable and compositionally compatible small compound stabilizing agent(s) to the solvent component, e.g., at least one C1-C6 alcohol characterizable as an alkyl alcohol, an aliphatic alcohol, or both, is not greater than about 1:350. ASPECT 170.

In aspects, the invention provides the composition of any one or more of aspects 115-170, wherein the ratio of the pharmaceutically acceptable and compositionally compatible small compound stabilizing agent(s) to the solvent component, e.g., at least one C1-C6 alcohol characterizable as an alkyl alcohol, and aliphatic alcohol, or both, is not greater than about 1:300, not greater than about 1:275, not greater than about 1:250, or not greater than about 1:225. ASPECT 171.

In aspects, the invention provides the composition of any one or more of aspects 115-171, wherein the composition does not comprise (e.g., is free of) any compound comprising at least about 12 contiguous carbons which demonstrates detectable or significant surfactant activity. ASPECT 172.

In aspects, the invention provides the composition of any one or more of aspects 115-172, wherein the composition is free of a compound comprising a ratio of carbon atoms to any non-hydrogen atom in the compound of at least about 1.5:1 which demonstrates detectable or significant surfactant activity. ASPECT 173.

In aspects, the invention provides the composition of any one or more of aspects 115-173, wherein the composition is free of a compound comprising a ratio of carbon atoms to any non-hydrogen atom in the compound of at least about 4:1 which demonstrates detectable or significant surfactant activity. ASPECT 174.

In aspects, the invention provides the composition of any one or more of aspects 115-174, wherein the composition does not comprise (e.g., is free of) any compound providing a significant level of surfactant activity. ASPECT 175.

In aspects, the invention provides the composition described in anyone or more of aspects 1-175, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 1 month. ASPECT 176.

In aspects, the invention provides the composition described in anyone or more of aspects 1-176, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 3 months. ASPECT 177.

In aspects, the invention provides the composition described in anyone or more of aspects 1-177, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 6 months. ASPECT 178.

In aspects, the invention provides the composition described in anyone or more of aspects 1-178, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 9 months. ASPECT 179.

In aspects, the invention provides the composition described in anyone or more of aspects 1-179, wherein the composition comprise less than about 0.2 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 12 months. ASPECT 180.

In aspects, the invention provides the composition described in anyone or more of aspects 176-180, wherein the composition comprises less than about 0.1 wt. % total impurities generated by the degradation of (a) any one API, (b) any combination of multiple API(s), or (c) both (a) and (b) based on the total weight of such one, more, or all API(s) in the composition and any associated impurity(ies), after storage at about 40° C. and about 75% relative humidity for a period of at least about 1 month, such as, e.g., a period of at least about 3 months, at least about 6 months, at least about 9 months, or, e.g., at least about 12 months. ASPECT 181.

In aspects, the invention provides a method of manufacturing any one or more of the composition(s) described in any one or more of aspects 1-181, wherein the method comprises (1) collecting and combining all API(s) of a composition; (2) adding non-volatile composition component(s) to the API(s); (3) mixing the combination of components until sufficiently mixed to facilitate addition of a propellant; (4) filling container(s) with the desired quantity of composition by a cold filling method comprising (a) adding the required amount of propellant to the mixed composition; (b) mixing the resulting combination of non-propellant components with the propellant until the resulting composition is completely dissolved or until the composition is uniformly suspended; (c) chilling the resulting composition to a temperature of between about −50° C.-about −60° C.; (d) adding the composition to a final packaging container (e.g., a canister); and (e) allowing the container to warm to ambient temperature, resulting in an increase in pressure within the container. ASPECT 182.

In aspects, the invention provides a method of manufacturing any one or more of the composition(s) described in any one or more of aspects 1-181, wherein the method comprises (1) collecting and combining all API(s) of a composition; (2) adding non-volatile composition component(s) to the API(s); (3) mixing the combination of components until sufficiently mixed to facilitate addition of a propellant; (4) filling container(s) with the desired quantity of composition by a pressure filling method comprising (a) adding a required amount of propellant to the mixed composition; (b) adding the mixture of non-propellant components and propellant to a final packaging container (e.g., canister) by way of applied pressure. ASPECT 183.

In aspects, the invention provides a method of directly treating or prophylactically treating in a mammal chronic obstructive pulmonary disease (COPD), asthma, one or more similar or related disorders, such as, e.g., disorders related to the obstruction of airflow to or from the lung(s); any one or more symptoms related to any one or more of such conditions; or any combination of any or all thereof, the method comprising administering a therapeutically effective amount of a composition described in any one or more of aspects 1-181. ASPECT 184.

In aspects, the invention provides a method of using a therapeutically effective amount of any one or more composition(s) described in any one or more of aspects 1-181 in the direct treatment or prophylactic treatment of a mammal suffering from or at risk of suffering from chronic obstructive pulmonary disease (COPD), asthma, one or more similar or related respiratory disorders, such as, e.g., disorders related to the obstruction of airflow to or from the lung(s); any one or more symptoms related to any one or more of such conditions; or any combination of any or all thereof. ASPECT 185.

In aspects, the invention provides a method of detectably or significantly expanding, e.g., dilating, one or more airway(s), e.g., bronchioles, in a recipient suffering from restriction of such one or more airways comprising administration of a therapeutically effective amount of a composition described in any one or more of aspects 1-181. ASPECT 186.

In aspects, the invention provides the method of any one or more of aspects 184-186, wherein the application of the method results in a detectably or significantly greater efficiency of delivery of API compared to a reference product, wherein the reference product is one or more of the reference products described herein. ASPECT 187.

In aspects, the invention provides a kit comprising at least one container comprising any composition described in any one or more of aspects 1-181. ASPECT 188.

In aspects, the invention provides the kit of aspect 188, wherein the kit comprises at least two or more containers each comprising a composition described in any one or more of aspects 1-180. ASPECT 189.

In aspects, the invention provides the kit of any one or both of aspect 188 and aspect 189, wherein the kit further comprises one or more delivery device(s) designed to effectively deliver the composition(s) provided by the kit. ASPECT 190.

In aspects, the invention provides the kit of aspect 190, wherein at least one of the one or more delivery device(s) is a device suitable for providing the composition(s) to a recipient by inhalation (e.g., an inhaler device). ASPECT 191.

In aspects, the invention provides the kit of aspect 191, wherein the inhaler device is a metered dose inhaler. ASPECT 192.

In aspects, the invention provides the kit of any one or more of aspects 188-192, wherein the kit is used in any one or more of the method(s) described in aspects 184-187. ASPECT 193.

In aspects, the invention provides any one or more of the methods described in this Exemplary Aspects of the Invention section, wherein the application of the method results in (1) a detectably or significantly greater efficiency of delivery of one or more API(s) compared to a reference product (wherein the reference product is one or more reference products such as those described herein); (2) a detectable or significant clinical improvement in one or more measures of chronic obstructive pulmonary disease, asthma, one or more similar or related disorders, such as, e.g., disorders related to the obstruction of airflow to or from the lung(s), any one or more symptoms related to any one or more of such condition(s), or any combination of any or all thereof; (3) a detectably or significantly greater clinical improvement in one or more measures of chronic obstructive pulmonary disease, asthma, one or more similar or related disorders, such as, e.g., disorders related to the obstruction of airflow to or from the lung(s), any one or more symptoms related to any one or more of such condition(s), or any combination of any or all thereof, compared to a reference product (wherein the reference product is one or more reference product(s) as described herein); or (4) any combination of any of (1)-(3), wherein efficacy, outcome(s), or clinical measure of the efficacy of such method(s) (such as, e.g., a performance characteristic, such as, e.g., a detectable or significant improvement in delivery efficiency; a detectable or significant reduction in frequency, severity, or frequency and severity of symptomatic attack (e.g., of acute asthmatic or bronchoconstrictor attack); detectable or significant improvement in lung function; a detectable or significant improvement in airway hyperreactivity; a detectable or significant reduction in the requirement for one or more other symptomatic therapy(ies), e.g., one or more therapy(ies) for or intended to restrict or abort symptomatic attack when it occurs; a detectable or significant reduction in morning dipping (treatment between about 4 AM and about 6 AM, or, e.g., other similar period of time generally most distant from any previous treatment/administration of composition(s)); or any combination thereof), is/are determined by an appropriately conducted and appropriately powered clinical study, such as an appropriately conducted clinical study recognized by an accepted regulatory authority such as, e.g., the U.S. FDA. or similar or related measure of composition efficacy/performance or other measure(s) of efficacy described herein or otherwise known in the art. ASPECT 194.

### Further Exemplary Facets

Additional exemplary facets of the invention include the following, which can be combined with any uncontradicted aspect, embodiment, facet, etc., whether from the preceding three subsections of this section or any other described aspect of the invention.

A first facet is a pharmaceutically acceptable composition comprising an active pharmaceutical ingredient component in solution, wherein each constituent of the active pharmaceutical ingredient component is at least substantially dissolved such that the solution is sufficiently homogeneous so as to allow light to at least substantially uniformly pass through the composition, wherein the active pharmaceutical ingredient component comprises an effective amount of at least one tiotropium compound (a tiotropium compound); at least one formoterol compound (a formoterol compound); and at least one pharmaceutically acceptable inhalable corticosteroid compound, and wherein the composition further comprises (1) an effective amount of at least one C1-C6 alcohol characterizable as an alkyl alcohol, an aliphatic alcohol, or both; (2) an effective amount of one or more pharmaceutically acceptable and compositionally compatible small compound stabilizing agent(s) each having a molecular weight of less than about 200 g/mol, providing detectable or significant chelating activity, or both, wherein the small compound stabilizing agent(s) is/are capable of detectably or significantly preventing or delaying any change in the homogeneity of the composition resulting in (a) the amount of light capable of passing through the composition to be reduced or (b) the composition no longer being characterizable as a solution; and (3) a propellant component comprising one or more pharmaceutically acceptable propellant compounds, wherein the ratio of the pharmaceutically compatible small compound stabilizing agent to the at least one C1-C6 alcohol characterizable as an alkyl alcohol, an aliphatic alcohol, or both is not greater than about 1:300. FACET 1

A pharmaceutically acceptable composition comprising an active pharmaceutical ingredient component wherein each constituent of the active pharmaceutical ingredient component is at least substantially dissolved and the solution sufficiently homogeneous mixed to allow light to at least substantially uniformly pass through the composition, wherein the an active pharmaceutical ingredient component comprises an effective amount of a tiotropium compound, such as tiotropium or a pharmaceutically acceptable salt or solvate thereof, and an effective amount of a formoterol compound, such as formoterol or a pharmaceutically acceptable salt or solvate thereof, and the composition further comprises (1) at least one C1-C6 alcohol characterizable as an alkyl alcohol, an aliphatic alcohol, or both, present in an amount of at least about 11.25 wt. % of the composition; (2) a pharmaceutically acceptable and compositionally compatible acid compound capable of detectably or significantly preventing or delaying any change in the homogeneity of the composition resulting in (a) the amount of light capable of passing through the composition to be reduced or (b) the composition no longer being characterizable as a solution; and (3) a propellant component comprising one or more pharmaceutically acceptable propellant compounds. FACET 2.

A pharmaceutically acceptable composition comprising an active pharmaceutical ingredient component in solution wherein each constituent of the active pharmaceutical ingredient component is at least substantially dissolved and the solution sufficiently homogeneous mixed to allows light to at least substantially uniformly pass through the composition, wherein the active pharmaceutical ingredient component comprises a tiotropium compound (i.e., at least one tiotropium compound) and a formoterol compound (at least one formoterol compound), and wherein the composition further comprises (1) at least one C1-C6 alcohol characterizable as an alkyl alcohol, an aliphatic alcohol, or both in an amount of at least about 6 wt. % of the composition; (2) at least one pharmaceutically acceptable and compositionally compatible organic acid; and (3) a propellant component comprising one or more pharmaceutically acceptable propellant compound(s), wherein the composition is free of any compound that provides/exhibits significant surfactant activity. FACET 3.

A pharmaceutically acceptable composition comprising an active pharmaceutical ingredient component in solution, wherein each constituent of the active pharmaceutical ingredient component is at least substantially dissolved such that the solution is sufficiently homogeneous so as to allow light to at least substantially uniformly pass through the composition, wherein the active pharmaceutical ingredient component comprises an effective amount of at least one tiotropium compound; at least one formoterol compound; and at least one pharmaceutically acceptable inhalable corticosteroid compound, and wherein the composition further comprises (1) at least one C1-C6 alcohol characterizable as an alkyl alcohol, an aliphatic alcohol, or both; (2) a pharmaceutically acceptable and compositionally compatible acid compound capable of detectably or significantly preventing or delaying any change in the homogeneity of the composition resulting in (a) the amount of light capable of passing through the composition to be reduced or (b) the composition no longer being characterizable as a solution; and (3) an at least substantially uniform propellant component, wherein the propellant component is at least about 95% composed of the same propellant compound. FACET 4.

A pharmaceutically acceptable composition comprising an active pharmaceutical ingredient component in solution, wherein each constituent of the active pharmaceutical ingredient component is at least substantially dissolved such that the solution is sufficiently homogeneous so as to allow light to at least substantially uniformly pass through the composition, wherein the active pharmaceutical ingredient component comprises an effective amount of at least one tiotropium compound; at least one formoterol compound; and at least one pharmaceutically acceptable inhalable corticosteroid compound, and wherein the composition further comprises (1) at least one C1-C6 alcohol characterizable as an alkyl alcohol, an aliphatic alcohol, or both; (2) a pharmaceutically acceptable and compositionally compatible small compound stabilizing agent having a molecular weight of less than about 200 g/mol, providing detectable or significant chelating activity, or both, wherein the small compound stabilizing agent is capable of detectably or significantly preventing or delaying any change in the homogeneity of the composition resulting in (a) the amount of light capable of passing through the composition to be reduced or (b) the composition no longer being characterizable as a solution, wherein (I) the pharmaceutically acceptable and compositionally compatible small compound stabilizing agent comprises an organic acid, (II) the composition further comprises a sugar alcohol, or both (I) and (II) are true; and (3) an at least substantially uniform propellant component, wherein the propellant component is at least about 95% composed of the same propellant compound. FACET 5.

A pharmaceutically acceptable composition comprising an active pharmaceutical ingredient component in solution, wherein each constituent of the active pharmaceutical ingredient component is at least substantially dissolved such that the solution is sufficiently homogeneous so as to allow light to at least substantially uniformly pass through the composition, wherein the active pharmaceutical ingredient component comprises an effective amount of at least one tiotropium compound; at least one formoterol compound; and at least one pharmaceutically acceptable inhalable corticosteroid compound, and wherein the composition further comprises (1) at least one C1-C6 alcohol characterizable as an alkyl alcohol, an aliphatic alcohol, or both; (2) a pharmaceutically acceptable and compositionally compatible organic acid; and (3) an at least substantially uniform and pharmaceutically acceptable propellant component, wherein the propellant component is at least about 96% composed of the same propellant compound establishing the percent of the propellant component represented by a dominant propellant, and wherein ratio of the percent of the propellant component represented by the dominant propellant to the pharmaceutically acceptable and compositionally compatible organic acid is at least about 320:1. FACET 6.

A method of directly or prophylactically treating chronic obstructive pulmonary disease (COPD), asthma, or a similar disorder related to either or both thereof involving the obstruction of airflow to or from the lung(s), in a mammal, wherein the method comprises administration of a therapeutically effective amount of the composition of any one or more of facets 1-6 for a therapeutically effective period of time, and wherein application of the method results in the detectable or significant expansion (dilation) of one or more airways in the recipient mammal. FACET 7.

A pharmaceutically acceptable composition comprising an active pharmaceutical ingredient component wherein at least a significant portion of one or more constituents of the active pharmaceutical ingredient component remain undissolved such that light cannot pass uniformly through the composition and the composition is characterizable as a suspension or hybrid solution-suspension, wherein the active pharmaceutical ingredient component comprises a tiotropium compound (at least one tiotropium compound) and a formoterol compound (at least one formoterol compound), and wherein the composition further comprises (1) one or more pharmaceutically acceptable and compositionally compatible small compound stabilizing agent(s) having a molecular weight of less than about 200 g/mol, providing detectable or significant chelating activity, or both, wherein the small compound stabilizing agent is capable of detectably or significantly preventing or delaying any change in the homogeneity of the composition and the pharmaceutically acceptable and compositionally compatible small compound stabilizing agent(s) is present in an amount of at least about 0.04 wt. % (readers will recognize that such a reference to an amount given in weight percent or similar unit can be alternatively characterized, uncontradicted, as a concentration); (2) a surfactant compound, wherein the surfactant compound does not meet the criteria of claim element (1) above; and (3) a propellant component comprising one or more pharmaceutically acceptable propellant compounds, wherein the propellant component does not comprise HFA-227. FACET 8.

A method of directly or prophylactically treating chronic obstructive pulmonary disease (COPD), asthma, or a similar disorder related to either or both thereof involving the obstruction of airflow to or from the lung(s), in a mammal, wherein the method comprises administration of a therapeutically effective amount of the composition of facet 8 for a therapeutically effective period of time, and wherein application of the method results in the detectable or significant expansion (dilation) of one or more airways in the recipient mammal. FACET 9.

A pharmaceutically acceptable composition comprising an active pharmaceutical ingredient component wherein each constituent of the active pharmaceutical ingredient component is at least substantially dissolved such that the solution is sufficiently homogeneous so as to allow light to at least substantially uniformly pass through the composition, wherein the an active pharmaceutical ingredient component comprises an effective amount of at least one tiotropium compound (a tiotropium compound); an effective amount of at least one formoterol compound (a formoterol compound); and an effective amount of at least one pharmaceutically acceptable inhalable corticosteroid compound (a corticosteroid compound), wherein the composition further comprises (1) a solvent component present in an amount representing at least about 11 wt. % of the composition; (2) pharmaceutically acceptable and compositionally compatible small compound stabilizing agent(s) having a molecular weight of less than about 200 g/mol, providing detectable or significant chelating activity, or both, wherein the small compound stabilizing agent is capable of detectably or significantly preventing or delaying any change in the homogeneity of the composition resulting in (a) the amount of light capable of passing through the composition to be reduced or (b) the composition no longer being characterizable as a solution; and (3) a propellant component comprising one or more pharmaceutically acceptable propellant compounds. FACET 10.

A pharmaceutically acceptable composition comprising an active pharmaceutical ingredient component wherein each constituent of the active pharmaceutical ingredient component is at least substantially dissolved such that the solution is sufficiently homogeneous so as to allow light to at least substantially uniformly pass through the composition, wherein the an active pharmaceutical ingredient component comprises an effective amount of at least one tiotropium compound (a tiotropium compound); an effective amount of at least one formoterol compound (a formoterol compound); and an effective amount of at least one pharmaceutically acceptable inhalable corticosteroid compound (a corticosteroid compound), wherein the composition further comprises (1) a solvent component present in an amount representing at least about 11 wt. % of the composition; (2) pharmaceutically acceptable and compositionally compatible small compound stabilizing agent(s) having a molecular weight of less than about 200 g/mol, providing detectable or significant chelating activity, or both, wherein the small compound stabilizing agent is capable of detectably or significantly preventing or delaying any change in the homogeneity of the composition resulting in (a) the amount of light capable of passing through the composition to be reduced or (b) the composition no longer being characterizable as a solution; and (3) a propellant component comprising one or more pharmaceutically acceptable propellant compounds. FACET 11.

A pharmaceutically acceptable composition comprising an effective amount of an active pharmaceutical ingredient component wherein each constituent of the active pharmaceutical ingredient component is at least substantially dissolved such that the solution is sufficiently homogeneous so as to allow light to at least substantially uniformly pass through the composition, wherein the an active pharmaceutical ingredient component comprises an effective amount of a tiotropium compound (a tiotropium compound); an effective amount of a formoterol compound (a formoterol compound); and at least one pharmaceutically acceptable inhalable corticosteroid or a pharmaceutically acceptable salt or solvate thereof, wherein the composition further comprises (1) a solvent component capable of detectably or significantly preventing or delaying any change in the homogeneity of the composition; (2) pharmaceutically acceptable and compositionally compatible small compound stabilizing agent(s) having a molecular weight of less than about 200 g/mol, providing detectable or significant chelating activity, or both, wherein the small compound stabilizing agent is capable of detectably or significantly preventing or delaying any change in the homogeneity of the composition resulting in (a) the amount of light capable of passing through the composition to be reduced or (b) the composition no longer being characterizable as a solution; and (3) an at least substantially homogeneous propellant component wherein the propellant component is at least about 98% composed of the same propellant compound. FACET 12.

A pharmaceutically acceptable composition comprising an effective amount of an active pharmaceutical ingredient component wherein each constituent of the active pharmaceutical ingredient component is at least substantially dissolved such that the solution is sufficiently homogeneous so as to allow light to at least substantially uniformly pass through the composition, wherein the an active pharmaceutical ingredient component comprises an effective amount of a tiotropium compound (a tiotropium compound); an effective amount of a formoterol compound (a formoterol compound); and an effective amount of at least one pharmaceutically acceptable inhalable corticosteroid or a pharmaceutically acceptable salt or solvate thereof, wherein the composition further comprises pharmaceutically acceptable and compositionally compatible small compound acid stabilizing agent(s) having a molecular weight of less than about 200 g/mol, providing detectable or significant chelating activity, or both, and a propellant component, wherein the propellant component does not comprise HFA-227. FACET 13.

A method of directly or prophylactically treating chronic obstructive pulmonary disease (COPD), asthma, or a similar disorder related to either or both thereof involving the obstruction of airflow to or from the lung(s), in a mammal, wherein the method comprises administration of a therapeutically effective amount of the composition of any one or more of facets 10-13 for a therapeutically effective period of time (readers will recognize that any reference to such a period implicitly discloses performing the method over such period for an effective number of times), and wherein application of the method results in the detectable or significant expansion (dilation) of one or more airways in the recipient mammal. FACET 14.

### DETAILED DESCRIPTION OF THE INVENTION

For convenience, both combinations of elements/steps and individual elements/steps may be described in this section of this disclosure. Despite the inclusion of passages focused on specific elements/steps, any aspect, facet, embodiment, or other description of particular step(s) or element(s) can be applied to any general description of the composition(s)/method(s) of the invention, or any other recited element(s)/step(s) thereof, which are provided in any part of this disclosure.

As used herein, the word “exemplary” means “serving as an example, instance, or illustration.” The following detailed description is merely exemplary in nature and is not intended to limit application and uses. Any embodiment described herein as “exemplary” is not necessarily to be construed as preferred or advantageous over other embodiments.

### Compositions

In aspects, the invention provides pharmaceutically acceptable composition(s) comprising one or more active pharmaceutical ingredients (API(s)) suitable for use in the prophylactic treatment, direct treatment, or both, of asthma, chronic pulmonary obstructive disorder (COPD), similar or related respiratory-related disorders, any combination thereof, or, e.g., or one or more symptoms related to any or all thereof. Composition(s) herein, are, in aspects, designed to be delivered by inhalation. In aspects, composition(s) herein are delivered via an inhalation device, such as, e.g., a metered dose inhaler (MDI) or breath actuated inhaler, or, e.g., a pressurized metered dose inhaler (pMDI).

In aspects, composition(s) provided by the invention comprise an API component comprising one or more API subcomponents or one or more API agents/constituents (e.g., one or more API compound(s)). In aspects, each such API component can comprise API constituent(s)/compound(s) which perform one or more detectably or significantly different function(s), e.g., demonstrate one or more detectably or significantly different activity(ies) than the API constituent(s)/compound(s) of one or more other API subcomponent(s). For example, in aspects, composition(s) can comprise, e.g., a bronchodilating component comprising one or more bronchodilating agent(s)/(compound(s)), as is described further elsewhere herein. Other examples described herein include, e.g., composition(s) comprising an anti-inflammatory component comprising one or more anti-inflammatory agent(s)/(compound(s)). In aspects, composition(s) herein comprise one or more other component(s) comprising one or more APIs effective in preventing or treating one or more respiratory-related, or lung-related condition(s) affecting breathing. In aspects, composition(s) of the invention can comprise any combination of such components, such as, e.g., composition(s) comprising both a bronchodilating component and an anti-inflammatory component. In aspects, each such component can comprise one or more further subcomponents, each subcomponent comprising one or more API(s) which share common characteristic(s) but perform one or more detectably or significantly different activities. As an example, as is further described elsewhere herein, in aspects, a bronchodilating component can comprise, e.g., a beta-adrenergic agonist component and, e.g., an anticholinergic component. In such scenarios, constituent(s)/compound(s) of the beta-adrenergic agonist component can share one or more characteristics with constituent(s)/compound(s) of the anticholinergic component, e.g., providing measurable bronchodilating effect by, for example, causing muscle relaxation. However, in such scenarios, the constituent(s)/compound(s) of each subcomponent can, e.g., provide such bronchodilating effect by different physiological activity (e.g., by blocking the action of the neurotransmitter acetylcholine in the case of an anticholinergic agent or, e.g., by mimicking the actions of, e.g., epinephrine in the case of a beta-adrenergic agonist.)

In exemplary aspects, composition(s) provided by the invention comprise a bronchodilating component, comprising one or more bronchodilation constituent(s)/compound(s), e.g., one or more long-acting bronchodilators. In aspects, such a long-acting bronchodilator can be, e.g., one or more anticholinergic (antimuscarinic) agent(s)/compound(s), one or more beta 2 agonist agent(s)/compound(s), or, e.g., combination(s) thereof. In other exemplary aspects, composition(s) provided by the invention comprise an anti-inflammatory component, comprising, e.g., one or more steroid constituent(s)/compound(s), e.g., one or more corticosteroid(s), e.g., one or more inhaled corticosteroids (ICS). In aspects, composition(s) comprise a combination therapy (e.g., two or more APIs). In aspects, composition(s) provided herein comprise one or more bronchodilating agents, e.g., one or more one or more anticholinergics, one or more beta 2 agonists, or both, and, e.g., one or more ICSs. In aspects, composition(s) herein provide for combination therapy, e.g., wherein composition(s) comprise at least one long-acting muscarinic (also referred to as an anticholinergic) antagonist (LAMA) constituent and at least one ICS constituent or, e.g., at least one long-acting beta agonist (LABA) constituent and at least one ICS constituent. In aspects, composition(s) herein provide a triple therapy, such as, e.g., comprising at least one LAMA constituent, at least one LABA constituent, and at least one ICS constituent.

Composition(s) provided herein, in aspects, further comprise a propellant component comprising one or more propellant compound(s). In aspects, composition(s) provided herein additionally comprise an excipient component comprising one or more pharmaceutically acceptable and inhalation-suitable excipient(s).

### Active Pharmaceutical Ingredient(s) (API(s))

A number of APIs are described herein as suitable for incorporation into composition(s) provided by the invention. Examples of such active pharmaceutical ingredients include, e.g., tiotropium, formoterol, arformoterol, fluticasone propionate, fluticasone furoate, fluticasone valerate, ciclesonide, beclomethasone, albuterol, salbutamol, levoalbuterol, levosalbutamol butaline, pirbuterol, procaterol, metaproterenol, fenoterol, isoproterenol, metaproterenol, terbutaline, isoetarine, bitolterol mesylate, ritodrine, salmeterol, carmoterol, bambuterol, clenbuterol, indacaterol, milveterol, vilanterol, olodaterol, mometasone, and, e.g., budesonide, R-budesonide. Herein, such terms are used for sake of simplicity and should be interpreted to include not only the active ingredient directly, but also all pharmaceutically acceptable derivatives thereof. Suitable derivatives include pharmaceutically acceptable salts, pharmaceutically acceptable solvates, pharmaceutically acceptable hydrates, pharmaceutically acceptable isomers, pharmaceutically acceptable esters, pharmaceutically acceptable anhydrates, pharmaceutically acceptable enantiomers, pharmaceutically acceptable polymorphs, pharmaceutically acceptable prodrugs, pharmaceutically acceptable tautomers, and/or pharmaceutically acceptable complexes thereof, or any combination of such derivatives.

For example, in aspects, reference to tiotropium or a tiotropium compound herein should be interpreted as encompassing, e.g., all pharmaceutically acceptable forms of tiotropium identified above, including, e.g., pharmaceutically acceptable salts of tiotropium, such as, e.g., tiotropium bromide, for example, e.g., tiotropium bromide monohydrate or tiotropium bromide anhydrate. In aspects, reference to formoterol or a formoterol compound herein should be interpreted as encompassing, e.g., all pharmaceutically acceptable forms of formoterol identified above, including, e.g., pharmaceutically acceptable salts of formoterol, such as, e.g., formoterol fumarate, for example, e.g., formoterol fumarate hydrate or formoterol fumarate anhydrate.

In common aspects, API(s) described herein are not complexed, e.g., do not form a complex with one or more component(s)/element(s) that are characterized in the art as adjuvant(s) or that exhibit similar effects/characteristics as such component(s). Alternatively, in aspects, one or more API(s) described herein can be complexed with, e.g., one or more adjuvant(s). In aspects, complexing agent(s) can be, e.g., any agent which detectably or significantly increases the solubility of API(s), detectably or significantly improves the suspension characteristic(s) of composition(s), e.g., specifically API(s), detectably or significantly increases the stability of composition(s) described herein, or any combination of any or all thereof. In aspects, composition(s) comprise an API component comprising no API compound in complexed form.

In certain aspects, composition(s) comprise an API component comprising at least two APIs (compounds that are chemically distinct from each other, often having significantly different chemical compositions/structures, such as a Tanimoto (or Jaccard) coefficient, T, of <0.85, 0.7, or <0.5, and usually belonging to different structural, functional, or both structural and functional classes of drugs from one another, such as, e.g., two bronchodilating compounds, or, e.g., one or more bronchodilating compound(s) and one or more corticosteroid compound(s)). Such two or more APIs are typically present in effective amounts (individually or only in combination with each other). In certain aspects, composition(s) comprising an API component comprising a bronchodilating component, the bronchodilating component comprises at least two bronchodilating compounds, which are chemically distinct from one another.

### Bronchodilating (Bronchodilator) Component

In aspects, the invention provides composition(s) delivered by inhalation comprising a bronchodilating component (also referred to herein as a bronchodilator component). In aspects, a bronchodilating component comprises one or more bronchodilating agent(s)/compound(s). In aspects, a bronchodilating agent/compound is any pharmaceutically acceptable compound suitable for inhalation which, upon administration, detectably or significantly relaxes muscle(s) of the lungs, detectably or significantly widens (dilates) airway(s) of the lung, e.g., bronchi, or both. In aspects, detectable or significant relaxation of muscle(s) of the lungs, widening/dilation of airway(s) of the lungs, or both detectably or significantly improves respiratory function (e.g., results in a detectable or significant improvement in respiratory function.)

### Short-Acting Bronchodilator Component

According to certain aspects, a bronchodilating component can comprise one or more subcomponents. In aspects, a bronchodilating component can comprise a short-acting bronchodilator component. In aspects, a short-acting bronchodilator component can comprise one or more short-acting bronchodilating agent(s)/compound(s). Such compounds are often referred to as, e.g., “quick acting”, “reliever”, or “rescue” compound(s). In aspects, short-acting bronchodilating agent(s)/compound(s) can be any pharmaceutically acceptable bronchodilating agent/compound which provide(s) detectable or significant relief from constricted airways, e.g., measurable expansion (dilation) of bronchioles as recognized in the art, as demonstrated by bioequivalence to an approved drug with similar properties, or as demonstrated in a statistically significant population of people in an appropriately conducted and powered clinical trial recognized by an applicable regulatory authority/body such as the United States Food and Drug Administration (US FDA) within about 20 minutes (min), e.g., within ˜18 min, ˜16 min, ˜14 min, ˜12 min, ˜10 min, ˜8 min, ˜6 min, ˜5 min, ˜4 min, ˜3 min, ˜2 min, or even within about 1 min of administration. In aspects, a short-acting bronchodilating agent/compound provides relief for a period of at least about 30 min, such as, e.g., ≥˜45 min, ≥˜1 hour, ≥˜1.25 hours, ≥˜1.5 hours, ≥˜1.75 hours, ≥˜2 hours, ≥˜2.25 hours, ≥˜2.5 hours, ≥˜2.75 hours, ≥˜3 hours, ≥˜3.25 hours, ≥˜3.5 hours, ≥˜3.75 hours, or, e.g., ≤˜4 hours. In aspects, a short-acting bronchodilating agent/compound is a compound recognized in the art or demonstrating the same in a statistically significant population of people in an appropriately conducted and powered clinical trial recognized by an applicable regulatory authority/body such as the US FDA as providing shorter term relief from respiratory distress compared to that of long-acting bronchodilating agent(s)/compound(s), such as, e.g., providing relief for a period of no more than about 10 hours, e.g., ≤˜9.5 hours, ≤˜9 hours, ≤˜8.5 hours, ≤˜8 hours, ≤˜7.5 hours, ≤˜7 hours, ≤˜6.5 hours, ≤˜6 hours, ≤˜5.5 hours, ≤˜5 hours, or, e.g., ≤˜4.5 hours.

Exemplary short-acting bronchodilating agent(s) include, e.g., albuterol, metaproterenol, ipratropium, levalbuterol, terbutaline, pirbuterol, etc.

### Short-Acting Bronchodilator Component (Amount)

In aspects, a short-acting bronchodilator component can be present in any therapeutically effective amount. In certain aspects, composition(s) comprise a single API component, e.g., comprise only a short-acting bronchodilator component. In aspects, the short-acting bronchodilator component comprises a single short-acting bronchodilating agent. In aspects, the short-acting bronchodilator component comprises two or more short-acting bronchodilating agents. In aspects, therefore, amount(s) of, e.g., bronchodilator component described herein can refer to: (a) the entirety of the API concentration within a composition when the API component of a composition comprises only a short-acting bronchodilator component; (b) the entirety of any bronchodilating component concentration of a composition when the bronchodilating component consists only of a short-acting bronchodilating component; (c) the entirety of any short-acting bronchodilator component concentration of a composition; (d) the specific concentration of any single short-acting bronchodilating agent/compound in the composition; or (e) any combination of (a)-(d).

In aspects, a short-acting bronchodilator component can be present in an amount of between about 5 μg and about 3000 μg, such as, e.g., between ˜5 μg and ˜2800 μg, ˜5 μg-˜2600 μg, ˜5 μg-˜2400 μg, ˜5 μg-˜2200 μg, ˜5 μg-˜2000 μg, ˜5 μg-˜1800 μg, ˜5 μg-˜1600 μg, ˜5 μg-˜1400 μg, ˜5 μg-˜1200 μg, ˜5 μg-˜1000 μg, ˜5 μg-˜800 μg, ˜5 μg-˜600 μg, ˜5 μg-˜400 μg, ˜5 μg-˜200 μg, or, e.g., ˜5 μg-˜100 μg.

In aspects, a short-acting bronchodilator component can be present in an amount of between ˜100 μg and ˜3000 μg, ˜200 μg-˜3000 μg, ˜400 μg-˜3000 μg, ˜600 μg-˜3000 μg, ˜800 μg-˜3000 μg, ˜1000 μg-˜3000 μg, ˜1200 μg-˜3000 μg, ˜1400 μg-˜3000 μg, ˜1600 μg-˜3000 μg, ˜1800 μg-˜3000 μg, ˜2000 μg-˜3000 μg, ˜2200 μg-˜3000 μg, ˜2400 μg-˜3000 μg, ˜2600 μg-˜3000 μg, or, e.g., ˜2800 μg-˜3000 μg.

In aspects, a short-acting bronchodilator component can be present in an amount of between ˜10 μg and ˜2800 μg, ˜50 μg-˜2600 μg, ˜100 μg-˜2400 μg, ˜200 μg-˜2200 μg, or, e.g., between ˜80 μg-˜400 μg, such as ˜90 μg-˜360 μg; ˜10 μg-˜100 μg, such as ˜17 μg-˜68 μg; ˜40 μg-˜210 μg, such as ˜45 μg-˜180 μg or ˜50 μg-˜200 μg; ˜150 μg-˜900 μg, such as ˜200 μg-˜800 μg; or ˜500 μg-˜2800 μg, such as ˜650 μg-˜2600 μg.

In certain aspects, a short-acting bronchodilator component can comprise, e.g., albuterol in an amount of ˜90 μg-˜360 μg; ipratropium bromide in an amount of ˜17 μg-˜68 μg; levalbuterol in an amount of ˜45 μg-˜180 μg; terbutaline in an amount of ˜50 μg-˜200 μg; pirbuterol in an amount of ˜200 μg-˜800 μg; metaproterenol in an amount of ˜650 μg-˜2600 μg; or any combination of any or all thereof.

### Long-Acting Bronchodilator Component

In aspects, a bronchodilating component can comprise a long-acting bronchodilator component. In aspects, a long-acting bronchodilator component can comprise one or more long-acting bronchodilating agent(s)/compound(s). In aspects, a long-acting bronchodilator component can comprise, e.g., one or more long-acting bronchodilator component subcomponents. In aspects, a long-acting bronchodilator component subcomponent can be, e.g., an anticholinergic component (anticholinergics also referred to as antimuscarinics, hence such a subcomponent can also be referred to as an antimuscarinic component). In aspects, a long-acting bronchodilator component subcomponent can be, e.g., a beta-adrenergic agonist component.

In aspects, a long-acting bronchodilating agent(s)/compound(s) can be any pharmaceutically acceptable bronchodilating agent/compound which provide(s) detectable or significant relief from constricted airways, e.g., measurable expansion (dilation) of bronchioles as recognized in the art, is bioequivalent to a similar, approved drug, or as demonstrated in a statistically significant population of people in an appropriately conducted and powered clinical trial recognized by an applicable regulatory authority/body such as the United States Food and Drug Administration (US FDA) for a period of at least about 5 hours, such as, e.g., ≥˜10 hours, ≥˜15 hours, ≥˜20 hours, ≥˜25 hours, ≥˜30 hours, ≥˜35 hours, ≥˜40 hours, ≥˜45 hours, or, e.g., ≥˜50 hours. In aspects, a long-acting bronchodilating agent/compound is a compound recognized in the art, or, e.g., is a compound having demonstrated in a statistically significant population of people in an appropriately conducted and powered clinical trial recognized by an applicable regulatory authority/body such as the US FDA, as providing detectably or significantly longer term relief from respiratory distress compared to that of short-acting bronchodilating agent(s)/compound(s) (described elsewhere herein).

### Long-Acting Bronchodilator Anticholinergic Component

According to aspects, composition(s) provided by the invention can comprise an anticholinergic component comprising one or more anticholinergic (antimuscarinic) constituent(s)/compound(s). In aspects, such an anticholinergic compound can be any pharmaceutically acceptable anticholinergic compound capable of detectably or significantly blocking and inhibiting the activity of acetylcholine (ACh) at central and peripheral nervous system synapse(s).

In aspects, exemplary anticholinergic component constituent(s)/compound(s) can comprise, e.g., aclidinium, glycopyrronium (also referred to as glycopyrrolate), synthetic quaternary ammonium congeners of atropine, e.g., ipratropium (such as ipratropium bromide), tiotropium (such as tiotropium bromide), oxitropium (e.g., oxitropium bromide), umeclindium, revefenacin, etc. In certain aspects, the anticholinergic compound can be, e.g., a tiotropium compound, an ipratropium compound, or a combination thereof. In aspects, the anticholinergic compound is a tiotropium compound. In aspects, the anticholinergic compound is an ipratropium compound. In aspects, the anticholinergic component comprises one or more compound(s) approved by a recognized regulatory authority/body, such as, e.g., a governmental agency such as, e.g., the United States Food and Drug Administration (US FDA) for use in the treatment of asthma, chronic obstructive pulmonary disease (COPD), or both.

In aspects, composition(s) herein are provided in the form of a solution and, accordingly, the anticholinergic component constituents, e.g., tiotropium compound(s), ipratropium compound(s), or combinations thereof, are provided in solution, e.g., are solubilized. In certain aspects, composition(s) provided herein comprise a bronchodilating component comprising, e.g., an anticholinergic sub-component in addition to one or more additional sub-components, e.g., a non-anticholinergic sub-component, such as, e.g., a beta-adrenergic agonist component. Accordingly, the present invention provides, in aspects, a pharmaceutical formulation comprising anticholinergic agent(s) such as, e.g., a tiotropium compound, an ipratropium compound, or both, which may further comprise one or more APIs selected from beta-adrenergic agonists. In aspects, one or more such compounds are administered simultaneously, sequentially, or are provided for separate use.

### Tiotropium

In aspects, composition(s) herein comprise an anticholinergic component comprising a tiotropium compound, e.g., a pharmaceutically acceptable salt of tiotropium, a pharmaceutically acceptable solvate of tiotropium, or a combination thereof. In aspects, the tiotropium compound is a pharmaceutically acceptable salt of tiotropium, such as, e.g., tiotropium bromide. In aspects, composition(s) comprise tiotropium bromide, wherein the tiotropium bromide compound is tiotropium bromide monohydrate. In aspects, the tiotropium bromide monohydrate is provided in crystalline form, as, e.g., described in US 2003/171586. In some aspects, composition(s) comprise tiotropium bromide present as anhydrous tiotropium bromide. Typically, the tiotropium compound is a cationic compound.

### Ipratropium

In certain aspects, composition(s) comprise an ipratropium compound as an alternative to tiotropium, either alone or in combination with tiotropium compound(s). In aspects, the ipratropium compound is a pharmaceutically acceptable salt of ipratropium. In aspects, the pharmaceutically acceptable salt of ipratropium is ipratropium bromide.

### Amounts of Long-Acting Bronchodilator Anticholinergic Component(s)

In certain aspects, composition(s) comprise a single API component, e.g., comprise only a bronchodilating component, e.g., only a long-acting bronchodilator component, such as, e.g., only a long-acting bronchodilator anticholinergic component. In aspects, the long-acting bronchodilator anticholinergic component comprises only an anticholinergic component. In aspects, the long-acting bronchodilator anticholinergic component comprises a single anticholinergic agent. In aspects, the long-acting bronchodilator anticholinergic component comprises two or more anticholinergic agents. In aspects, therefore, amounts described within this section can refer to: (a) the entirety of the API concentration within a composition when the API component of a composition comprises only a bronchodilator component, and the bronchodilator component comprises only a long-acting bronchodilator component comprising only an anticholinergic component; (b) the entirety of any bronchodilating component concentration of a composition when the bronchodilating component consists only of a long-acting bronchodilating component comprising only an anticholinergic component; (c) the entirety of any long-acting bronchodilator component concentration of a composition when a composition comprises a long-acting bronchodilator component comprising only an anticholinergic component; (d) the entirety of an anticholinergic component concentration of a composition when the composition comprises an anticholinergic component comprising only long-acting bronchodilator anticholinergic agents; (e) the specific concentration of any single long-acting bronchodilator anticholinergic agent/compound in the composition; (f) the specific concentration of tiotropium compound(s) within the composition; (g) the specific concentration of ipratropium compound(s) within the composition; (h) the concentration of tiotropium and ipratropium compound(s) together in the composition; or (i) any combination of (a)-(h).

### Long-Acting Bronchodilator Anticholinergic Component Concentrations

In aspects, any therapeutically effective amount of an anticholinergic component can be used. In aspects, the concentration of an anticholinergic component in composition(s) when expressed as a mg/mL concentration can be, e.g., between about 0.01 mg/mL and about 0.5 mg/mL, such as, e.g., ˜0.01 mg/mL-˜0.45 mg/mL, ˜0.01 mg/mL-˜0.4 mg/mL, ˜0.01 mg/mL-˜0.35 mg/mL, ˜0.01 mg/mL-˜0.3 mg/mL, ˜0.01 mg/mL-˜0.25 mg/mL, ˜0.01 mg/mL-˜0.2 mg/mL, ˜0.01 mg/mL-˜0.15 mg/mL, or, e.g., ˜0.01 mg/mL-˜0.1 mg/mL anticholinergic component, e.g., a tiotropium compound, an ipratropium compound, or, e.g., a combination of a tiotropium compound and an ipratropium compound.

In aspects, the concentration of an anticholinergic component in composition(s) when expressed as a mg/mL concentration can be, e.g., between about 0.015 mg/mL and about 0.5 mg/mL, such as, e.g., ˜0.02 mg/mL-˜0.5 mg/mL, ˜0.025 mg/mL-˜0.5 mg/mL, ˜0.03 mg/mL-˜0.5 mg/mL, ˜0.035 mg/mL-˜0.5 mg/mL, ˜0.04 mg/mL-˜0.5 mg/mL, ˜0.045 mg/mL-˜0.5 mg/mL, or, e.g., ˜0.05 mg/mL-˜0.5 mg/mL anticholinergic component, e.g., a tiotropium compound, an ipratropium compound, or, e.g., a combination of a tiotropium compound and an ipratropium compound.

In aspects, the concentration of an anticholinergic component in composition(s) when expressed as a mg/mL concentration can be, e.g., between about 0.015 mg/mL and about 0.45 mg/mL, such as, e.g., ˜0.02 mg/mL-˜0.4 mg/mL, ˜0.025 mg/mL-˜0.35 mg/mL, ˜0.03 mg/mL-˜0.3 mg/mL, ˜0.035 mg/mL-˜0.25 mg/mL, ˜0.04 mg/mL-˜0.2 mg/mL, ˜0.045 mg/mL-˜0.15 mg/L, or, e.g., ˜0.05 mg/mL-˜0.1 mg/mL, such as, e.g., ˜0.06 mg/mL, ˜0.07 mg/mL, ˜0.08 mg/mL, ˜0.09 mg/mL, or, e.g., ˜0.1 mg/mL anticholinergic component, e.g., a tiotropium compound, an ipratropium compound, or, e.g., a combination of a tiotropium compound and an ipratropium compound.

In aspects, the concentration of an anticholinergic component in composition(s), e.g., a tiotropium compound, an ipratropium compound, or, e.g., a combination of tiotropium and ipratropium compound(s), is, e.g., not less than an effective amount of such compound(s), e.g., is not less than an amount capable of demonstrating a detectably or significantly therapeutically beneficial effect. In aspects, the concentration of an anticholinergic component in composition(s) when expressed as a mg/mL concentration can be, e.g., no less than about 0.05 mg/mL, ≥˜0.055 mg/mL, ≥˜0.06 mg/mL, ≥˜0.065 mg/mL, ≥˜0.07 mg/mL, ≥˜0.075 mg/mL, ≥˜0.08 mg/mL, ≥˜0.085 mg/mL, ≥˜0.09 mg/mL, ≥˜0.095 mg/mL, or, e.g., ≥˜0.1 mg/mL, anticholinergic component, e.g., a tiotropium compound, an ipratropium compound, or, e.g., a combination of a tiotropium compound and an ipratropium compound.

In aspects, the concentration of an anticholinergic component in composition(s), e.g., a tiotropium compound, an ipratropium compound, or, e.g., a combination of tiotropium and ipratropium compound(s), is, e.g., not more than an amount typically suitable, safe, efficacious, or otherwise necessary of demonstrating a sufficiently beneficial therapeutic effect. In aspects, the concentration of an anticholinergic component in composition(s) when expressed as a mg/mL concentration can be, e.g., no more than about 0.1 mg/mL, such as, e.g., ≤˜0.09 mg/mL, ≤˜0.085 mg/mL, ≤˜0.08 mg/mL, ≤˜0.075 mg/mL, ≤˜0.07 mg/mL, ≤˜0.065 mg/mL, ≤˜0.06 mg/mL, ≤˜0.055 mg/mL, or, e.g., ≤˜0.05 mg/mL anticholinergic component, e.g., a tiotropium compound, an ipratropium compound, or, e.g., a combination of a tiotropium compound and an ipratropium compound.

In aspects, the concentration of an anticholinergic component in composition(s) when expressed as a mg/mL concentration can be, e.g., between about 0.05 mg/mL and about 5 mg/mL, such as, e.g., ˜0.05 mg/L-˜4.5 mg/mL, ˜0.05 mg/L-˜4 mg/mL, ˜0.05 mg/L-˜3.5 mg/mL, ˜0.05 mg/L-˜3 mg/mL, ˜0.05 mg/L-˜2.5 mg/mL, ˜0.05 mg/L-˜2 mg/mL, ˜0.05 mg/L-˜1.5 mg/mL, or, e.g., ˜0.05 mg/L-˜1 mg/mL anticholinergic component, e.g., a tiotropium compound, an ipratropium compound, or, e.g., a combination of a tiotropium compound and an ipratropium compound.

In aspects, the concentration of an anticholinergic component in composition(s) when expressed as a mg/mL concentration can be, e.g., between about 0.055 mg/mL and about 5 mg/mL, such as, e.g., ˜0.06 mg/L-˜5 mg/mL, ˜0.065 mg/L-˜5 mg/mL, ˜0.07 mg/L-˜5 mg/mL, ˜0.075 mg/L-˜5 mg/mL, ˜0.08 mg/L-˜5 mg/mL, ˜0.085 mg/L-˜5 mg/mL, ˜0.09 mg/L-˜5 mg/mL, ˜0.095 mg/L-˜5 mg/mL, or, e.g., ˜0.01 mg/mL-˜5 mg/mL anticholinergic component, e.g., a tiotropium compound, an ipratropium compound, or, e.g., a combination of a tiotropium compound and an ipratropium compound.

In aspects, the concentration of an anticholinergic component in composition(s) when expressed as a mg/mL concentration can be, e.g., between about 0.055 mg/mL and about 4.5 mg/mL, such as ˜0.06 mg/mL-˜4 mg/mL, ˜0.065 mg/mL-˜3.5 mg/mL, ˜0.07 mg/mL-˜3 mg/mL, ˜0.075 mg/mL-˜2.5 mg/mL, ˜0.08 mg/mL-˜2 mg/mL, ˜0.085 mg/mL-˜1.5 mg/mL, ˜0.09 mg/mL-˜1.5 mg/mL, or, e.g., ˜0.1 mg/mL-˜1 mg/mL, such as, e.g., ˜0.2 mg/mL, ˜0.3 mg/mL, ˜0.4 mg/mL, ˜0.5 mg/mL, ˜0.6 mg/mL, ˜0.7 mg/mL, ˜0.8 mg/mL, ˜0.9 mg/mL, or, e.g., ˜1 mg/mL anticholinergic component, e.g., a tiotropium compound, an ipratropium compound, or, e.g., a combination of a tiotropium compound and an ipratropium compound.

In aspects, the concentration of an anticholinergic component in composition(s) when expressed as a mg/mL concentration can be, e.g., no less than about 0.1 mg/mL, ≥˜0.15 mg/mL, ≥˜0.2 mg/mL, ≥˜0.25 mg/mL, ≥˜0.3 mg/mL, ≥˜0.35 mg/mL, ≥˜0.4 mg/mL, ≥˜0.45 mg/mL, ≥˜0.5 mg/mL, ≥˜0.55 mg/mL, ≥˜0.6 mg/mL, ≥˜0.65 mg/mL, ≥˜0.7 mg/mL, ≥˜0.75 mg/mL, ≥˜0.8 mg/mL, ≥˜0.85 mg/mL, ≥˜0.9 mg/mL, ≥˜0.95 mg/mL, or, e.g., ≥˜1 mg/mL anticholinergic component, e.g., a tiotropium component, an ipratropium compound, or, e.g., a combination of a tiotropium compound and an ipratropium compound.

In aspects, the concentration of an anticholinergic component in composition(s) when expressed as a mg/mL concentration can be, e.g., no more than about 1 mg/mL, such as, e.g., ≥˜0.95 mg/mL, ≤˜0.9 mg/mL, ≥˜0.85 mg/mL, ≤˜0.8 mg/mL, ≥˜0.75 mg/mL, ≤˜0.7 mg/mL, ≥˜0.65 mg/mL, ≤˜0.6 mg/mL, ≥˜0.55 mg/mL, ≤˜0.5 mg/mL, ≥˜0.45 mg/mL, ≤˜0.4 mg/mL, ≥˜0.35 mg/mL, ≤˜0.3 mg/mL, ≥˜0.25 mg/mL, ≤˜0.2 mg/mL, ≥˜0.15 mg/mL, or, e.g., ≤˜0.1 mg/mL anticholinergic component, e.g., a tiotropium component, an ipratropium compound, or, e.g., a combination of a tiotropium compound and an ipratropium compound.

In certain aspects, the concentration of an anticholinergic component in composition(s) provided as a solution when expressed as a mg/mL concentration can be, e.g., between about 0.01 mg/mL and about 0.5 mg/mL, such as, e.g., between about 0.05 mg/mL and about 0.1 mg/mL anticholinergic component, e.g., a tiotropium compound, an ipratropium compound, or, e.g., a combination of a tiotropium compound and an ipratropium compound.

In certain aspects, the concentration of an anticholinergic component in composition(s) provided as a suspension when expressed as a mg/mL concentration can be, e.g., between about 0.05 mg/mL and about 5 mg/mL, such as, e.g., between about 0.1 mg/mL and about 1 mg/mL anticholinergic component, e.g., a tiotropium compound, an ipratropium compound, or, e.g., a combination of a tiotropium compound and an ipratropium compound.

### Long-Acting Bronchodilator Anticholinergic Component Weight Percent Concentration

In aspects, any effective amount of an anticholinergic component can be used. In aspects, the concentration of an anticholinergic component in composition(s) when expressed as a weight percent (wt. %) concentration can be, e.g., between about 0.001 wt. % and about 0.05 wt. %, such as, e.g., ˜0.001 wt. %-˜0.045 wt. %, ˜0.001 wt. %-˜0.04 wt. %, ˜0.001 wt. %-˜0.035 wt. %, ˜0.001 wt. %-˜0.03 wt. %, ˜0.001 wt. %-˜0.025 wt. %, ˜0.001 wt. %-˜0.02 wt. %, ˜0.001 wt. %-˜0.015 wt. %, or, e.g., ˜0.001 wt. %-˜0.01 wt. % anticholinergic component, e.g., a tiotropium compound, an ipratropium compound, or, e.g., a combination of a tiotropium compound and an ipratropium compound.

In aspects, the concentration of an anticholinergic component in composition(s) when expressed as a wt. % concentration can be, e.g., between about 0.0015 wt. % and about 0.05 wt. %, such as, e.g., ˜0.002 wt. %-˜0.05 wt. %, ˜0.0025 wt. %-˜0.05 wt. %, ˜0.003 wt. %-˜0.05 wt. %, ˜0.0035 wt. %-˜0.05 wt. %, ˜0.004 wt. %-˜0.05 wt. %, ˜0.0045 wt. %-˜0.05 wt. %, or, e.g., ˜0.005 wt. %-˜0.05 wt. % anticholinergic component, e.g., a tiotropium compound, an ipratropium compound, or, e.g., a combination of a tiotropium compound and an ipratropium compound.

In aspects, the concentration of an anticholinergic component in composition(s) when expressed as a wt. % concentration can be, e.g., between about 0.0015 wt. % and about 0.045 wt. %, such as, e.g., ˜0.002 wt. %-˜0.04 wt. %, ˜0.0025 wt. %-˜0.035 wt. %, ˜0.003 wt. %-˜0.03 wt. %, ˜0.0035 wt. %-˜0.025 wt. %, ˜0.004 wt. %-˜0.02 wt. %, ˜0.0045 wt. %-˜0.015 wt. %, or, e.g., ˜0.005 wt. %-˜0.01 wt. %, such as, e.g., ˜0.006 wt. %, ˜0.007 wt. %, ˜0.008 wt. %, ˜0.009 wt. %, or, e.g., ˜0.01 wt. % anticholinergic component, e.g., a tiotropium compound, an ipratropium compound, or, e.g., a combination of a tiotropium compound and an ipratropium compound.

In aspects, the concentration of an anticholinergic component in composition(s), e.g., a tiotropium compound, an ipratropium compound, or, e.g., a combination of tiotropium and ipratropium compound(s), is, e.g., not less than an effective amount of such compound(s), e.g., is not less than an amount capable of demonstrating a detectably or significantly therapeutically beneficial effect. In aspects, the concentration of an anticholinergic component in composition(s) when expressed as a wt. % concentration can be, e.g., no less than (e.g., greater than or equal to) about 0.005 wt. %, ≥˜0.0055 wt. %, ≥˜0.006 wt. %, ≥˜0.0065 wt. %, ≥˜0.007 wt. %, ≥˜0.0075 wt. %, ≥˜0.008 wt. %, ≥˜0.0085 wt. %, ≥˜0.009 wt. %, ≥˜0.0095 wt. %, or, e.g., ≥˜0.01 wt. %, anticholinergic component, e.g., a tiotropium component, an ipratropium compound, or, e.g., a combination of a tiotropium compound and an ipratropium compound.

In aspects, the concentration of an anticholinergic component in composition(s), e.g., a tiotropium compound, an ipratropium compound, or, e.g., a combination of tiotropium and ipratropium compound(s), is, e.g., not more than an amount typically suitable, safe, efficacious, or otherwise necessary of demonstrating a sufficiently beneficial therapeutic effect. In aspects, the concentration of an anticholinergic component in composition(s) when expressed as a wt. % concentration can be, e.g., no more than about 0.01 wt. %, such as, e.g., ≤˜0.009 wt. %, ≤˜0.0085 wt. %, ≤˜0.008 wt. %, ≤˜0.0075 wt. %, ≤˜0.007 wt. %, ≤˜0.0065 wt. %, ≤˜0.006 wt. %, ≤˜0.0055 wt. %, or, e.g., ≤˜0.005 wt. % anticholinergic component, e.g., a tiotropium component, an ipratropium compound, or, e.g., a combination of a tiotropium compound and an ipratropium compound.

In aspects, the concentration of an anticholinergic component in composition(s) when expressed as a wt. % concentration can be, e.g., between about 0.005 wt. % and about 0.5 wt. %, such as, e.g., ˜0.005 wt. %-˜0.45 wt. %, ˜0.005 wt. %-˜0.4 wt. %, ˜0.005 wt. %-˜0.35 wt. %, ˜0.005 wt. %-˜0.3 wt. %, ˜0.005 mg/L-˜0.25 wt. %, ˜0.005 wt. %-˜0.2 wt. %, ˜0.005 wt. %-˜0.15 wt. %, or, e.g., ˜0.005 wt. %-˜0.1 wt. % anticholinergic component, e.g., a tiotropium compound, an ipratropium compound, or, e.g., a combination of a tiotropium compound and an ipratropium compound.

In aspects, the concentration of an anticholinergic component in composition(s) when expressed as a wt. % concentration can be, e.g., between about 0.0055 wt. % and about 0.5 wt. %, such as, e.g., ˜0.006 wt. %-˜0.5 wt. %, ˜0.0065 wt. %-˜0.5 wt. %, ˜0.007 wt. %-˜0.5 wt. %, ˜0.0075 wt. %-˜0.5 wt. %, ˜0.008 wt. %-˜0.5 wt. %, ˜0.0085 wt. %-˜0.5 wt. %, ˜0.009 wt. %-˜0.5 wt. %, ˜0.0095 wt. %-˜0.5 wt. %, or, e.g., ˜0.001 wt. %-˜0.5 wt. % anticholinergic component, e.g., a tiotropium compound, an ipratropium compound, or, e.g., a combination of a tiotropium compound and an ipratropium compound.

In aspects, the concentration of an anticholinergic component in composition(s) when expressed as a wt. % concentration can be, e.g., between about 0.0055 wt. % and about 0.45 wt. %, such as ˜0.006 wt. %-˜0.4 wt. %, ˜0.0065 wt. %-˜0.35 wt. %, ˜0.007 wt. %-˜0.3 wt. %, ˜0.0075 wt. %-˜0.25 wt. %, ˜0.008 wt. %-˜0.2 wt. %, ˜0.0085 wt. %-˜0.15 wt. %, ˜0.009 wt. %-˜0.15 wt. %, or, e.g., ˜0.01 wt. %-˜0.1 wt. %, such as, e.g., ˜0.02 wt. %, ˜0.03 wt. %, ˜0.04 wt. %, ˜0.05 wt. %, ˜0.06 wt. %, ˜0.07 wt. %, ˜0.08 wt. %, ˜0.09 wt. %, or, e.g., ˜0.1 wt. % anticholinergic component, e.g., a tiotropium compound, an ipratropium compound, or, e.g., a combination of a tiotropium compound and an ipratropium compound.

In aspects, the concentration of an anticholinergic component in composition(s) when expressed as a wt. % concentration can be, e.g., no less than about 0.01 wt. %, ≥˜0.015 wt. %, ≥˜0.02 wt. %, ≥˜0.025 wt. %, ≥˜0.03 wt. %, ≥˜0.035 wt. %, ≥˜0.04 wt. %, ≥˜0.045 wt. %, ≥˜0.05 wt. %, ≥˜0.055 wt. %, ≥˜0.06 wt. %, ≥˜0.065 wt. %, ≥˜0.07 wt. %, ≥˜0.075 wt. %, ≥˜0.08 wt. %, ≥˜0.085 wt. %, ≥˜0.09 wt. %, ≥˜0.095 wt. %, or, e.g., ≥˜0.1 wt. % anticholinergic component, e.g., a tiotropium component, an ipratropium compound, or, e.g., a combination of a tiotropium compound and an ipratropium compound.

In aspects, the concentration of an anticholinergic component in composition(s) when expressed as a wt. % concentration can be, e.g., no more than about 0.1 wt. %, such as, e.g., ≥˜0.095 wt. %, ≤˜0.09 wt. %, ≥˜0.085 wt. %, ≤˜0.08 wt. %, ≥˜0.075 wt. %, ≤˜0.07 wt. %, ≥˜0.065 wt. %, ≤˜0.06 wt. %, ≥˜0.055 wt. %, ≤˜0.05 wt. %, ≥˜0.045 wt. %, ≤˜0.04 wt. %, ≥˜0.035 wt. %, ≤˜0.03 wt. %, ≥˜0.025 wt. %, ≤˜0.02 wt. %, ≥˜0.015 wt. %, or, e.g., ≤˜0.01 wt. % anticholinergic component, e.g., a tiotropium component, an ipratropium compound, or, e.g., a combination of a tiotropium compound and an ipratropium compound.

In certain aspects, the concentration of an anticholinergic component in composition(s) provided as a solution when expressed as a wt. % concentration can be, e.g., between about 0.001 wt. % and about 0.05 wt. %, such as, e.g., between about 0.005 wt. % and about 0.01 wt. % anticholinergic component, e.g., a tiotropium compound, an ipratropium compound, or, e.g., a combination of a tiotropium compound and an ipratropium compound.

In certain aspects, the concentration of an anticholinergic component in composition(s) provided as a suspension when expressed as a wt. % concentration can be, e.g., between about 0.005 wt. % and about 0.5 wt. %, such as, e.g., between about 0.01 wt. % and about 0.1 wt. % anticholinergic component, e.g., a tiotropium compound, an ipratropium compound, or, e.g., a combination of a tiotropium compound and an ipratropium compound.

### Principles Related to Amount/Concentration Conversions Among Compounds

Readers will recognize that disclosures of amounts of anticholinergic component concentrations herein, e.g., the concentration of tiotropium compound(s), amounts also can be representative of or reflect typical corresponding/related concentration of other compound(s), e.g., tiotropium, e.g., in their/its pure form. For example, amounts of anticholinergic component concentrations can represent the concentration of tiotropium or vice versa in this disclosure, wherever suitable. In alternative aspects, provided values including ranges can be adjusted as necessary to address alternative forms of such constituents. For example, concentrations provided herein can be representative of, e.g., compound(s) in salt form. In aspects, anticholinergic component concentrations provided herein represent the concentration of tiotropium compound(s) in composition(s). In aspects, when tiotropium is present in composition(s) in the form of salts or solvates of tiotropium, readers will recognize that corresponding amounts of related compounds are also implicitly disclosed based on knowledge in the art, e.g., tiotropium bromide, 0.1 mg/mL of tiotropium corresponds to 0.1204 mg/mL tiotropium bromide. If a different tiotropium salt is used, a person of ordinary skill in the art is able to calculate the concentration of the particular tiotropium salt used in terms of tiotropium or tiotropium bromide using the ratio of the molar mass of the tiotropium salt being used to the molar mass of tiotropium or tiotropium bromide as applicable. Approximate values also can be used where suitable to cover a number of different types of related compounds in aspects. Readers and those skilled in the art can apply such conversion factors. E.g., multiplying provided values provided above by 1.204 to convert such values specifically to that which may further encapsulate use of tiotropium bromide can, in aspects, expand upon cited values and ranges so as to establish values incorporating alternative forms of compound(s) provided herein as applicable. Exemplary such conversions are provided here but should not be interpreted as limiting; that is, similar such conversions should be applied elsewhere herein or to other compound(s) or form(s) of compound(s) even where not specifically exemplified as would be understood by readers.

### Specific Examples of Tiotropium Bromide Concentrations

For example, in aspects, composition(s) provided by the invention can comprise a tiotropium compound, e.g. tiotropium bromide, wherein the concentration of tiotropium, expressed in terms of tiotropium bromide, can be, e.g., no less than about 0.01 mg/mL, ≥˜0.02 mg/mL, ≥˜0.03 mg/mL, ≥˜0.04 mg/mL, ≥˜0.05 mg/mL, ≥˜0.06 mg/mL, ≥˜0.07 mg/mL, ≥˜0.08 mg/mL, ≥˜0.09, ≥˜0.1 mg/mL, ≥˜0.2 mg/mL, ≥˜0.3 mg/mL, ≥˜0.4 mg/mL, ≥˜0.5 mg/mL, ≥˜0.6 mg/mL, ≥˜0.7 mg/mL, ≥˜0.8 mg/mL, ≥˜0.9 mg/mL, ≥˜1 mg/mL, ≥˜1.2 mg/mL, ≥˜1.4 mg/mL, ≥˜1.6 mg/mL, ≥˜1.8 mg/mL, ≥˜2 mg/mL, ≥˜2.2 mg/mL, ≥˜2.4 mg/mL, ≥˜2.6 mg/mL, ≥˜2.8 mg/mL, ≥˜3 mg/mL, ≥˜3.2 mg/mL, ≥˜3.4 mg/mL, ≥˜3.6 mg/mL, ≥˜3.8 mg/mL, ≥˜4 mg/mL, ≥˜4.2 mg/mL, ≥˜4.4 mg/mL, ≥˜4.6 mg/mL, ≥˜4.8 mg/mL, or, e.g., ≥˜5 mg/mL.

In another example, in aspects, composition(s) provided by the invention can comprise a tiotropium compound, e.g. tiotropium bromide, wherein the concentration of tiotropium, expressed in terms of tiotropium bromide, can be, e.g., no greater than about 5 mg/mL, ≤˜4.8 mg/mL, ≤˜4.6 mg/mL, ≤˜4.4 mg/mL, ≤˜4.2 mg/mL, ≤˜4 mg/mL, ≤˜3.8 mg/mL, ≤˜3.6 mg/ml, ≤˜3.4, ≤˜3.2 mg/mL, ≤˜3 mg/ml, ≤˜2.8 mg/mL, ≤˜2.6 mg/ML, ≤˜2.4 mg/mL, ≤˜2.2 mg/mL, ≤˜2 mg/mL, ≤˜1.8 mg/mL, ≤˜1.6 mg/mL, ≤˜1.4 mg/mL, ≤˜1.2 mg/mL, ≤˜1 mg/mL, ≤˜0.9 mg/mL, ≤˜0.8 mg/mL, ≤˜0.7 mg/mL, ≤˜0.6 mg/mL, ≤˜0.5 mg/mL, ≤˜0.4 mg/mL, ≤˜0.3 mg/mL, ≤˜0.2 mg/mL, ≤˜0.1 mg/mL, ≤˜0.09 mg/mL, ≤˜0.08 mg/mL, ≤˜0.07 mg/mL, ≤˜0.06 mg/mL, ≤˜0.05 mg/mL, ≤˜0.04 mg/mL, ≤˜0.03 mg/mL, ≤˜0.02 mg/mL, or, e.g., ≤˜0.01 mg/mL.

In other examples, in aspects, composition(s) comprise a tiotropium compound, e.g., a salt of tiotropium, e.g., tiotropium bromide, wherein the concentration of tiotropium, expressed in terms of tiotropium bromide, can be, e.g., about 0.08 mg/mL to about 0.12 mg/mL, or, e.g., about 0.09 mg/mL to about 0.11 mg/mL, e.g., about 0.1 mg/mL or, e.g., 0.1 mg/mL. In aspects, the concentration of tiotropium, expressed in terms of tiotropium bromide, can be, e.g., about 0.1 mg/mL to about 1.3 mg/mL, such as, e.g., about 0.3 mg/mL to about 1.2 mg/mL, e.g., about 0.5 mg/mL to about 1 mg/mL.

In still further examples, in aspects, composition(s) comprise a tiotropium compound, e.g., a salt of tiotropium, e.g., tiotropium bromide, wherein the concentration of tiotropium, expressed in terms of tiotropium bromide, can be, e.g., about 0.001 wt. % to about 0.1 wt. % based on the weight of tiotropium bromide. In aspects, the concentration of tiotropium, expressed in terms of tiotropium bromide can be, e.g., a concentration of about 0.005 wt. % to about 0.01 wt. % based on the weight of tiotropium bromide.

As stated above, in aspects, the amounts referenced here are amounts representing the concentration of a bronchodilator component in composition(s), wherein such a bronchodilator component comprises one or more subcomponents (e.g., comprises one class, group, or otherwise defined group of active(s) or more than one class, group, or otherwise defined type of active(s)), one or more agent(s)/compound(s), or any combination thereof. In aspects, the amounts referenced here are amounts representing the concentration of a long-acting bronchodilator component in composition(s), wherein such a long-acting bronchodilator component comprises one or more subcomponents (e.g., comprises one class, group, or otherwise defined type of actives or more than one class, group, or otherwise defined type of active(s)), one or more agent(s)/compound(s), or any combination thereof. In aspects, the amounts referenced here are amounts representing the concentration of an anticholinergic component in composition(s), wherein the anticholinergic component comprises one or more compounds. In aspects, the amounts referenced here are amounts representing the concentration of an anticholinergic agent in composition(s), wherein the anticholinergic agent is, e.g., a tiotropium compound or, e.g., an ipratropium compound.

### Long-Acting Bronchodilator Non-Anticholinergic Component(s)

According to certain aspects, composition(s) provided by the invention comprise a long-acting bronchodilator component comprising a non-anticholinergic component. According to aspects, composition(s) provided by the invention can comprise a long-acting bronchodilator non-anticholinergic component comprising one or more non-anticholinergic (non-antimuscarinic) constituent(s)/compound(s). In aspects, such a non-anticholinergic compound can be any pharmaceutically acceptable long-acting bronchodilator, non-anticholinergic compound not capable of detectably or significantly blocking and inhibiting the activity of acetylcholine (ACh) at central and peripheral nervous system synapse(s). In aspects, composition(s) do not comprise a non-anticholinergic component. In aspects, composition(s) comprise a long-acting bronchodilator anticholinergic component in addition to a long-acting bronchodilator non-anticholinergic component.

In aspects, the non-anticholinergic component or subcomponent thereof can comprise any one or more pharmaceutically acceptable compounds suitable for delivery by inhalation. In aspects, such a non-anticholinergic component can comprise, e.g., a beta-adrenergic agonist component, an inhaled corticosteroid component, or both. Other exemplary non-anticholinergic component constituents can include, e.g., a leukotriene receptor antagonist component, a leukotriene inhibitor component, a methylxanthine component, etc. Exemplary leukotriene receptor antagonists can include, e.g., monelukast and zafirlukast. Exemplary leukotriene inhibitors can include, e.g., zileuton. Exemplary methylxanthines can include, e.g., theophylline. In aspects, a non-anticholinergic component is provided for simultaneous administration with an anticholinergic component, e.g., both components are present within the same composition. In aspects, an anticholinergic component and a non-anticholinergic component are co-administered. In aspects, a non-anticholinergic component is provided for sequential administration with an anticholinergic component. In aspects, a non-anticholinergic component is provided for separate administration.

### Beta-Adrenergic Agonist Component(s)

In aspects, exemplary non-anticholinergic component constituent(s)/compound(s) can comprise, e.g., one or more long-acting bronchodilators, e.g., a long-acting beta-adrenergic agonist (LABA) such as a beta 2 agonist. In aspects, such a constituent/compound can be any pharmaceutically acceptable beta-adrenergic agonist. In aspects, the beta-adrenergic agonist is a compound suitable for delivery by inhalation. Exemplary beta-adrenergic agonists include, e.g., salbutamol, levoalbuterol, levosalbutamol butaline, pirbuterol, procaterol, metaproterenol, fenoterol, isoproterenol (beta1 and beta2), metaproterenol, terbutaline, isoetarine, bitolterol mesylate, ritodrine, salmeterol, formoterol, arformoterol, carmoterol, bambuterol, clenbuterol, indacaterol, milveterol, vilanterol, olodaterol, etc. In certain aspects, composition(s) herein comprise at least one long-acting beta 2 agonist selected from a group comprising formoterol fumarate, formoterol fumarate dihydrate, salmeterol xinafoate, and olodaterol. Uncontradicted, the phrase “selected from a group” is used for convenience only and is not meant to imply that there is any type of equivalence between members of the “group” of elements associated with the usage of the phrase and any such group can be divided into smaller groups or elements of the group as suitable in corresponding/related aspects.

In certain aspects, the non-anticholinergic compound, e.g., beta-adrenergic agonist, is a beta 2 adrenergic agonist, e.g., a formoterol compound.

In aspects, composition(s) herein are provided in the form of a solution and, accordingly, the non-anticholinergic component constituents, e.g., the beta-adrenergic agonist component, e.g., beta 2 agonist(s), e.g., formoterol compound(s) are provided in solution, e.g., are solubilized. In certain aspects, composition(s) provided herein comprise a bronchodilating component comprising, e.g., a non-anticholinergic sub-component in addition to one or more additional sub-components, e.g., an anticholinergic sub-component, such as, e.g., a component comprising a tiotropium compound, an ipratropium compound or a component comprising both compounds. Accordingly, the present invention provides, in aspects, a pharmaceutical formulation comprising anticholinergic agent(s) such as, e.g., a tiotropium compound, an ipratropium compound, or both, which may further comprise one or more APIs selected from non-anticholinergic agent(s), such as, e.g., beta-adrenergic agonist(s), e.g., beta 2 agonist(s). In aspects, one or more such compounds are administered simultaneously, sequentially, or are provided for separate use.

### Formoterol

In aspects, composition(s) provided by the invention comprise a bronchodilating component further comprising a long-acting bronchodilating component which further comprises a non-anticholinergic component comprising a formoterol compound, e.g., formoterol or a pharmaceutically acceptable salt or solvate thereof. In aspects, the salt or solvate of formoterol can be any pharmaceutically acceptable salt or solvate thereof suitable for delivery by inhalation. In aspects, comprise a salt of formoterol, such as, e.g., formoterol fumarate. In aspects, the formoterol fumarate compound is provided as formoterol fumarate dihydrate.

### Long-Acting Bronchodilator Non-Anticholinergic Component (e.g., Beta-Adrenergic Component) Amounts

In certain aspects, composition(s) comprise a single API component, e.g., comprising only a bronchodilating component, e.g., comprising only a long-acting bronchodilating component, e.g., comprising only a non-anticholinergic component. In aspects, the non-anticholinergic subcomponent of a long-acting bronchodilating component comprises a single non-anticholinergic agent. In aspects, the non-anticholinergic subcomponent of a long-acting bronchodilating component comprises two or more non-anticholinergic agents. In aspects, therefore, amounts described within this section can refer to: (a) the entirety of the API concentration within a composition when the API component of a composition comprises only a bronchodilating component consisting only of a long-acting bronchodilating component comprising only a non-anticholinergic component; (b) the entirety of any bronchodilating component concentration of a composition when the bronchodilating component consists only of a long-acting bronchodilating component comprising only a non-anticholinergic component; (c) the entirety of any long-acting bronchodilator component concentration of a composition when a composition comprises a long-acting bronchodilator component consisting only of a non-anticholinergic component; (d) the entirety of a non-anticholinergic component concentration of a composition; (e) the specific concentration of any single non-anticholinergic agent/compound in the composition; (f) the specific concentration of formoterol compound(s) within the composition; or (g) any combination of (a)-(f).

Long-Acting Bronchodilating Component Non-Anticholinergic Component—mg/mL

In aspects, any effective amount of a non-anticholinergic component can be used. In aspects, the concentration of a non-anticholinergic component in composition(s) when expressed as a mg/mL concentration can be, e.g., between about 0.01 mg/mL and about 0.5 mg/mL, such as, e.g., ˜0.01 mg/mL-˜0.45 mg/mL, ˜0.01 mg/mL-˜0.4 mg/mL, ˜0.01 mg/mL-˜0.35 mg/mL, ˜0.01 mg/mL-˜0.3 mg/mL, ˜0.01 mg/mL-˜0.25 mg/mL, ˜0.01 mg/mL-˜0.2 mg/mL, ˜0.01 mg/mL-˜0.15 mg/mL, or, e.g., ˜0.01 mg/mL-˜0.1 mg/mL non-anticholinergic component, e.g., a formoterol compound.

In aspects, the concentration of a non-anticholinergic component in composition(s) when expressed as a mg/mL concentration can be, e.g., between about 0.015 mg/mL and about 0.5 mg/mL, such as, e.g., ˜0.02 mg/mL-˜0.5 mg/mL, ˜0.025 mg/mL-˜0.5 mg/mL, ˜0.03 mg/mL-˜0.5 mg/mL, ˜0.035 mg/mL-˜0.5 mg/mL, ˜0.04 mg/mL-˜0.5 mg/mL, ˜0.045 mg/mL-˜0.5 mg/mL, or, e.g., ˜0.05 mg/mL-˜0.5 mg/mL non-anticholinergic component, e.g., a formoterol compound.

In aspects, the concentration of a non-anticholinergic component in composition(s) when expressed as a mg/mL concentration can be, e.g., between about 0.015 mg/mL and about 0.45 mg/mL, such as, e.g., ˜0.02 mg/mL-˜0.4 mg/mL, ˜0.025 mg/mL-˜0.35 mg/mL, ˜0.03 mg/mL-˜0.3 mg/mL, ˜0.035 mg/mL-˜0.25 mg/mL, ˜0.04 mg/mL-˜0.2 mg/mL, ˜0.045 mg/mL-˜0.15 mg/L, or, e.g., ˜0.05 mg/mL-˜0.1 mg/mL, such as, e.g., ˜0.06 mg/mL, ˜0.07 mg/mL, ˜0.08 mg/mL, ˜0.09 mg/mL, or, e.g., ˜0.1 mg/mL non-anticholinergic component, e.g., a formoterol compound.

In aspects, the concentration of a non-anticholinergic component in composition(s), e.g., a formoterol compound, is, e.g., not less than an effective amount of such compound(s), e.g., is not less than an amount capable of demonstrating a detectably or significantly therapeutically beneficial effect. In aspects, the concentration of a non-anticholinergic component in composition(s) when expressed as a mg/mL concentration can be, e.g., no less than about 0.05 mg/mL, ≥˜0.055 mg/mL, ≥˜0.06 mg/mL, ≥˜0.065 mg/mL, ≥˜0.07 mg/mL, ≥˜0.075 mg/mL, ≥˜0.08 mg/mL, ≥˜0.085 mg/mL, ≥˜0.09 mg/mL, ≥˜0.095 mg/mL, or, e.g., ≥˜0.1 mg/mL, non-anticholinergic component, e.g., a formoterol compound.

In aspects, the concentration of a non-anticholinergic component in composition(s), e.g., a formoterol compound, is, e.g., not more than an amount typically suitable, safe, efficacious, or otherwise necessary of demonstrating a sufficiently beneficial therapeutic effect. In aspects, the concentration of a non-anticholinergic component in composition(s) when expressed as a mg/mL concentration can be, e.g., no more than about 0.1 mg/mL, such as, e.g., ≤˜0.09 mg/mL, ≤˜0.085 mg/mL, ≤˜0.08 mg/mL, ≤˜0.075 mg/mL, ≤˜0.07 mg/mL, ≤˜0.065 mg/mL, ≤˜0.06 mg/mL, ≤˜0.055 mg/mL, or, e.g., ≤˜0.05 mg/mL non-anticholinergic component, e.g., a formoterol compound.

In certain aspects, the concentration of a non-anticholinergic component in composition(s) provided as a solution when expressed as a mg/mL concentration can be, e.g., between about 0.01 mg/mL and about 0.5 mg/mL, such as, e.g., between about 0.05 mg/mL and about 0.1 mg/mL non-anticholinergic component, e.g., a formoterol compound, such as, e.g., about 0.08 mg/mL formoterol compound, e.g., a salt of formoterol, e.g., formoterol fumarate, e.g., formoterol fumarate dihydrate.

In certain aspects, the concentration of a non-anticholinergic component in composition(s) provided as a suspension when expressed as a mg/mL concentration can be, e.g., between about 0.01 mg/mL non-anticholinergic component, e.g., a formoterol compound, such as, e.g., about 0.08 mg/mL formoterol compound, e.g., a salt of formoterol, e.g., formoterol fumarate, e.g., formoterol fumarate dihydrate.

### Long-Acting Bronchodilating Component Non-Anticholinergic Component—%

In aspects, any effective amount of a non-anticholinergic component can be used. In aspects, the concentration of a non-anticholinergic component in composition(s) when expressed as a weight percent (wt. %) concentration can be, e.g., between about 0.001 wt. % and about 0.05 wt. %, such as, e.g., ˜0.001 wt. %-˜0.045 wt. %, ˜0.001 wt. %-˜0.04 wt. %, ˜0.001 wt. %-˜0.035 wt. %, ˜0.001 wt. %-˜0.03 wt. %, ˜0.001 wt. %-˜0.025 wt. %, ˜0.001 wt. %-˜0.02 wt. %, ˜0.001 wt. %-˜0.015 wt. %, or, e.g., ˜0.001 wt. %-˜0.01 wt. % non-anticholinergic component, e.g., a formoterol compound.

In aspects, the concentration of a non-anticholinergic component in composition(s) when expressed as a wt. % concentration can be, e.g., between about 0.0015 wt. % and about 0.05 wt. %, such as, e.g., ˜0.002 wt. %-˜0.05 wt. %, ˜0.0025 wt. %-˜0.05 wt. %, ˜0.003 wt. %-˜0.05 wt. %, ˜0.0035 wt. %-˜0.05 wt. %, ˜0.004 wt. %-˜0.05 wt. %, ˜0.0045 wt. %-˜0.05 wt. %, or, e.g., ˜0.005 wt. %-˜0.05 wt. % non-anticholinergic component, e.g., a formoterol compound.

In aspects, the concentration of a non-anticholinergic component in composition(s) when expressed as a wt. % concentration can be, e.g., between about 0.0015 wt. % and about 0.045 wt. %, such as, e.g., ˜0.002 wt. %-˜0.04 wt. %, ˜0.0025 wt. %-˜0.035 wt. %, ˜0.003 wt. %-˜0.03 wt. %, ˜0.0035 wt. %-˜0.025 wt. %, ˜0.004 wt. %-˜0.02 wt. %, ˜0.0045 wt. %-˜0.015 wt. %, or, e.g., ˜0.005 wt. %-˜0.01 wt. %, such as, e.g., ˜0.006 wt. %, ˜0.007 wt. %, ˜0.008 wt. %, ˜0.009 wt. %, or, e.g., ˜0.01 wt. % non-anticholinergic component, e.g., a formoterol compound.

In aspects, the concentration of a non-anticholinergic component in composition(s), e.g., a formoterol compound, is, e.g., not less than an effective amount of such compound(s), e.g., is not less than an amount capable of demonstrating a detectably or significantly therapeutically beneficial effect. In aspects, the concentration of an anticholinergic component in composition(s) when expressed as a wt. % concentration can be, e.g., no less than about 0.005 wt. %, ≥˜0.0055 wt. %, ≥˜0.006 wt. %, ≥˜0.0065 wt. %, ≥˜0.007 wt. %, ≥˜0.0075 wt. %, ≥˜0.008 wt. %, ≥˜0.0085 wt. %, ≥˜0.009 wt. %, ≥˜0.0095 wt. %, or, e.g., ≥˜0.01 wt. %, non-anticholinergic component, e.g., a formoterol compound.

In aspects, the concentration of a non-anticholinergic component in composition(s), e.g., a formoterol compound, is, e.g., not more than an amount typically suitable, safe, efficacious, or otherwise necessary of demonstrating a sufficiently beneficial therapeutic effect. In aspects, the concentration of a non-anticholinergic component in composition(s) when expressed as a wt. % concentration can be, e.g., no more than about 0.01 wt. %, such as, e.g., ≤˜0.009 wt. %, ˜0.0085 wt. %, ≤˜0.008 wt. %, ≤˜0.0075 wt. %, ≤˜0.007 wt. %, ≤˜0.0065 wt. %, ≤˜0.006 wt. %, ≤˜0.0055 wt. %, or, e.g., ≤˜0.005 wt. % non-anticholinergic component, e.g., a formoterol compound.

In certain aspects, the concentration of a non-anticholinergic component in composition(s) provided as a solution when expressed as a wt. % concentration can be, e.g., between about 0.001 wt. % and about 0.05 wt. %, such as, e.g., between about 0.005 wt. % and about 0.01 wt. % non-anticholinergic component, e.g., a formoterol compound, e.g., a salt of formoterol, e.g., formoterol fumarate, e.g., formoterol fumarate dihydrate.

In certain aspects, the concentration of a non-anticholinergic component in composition(s) provided as a suspension when expressed as a wt. % concentration can be, e.g., between about 0.001 wt. % and about 0.05 wt. %, such as, e.g., between about 0.005 wt. % and about 0.01 wt. % non-anticholinergic component, e.g., a formoterol compound, e.g., a salt of formoterol, e.g., formoterol fumarate, e.g., formoterol fumarate dihydrate.

### Salts and Applicable Value Conversions—Formoterol Fumarate-Specific Examples

As is discussed elsewhere herein, in discussing amounts of non-anticholinergic component concentrations, e.g., the concentration of, for example, formoterol compound(s), amounts can be representative of compound(s), e.g., formoterol, in their/its pure form. For example, amounts of non-anticholinergic component concentrations can represent the concentration of formoterol. In alternative aspects, provided values including ranges can be adjusted as necessary to address alternative forms of such constituents. For example, concentrations provided herein can be representative of, e.g., compound(s) in salt form. In aspects, non-anticholinergic component concentrations provided herein represent the concentration of formoterol compound(s) in composition(s) based on the weight of a specific form of the compound, e.g., a salt of formoterol, e.g., formoterol fumarate, e.g., formoterol dihydrate. Examples of the same are provided here but should not be interpreted as limiting.

For example, in aspects, composition(s) provided by the invention can comprise a formoterol compound, e.g. formoterol fumarate, wherein the concentration of formoterol, expressed in terms of formoterol fumarate dihydrate, can be, e.g., no less than about 0.01 mg/mL, ≥˜0.02 mg/mL, ≥˜0.03 mg/mL, ≥˜0.04 mg/mL, ≥˜0.05 mg/mL, ≥˜0.06 mg/mL, ≥˜0.07 mg/mL, ≥˜0.08 mg/mL, ≥˜0.09, ≥˜0.1 mg/mL, ≥˜0.2 mg/mL, ≥˜0.3 mg/mL, ≥˜0.4 mg/mL, or, e.g., ≥˜0.5 mg/mL.

In another example, in aspects, composition(s) provided by the invention can comprise a formoterol compound, e.g. formoterol fumarate, wherein the concentration of formoterol, expressed in terms of formoterol fumarate dihydrate, can be, e.g., no greater than about 0.5 mg/mL, such as, e.g., ≤˜0.4 mg/mL, ≤˜0.3 mg/mL, ≤˜0.2 mg/mL, ≤˜0.1 mg/mL, ≤˜0.09 mg/mL, ≤˜0.08 mg/mL, ≤˜0.07 mg/mL, ≤˜0.06 mg/mL, ≤˜0.05 mg/mL, ≤˜0.04 mg/mL, ≤˜0.03 mg/mL, ≤˜0.02 mg/mL, or, e.g., ≤˜0.01 mg/mL.

In other examples, in aspects, composition(s) comprise a formoterol compound, e.g., a salt of formoterol, e.g., formoterol fumarate, e.g., formoterol fumarate dihydrate, wherein the concentration of formoterol, expressed in terms of formoterol fumarate dihydrate, can be, e.g., about 0.01 mg/mL to about 0.5 mg/mL, or, e.g., about 0.05 mg/mL to about 0.1 mg/mL.

In other examples, in aspects, composition(s) comprise a formoterol compound, e.g., a salt of formoterol, e.g., formoterol fumarate, e.g., formoterol fumarate dihydrate, wherein the concentration of formoterol, expressed in terms of formoterol fumarate dihydrate, can be, e.g., about 0.001 wt. % to about 0.05 wt. %, such as, e.g., about 0.005 wt. % to about 0.01 wt. % based on the weight of formoterol fumarate dihydrate.

### Long-Acting Bronchodilator Component (Amount)

In aspects, composition(s) comprise a bronchodilating component which comprises only a long-acting bronchodilator sub-component. In aspects, composition(s) lack a short-acting bronchodilating component. In aspects, amounts provided in this section can represent the amount of bronchodilating component in composition(s) when such composition(s) comprise a bronchodilating component which comprises only long-acting bronchodilating constituent(s)/compound(s)/agent(s).

In aspects, any effective amount of a long-acting bronchodilator component can be used. In aspects, the concentration of a long-acting bronchodilator component in composition(s) when expressed as a mg/mL concentration can be, e.g., between about 0.01 mg/mL and about 1 mg/mL, such as, e.g., ˜0.01 mg/mL-˜0.9 mg/mL, ˜0.01 mg/mL-˜0.8 mg/mL, ˜0.01 mg/mL-˜0.7 mg/mL, ˜0.01 mg/mL-˜0.6 mg/mL, ˜0.01 mg/mL-˜0.5 mg/mL, ˜0.01 mg/mL-˜0.4 mg/mL, ˜0.01 mg/mL-˜0.3 mg/mL, ˜0.01 mg/mL-˜0.2 mg/mL, ˜0.01 mg/mL-˜0.1 mg/mL, ˜0.01 mg/mL-˜0.08 mg/mL, or, e.g., ˜0.01 mg/mL-˜0.06 mg/mL long-acting bronchodilator component.

In aspects, the concentration of a long-acting bronchodilator component in composition(s) when expressed as a mg/mL concentration can be, e.g., between about 0.015 mg/mL and about 1 mg/mL, such as, e.g., ˜0.02 mg/mL-˜1 mg/mL, ˜0.025 mg/mL-˜0.1 mg/mL, ˜0.3 mg/mL-˜1 mg/mL, ˜0.35 mg/mL-˜1 mg/mL, ˜0.4 mg/mL-˜1 mg/mL, ˜0.45 mg/mL-˜1 mg/mL, ˜0.5 mg/mL-˜1 mg/mL, ˜0.55 mg/mL-˜1 mg/mL, ˜0.6 mg/mL-˜1 mg/mL, ˜0.65 mg/mL-˜1 mg/mL, ˜0.7 mg/mL-˜1 mg/mL, ˜0.75 mg/mL-˜1 mg/mL, or, e.g., ˜0.8 mg/mL-˜1 mg/mL long-acting bronchodilator component.

In aspects, the concentration of a long-acting bronchodilator component in composition(s) when expressed as a mg/mL concentration can be, e.g., between about 0.02 mg/mL and about 0.9 mg/mL, such as, e.g., ˜0.03 mg/mL-˜0.8 mg/mL, ˜0.04 mg/mL-˜0.7 mg/mL, ˜0.05 mg/mL-˜0.6 mg/mL, ˜0.06 mg/mL-˜0.5 mg/mL, ˜0.07 mg/mL-˜0.4 mg/mL, ˜0.08 mg/mL-˜0.3 mg/mL, or, e.g., ˜0.09 mg/mL-˜0.2 mg/mL or ˜0.1 mg/mL-˜0.2 mg/mL long-acting bronchodilator component.

In aspects, the concentration of a long-acting bronchodilator component in composition(s) is, e.g., not less than an effective amount of such compound(s), e.g., is not less than an amount capable of demonstrating a detectably or significantly therapeutically beneficial effect. In aspects, the concentration of a long-acting bronchodilator component in composition(s) when expressed as a mg/mL concentration can be, e.g., no less than about 0.01 mg/mL, ≥˜0.02 mg/mL, ≥˜0.04 mg/mL, ≥˜0.06 mg/mL, ≥˜0.08 mg/mL, 0.1 mg/mL, ≥˜0.2 mg/mL, ≥˜0.3 mg/mL, ≥˜0.4 mg/mL, ≥˜0.5 mg/mL, ≥˜0.6 mg/mL, ≥˜0.7 mg/mL, ≥˜0.8 mg/mL, ≥˜0.9 mg/mL, or, e.g., ≥˜1 mg/mL long-acting bronchodilator component.

In aspects, the concentration of a long-acting bronchodilator component in composition(s) is, e.g., not more than an amount typically suitable, safe, efficacious, or otherwise necessary of demonstrating a sufficiently beneficial therapeutic effect. In aspects, the concentration of a long-acting bronchodilator component in composition(s) when expressed as a mg/mL concentration can be, e.g., no more than about 1 mg/mL, such as, e.g., ≤˜0.9 mg/mL, ≤˜0.8 mg/mL, ≤˜0.7 mg/mL, ≤˜0.6 mg/mL, ≤˜0.5 mg/mL, ≤˜0.4 mg/mL, ≤˜0.3 mg/mL, ≤˜0.2 mg/mL, ≤˜0.1 mg/mL, ≤˜0.08 mg/mL, ≤˜0.06 mg/mL, ≤˜0.04 mg/mL, or, e.g., ≤˜0.02 mg/mL or ≤˜0.01 mg/mL long-acting bronchodilator component.

In aspects, the concentration of a long-acting bronchodilator component in composition(s) when expressed as a mg/mL concentration can be, e.g., between about 0.01 mg/mL and about 5.5 mg/mL, such as, e.g., ˜0.01 mg/L-˜5 mg/mL, ˜0.01 mg/L-˜4.5 mg/mL, ˜0.01 mg/L-˜4 mg/mL, ˜0.01 mg/L-˜3.5 mg/mL, ˜0.01 mg/L-˜3 mg/mL, ˜0.01 mg/L-˜2.5 mg/mL, ˜0.01 mg/L-˜2 mg/mL, ˜0.01 mg/L-˜1.5 mg/mL, or, e.g., ˜0.01 mg/L-˜1 mg/mL long-acting bronchodilator component.

In aspects, the concentration of a long-acting bronchodilator component in composition(s) when expressed as a mg/mL concentration can be, e.g., between about 0.02 mg/mL and about 5.5 mg/mL, such as, e.g., ˜0.04 mg/mL-˜5.5 mg/mL, ˜0.06 mg/mL-˜5.5 mg/mL, ˜0.08 mg/mL-˜5.5 mg/mL, ˜0.1 mg/mL-˜5.5 mg/mL, ˜0.15 mg/mL-˜5.5 mg/mL, ˜0.2 mg/mL-˜5.5 mg/mL, ˜0.25 mg/mL-˜5.5 mg/mL, ˜0.3 mg/mL-˜5.5 mg/mL, ˜0.4 mg/mL-˜5.5 mg/mL, ˜0.5 mg/mL-˜5.5 mg/mL, ˜0.6 mg/mL-˜5.5 mg/mL, ˜0.7 mg/mL-˜5.5 mg/mL, ˜0.8 mg/mL-˜5.5 mg/mL, ˜0.9 mg/mL-˜5.5 mg/mL, or, e.g., ˜1 mg/mL-˜5.5 mg/mL long-acting bronchodilator component.

In aspects, the concentration of a long-acting bronchodilator component in composition(s) when expressed as a mg/mL concentration can be, e.g., between about 0.01 mg/mL and about 5 mg/mL, such as, e.g., ˜0.05 mg/mL-˜4.5 mg/mL, ˜0.1 mg/mL-˜4 mg/mL, ˜0.2 mg/mL-˜3.5 mg/mL, ˜0.3 mg/mL-˜3 mg/mL, ˜0.4 mg/mL-˜2.5 mg/mL, ˜0.5 mg/mL-˜2 mg/mL, ˜0.6 mg/mL-˜1.5 mg/mL, ˜0.7 mg/mL-˜1.3 mg/mL, or, e.g., ˜0.8 mg/mL-˜1.1 mg/mL, e.g., ˜0.9 mg/mL-˜1 mg/mL long-acting bronchodilator component.

In aspects, the concentration of a long-acting bronchodilator component in composition(s) when expressed as a mg/mL concentration can be, e.g., no less than about 0.01 mg/mL, ≥˜0.02 mg/mL, ≥˜0.03 mg/mL, ≥˜0.04 mg/mL, ≥˜0.05 mg/mL, ≥˜0.06 mg/mL, ≥˜0.07 mg/mL, ≥˜0.08 mg/mL, ≥˜0.09 mg/mL, ≥˜0.1 mg/mL, ≥˜0.2 mg/mL, ≥˜0.3 mg/mL, ≥˜0.4 mg/mL, ≥˜0.5 mg/mL, ≥˜0.6 mg/mL, ≥˜0.7 mg/mL, ≥˜0.8 mg/mL, ≥˜0.9 mg/mL, ≥˜1 mg/mL, ≥˜1.5 mg/mL, ≥˜2 mg/mL, ≥˜2.5 mg/mL, ≥˜3 mg/mL, ≥˜3.5 mg/mL, ≥˜4 mg/mL, ≥˜4.5 mg/mL, ≥˜5 mg/mL, or, e.g., ≥˜5.5 mg/mL long-acting bronchodilator component.

In aspects, the concentration of a long-acting bronchodilator component in composition(s) when expressed as a mg/mL concentration can be, e.g., no more than about 5.5 mg/mL, such as, e.g., ≥˜5 mg/mL, ≥˜4.5 mg/mL, ≥˜4 mg/mL, ≥˜3.5 mg/mL, ≥˜3 mg/mL, ≥˜2.5 mg/mL, ≥˜2 mg/mL, ≥˜1.5 mg/mL, ≥˜1 mg/mL, ≥˜0.9 mg/mL, ≥˜0.8 mg/mL, ≥˜0.7 mg/mL, ≥˜0.6 mg/mL, ≥˜0.5 mg/mL, ≥˜0.4 mg/mL, ≥˜0.3 mg/mL, ≥˜0.2 mg/mL, ≥˜0.1 mg/mL, ≥˜0.09 mg/mL, ≥˜0.08 mg/mL, ≥˜0.07 mg/mL, ≥˜0.06 mg/mL, ≥˜0.05 mg/mL, ≥˜0.04 mg/mL, ≥˜0.03 mg/mL, ≥˜0.02 mg/mL, or, e.g., ≥˜0.01 mg/mL long-acting bronchodilator component.

In certain aspects, the concentration of a long-acting bronchodilator component in composition(s) provided as a solution when expressed as a mg/mL concentration can be, e.g., between about 0.01 mg/mL and about 1 mg/mL, such as, e.g., between about 0.05 mg/mL and about 0.2 mg/mL long-acting bronchodilator component.

In certain aspects, the concentration of a long-acting bronchodilator component in composition(s) provided as a suspension when expressed as a mg/mL concentration can be, e.g., between about 0.01 mg/mL and about 5.5 mg/mL, such as, e.g., between about 0.05 mg/mL and about 1.1 mg/mL long-acting bronchodilator component.

### Long-Acting Bronchodilator Component—%

In aspects, any effective amount of a long-acting bronchodilator component can be used. In aspects, the concentration of a long-acting bronchodilator component in composition(s) when expressed as a weight percent (wt. %) concentration can be, e.g., between about 0.001 wt. % and about 0.1 wt. %, such as, e.g., ˜0.001 wt. %-˜0.095 wt. %, ˜0.001 wt. %-˜0.09 wt. %, ˜0.001 wt. %-˜0.085 wt. %, ˜0.001 wt. %-˜0.08 wt. %, ˜0.001 wt. %-˜0.075 wt. %, ˜0.001 wt. %-˜0.07 wt. %, ˜0.001 wt. %-˜0.065 wt. %, ˜0.001 wt. %-˜0.06 wt. %, ˜0.001 wt. %-˜0.055 wt. %, ˜0.001 wt. %-˜0.05 wt. %, ˜0.001 wt. %-˜0.045 wt. %, ˜0.001 wt. %-˜0.04 wt. %, ˜0.001 wt. %-˜0.035 wt. %, ˜0.001 wt. %-˜0.03 wt. %, ˜0.001 wt. %-˜0.025 wt. %, or, e.g., ˜0.001 wt. %-˜0.02 wt. % long-acting bronchodilator component.

In aspects, the concentration of a long-acting bronchodilator component in composition(s) when expressed as a wt. % concentration can be, e.g., between about 0.0015 wt. % and about 0.1 wt. %, such as, e.g., ˜0.002 wt. %-˜0.1 wt. %, ˜0.0025 wt. %-˜0.1 wt. %, ˜0.003 wt. %-˜0.1 wt. %, ˜0.0035 wt. %-˜0.1 wt. %, ˜0.004 wt. %-˜0.1 wt. %, ˜0.0045 wt. %-˜0.1 wt. %, or, e.g., ˜0.005 wt. %-˜0.1 wt. %, such as, e.g., 0.01 wt. %-˜0.1 wt. %, or, e.g., ˜0.02 wt. %-˜0.1 wt. % long-acting bronchodilator component.

In aspects, the concentration of a long-acting bronchodilating component in composition(s) when expressed as a wt. % concentration can be, e.g., between about 0.0015 wt. % and about 0.095 wt. %, such as, e.g., ˜0.002 wt. %-˜0.09 wt. %, ˜0.0025 wt. %-˜0.085 wt. %, ˜0.003 wt. %-˜0.08 wt. %, ˜0.0035 wt. %-˜0.075 wt. %, ˜0.004 wt. %-˜0.07 wt. %, ˜0.0045 wt. %-˜0.065 wt. %, or, e.g., ˜0.005 wt. %-˜0.06 wt. %, such as, e.g., ˜0.005 wt. %, ˜0.05 wt. %, ˜0.005 wt. %, ˜0.04 wt. %, ˜0.05 wt. %-˜0.03 wt. %, or, e.g., ˜0.05 wt. %-˜0.02 wt. % long-acting bronchodilating component.

In aspects, the concentration of a long-acting bronchodilating component in composition(s) is, e.g., not less than an effective amount of such compound(s), e.g., is not less than an amount capable of demonstrating a detectably or significantly therapeutically beneficial effect. In aspects, the concentration of a long-acting bronchodilating component in composition(s) when expressed as a wt. % concentration can be, e.g., no less than about 0.001 wt. %, ≥˜0.002 wt. %, ≥˜0.004 wt. %, ≥˜0.006 wt. %, ≥˜0.008 wt. %, ≥˜0.01 wt. %, ≥˜0.02 wt. %, ≥˜0.03 wt. %, ≥˜0.04 wt. %, ≥˜0.05 wt. %, ≥˜0.06 wt. %, ≥˜0.07 wt. %, ≥˜0.08 wt. %, ≥˜0.09 wt. %, or, e.g., ≥˜0.1 wt. %, long-acting bronchodilating component.

In aspects, the concentration of a long-acting bronchodilating component in composition(s) is, e.g., not more than an amount typically suitable, safe, efficacious, or otherwise necessary of demonstrating a sufficiently beneficial therapeutic effect. In aspects, the concentration of a long-acting bronchodilating component in composition(s) when expressed as a wt. % concentration can be, e.g., no more than about 0.1 wt. %, such as, e.g., ≤˜0.09 wt. %, ≤˜0.08 wt. %, ≤˜0.07 wt. %, ˜0.06 wt. %, ≤˜0.05 wt. %, ˜0.04 wt. %, ˜0.03 wt. %, ≤˜0.02 wt. %, or, e.g., ≤˜0.01 wt. %, such as ≤˜0.008 wt. %, ≤˜0.006 wt. %, ≤˜0.004 wt. %, ≤˜0.002 wt. %, or, e.g., ≤˜0.001 wt. % long-acting bronchodilating component.

In aspects, the concentration of a long-acting bronchodilator component in composition(s) when expressed as a wt. % concentration can be, e.g., between about 0.001 wt. % and about 0.6 wt. %, such as, e.g., ˜0.001 wt. %-˜0.55 wt. %, ˜0.001 wt. %-˜0.5 wt. %, ˜0.001 wt. %-˜0.45 wt. %, ˜0.001 wt. %-˜0.4 wt. %, ˜0.001 wt. %-˜0.35 wt. %, ˜0.001 wt. %-˜0.3 wt. %, ˜0.001 mg/L-˜0.25 wt. %, ˜0.001 wt. %-˜0.2 wt. %, ˜0.001 wt. %-˜0.15 wt. %, or, e.g., ˜0.001 wt. %-˜0.1 wt. % long-acting bronchodilating component.

In aspects, the concentration of a long-acting bronchodilating component in composition(s) when expressed as a wt. % concentration can be, e.g., between about 0.002 wt. % and about 0.6 wt. %, such as, e.g., ˜0.004 wt. %-˜0.6 wt. %, ˜0.006 wt. %-˜0.6 wt. %, ˜0.008 wt. %-˜0.6 wt. %, ˜0.01 wt. %-˜0.6 wt. %, ˜0.02 wt. %-˜0.6 wt. %, ˜0.03 wt. %-˜0.6 wt. %, ˜0.04 wt. %-˜0.6 wt. %, ˜0.05 wt. %-˜0.6 wt. %, ˜0.06 wt. %-˜0.6 wt. %, ˜0.07 wt. %-˜0.6 wt. %, ˜0.08 wt. %-˜0.6 wt. %, ˜0.09 wt. %-˜0.6 wt. %, ˜0.1 wt. %-˜0.6 wt. %, ˜0.011 wt. %-˜0.6 wt. %, or, e.g., ˜0.012 wt. %-˜0.6 wt. % long-acting bronchodilating component.

In aspects, the concentration of an anticholinergic component in composition(s) when expressed as a wt. % concentration can be, e.g., between about 0.0015 wt. % and about 0.55 wt. %, such as ˜0.002 wt. %-˜0.5 wt. %, ˜0.004 wt. %-˜0.45 wt. %, ˜0.006 wt. %-˜0.4 wt. %, ˜0.008 wt. %-˜0.35 wt. %, ˜0.01 wt. %-˜0.3 wt. %, ˜0.01 wt. %-˜0.2 wt. %, or, e.g., ˜0.01 wt. %-˜0.1 wt. % such as, e.g., ˜0.01 wt. %, ˜0.15 wt. %, ˜0.02 wt. %, ˜0.03 wt. %, ˜0.04 wt. %, ˜0.05 wt. %, ˜0.06 wt. %, ˜0.07 wt. %, ˜0.08 wt. %, ˜0.09 wt. %, ˜0.1 wt. %, or, e.g., ˜0.11 wt. % long-acting bronchodilator component.

In aspects, the concentration of a long-acting bronchodilator component in composition(s) when expressed as a wt. % concentration can be, e.g., no less than about 0.001 wt. %, ≥˜0.002 wt. %, ≥˜0.004 wt. %, ≥˜0.006 wt. %, ≥˜0.008 wt. %, ≥˜0.01 wt. %, ≥˜0.02 wt. %, ≥˜0.04 wt. %, ≥˜0.06 wt. %, ≥˜0.08 wt. %, ≥˜0.1 wt. %, ≥˜0.15 wt. %, ≥˜0.2 wt. %, ≥˜0.25 wt. %, ≥˜0.3 wt. %, ≥˜0.35 wt. %, ≥˜0.4 wt. %, ≥˜0.45 wt. %, or, e.g., ≥˜0.5 wt. % or ≥˜0.55 wt. % long-acting bronchodilator component.

In aspects, the concentration of a long-acting bronchodilator component in composition(s) when expressed as a wt. % concentration can be, e.g., no more than about 0.55 wt. %, such as, e.g., ≥˜0.5 wt. %, ≤˜0.45 wt. %, ≥˜0.4 wt. %, ≤˜0.35 wt. %, ≥˜0.3 wt. %, ≤˜0.25 wt. %, ≥˜0.2 wt. %, ≤˜0.15 wt. %, ≥˜0.1 wt. %, ≤˜0.08 wt. %, ≥˜0.06 wt. %, ≤˜0.04 wt. %, ≥˜0.02 wt. %, ≤˜0.01 wt. %, ≥˜0.008 wt. %, ≤˜0.006 wt. %, ≥˜0.004 wt. %, ≥˜0.002 wt. %, or, e.g., ≤˜0.001 wt. % long-acting bronchodilator component.

In certain aspects, the concentration of a long-acting bronchodilator component in composition(s) provided as a solution when expressed as a wt. % concentration can be, e.g., between about 0.001 wt. % and about 0.1 wt. %, such as, e.g., between about 0.005 wt. % and about 0.02 wt. % along-acting bronchodilator component.

In certain aspects, the concentration of a long-acting bronchodilator component in composition(s) provided as a suspension when expressed as a wt. % concentration can be, e.g., between about 0.001 wt. % and about 0.55 wt. %, such as, e.g., between about 0.005 wt. % and about 0.11 wt. % long-acting bronchodilator component.

### Bronchodilating Component—Amounts

In aspects, as stated above, composition(s) comprise a bronchodilating component comprising only a long-acting bronchodilator sub-component. Therefore, in aspects, composition(s) can comprise a bronchodilating component in amount(s) represented above as described for a long-acting bronchodilating component. In aspects, a bronchodilating component can comprise both long- and short-acting bronchodilating sub-components. Therefore, in aspects, composition(s) can comprise a bronchodilating component in amount(s) established by the combination of (e.g., sum of) amount(s) represented above/herein of a short-acting bronchodilating component and a long-acting bronchodilating component.

### Anti-Inflammatory Component

In aspects, the invention provides composition(s) delivered by inhalation comprising an anti-inflammatory component. In aspects an anti-inflammatory component comprises one or more anti-inflammatory agent(s)/compound(s). In aspects, an anti-inflammatory agent/compound is any pharmaceutically acceptable compound suitable for inhalation which, upon administration, detectably or significantly reduces inflammation within air passageway(s), e.g., bronchi, of the lung. In aspects, a detectable or significant reduction in air passageway inflammation detectably or significantly improves respiratory function, e.g., results in a detectable or significant improvement in respiratory function.

According to certain aspects, composition(s) herein comprise an API component comprising a bronchodilating component, wherein the bronchodilating component comprises, e.g., a long-acting bronchodilating subcomponent, wherein, e.g., the long-acting bronchodilating subcomponent comprises an anticholinergic component comprising, e.g., a tiotropium compound. In aspects, the composition further comprises a non-anticholinergic component comprising, e.g., a beta-adrenergic agent agonist component, comprising, e.g., a beta 2 agonist such as, e.g., a formoterol compound. In aspects, any such composition of this paragraph can comprise an anti-inflammatory component (such as, e.g., an anti-inflammatory component comprising one or more inhaled corticosteroid(s) as described herein). In aspects, anti-inflammatory agent(s) can be administered simultaneously with, sequentially with, or provided separately from any one or more other API(s) of the composition (such as, e.g., any one or more bronchodilating agent(s)/compound(s), e.g., a tiotropium compound or a formoterol compound. In aspects, all such APIs, including APIs of an anti-inflammatory component, are co-administered, e.g., are provided within the same (a single) composition.

According to certain exemplary aspects, composition(s) herein comprise at least one anticholinergic agent, e.g., a tiotropium compound, at least one long-acting beta-2-agonist (LABA), e.g., a formoterol compound, and at least one corticosteroid.

### Steroid Anti-Inflammatory Component: Inhaled Steroid(s) (Corticosteroid(s)/Glucocorticosteroid(s)

In aspects, composition(s) provided herein comprise an anti-inflammatory component comprising one or more sub-components. In aspects, an anti-inflammatory component subcomponent is a steroid anti-inflammatory component. In aspects, the steroid anti-inflammatory component comprises one or more inhalable steroid compound(s)/agent(s), such as corticosteroid(s) or glucocorticosteroid(s). According to some aspects, composition(s) comprising an anti-inflammatory component comprise at least a steroid anti-inflammatory component. In aspects, a steroid anti-inflammatory component comprises at least one inhalable (inhaled) corticosteroid agent/compound/constituent.

In aspects, a steroid anti-inflammatory component can comprise any one or more pharmaceutically acceptable steroid anti-inflammatory constituent(s)/compound(s) capable of detectably or significantly reducing inflammation as described above. In aspects, exemplary anti-inflammatory component constituent(s) can comprise, e.g., inhaled corticosteroids such as, e.g., mometasone, mometasone furoate, beclomethasone, beclomethasone dipropionate, fluticasone, fluticasone propionate, fluticasone furoate, fluticasone valerate, ciclesonide, budesonide, R-budesonide etc. In aspects, a corticosteroid is selected from one or more of ciclesonide, beclomethasone, and a fluticasone compound, e.g., fluticasone furoate.

### Steroidal Anti-Inflammatory Component—Amount

In certain aspects, composition(s) comprise a single API component, e.g., comprising only an anti-inflammatory component, e.g., comprising only a steroidal anti-inflammatory component. In aspects, an anti-inflammatory component comprises only a steroidal anti-inflammatory component. In aspects, therefore, amounts described within this section can refer to (a) the entirety of the API concentration within a composition when the API component of a composition comprises only an anti-inflammatory component consisting only of a steroidal anti-inflammatory component; (b) the entirety of any anti-inflammatory component concentration of a composition when the anti-inflammatory component only comprises a steroidal anti-inflammatory component; (c) the concentration of the steroidal anti-inflammatory component itself; (d) the concentration of any single steroidal anti-inflammatory agent/compound if the anti-inflammatory component comprises a single API, or (e) any combination of (a)-(d).

Steroidal Anti-Inflammatory Component—Amount (mg/mL)

In aspects, any effective amount of a steroidal anti-inflammatory component can be used. In aspects, the concentration of a steroidal anti-inflammatory component, e.g., corticosteroid(s) in composition(s) when expressed as a mg/mL concentration can be, e.g., between about 1 mg/mL and about 5 mg/mL, such as, e.g., ˜1 mg/mL-˜4.5 mg/mL, ˜1 mg/mL-˜4 mg/mL, ˜1 mg/mL-˜3.5 mg/mL, ˜1 mg/mL-˜3 mg/mL, ˜1 mg/mL-˜2.5 mg/mL, ˜1 mg/mL-˜2 mg/mL, or, e.g., ˜1 mg/mL-˜1.5 mg/mL steroidal anti-inflammatory component, e.g., corticosteroid(s), e.g., ciclesonide, beclomethasone, or fluticasone compound, e.g., fluticasone furoate.

In aspects, the concentration of a steroidal anti-inflammatory component, e.g., corticosteroid(s) in composition(s) when expressed as a mg/mL concentration can be, e.g., between about 1.5 mg/mL and about 5 mg/mL, such as, e.g., ˜2 mg/mL-˜5 mg/mL, ˜2.5 mg/mL-˜5 mg/mL, ˜3 mg/mL-˜5 mg/mL, ˜3.5 mg/mL-˜5 mg/mL, ˜4 mg/mL-˜5 mg/mL, or, e.g., ˜4.5 mg/mL-˜5 mg/mL steroidal anti-inflammatory component, e.g., corticosteroid(s), e.g., ciclesonide, beclomethasone, or fluticasone compound, e.g., fluticasone furoate.

In aspects, the concentration of a steroidal anti-inflammatory component, e.g., corticosteroid(s) in composition(s) when expressed as a mg/mL concentration can be, e.g., between about 1.2 mg/mL and about 4.8 mg/mL, such as, e.g., ˜1.3 mg/mL-˜4.6 mg/mL, ˜1.4 mg/mL-˜4.4 mg/mL, ˜1.5 mg/mL-˜4.2 mg/mL, ˜1.6 mg/mL-˜4 mg/mL, ˜1.7 mg/mL-˜3.8 mg/mL, or, e.g., ˜1.8 mg/mL-˜3.6 mg/ml steroidal anti-inflammatory component, e.g., corticosteroid(s), e.g., ciclesonide, beclomethasone, or fluticasone compound, e.g., fluticasone furoate.

In aspects, the concentration of a steroidal anti-inflammatory component in composition(s) is, e.g., not less than an effective amount of such compound(s), e.g., is not less than an amount capable of demonstrating a detectably or significantly therapeutically beneficial effect. In aspects, the concentration of a steroidal anti-inflammatory component, e.g., corticosteroid(s) in composition(s) when expressed as a mg/mL concentration can be, e.g., no less than about 1 mg/mL, ≥˜1.5 mg/mL, ≥˜2 mg/mL, ≥˜2.5 mg/mL, ≥˜3 mg/mL, ≥˜3.5 mg/mL, ≥˜4 mg/mL, ≥˜4.5 mg/mL, or, e.g., ≥˜5 mg/mL, or, e.g., ≥˜1.8 mg/mL, ≥˜2.2 mg/mL, ≥˜2.6 mg/mL, or, ≥˜3.2 mg/mL steroidal anti-inflammatory component, e.g., corticosteroid(s), e.g., ciclesonide, beclomethasone, or fluticasone compound, e.g., fluticasone furoate.

In aspects, the concentration of a steroidal anti-inflammatory component in composition(s) is, e.g., not more than an amount typically suitable, safe, efficacious, or otherwise necessary of demonstrating a sufficiently beneficial therapeutic effect. In aspects, the concentration of a steroidal anti-inflammatory component, e.g., corticosteroid(s), in composition(s) when expressed as a mg/mL concentration can be, e.g., no more than about 5 mg/mL, such as, e.g., ≤˜4.5 mg/mL, ≤˜4 mg/mL, ≤˜3.5 mg/mL, ≤˜3 mg/mL, ≤˜2.5 mg/ml, ≤˜2 mg/mL, ≤˜1.5 mg/mL, or, e.g., ≤˜1 mg/mL, such as, for example, ≤˜1.8 mg/mL, ≤˜2.2 mg/mL, ≤˜2.6 mg/mL, or, e.g., ≤˜3.2 mg/mL mg/mL steroidal anti-inflammatory component, e.g., corticosteroid(s), e.g., ciclesonide, beclomethasone, or fluticasone compound, e.g., fluticasone furoate.

### Steroidal Anti-Inflammatory Component—Amount (wt. %)

In aspects, any effective amount of a steroidal anti-inflammatory component can be used. In aspects, the concentration of a steroidal anti-inflammatory component, e.g., corticosteroid(s) in composition(s) when expressed as a weight percent (wt. %) concentration can be, e.g., about 0.1 wt. % and about 0.5 wt. %, such as, e.g., ˜0.1 wt. %-˜0.45 wt. %, ˜0.1 wt. %-˜0.4 wt. %, ˜0.1 wt. %-˜0.35 wt. %, ˜0.1 wt. %-˜0.3 wt. %, ˜0.1 wt. %-˜0.25 wt. %, or, e.g., ˜0.1 wt. %-˜0.15 wt. % steroidal anti-inflammatory component, e.g., corticosteroid(s), e.g., ciclesonide, beclomethasone, or fluticasone compound, e.g., fluticasone furoate. In aspects, compositions can comprise a steroidal anti-inflammatory component, e.g., corticosteroid(s), e.g., ciclesonide, beclomethasone, or fluticasone compound (e.g., fluticasone furoate), in an amount of between about 0.05 wt. % and about 0.4 wt. %, such as, e.g., ˜0.05 wt. %-˜0.35 wt. %, ˜0.05 wt. %-˜0.3 wt. %, or ˜0.05 wt. %-˜2.5 wt. %, e.g., ˜0.1 wt. %-˜0.4 wt. %, ˜0.15 wt. %-˜0.4 wt. %, or, e.g., ˜0.1 wt. %-˜0.3 wt. %, ˜0.15 wt. %-˜0.25 wt. %, such as, e.g., 0.2 wt. % of the composition.

In aspects, the concentration of a steroidal anti-inflammatory component, e.g., corticosteroid(s) in composition(s) when expressed as a wt. % concentration can be, e.g., between about 0.15 wt. % and about 0.5 wt. %, such as, e.g., ˜0.2 wt. %-˜0.5 wt. %, ˜0.25 wt. %-˜0.2 wt. %, ˜0.3 wt. %-˜0.5 wt. %, ˜0.35 wt. %-˜0.5 wt. %, ˜0.4 wt. %-˜0.5 wt. %, or, e.g., ˜0.45 wt. %-˜0.5 wt. % steroidal anti-inflammatory component, e.g., corticosteroid(s), e.g., ciclesonide, beclomethasone, or fluticasone compound, e.g., fluticasone furoate.

In aspects, the concentration of a steroidal anti-inflammatory component, e.g., corticosteroid(s) in composition(s) when expressed as a wt. % concentration can be, e.g., between about 0.12 wt. % and about 0.48 wt. %, such as, e.g., ˜0.14 wt. %-˜0.46 wt. %, ˜0.16 wt. %-˜0.44 wt. %, ˜0.18 wt. %-˜0.42 wt. %, ˜0.2 wt. %-˜0.4 wt. %, ˜0.22 wt. %-˜0.38 wt. %, ˜0.24 wt. %-˜0.36 wt. %, ˜0.26 wt. %-˜0.34 wt. %, or, e.g., ˜0.28 wt. %-˜0.32 wt. % steroidal anti-inflammatory component, e.g., corticosteroid(s), e.g., ciclesonide, beclomethasone, or fluticasone compound, e.g., fluticasone furoate.

In aspects, the concentration of a steroidal anti-inflammatory component in composition(s) is, e.g., not less than an effective amount of such compound(s), e.g., is not less than an amount capable of demonstrating a detectably or significantly therapeutically beneficial effect. In aspects, the concentration of a steroidal anti-inflammatory component, e.g., corticosteroid(s) in composition(s) when expressed as a wt. % concentration can be, e.g., no less than about 0.01 wt. %, ≥˜0.15 wt. %, ≥˜0.2 wt. %, ≥˜0.25 wt. %, ≥˜0.3 wt. %, ≥˜0.35 wt. %, ≥˜0.4 wt. %, ≥˜0.45 wt. %, or, e.g., ≥˜0.5 wt. % steroidal anti-inflammatory component, e.g., corticosteroid(s), e.g., ciclesonide, beclomethasone, or fluticasone compound, e.g., fluticasone furoate.

In aspects, the concentration of a steroidal anti-inflammatory component in composition(s) is, e.g., not more than an amount typically suitable, safe, efficacious, or otherwise necessary of demonstrating a sufficiently beneficial therapeutic effect. In aspects, the concentration of a steroidal anti-inflammatory component, e.g., corticosteroid(s) in composition(s) when expressed as a wt. % concentration can be, e.g., no more than about 0.5 wt. %, such as, e.g., ≤˜0.45 wt. %, ≤˜0.4 wt. %, ≤˜0.35 wt. %, ≤˜0.3 wt. %, ≤˜0.25 wt. %, ≤˜0.2 wt. %, ≤˜0.15 wt. %, or, e.g., ≤˜0.1 wt. % steroidal anti-inflammatory component, e.g., corticosteroid(s), e.g., ciclesonide, beclomethasone, or fluticasone compound, e.g., fluticasone furoate.

### Non-Steroid Anti-Inflammatory Component

In aspects, an anti-inflammatory component subcomponent is a non-steroid anti-inflammatory component. In aspects, the non-steroid anti-inflammatory component comprises one or more inhalable non-steroid compound(s)/agent(s) (NSAID(s)).

In aspects, a non-steroid anti-inflammatory component can comprise any one or more pharmaceutically acceptable non-steroid anti-inflammatory constituent(s)/compound(s) capable of detectably or significantly reducing inflammation as described above. In aspects, exemplary anti-inflammatory component constituent(s) can comprise, for example, cromolyn sodium (SCG), nedocromil sodium (NS), flurbiprofen axetil, parecoxib sodium, lysine aspirin (L-aspirin), indomethacin, sodium salicylate, etc.

### Non-Steroid Anti-Inflammatory Component—Amount

In aspect, non-steroid anti-inflammatory component constituent(s) can be present in any therapeutically effective amount. In certain aspects, composition(s) comprise a single API component, e.g., comprising only an anti-inflammatory component, e.g., comprising only a non-steroidal anti-inflammatory component. In aspects, an anti-inflammatory component comprises mostly, substantially only, or only a non-steroidal anti-inflammatory component. In aspects, therefore, amounts described within this section can refer to (a) the entirety of the API concentration within a composition when the API component of a composition comprises only an anti-inflammatory component consisting only of a non-steroidal anti-inflammatory component; (b) the entirety of any anti-inflammatory component concentration of a composition when the anti-inflammatory component only comprises a non-steroidal anti-inflammatory component; (c) the concentration of the non-steroidal anti-inflammatory component itself; (d) the concentration of any single non-steroidal anti-inflammatory agent/compound if the anti-inflammatory component comprises a single API, or (e) any combination of (a)-(d).

### Anti-Inflammatory Component—Amount

In aspects, as stated above, composition(s) comprise an anti-inflammatory component comprising only a steroidal anti-inflammatory, e.g., corticosteroid, sub-component. Therefore, in aspects, composition(s) can comprise an anti-inflammatory component in amount(s) represented above as described for a steroidal anti-inflammatory component.

### Other API Component

In certain aspects, composition(s) provided herein can comprise one or more additional API components, such as, e.g., a cromolyn component, a monoclonal (inhaled) component, or other known or recognized classification of inhalable compound(s) suitable for the prevention or treatment of respiratory-related disorders such as, e.g., asthma, COPD, or both, or symptom(s) related to such disorder(s). In aspects, composition(s) can comprise antibiotics, such as, e.g., aminoglycosides including gentamycin, colomycin, amikacin, liposomal amikacin, neomycin, sisomycin, or, e.g., tobramycin; polymyxins such as, e.g., colistin/polymyxin B; glycopeptides such as, e.g., vancomycin; monobactams such as, e.g., aztreonam lysine; beta-lactams such as, e.g., ceftazidime; a penicillin antibiotic such as, e.g., amoxicillin, etc. In aspects, composition(s) can comprise one or more pulmonary hypertension reducing agent(s) such as, e.g., prostacyclins such as iloprost, treprosinil, epoprostenol, etc., heparin, etc. In aspects, composition(s) can comprise one or more mucolytic agent(s) such as, e.g., acetylcysteine, bromhexine, ambroxol, carbocisteine, dithiothreitol, or, e.g., P3001 class mucolytic agents as described by Here, et. al. in, “An Improved Inhaled Mucolytic to Treat Airway Muco-obstructive Diseases,” Am J Respir Crit Care Med., 2019 Jan. 15; 199(2): 171-180. In aspects, composition(s) can comprise one or more MABA (muscarinic antagonist ß2 agonist) molecule(s) such as, e.g., compounds described by Hughes, et. al, in, “Dual-pharmacology muscarinic antagonist and 2 agonist molecules for the treatment of chronic obstructive pulmonary disease,” in Future Med Chem. 2011 October; 3(13):1585-605; Rancati, et. al., in, “Discovery of a novel class of inhaled dual pharmacology muscarinic antagonist and B2 agonist (MABA) for the treatment of chronic obstructive pulmonary disease (COPD),” in Bioorg Med Chem Lett. 2021 Jun. 1; 41:127975; and, e.g., batefenterol, navafeneraol, and others such as those described by Ora, et. al., in, “Long-Acting Muscarinic Antagonists Under Investigational [sic] to Treat Chronic Obstructive Pulmonary Disease,” J Exp Pharmacol. 2020; 12:559-574. Such other/additional API are included in the formulation in addition to the amount(s) of other APIs described herein. Such APIs are typically present in effective amount (individually or in combination with other APIs).

### Combinations of Components/Sub-Components

In aspects, components of composition(s) described herein can be described in alternative ways, such as, e.g., describing composition(s) as comprising two components, e.g., an anticholinergic API component and a non-anticholinergic API component, wherein, e.g., the non-anticholinergic API component can comprise, e.g., any agent/API characterizable as something other than an anticholinergic agent, such as, e.g., a beta-adrenergic agonist, an anti-inflammatory agent, e.g., an inhaled corticosteroid, or, e.g., any combination thereof. Accordingly, disclosure herein should be interpreted to encompass the description of composition(s) encompassing any combination of components or subcomponents. Disclosure herein further encompasses the amounts and relationship(s) between such combination(s) of components, as is disclosed at least in exemplary part elsewhere herein.

### Propellant Component(s)

In aspects, composition(s) provided by the invention are delivered by inhalation. In aspects, composition(s) are provided as an inhaled aerosol. In aspects, composition(s) provided herein comprise a means for aerosolizing (dispersing composition(s) as an aerosol) composition(s) upon activation (e.g., upon the triggering of an inhalation device, such as, e.g., an MDI comprising a composition). In aspects, such a means can be, e.g., any mechanism resulting in the formation of an aerosol, such as, e.g., by the application of oxygen, compressed air, or ultrasonic power to sufficiently break up composition(s) (e.g., in the form of suspensions, solutions, or both) into aerosol droplets, or e.g., by providing a propellant component.

In aspects, composition(s) provided herein comprise a propellant component to aid in the aerosolization of, or to form an aerosol of, composition(s) to facilitate delivery by inhalation. A propellant component can comprise any one or more pharmaceutically acceptable propellant(s) in any suitable amount(s)/concentration(s). Propellants are known in the art and, in general, any suitable propellant can be incorporated, but certain types of propellants that are particularly suitable are described below and can serve to define, along with possibly other components/characteristics, steps, etc., particular aspects of the invention. In aspects, a propellant compound present in composition(s) herein has been demonstrated as safe for use in inhaled composition(s), such as being approved for such use by a recognized regulatory authority such as, for example, the United States Food and Drug Administration (US FDA). Exemplary propellant component compound(s)/constituent(s)/agent(s) include, e.g., chlorofluorocarbon(s) (CFC(s)), hydrofluoroalkane(s) (HFA(s)), and, surprisingly, hydrofluoropropenes (HFO(s)), especially, for example, tetrafluoropropenes. In aspects, hydrocarbons, such as n-propane, n-butane, or isobutane; halogen-substituted hydrocarbons, e.g., fluorine-substituted methanes, ethanes, propanes, butanes, cyclopropanes, or cyclobutanes are present in propellant system(s) suitable for use in composition(s) provided herein. Propellants typically are characterized by the ability to detectably or significantly aid in the propulsion of composition(s) in delivering the composition(s) by inhalation. In aspects, propellants are characterized by the primary function of the composition, that being to aid in such delivery by inhalation (as compared to imparting other functions).

In aspects, composition(s) herein comprise a propellant component comprising one or more propellant compound(s), wherein the compound(s) and thus the propellant component have/has a suitable boiling point and vapor pressure, allowing for liquification of the compound(s) in a closed system, e.g., closed container, when maintained at room temperature (e.g., about 25° C.)

In aspects, composition(s) herein comprise a propellant component comprising one or more propellant compound(s), wherein the propellant component is capable of being maintained at a sufficiently high pressure such that when released, e.g., when an MDI is activated, the pressure is sufficient to deliver composition(s), e.g., API(s) of composition(s) in atomized for, even at low ambient temperatures.

In aspects, composition(s) herein comprise a propellant component comprising one or more propellant compound(s), wherein the compound(s) and thus the propellant component demonstrate/demonstrates (e.g., as determined by method(s) known in the art or as recognized by an authoritative regulatory body such as, e.g., the US FDA) a sufficiently low acute toxicity, a sufficiently low chronic toxicity, or both a sufficiently low acute and chronic toxicity to render the composition safe for human use over a period of at least about 1 week, at least about 1 month, ≥˜3 months, ≥˜6 months, ≥˜9 months, ≥˜12 months, ≥˜18 months, ≥˜2 years, ≥˜3 years, ≥˜5 years, ≥˜10 years, ≥˜15 years, ≥˜20 years, ≥˜25 years, ≥˜30 years or for a period of time longer than 40 or 50 years when administered at least once daily, or, e.g., in aspects, when administered about twice or three times daily, e.g., ˜4 or ˜5 times daily, for such periods.

In aspects, composition(s) herein comprise a propellant component comprising one or more propellant compound(s) wherein the compound(s) and thus the propellant component comprise/comprises a suitable cardiac sensitization threshold (increased sensitivity of the heart to adrenaline brought about by exposure to high concentration of certain organic compounds) as measured by cardiac sensitization method(s) known in the art.

In aspects, composition(s) herein comprise a propellant component comprising one or more propellant compound(s) wherein the compound(s) and thus the propellant component demonstrate/demonstrates chemical stability (e.g., lack of detectable or significant breakdown of the compound(s) rendering the compound(s) detectably or significantly less effective, unsuitable, or inappropriate for administration to a recipient) when in contact with one or more API(s) of composition(s) or one or more component(s) of the delivery system (e.g., MDI) by which composition(s) are delivered, e.g., one or more metallic or non-metallic component(s) of such device(s).

In aspects, composition(s) herein comprise a propellant component comprising one or more propellant compounds wherein the compound(s) and thus the propellant component demonstrate/demonstrates a low propensity to extract low molecular weight substances from any elastomeric material(s) present in any one or more component(s) of the delivery system (e.g., MDI) by which composition(s) are delivered.

In aspects, composition(s) herein comprise a propellant component comprising one or more propellant compounds wherein the propellant component maintains one or more API(s) present in the composition in homogeneous solution, stable suspension, or stable dispersion sufficient to permit reproducible drug delivery.

In aspects, composition(s) herein, when provided as a suspension, comprise a propellant component comprising one or more propellant compound(s) wherein the propellant component is a liquid having a density at least substantially similar to that of solid API(s) maintained therein, such that detectable or significant sinking, floating, or both of drug particles within the liquid is at least substantially avoided or any detectably sinking, floating, or both can be overcome by simple, short-term (e.g., 1-5 second) shaking of the composition prior to use.

In aspects, composition(s) herein, comprise a propellant component comprising one or more propellant compound(s) wherein the compound(s) and thus the propellant component are non-flammable or comprise a sufficiently low flammability when mixed with air, e.g., when mixed with air in the respiratory tract to render the propellant component safe and suitable for administration to a mammalian patient via, e.g., a delivery device such as, e.g., an MDI.

In aspects, a propellant component can comprise any one or more such propellant compound(s), described above, such as comprising a single propellant compound (or e.g., at least substantially consists of a single propellant compound), or, e.g., comprising a combination of two or more propellant compound(s). In certain aspects, a propellant component is at least about 90%, such as, e.g., ≥˜91%, ≥˜92%, ≥˜93%, ≥˜94%, ≥˜95%, ≥˜96%, ≥˜97%, ≥˜98%, ≥˜99%, or, e.g., is even, in aspects 100%, composed of the same propellant compound. In aspects, composition(s) comprise two or more propellant compounds wherein each compound belongs to the same class of propellant, e.g., each propellant compound is a CFC, each propellant compound is an HFA, or, e.g., each propellant compound is an HFO. Alternatively, in aspects, composition(s) comprise two or more propellant compounds wherein at least two of the at least two propellant compounds of a propellant component belong to different classes of propellant; for example, at least one propellant is an HFA and at least one propellant is an HFO. In certain aspects, it may be advantageous for composition(s) to comprise a propellant component at least substantially consisting of an effective amount of a single propellant compound. In other aspects, it may be advantageous for composition(s) to comprise a propellant component comprising an effective amount of each of at least two propellant compound(s). In aspects, an advantage of a single propellant, or, e.g., in other scenarios/embodiments two or more propellants, can be, e.g., that such a propellant system is one in which (a) an effective amount of one or more API(s) can be dissolved (e.g., the composition can be effectively delivered as a solution), (b) a suitable composition can be obtained which does not require delivery via a device having modified material characteristics (e.g., requiring component(s) of the device to be manufactured using a particular material or to be coated with a protective coating) as is described elsewhere herein, or (c) both (a) and (b) are true.

In aspects, a propellant component detectably or significantly provides a medium in which a detectable or significant amount of one or more API(s), such as, e.g., a therapeutically effective amount of API(s) can be dissolved. In aspects, a propellant component is present with one or more excipients which aids in the detectable or significant dissolution of the API(s), such as, e.g., one or more co-solvent compound(s). Such excipients are described elsewhere herein. In certain aspects, API(s) of a composition herein are held in suspension within a propellant, e.g., in aspects, a propellant component can participate in the suspension of or provide a suspension medium for some, most, generally all, or all API(s) or portions of such API(s) in composition(s) herein.

In aspects, it may be advantageous for composition(s) to comprise an HFO. In aspects, it may be advantageous for composition(s) to comprise one or more HFOs and lack any propellant compound(s) not classifiable as an HFO.

Exemplary propellant compound(s) are discussed in detail below. In certain exemplary aspects, composition(s) herein comprise a propellant component comprising one or more of HFO-1234ze, HFA-134a, and HFA-152a. In particular, in one aspect, the inventive composition(s) provided herein provide tiotropium compound-based composition(s) deliverable by MDI(s) wherein the composition(s) comprise a propellant component comprising HFO-1234ze, HFA-134a, HFA-152a, or mixture thereof. In aspects, composition(s) herein exhibit (a) detectably or significantly improved chemical stability; (b) detectably or significantly improved aerosolization performance; (c) detectably or significantly improved drug delivery; (d) detectably or significantly greater formulation stability; (e) detectably or significantly reduced environmental impact, (e.g., reduced GWP, reduced ODP, or both); (f) suitable compatibility with MDI component(s) manufactured using standard technique(s) and material(s) without having to modify such technique(s) or material(s) to address compositional compatibility issues; or (g) any combination of any or all of (a)-(f), e.g., as compared to other tiotropium compound-based composition(s) comprising, at a minimum, the same or at least substantially the same amount of tiotropium, the same one or more additional API(s) as applicable, the same or similar excipient(s) as applicable, the same or at least substantially the same amount of such excipient(s) as applicable, or any combination thereof.

### CFC Propellants

According to certain aspects, composition(s) provided by the invention comprise a propellant component comprising one or more chlorofluorocarbon (CFC) propellant compound(s). In aspects, such a CFC compound can be any non-toxic (or, e.g., toxicologically safe), non-flammable CFC compound having suitable characteristic(s) for use in delivering composition(s) herein in aerosolized form, such as, e.g., by an inhaler device such as a metered dose inhaler (MDI) (e.g., a pressurized metered dose inhaler). For example, such CFC compound(s) have a vapor pressure which is suitable to enable the composition(s) herein to be administered via a pressurized MDI. Further, in aspects, CFC propellant compound(s) suitable for use in composition(s) herein are compatible with the components of the MDI device (such as containers, valves, and sealing gaskets, etc.) employed to administer the medicament. In aspects, such components are those manufactured by traditional techniques. In aspects, such components do not require use of non-traditional materials or coatings to facilitate compatibility with the CFC propellant compound(s). This is discussed further elsewhere herein. In aspects, such a CFC is a CFC compound approved for use by a recognized regulatory body, such as, e.g., the US FDA.

In aspects, exemplary chlorofluorocarbon propellant compounds include but are not limited to, e.g., trichlorofluoromethane (CFC 11 or R-11), dichlorodifluoromethane (CFC 12 or R-12), or, e.g., dichlorotetrafluoroethane (CFC 114 or R-114), etc., or mixtures of any or all thereof, such as, e.g., a blend of R-12 and R-11.

### HFA Propellants

In certain aspects, composition(s) provided by the invention comprise a propellant component comprising propellant compound(s) characterizable as a hydrofluoroalkane hydrofluorocarbon, herein referred to as an HFA. An HFA compound is an alkane (saturated) compound comprising hydrogen, fluorine, and carbon atoms. Herein, use of the term HFA is intended to specifically specify such a saturated state of the hydrofluorocarbon propellant compound. This characteristic is, in aspects, important with regard to differentiating such compounds from hydrofluoroolefin compounds further referenced herein. In aspects, HFA propellant(s) have one or more advantageous properties over traditional CFC compound(s) such as those listed herein. In aspects, propellant compound(s) provided by the invention comprise any environmentally safe propellant compound(s). In aspects, HFA compound(s) of a propellant component have a detectably or significantly reduced adverse effect on global warming (e.g., have a detectably or significantly lower global warming potential (GWP)), have a detectably or significantly reduced adverse effect on stratospheric ozone (e.g., have a detectably or significantly lower ozone depletion potential (ODP)), or both than that of traditional CFC compound(s). Accordingly, in aspects, composition(s) herein comprise a propellant component which has a detectably or significantly lower environmental impact than that of traditional CFC compound(s). In aspects, such compounds comprise one or more HFA compounds.

In aspects, such HFA compound(s) can be any non-toxic (or, e.g., toxicologically safe), non-flammable HFA compound having suitable characteristic(s) for use in delivering composition(s) herein in aerosolized form, such as, e.g., by an inhaler device such as a metered dose inhaler (e.g., a pressurized metered dose inhaler). For example, such HFA compound(s) have a vapor pressure which is suitable to enable the composition(s) herein to be administered via a pressurized MDI. Further, in aspects, HFA propellant compound(s) suitable for use in composition(s) herein are compatible with the components of the MDI device (such as containers, valves, and sealing gaskets, etc.) employed to administer the medicament. In aspects, such components are those manufactured by traditional techniques. In aspects, such components do not require use of non-traditional materials or coatings to facilitate compatibility with the HFA propellant compound(s). This is discussed further elsewhere herein. In aspects, such an HFA is an HFA compound approved for use by a recognized regulatory body, such as, e.g., the US FDA.

In aspects, any pharmaceutically acceptable hydrofluorocarbonated propellants can be used. In aspects, exemplary hydrofluoroalkane propellant compounds include but are not limited to, e.g., difluoromethane (HFA-32), tetrafluoroethane (HFA-134) (e.g., 1,1,2,2-tetrafluoroethane or, e.g., 1,1,1,2-tetrafluoroethane (HFA-134(a)), heptafluoropropane (HFA-227) (e.g., 1,1,1,2,3,3,3,-heptafluoropropane (HFA-227ea)), trifluoroethane (e.g., 1.1.1-trifluoroethane (HFA-143a), HFA-152a (1,1-difluoroethane), etc., and mixtures of any or all thereof. In certain aspects, composition(s) herein do not comprise, e.g., composition(s) lack a propellant component comprising HFA-227. In certain aspects, composition(s) herein do not comprise, e.g., composition(s) lack a propellant component comprising HFA-32. In certain aspects, composition(s) herein do not comprise, e.g., composition(s) lack a propellant component comprising HFA-143. In certain aspects, composition(s) herein lack any propellant component characterized as an HFA (such as, e.g., composition(s) comprising only one or more HFO propellant compound(s)).

In particular aspects, composition(s) comprising one or more HFA compounds, such as, e.g., HFA-134a, HFA-152a, or, e.g., a mixture thereof, with or without one or more additional propellants such as one or more HFO compounds, e.g., one or more tetrafluoropropenes, for example one or both of 2,3,3,3-tetrafluoropropene (HFO-1234yl) and 1,3,3,3-tetrafluoropropene (HFO-1234ze), demonstrate detectably or significantly greater stability compared to composition(s) comprising the same or similar components, e.g., sharing one or more active pharmaceutical ingredients, sharing one or more APIs in the same amount(s), sharing one or more excipients, sharing one or more excipients in the same amount(s), or any combination thereof, but lacking such propellant(s), such as stability when stored under normal storage conditions (normal storage conditions being, e.g., about room temperature or between about 2° C. and about 8° C. as is discussed elsewhere herein) for a period of at least about 1 week, such as, e.g., ≥˜2 weeks, ≥˜3 weeks, ≥˜1 months, ≥˜6 weeks, ≥˜2 months, ≥˜10 weeks, ≥˜3 months, ≥˜4 months, ≥˜5 months, ≥˜6 months, ≥˜9 months, ≥˜12 months, ≥˜18 months, ≥˜24 months, ≥˜30 months, or, e.g., ≥˜36 months. Stability of composition(s) provided by the invention is discussed in greater detail elsewhere herein.

### HFO Propellants

In aspects, composition(s) provided herein comprise a propellant component wherein the propellant component does not comprise a propellant compound characterizable as a saturated hydrocarbon. In certain aspects, composition(s) provided by the invention comprise a propellant component comprising propellant compound(s) characterizable as a hydrofluoroolefin hydrofluorocarbon compound, herein referred to as an HFO. An HFO compound is an alkene (unsaturated) compound comprising hydrogen, fluorine, and carbon atoms. Herein, use of the term HFO is intended to specifically specify such an unsaturated state of the hydrofluorocarbon propellant compound. In certain aspects, composition(s) provided herein comprise a propellant component wherein the propellant component comprises at least one propellant compound which is characterizable as an unsaturated hydrocarbon. In aspects, composition(s) provided herein comprise halogen-substituted propene hydrocarbon propellant compound(s). This characteristic is, in aspects, important with regard to differentiating such compounds from hydrofluoroalkane compounds further referenced herein. In aspects, HFO propellant(s) have one or more advantageous properties over traditional CFC compound(s) and HFA compound(s) such as those listed herein. In aspects, propellant compound(s) provided by the invention comprise any environmentally safe propellant compound(s). In aspects, HFO compound(s) of a propellant component have a detectably or significantly reduced adverse effect on global warming (e.g., have a detectably or significantly lower global warming potential (GWP)), have a detectably or significantly reduced adverse effect on stratospheric ozone (e.g., have a detectably or significantly lower ozone depletion potential (ODP)), or both than that of traditional CFC compound(s), HFA compound(s), or both. Accordingly, in aspects, composition(s) herein comprise a propellant component which has a detectably or significantly lower environmental impact than that of traditional CFC compound(s), HFA compound(s), or both. In aspects, such compounds comprise one or more HFO compounds.

In aspects, HFO compound(s) can be any non-toxic (or, e.g., toxicologically safe), non-flammable hydrofluoroolefin compound having suitable characteristic(s) for use in delivering composition(s) herein in aerosolized form, such as, e.g., by an inhaler device such as a metered dose inhaler (e.g., a pressurized metered dose inhaler). For example, such HFO compound(s) have a vapor pressure which is suitable to enable the composition(s) herein to be administered via a pressurized MDI. Further, in aspects, HFO propellant compound(s) suitable for use herein are compatible with the components of the MDI device (such as containers, valves, and sealing gaskets, etc.) employed to administer the medicament. In aspects, such components are those manufactured by traditional techniques. In aspects, such components do not require use of non-traditional materials or coatings to facilitate compatibility with the HFO propellant compound(s). This is discussed further elsewhere herein. In aspects, such an HFO is an HFO compound approved for use by a recognized regulatory body, such as, e.g., the US FDA.

In aspects, suitable HFO compounds are hydrofluoropropenes, such as, e.g., tetrafluoropropenes. Exemplary tetrafluoropropenes include, e.g., 2,3,3,3-tetrafluoropropene (HFO-1234yl) and 1,3,3,3-tetrafluoropropene (HFO-1234ze). In aspects, a propellant component of a composition herein can comprise a mixture of any two or more suitable HFO propellant compound(s).

In particular aspects, composition(s) comprising one or more HFO compounds, such as, e.g., one or more tetrafluoropropenes, for example one or both of 2,3,3,3-tetrafluoropropene (HFO-1234yl) and 1,3,3,3-tetrafluoropropene (HFO-1234ze) demonstrate detectably or significantly greater stability compared to composition(s) comprising the same or similar components, e.g., sharing one or more active pharmaceutical ingredients, sharing one or more APIs in the same amount(s), sharing one or more excipients, sharing one or more excipients in the same amount(s), or any combination thereof, but lacking such propellant(s), such as stability when stored under normal storage conditions (normal storage conditions being, e.g., about room temperature or between about 2° C. and about 8° C. as is discussed elsewhere herein) for a period of at least about 1 week, such as, e.g., ≥˜2 weeks, ≥˜3 weeks, ≥˜1 months, ≥˜6 weeks, ≥˜2 months, ≥˜10 weeks, ≥˜3 months, ≥˜4 months, ≥˜5 months, ≥˜6 months, ≥˜9 months, ≥˜12 months, ≥˜18 months, ≥˜24 months, ≥˜30 months, or, e.g., ≥˜36 months. Stability of composition(s) provided by the invention is discussed in greater detail elsewhere herein.

### Miscellaneous Propellants

In aspects, composition(s) can comprise a propellant component comprising one or more propellant compound(s) not otherwise characterized here or which may be described in one or more manners other than that which may be clearly characterized elsewhere herein. In aspects, a propellant compound can be, e.g., a non-chlorinated propellant compound. In aspects, composition(s) herein can comprise non-halogenated hydrocarbon propellant compound(s). In aspects, composition(s) herein can comprise saturated hydrocarbon propellant compound(s) including propane, n-butane, and isobutane, etc. and ether(s), e.g., diethyl ether. In aspects, composition(s) can comprise, e.g., halogen-substituted hydrocarbons, for example fluorine-substituted methanes, ethanes, propanes, butanes, cyclopropanes, or cyclobutanes.

In aspects, composition(s) herein comprise a propellant component comprising one or more propellant compounds which has a global warming potential (GWP) equal to or less than that of tetrafluoropropenes such as, e.g., 2,3,3,3-tetrafluoropropene (HFO-1234yl), 1,3,3,3-tetrafluoropropene (HFO-1234ze), or both. In aspects, a propellant component of composition(s) herein can comprise one or more propellant compounds having a GWP which is at least about 0.1%, ≥0.5%, ≥1%, ≥1.5%, ≥2%, ≥2.5%, ≥3%, ≥3.5%, ≥4%, ≥4.5%, ≥5%, ≥10%, ≥15%, ≥20%, ≥25%, ≥30%, ≥35%, ≥40%, ≥45%, or, even ≥50% less than that of, 3,3,3-tetrafluoropropene (HFO-1234yl), 1,3,3,3-tetrafluoropropene (HFO-1234ze), or both.

In aspects, composition(s) herein comprise a propellant component comprising one or more propellant compounds which has an ozone depletion potential (ODP) no greater than that of tetrafluoropropenes such as, e.g., 2,3,3,3-tetrafluoropropene (HFO-1234yl), 1,3,3,3-tetrafluoropropene (HFO-1234ze), or both. In certain aspects, a propellant component of composition(s) herein can comprise one or more propellant compounds having an ODP equal to or no greater than 0.

In aspects, composition(s) herein can comprise a propellant component wherein the propellant component comprises two or more propellant compounds described within the entirety of this disclosure.

### Global Warming Potential (GWP)

In aspects, composition(s) provided by the invention comprise a propellant component wherein one or more propellant compound(s)/agent(s) comprise a global warming potential (GWP) which is detectably or significantly less than comparable composition(s), e.g., having one or more shared API(s), having one or more API(s) in similar or the same amount, having one or more shared excipient(s), having one or more shared excipient(s) in similar or the same amount, or any combination thereof. In aspects, a particular advantage of composition(s) herein is the ability to provide composition(s) having a detectably or significantly reduced environmental impact compared to composition(s) comprising propellant(s) common in the art. In specific aspects, the invention provides composition(s) comprising a bronchodilator component, e.g. comprising a long-acting bronchodilator component, comprising, e.g., an anticholinergic component, a non-anticholinergic agent, or both (e.g., comprising a tiotropium compound, e.g., tiotropium or a pharmaceutically acceptable salt or solvate thereof, a formoterol compound, e.g., formoterol or a pharmaceutically acceptable salt or solvate thereof, or both), and optionally further comprising an anti-inflammatory component comprising, e.g., a steroid component, e.g., an inhaled corticosteroid component (e.g., comprising one or more of beclomethasone, ciclesonide, and a fluticasone compound), wherein the composition is deliverable via a metered dose inhaler (MDI) and comprises a propellant having a reduced GWP compared to CFC(s), many HFA(s), or both.

In aspects, a propellant component of a composition herein can comprise one or more propellant compounds having a GWP less than about 2000, such as, e.g., ≤˜1900, ≤˜1800, ≤˜1700, ≤˜1600, or, e.g., ≤˜1500. In aspects, composition(s) herein comprise a propellant component comprising one or more compounds having a GWP of less than about 1400, such as, e.g., ≤˜1300, ≤˜1200, ≤˜1100, or, e.g., ≤˜1000. In certain further aspects, composition(s) herein comprise a propellant component comprising one or more compounds having a GWP of less than about 900, such as, e.g., ≤˜800, ≤˜700, ≤˜600, or, e.g., ≤˜500. In some aspects, composition(s) herein comprise a propellant component comprising one or more compounds having a GWP of less than about 400, such as, e.g., ≤˜300, ≤˜200, or, e.g., ≤˜100. In certain other aspects, composition(s) herein comprise a propellant component comprising one or more compounds having a GWP of less than about 90, such as, e.g., ≤˜80, ≤˜70, ≤˜60, ≤˜50, ≤˜40, ≤˜30, ≤˜20, or ≤˜10, such as, e.g., ≤˜9, ≤˜8, ≤˜7, ≤˜6, ≤˜5, ≤˜4, ≤˜3, ≤˜2, or, for example, ≤˜1. In aspects, a propellant component of a composition comprises one or more propellant compounds each having a GWP of less than about the GWP values provided in this paragraph.

In aspects, a propellant component of a composition herein comprises no propellant compound having a GWP greater than about 2000, ˜1900, ˜1800, ˜1700, ˜1600, or, e.g., ˜1500. In aspects, composition(s) herein comprise no propellant compound having a GWP of greater than about 1400, ˜1300, ˜1200, ˜1100, or, e.g., ˜1000. In certain further aspects, composition(s) herein comprise no propellant compound having a GWP of greater than about 900, ˜800, ˜700, ˜600, or, e.g., ˜500. In some aspects, composition(s) herein comprise no propellant compound having a GWP of greater than about 400, ˜300, ˜200, or, e.g., ˜100. In certain other aspects, composition(s) herein comprise no propellant compound having a GWP of greater than about 90, ˜80, ˜70, ˜60, ˜50, ˜40, ˜30, ˜20, or ˜10, such as, e.g., no greater than ˜9, ˜8, ˜7, ˜6, ˜5, ˜4, ˜3, ˜2, or, for example, no greater than ˜1.

### Ozone Depletion Potential (ODP)

According to certain embodiments, composition(s) provided by the invention, e.g., the propellant component of such composition(s), e.g., any one or more propellant compound(s)/agent(s) in such composition(s), comprise an ODP potential of no greater than about 5, no greater than about 4, no greater than about 3, no greater than about 2, or no greater than about 1. In aspects, composition(s) comprise an ODP equal to or less than that of HFA-134a. In common aspects, composition(s) comprise propellant compound(s) having an ODP no greater than 1, such as, e.g., an ODP of less than 1 or an ODP of zero. In one exemplary aspect, the propellant(s) is/are selected from the group consisting of HFO-1234yl, HFO-1234ze, HFA-134a, HFA-152a, including mixture thereof.

### Propellant Component Amount

According to certain aspects, a propellant component can be present in any amount suitable and effective for the aerosolization of composition(s) upon activation of a/the delivery device within which such a composition is provided.

In aspects, a composition comprises a single propellant component. In aspects, the propellant component comprises a single propellant compound/agent. In aspects, the propellant component comprises two or more propellant compounds/agents. In aspects, amounts provided here for the propellant component can represent, e.g., the amount of the propellant component itself, e.g., the sum total of all propellant compound(s) making up the propellant component, or, e.g., the amount of a single propellant compound if the propellant component comprises a single propellant compound or is at least substantially comprised of a single propellant compound.

In aspects, any single propellant can represent, e.g., at least about 1 wt. %, such as, e.g., ≥˜2 wt. %, ≥˜3 wt. %, ≥˜4 wt. %, ≥˜5 wt. %, ≥˜8 wt. %, ≥˜10 wt. %, ≥˜15 wt. %, ≥˜20 wt. %, ≥˜25 wt. %, ≥˜30 wt. %, ≥˜35 wt. %, ≥˜40 wt. %, ≥˜45 wt. %, ≥˜50 wt. %, ≥˜55 wt. %, ≥˜60 wt. %, ≥˜65 wt. %, ≥˜70 wt. %, ≥˜75 wt. %, ≥˜80 wt. %, ≥˜85 wt. %, ≥˜90 wt. %, or, e.g., ≥˜95 wt. %, ≥˜96 wt. %, ≥˜97 wt. %, ≥˜98 wt. %, ≥˜99 wt. %, or, e.g., can make up 100 wt. % of a propellant component. In aspects, one propellant compound/agent represents at least about most, at least about essentially all, at least about substantially all, or all of a propellant component.

In certain aspects, a propellant component is comprised of at least two propellant compounds/agents, and at least about 95%, at least about 96%, at least about 97%, at least about 98%, or, e.g., at least about 99%, such as about 100% of the propellant component is represented by a single propellant compound, establishing the percent of the propellant component represented by a dominant propellant. In aspects, composition(s) can further comprise an acid compound, such as, e.g., an organic acid compound, and the ratio of the dominant propellant to the organic acid can be a ratio as described elsewhere herein.

For example, in aspects, a propellant component can comprise two propellant compounds/agents where the first and second propellant compound(s)/agent(s) represent, e.g., ˜1 wt. % and ˜99 wt. %, ˜2 wt. wt. % and ˜98 wt. %, ˜3 wt. % and ˜97 wt. %, ˜4 wt. % and ˜96 wt. %, ˜5 wt. % and ˜95 wt. %, ˜10 wt. % and ˜90 wt. %, ˜20 wt. % and ˜80 wt. %, ˜30 wt. % and ˜70 wt. %, ˜40 wt. % and ˜60 wt. %, ˜50 wt. % and ˜50 wt. % of a propellant component, respectively. It should be made clear to the reader that such percentages are exemplary, and disclosure should be interpreted as encompassing all values in between such exemplified ranges: for example, a first propellant compound/agent representing about 6 wt. % of a propellant component and a second propellant compound/agent representing about 94 wt. % of the propellant component, or, e.g., a first propellant compound/agent representing about 15 wt. % of a propellant component and a second propellant compound/agent representing about 85 wt. % of the propellant component, or, e.g., a first propellant compound/agent representing about 26 wt. % of a propellant component and a second propellant compound/agent representing about 74 wt. % of the propellant component, or, further, e.g., a first propellant compound/agent representing about 38 wt. % of a propellant component and a second propellant compound/agent representing about 62 wt. % of a propellant component, etc.

In aspects, a propellant component can comprise two propellant compounds/agents wherein the ratio of one propellant compound/agent to the second propellant compound agent is between about 1: about 99 and about 1: about 1 (between ˜1:˜99 to ˜1:˜1), such as, for example, ˜1:˜99, ˜1:˜49, ˜1:˜24, ˜1:˜19, ˜1:˜9, ˜1:˜4, ˜1:˜3, ˜1:˜2, or, e.g., ˜1:˜1, such as, for example, ˜1:˜90, ˜1:˜80, ˜1:˜70, ˜1:˜60, ˜1:˜50, ˜1:40, ˜1:˜30, ˜1:˜20, ˜1:˜10, or, e.g., ˜1:˜1, such as between ˜1-˜99 and ˜1:˜10, ˜1:˜99 and ˜1:˜20, ˜1:˜99 and ˜1:˜30, ˜1:˜99 and ˜1:˜40, ˜1:˜99 and ˜1:˜50, ˜1:˜99 and ˜1:˜60, ˜1:˜99 and ˜1:˜70, ˜1:˜99 and ˜1:˜80, or, e.g., between ˜1:˜99 and ˜1:˜90, as in, e.g., ˜1:˜99 and ˜1:˜1, ˜1:˜90 and ˜1:˜1, ˜1:˜80 and ˜1:˜1, ˜1:˜70 and ˜1:˜1, ˜1:˜60 and ˜1:˜1, ˜1:˜50 and ˜1:˜1, ˜1:˜40 and ˜1:˜1, ˜1:˜30 and ˜1:˜1, ˜1:˜20 and ˜1:˜1, or, e.g., between about ˜1:˜10 and ˜1:˜1.

Similarly, a propellant component can comprise three or more propellant compounds/agents wherein each of the propellant compounds/agents make up at least about 0.1 wt. % of the propellant component, such as, e.g., ≥0.1 wt. %, ≥0.2 wt. %, ≥0.4 wt. %, ≥0.6 wt. %, ≥0.8 wt. %, or, e.g., ≥1 wt. % of the propellant component, and wherein no propellant makes up more than about 99.8 wt. % of the propellant component, such as, e.g., no propellant makes up more than about 99.6 wt. %, 99.2 wt. %, 98.8 wt. %, 98.4 wt. %, or, e.g., no more than about 98 wt. % of a propellant component.

According to aspects, composition(s) provided by the invention comprise a propellant component, wherein the propellant component is present in an amount representing about at least 80 wt. % of the composition, such as, e.g., ≥˜82 wt. %, ≥˜84 wt. %, ≥˜86 wt. %, ≥˜88 wt. %, or, ≥˜90 wt. % of the composition, as in, e.g., ≥˜91 wt. %, ≥˜92 wt. %, ≥˜93 wt. %, ≥˜94 wt. %, ≥˜95 wt. %, ≥˜96 wt. %, ≥˜97 wt. %, ≥˜98 wt. %, ≥˜99 wt. %, ≥˜99.2 wt. %, ≥˜99.4 wt. %, ≥˜99.5 wt. %, ≥˜99.6 wt. %, ≥˜99.7 wt. %, ≥˜99.8 wt. %, or, e.g., ≥˜99.8 wt. % of the composition.

In aspects, composition(s) comprise a propellant component, wherein the propellant component is present in an amount representing between about 80 wt. % and about 99.9 wt. % of the composition, ˜82 wt. %-˜99.9 wt. %, ˜84 wt. %-˜99.9 wt. %, ˜86 wt. %-˜99.9 wt. %, ˜88 wt. %-˜99.9 wt. %, ˜90 wt. %-˜99.9 wt. %, ˜92 wt. %-˜99.9 wt. %, ˜94 wt. %-˜99.9 wt. %, ˜96 wt. %-˜99.9 wt. %, or, e.g., between ˜98 wt. %-˜99.9 wt. % of the composition.

In aspects, composition(s) comprise a propellant component, wherein the propellant component is present in an amount representing between about 80 wt. % and about 99.8 wt. % of the composition, ˜80 wt. %-˜99.6 wt. %, ˜80 wt. %-˜99.4 wt. %, ˜80 wt. %-˜99.2 wt. %, ˜80 wt. %-˜99 wt. %, ˜80 wt. %-˜98 wt. %, ˜80 wt. %-˜97 wt. %, ˜80 wt. %-˜96 wt. %, ˜80 wt. %-˜95 wt. %, ˜80 wt. %-˜94 wt. %, ˜80 wt. %-˜93 wt. %, ˜80 wt. %-˜92 wt. %, ˜80 wt. %-˜91 wt. %, or, e.g., between ˜80 wt. %-˜90 wt. % of the composition.

### Aerosolization Means

In this and other component, compound/agent/constituent, or method aspect(s) of the invention, the invention also can be characterized as comprising a “means” for aerosolizing composition(s), e.g., a means for providing API(s) within a composition in aerosolized form. In such a respect, any known equivalents of such named aerosolization component(s), compound(s), or constituent(s), such as, e.g., is exemplified in one aspect as propellant compound(s) or a propellant component, can also be, e.g., are, incorporated into composition(s) or method(s) of the invention. As with other sections similarly described herein, any of the component(s), compound(s), agent(s), constituent(s), or, e.g., method(s) of the invention can be, where suitable, described as means (e.g., the above-described aerosolization component(s), compound(s), or constituent(s) can be described as aerosolization means or means for aerosolization).

### Excipient Component

In aspects, composition(s) provided herein can comprise an excipient component. In aspects, an excipient component can comprise one or more subcomponents, e.g., characterized by the type of compound(s) or activity of compound(s) therein. In aspects, an excipient component, an excipient component subcomponent, or both, can comprise one or more excipient compound(s)/agent(s). In aspects, an excipient compound/agent is any agent not otherwise characterized herein as an active pharmaceutical ingredient or a propellant compound/agent. In aspects, a propellant compound/agent can also be considered an excipient.

In certain aspects, an excipient compound/agent or group(s) of excipient compound(s)/agent(s) can detectably or significantly stabilize a composition, such as, e.g., detectably or significantly physically stabilize a suspension (e.g., maintain the composition in a stabile suspension for a detectably or significantly longer period of time than such a composition without such component(s) or compound(s)/agent(s) or group(s) thereof).

In certain aspects, an excipient compound/agent or group(s) of excipient compound(s)/agent(s) can detectably or significantly stabilize a composition, such as, e.g., detectably or significantly chemically stabilize one or more APIs (e.g., prevent the detectable or significant breakdown of one or more APIs, prevent the detectable or significant formation of one or more degradation products, or, e.g., any combination thereof) for a detectably or significantly longer period of time than such a composition without such component(s) or compound(s)/agent(s) or group(s) thereof.

In aspects, suitable excipients used in the formulation of pharmaceutical composition(s) herein can comprise any pharmaceutically acceptable, compositionally compatible compound(s), such as, for example, one or more, but not limited to, solvent(s)/co-solvent(s), low volatility component(s)/compound(s)/agent(s), stabilizer(s), dispersing agent(s), suspension agent(s), pH adjusting agent(s), preservative(s), antioxidant(s), surface active agent(s), surfactant(s), viscosity-enhancing agent(s), bulking agent(s), absorbent(s), performance modulating agent(s), carrier(s), performance modulating agent(s), etc. and mixtures of any or all thereof. In certain aspects, an excipient may be characterizable according to the type of compound as opposed to or in addition to its functional activity, such as, e.g., characterizing an excipient as an organic acid, mineral acid, etc. In aspects, one or more compound(s)/agent(s) can provide one or more detectable or significant activities, thus can be characterized in a plurality of ways, e.g., providing both solvent and suspension activity or, e.g., providing both solvent and viscosity enhancement activity. In aspects, uncontradicted, any compound provided under any specific section here can be interpreted to be incorporated under other sections as applicable. Amounts disclosed, uncontradicted or unless stated otherwise, can be interpreted to apply to such compound when such compound is described as or known in the art as providing a different activity.

Herein, use of the term “performance modulating agent” in describing an excipient can be used to refer to a compound/agent (or, e.g., molecule) which provides a detectable or significant increase in the dissolution of one or more API(s) in composition(s), or which provides a detectable or significant increase in the suspension of one or more API(s) in composition(s). For example, in aspects, polyethylene glycol (e.g., PEG-600 or, e.g., PEG-1000), glycerol, or both, can be referred to herein as performance modulating molecules/compounds. As stated above, such a compound/agent/molecule can, in aspects, belong to one or more excipient group(s) herein. Aspects of the invention include formulations/compositions comprising effective amount(s) of one or more of such agent(s).

### Surfactant(s)

In aspects, composition(s) herein can comprise a surfactant component comprising one or more surfactant compound(s)/agent(s). In aspects, composition(s) comprise one or more surfactant compound(s)/agent(s). In aspects, a surfactant is a composition agent/constituent which detectably or significantly reduces surface tension, detectably or significantly reduces particle aggregation (e.g., detectably or significantly improves upon the separation of particles to detectably or significantly prevent their settling or clumping), detectably or significantly reduces particulate sticking, clogging, or other interference with delivery device operation or successful delivery of an appropriate dose of composition, detectably or significantly increases spreading or wetting of one or more component(s)/compound(s)/agent(s), or any or all thereof. In aspects, surfactant compound(s) present in composition(s) herein are surface active compound(s). In certain aspects, surfactant compound(s) can, e.g., detectably or significantly improve physical stability of a composition by, e.g., minimizing particle-particle interaction(s). In certain aspects, surfactant compound(s) may, e.g., detectably or significantly increase the absorption of composition(s), component(s) of composition(s), compound(s) of compositions, or any or all thereof, into lung tissue(s). In aspects, surfactant compound(s) present in composition(s) herein address the interface between two compositional phases. In aspects, a compound of a composition described herein as a surfactant comprises both a hydrophilic group and a hydrophobic group interacting via electrostatic interactions with surface(s) of, e.g., particle(s), a suspension medium, a container, etc.

In aspects, a surfactant component/constituent can provide detectable or significant stabilization to composition(s), e.g., physical stabilization. For example, in aspects composition(s) comprising surfactant(s) demonstrate detectably or significantly increased stability compared to at least substantially similar composition(s) lacking such surfactant(s). In aspects, composition constituent(s) described as stabilizer(s) may or may not demonstrate detectable or significant surfactant capability(ies). For example, in aspects a stabilizer may provide a detectable or significant increase in composition stability compared to composition(s) lacking such a stabilizing agent, and, e.g., in aspects, such a stabilizing agent may provide detectable or significant pH modulation activity, however, in aspects, such a stabilizer may not provide detectable or significant surfactant activity.

In aspects, one or more surfactant compound(s)/agents(s) detectably or significantly increase the stability the pharmaceutical composition. In aspects, one or more surfactant compound(s) agents provide lubrication to one or more delivery device components, e.g., provide lubrication to a valve system of an MDI. According to certain aspects, composition(s) herein are provided as a suspension, wherein surfactant(s) are present in the suspension and such surfactant(s) detectably or significantly prevent detectable or significant particle agglomeration, crystal growth, adhesion to the container (e.g., container closure), or any combination thereof. In aspects, surfactant(s) present in such composition(s) detectably or significantly reduce the rate of separation between API(s) and propellant(s) (or, e.g., API(s) and the propellant system). According to certain aspects, compositions) herein are provided as a solution, wherein surfactant(s) are present in the solution and such surfactant(s) detectably or significantly increase the solubility of API(s). In certain aspects, the presence of surfactant(s) detectably or significantly prevents, or, e.g., decreases the frequency of, the sticking of two or more components of a delivery device, such as, e.g., prevents or decreases the frequency of valve sticking in an MDI.

In aspects, a constituent of a surfactant component can comprise, e.g., one or more compound(s) recognized in the art as being capable of providing one or more activity(ies) aside from that of a surfactant. In aspects, a surfactant compound(s) may further be characterizable as a dispersing agent, and, in aspects, uncontradicted, can encompass dispersing agent(s) described herein.

In aspects, composition(s) herein lack compound(s)/agent(s) which demonstrate detectable or significant surfactant activity. In aspects, any surfactant compound(s)/agent(s) of composition(s) herein are not characterizable as pharmaceutically acceptable and compositionally compatible small compound stabilizing agent(s) having a molecular weight of less than about 200 g/mol, providing detectable or significant chelating activity, or both. In aspects, any surfactant compound(s)/agent(s) of composition(s) herein are not characterizable as a pharmaceutically acceptable and compositionally compatible small compound stabilizing agent(s) having a molecular weight of less than about 200 g/mol, providing detectable or significant chelating activity, or both, capable of detectably or significantly preventing or delaying any change in the homogeneity of the composition resulting in (a) the amount of light capable of passing through the composition to be reduced or (b) the composition no longer being characterizable as a solution. In aspects, composition(s) do not comprise a compound recognized in the art as a common surfactant. In aspects, composition(s) do not comprise a compound having an oil affinity greater than or equal to that of oleic acid, e.g., do not have an HLB value equal to or less than 1. In aspects, composition(s) do not comprise a compound wherein the amount of compound required to reduce the maximum surface tension of water is less than that of oleic acid. In certain aspects, composition(s) provided herein do not comprise (e.g., composition(s) are free of) any compound(s)/agent(s) providing significant surfactant activity.

In further aspects, a surfactant herein is a compound comprising one or more series of carbons, e.g., carbon chains, comprising at least 12 contiguous carbon atoms. For example, in aspects, a surfactant compound present in composition(s) can comprise one or more series of carbons, e.g., carbon chain(s), comprising at least about 12, ˜13, ˜14, ˜15, ˜16, ˜17, ˜18, or, even more, contiguous carbon atoms.

In an additional aspect, a surfactant herein is a compound comprising a ratio of carbon atoms to any non-hydrogen atom present in the compound of at least about 1.5:1, such as, e.g., at least about 2:1, at least about 2.5:1, at least about 3:1, at least about 3.5:1, or, e.g., at least about 4:1. In certain aspects, a surfactant herein is a compound comprising a ratio of carbon atoms present in the compound to any non-hydrogen atom in the compound is at least about 4:1, such as e.g., ≥˜5:1, ≥˜6:1, ≥˜7:1, ≥˜8:1, ≥˜9:1, ≥˜10:1, ≥˜11:1, ≥˜12:1, ≥˜13:1, ≥˜14:1, ≥˜15:1, ≥˜16:1, ≥˜17:1, ≥˜18:1, ≥˜19:1, ≥˜20:1, or, e.g., ≥˜21:1. In certain aspects, a surfactant herein is a compound comprising a ratio of carbon atoms present in the compound to any non-hydrogen atom in the compound is about 4:1, such as, e.g., ˜5:1, ˜6:1, ˜7:1, ˜8:1, ˜9:1, ˜10:1, ˜11:1, ˜12:1, ˜13:1, ˜14:1, ˜15:1, ˜16:1, ˜17:1, ˜18:1, ˜19:1, ˜20:1, or, e.g., ˜21:1.

In aspects, composition(s) can comprise a surfactant component comprising any pharmaceutically acceptable and compositionally compatible surfactant compound(s)/agent(s). According to certain aspects, a surfactant component of composition(s) provided herein can comprise, e.g., one or more ionic surfactants. In aspects, a surfactant component of composition(s) provided herein can comprise, e.g., one or more non-ionic surfactants. In aspects, a surfactant component of composition(s) provided herein can comprise, e.g., salt(s)s of stearic acid(s) such as magnesium stearate, ester(s) such as ascorbyl palmitate, isopropyl myristate, tocopherol ester(s) such as oleic acid, sorbitan trioleate, lecithin, isopropyl myristate, tyloxapol, polysorbate(s) such as polysorbate 80, polysorbate 20, polysorbate 40, vitamin E-TPGS, macrogol hydroxystearate(s) such as macrogol-15-hydroxystearate, acetylated monoglycerides such as Myvacet 9-45 and Myvacet 9-08, polyoxyethylene ethers, ethyloleate, glyceryl trioleate, glyceryl monolaurate, glyceryl monooleate, glyceryl monostearate, glyceryl monoricinoleate, cetylalcohol, sterylalcohol, cetyl pyridinium chloride, block polymers, natural oils, sorbitan fatty acid esters such as sorbitan trioleate, polyethoxylated sorbitan fatty acid esters such as polyethoxylated sorbitan trioleate, sorbimacrogol oleate, synthetic amphotensides (tritons), ethylene oxide ethers of octylphenolformaldehyde condensation products, phosphatides such as lecithin, polyethoxylated fats, polyethoxylated oleotriglycerides, polyethoxylated fatty alcohols, etc.

In aspects, a surfactant component can comprise, e.g., one or more surfactant compound(s)/agent(s)/constituent(s) selected from the vast class like oils known in the art such as, but not limited to, corn oil, olive oil, cottonseed oil and sunflower seed oil, mineral oils (e.g., paraffin), oleic acid, phospholipids such as lecithin, or sorbitan fatty acid esters such as, e.g., sorbitan trioleate, or Tween 20, Tween 60, Tween 80, polyethylene glycols such as PEG-25, PEG-100, PEG-600, PEG-1000, glyceryl trioleate, PVP (polyvinylpyrrolidone), PVP VA64, lysine, glycine, leucine, phospholipid, cyclodextrin, cremophor, etc. In aspects, composition(s) can comprise a mixture of any two or more compound(s)/agent(s)/surfactant component constituent(s) described in this paragraph or elsewhere herein.

According to certain aspects, a surfactant component constituent is selected from oleic acid, span 85, Tween 80, cetylpyridinium chloride, lecithin, palmitic acid, or a combination of any or all thereof.

### Surfactant Amount

In certain aspects, composition(s) herein lack any component or compound/agent which is characterizable as a surfactant. In aspects, composition(s) herein are characterizable as surfactant-free.

Here and in similar sections herein, amounts presented can, in aspects, represent the amount of a single surfactant compound/agent, the amount of a surfactant component (e.g., representing the total amount of surfactant compound(s) in a composition, or both. In aspects, a surfactant component of a composition can comprise two or more compounds each present in a composition in amount(s) described here.

In aspects, composition(s) herein comprise an effective amount of a pharmaceutically acceptable and compositionally compatible surfactant component. According to aspects, a surfactant component can be present in an amount representing between about 0.0001 wt. % and about 0.5 wt. %, such as, e.g., ˜0.0001 wt. %-˜0.45 wt. %, ˜0.0001 wt. %-˜0.4 wt. %, ˜0.0001 wt. %-˜0.35 wt. %, ˜0.0001 wt. %-˜0.3 wt. %, ˜0.0001 wt. %-˜0.25 wt. %, ˜0.0001 wt. %-˜0.2 wt. %, ˜0.0001 wt. %-˜0.15 wt. %, or, e.g., ˜0.0001 wt. %-˜0.45 wt. %.

In aspects, a surfactant component can be present in an amount representing between about 0.0002 wt. % and about 0.5 wt. %, such as, e.g., ˜0.0003 wt. %-˜0.5 wt. %, ˜0.0004 wt. %-˜0.5 wt. %, ˜0.0005 wt. %-˜0.5 wt. %, ˜0.0006 wt. %-˜0.5 wt. %, ˜0.0007 wt. %-˜0.5 wt. %, ˜0.0008 wt. %-˜0.5 wt. %, ˜0.0009 wt. %-˜0.5 wt. %, or, e.g., ˜0.001 wt. %-˜0.5 wt. %.

In aspects, a surfactant component can be present in an amount representing between about 0.0002 wt. % and about 0.45 wt. %, such as, e.g., ˜0.0003 wt. % and about 0.4 wt. %, ˜0.0004 wt. % and about 0.35 wt. %, ˜0.0005 wt. %-˜0.3 wt. %, ˜0.0006 wt. %-˜0.25 wt. %, ˜0.0007 wt. %-˜0.2 wt. %, ˜0.0008 wt. %-˜0.15 wt. %, ˜0.0009 wt. %-˜0.1 wt. %, or, e.g., ˜0.001 wt. %-˜0.1 wt. %.

In certain aspects, composition(s) comprise no more than about 0.3 wt. % of a surfactant component, such as, e.g., ≤˜0.25 wt. %, ≤˜0.2 wt. %, ≤˜0.15 wt. %, or, e.g., ≤˜0.1 wt. % of a surfactant component.

In some aspects, composition(s) can comprise a surfactant, such as, e.g., PEG-1000, in an amount of between about 0.005 wt. % and about 0.15 wt. %, such as, e.g., about 0.001 wt. %. In certain aspects, such a composition can be provided as a suspension. In certain aspects, such a composition can be provided as a solution-suspension hybrid. In certain specific aspects, composition(s) can comprise a surfactant such as, e.g., oleic acid, present in an amount of between about 0.001 wt. % and about 0.1 wt. %. In certain aspects, such composition(s) are provided as a suspension.

According to certain aspects, wherein it is appropriate or, e.g., recognizable by those in the art, to characterize a particular surfactant as having one or more functional activities which is/are characterizable as an activity other than that of a surfactant, e.g., characterizable as a dispersing agent or other identifiable characteristic (though, in aspects, a “surfactant” and a “dispersing agent” may in fact share some, most, generally all, or all functional activity(ies)), amount(s) disclosed herein as representing such activity, e.g., the amount of a dispersing compound/agent or component present in composition(s), can be applied to any one or more surfactant compound(s)/agent(s)/constituent(s) described here or can be applied to the surfactant component described here.

### Surfactant Means

In this and other component, compound/agent/constituent, or method aspect(s) of the invention, the invention also can be characterized as comprising a “means” for providing one or more surfactant characteristic(s), e.g., detectably or significantly increasing the stability of compound(s) or composition(s), lubricating one or more delivery device (e.g., MDI) component(s) (e.g., valve of an MDI), reducing surface tension, detectably or significantly reducing particle aggregation, detectably or significantly reducing particulate sticking, clogging, or other interference with delivery device operation or successful delivery of an appropriate dose of composition, detectably or significantly increasing spreading or wetting of one or more component(s)/compound(s)/agent(s), or any or all thereof. In such a respect, any known equivalents of such named surfactants are incorporated into composition(s) or method(s) of the invention. As with other sections similarly described herein, any of the component(s), compound(s), agent(s), constituent(s), or, e.g., method(s) of the invention can be, where suitable, described as means (e.g., the above-described surfactant component(s), compound(s), or constituent(s) can be described as surfactant means or means for providing a detectable or significant increase in the stability of compound(s) or composition(s), lubricating one or more delivery device (e.g., MDI) component(s) (e.g., valve of an MDI), reducing surface tension, detectably or significantly reducing particle aggregation (e.g., detectably or significantly improving the separation of particles and preventing their settling or clumping), detectably or significantly reducing particulate sticking, clogging, or other interference with delivery device operation or successful delivery of an appropriate dose of composition, detectably or significantly increasing spreading or wetting of one or more component(s)/compound(s)/agent(s), or any or all thereof.)

### Solvent(s)

In aspects, composition(s) herein can comprise a solvent component comprising one or more solvent compound(s)/agent(s). In aspects, a solvent compound/agent is any compound/agent which detectably or significantly increases the compatibility between any one or more API(s) and any one or more propellant(s). In aspects, a solvent compound/agent can be characterizable as a co-solvent. In aspects, a solvent compound/agent can be characterizable as a low volatility compound. In aspects, a solvent agent/compound detectably or significantly increases the amount of time that an API(s) are capable of remaining solubilized in the pharmaceutical composition, such that the composition is characterizable as a solution.

In aspects, a constituent of a solvent component can comprise, e.g., one or more compound(s) recognized in the art as being capable of providing one or more activity(ies) aside from that of a solvent, or, e.g., co-solvent. In aspects, solvent compound(s) may further be characterizable as, e.g., a viscosity enhancing agent. In aspects, a solvent compound can further be characterizable as such a compound and, uncontradicted, in aspects, can encompass such compounds and be present in such amounts described herein.

In aspects, composition(s) can comprise a solvent component comprising any pharmaceutically acceptable and compositionally compatible solvent compound(s)/agent(s). According to certain aspects, a solvent component of composition(s) provided herein can comprise, e.g., one or more C1-C6 alcohol compound(s)/agent(s). In aspects, the C1-C6 alcohol compound(s)/agent(s) is characterizable as an alkyl alcohol. In aspects, the C1-C6 alcohol compound(s)/agent(s) is characterizable as an aliphatic alcohol. In aspects, composition(s) comprise one or more C1-C6 alcohols, such as, e.g., C2-C6 aliphatic alcohols such as, but not limited to, ethyl alcohol (ethanol) and isopropyl alcohol. In aspects, a solvent component is characterizable as a lower branched or linear alkyl (C1-C4) alcohol. In aspects, a solvent is characterizable as an organic alcohol (such as, e.g., methanol, ethanol, propanol, butanol, etc.) In aspects, composition(s) comprise a solvent component comprising one or more sugar alcohol(s). In aspects, composition(s) comprise a solvent component comprising glycol(s), such as, but not limited to, propylene glycol, polyethylene glycols, polypropylene glycols, glycol ethers, block copolymers of oxyethylene and oxypropylene, other substances such as, but not limited to glycerol, isopropyl myristate polyoxyethylene alcohols, polyoxyethylene fatty acid esters, hydrocarbons such as, but not limited to, n-propane, n-butane, isobutane, n-pentane, iso-pentane, neo pentane, n-hexane, ethers such as but not limited to diethyl ether, glycerol, glycerin, etc. In aspects, a solvent component comprises a compound having a higher polarity than that of one or more propellant compound(s). In aspects composition(s) can comprise a mixture of any two or more compound(s)/agent(s)/surfactant component constituent(s) described in this paragraph or elsewhere herein. In certain aspects, composition(s) comprise a pharmaceutically acceptable and compositionally compatible small compound stabilizing agent comprising an organic acid, a sugar alcohol, or both. In aspects, for example, composition(s) can comprise ethanol, glycol, or both ethanol and glycol.

In certain aspects, one or more solvent compound(s) is an ex-actuator size affecting compound. In aspects, an ex-actuator size affecting compound can be, e.g., glycerol. In certain aspects, other ex-actuator size affecting compound(s) can be, e.g., ethanol, propylene glycol, polyethylene glycol (e.g., polyethylene glycol compound(s) having a molecular weight of between about 400 and about 4000 g/mol), oleic acid, etc. In aspects, other ex-actuator size affecting compound(s) can be, e.g., any one or more compound(s) which detectably or significantly modulate composition viscosity, composition density, surface property(ies) of the composition, pressure of the composition, or combination(s) of any or all thereof.

According to certain aspects, a solvent component constituent is selected from ethanol, glycerin, glycerol (e.g., pure compound (glycerol) or commercial grade compound(s) as glycerin/glycerine) water, menthol, or a combination of any or all thereof. In aspects, composition(s) comprise ethanol. In aspects, a solvent component can comprise, e.g., ethanol (95%), anhydrous ethanol.

### Solvent Amount

According to certain aspects, composition(s) herein lack any component or compound/agent which is characterizable as a solvent or co-solvent. In aspects, composition(s) are characterizable as ethanol free.

Here and in similar sections herein, amounts presented can, in aspects, represent the amount of a single solvent compound/agent, the amount of a solvent component (e.g., representing the total amount of solvent (or, e.g., co-solvent) compound(s) in a composition), or both. In aspects, a solvent component of a composition can comprise two or more compounds each present in a composition in amount(s) described here.

In aspects, a sufficient amount of a solvent agent/compound or, e.g., solvent component, is present to ensure adequate that an adequate concentration of drug (e.g., a therapeutically effective amount an API or API component) can be dissolved in the propellant/propellant system. In aspects, a sufficient amount of ethanol is present to ensure adequate that an adequate concentration of drug (e.g., a therapeutically effective amount an API or API component) can be dissolved in the propellant/propellant system.

In aspects, composition(s) herein comprise an effective amount of a pharmaceutically acceptable and compositionally compatible solvent component. According to certain aspects, composition(s) provided herein can comprise a solvent component wherein the solvent component is present in an amount representing between about 0.1 wt. % and about 25 wt. % of the composition, such as, e.g., ˜0.1 wt. %-˜22.5 wt. %, ˜0.1 wt. %-˜20 wt. %, ˜0.1 wt. %-˜17.5 wt. %, ˜0.1 wt. %-˜15 wt. %, ˜0.1 wt. %-˜12.5 wt. %, ˜0.1 wt. %-˜10 wt. %, ˜0.1 wt. %-˜7.5 wt. %, or, e.g., ˜0.1 wt. %-˜5 wt. % of the composition.

In aspects, composition(s) provided herein can comprise a solvent component wherein the solvent component is present in an amount representing between about 0.5 wt. %-˜25 wt. %, such as, e.g., ˜1 wt. %-˜25 wt. %, ˜1.5 wt. %-˜25 wt. %, ˜2 wt. %-˜25 wt. %, ˜2.5 wt. %-˜25 wt. %, ˜3 wt. %-˜25 wt. %, ˜3.5 wt. %-˜25 wt. %, ˜4 wt. %-˜25 wt. %, ˜4.5 wt. %-˜25 wt. %, or, e.g., ˜5 wt. %-˜25 wt. % of the composition.

In aspects, composition(s) provided herein can comprise a solvent component wherein the solvent component is present in an amount representing ˜0.2 wt. %-˜22.5 wt. %, ˜0.25 wt. %-˜20 wt. %, ˜0.3 wt. %-˜17.5 wt. %, ˜0.35 wt. %-˜15 wt. %, ˜0.4 wt. %-˜12.5 wt. %, or, e.g., ˜0.5 wt. %-˜10 wt. %, such as, e.g., ˜1 wt. %-˜7.5 wt. %, or ˜1.5 wt. %-˜5 wt. % of the composition. In aspects, composition(s) comprise, e.g., glycerol, ethanol, or both, in an amount of between about 0.1 wt. % and about 25 wt. % of the composition.

In certain aspects, composition(s) comprise a solvent component present in an amount of between about 0 wt. % and about 2.5 wt. %, such as, e.g., ˜0.1 wt. %-2.5 wt. %, ˜0.2 wt. %-2.5 wt. %, ˜0.3 wt. %-2.5 wt. %, ˜0.4 wt. %-2.5 wt. %, ˜0.5 wt. %-2.5 wt. %, ˜0.6 wt. %-2.5 wt. %, ˜0.7 wt. %-2.5 wt. %, ˜0.8 wt. %-2.5 wt. %, ˜0.9 wt. %-2.5 wt. %, or, e.g., ˜1 wt. %-2.5 wt. % of the composition.

In aspects, composition(s) comprise a solvent component present in an amount of between about 0 wt. % and about 2.25 wt. %, such as, e.g., ˜0 wt. %-˜2 wt. %, ˜0 wt. %-˜1.75 wt. %, ˜0 wt. %-˜1.5 wt. %, ˜0 wt. %-˜1.25 wt. %, or, e.g., ˜0 wt. %-˜1 wt. % of the composition.

In aspects, composition(s) comprise a solvent component present in an amount of between about 0.1 wt. % and about 2.25 wt. %, such as, e.g., ˜0.2 wt. %-˜2 wt. %, ˜0.3 wt. %-˜1.75 wt. %, ˜0.4 wt. %-˜1.5 wt. %, ˜0.5 wt. %-˜1.5 wt. %, ˜0.6 wt. %-˜1.5 wt. %, ˜0.7 wt. %-˜1.5 wt. %, ˜0.8 wt. %-˜1.5 wt. %, ˜0.9 wt. %-˜1.5 wt. %, or, e.g., ˜1 wt. %-˜1.5 wt. %, or, e.g., ˜0.6 wt. %-˜1 wt. % of the composition. In aspects, composition(s) can comprise, e.g., glycerol, in an amount of between about 0 wt. %-about 2.5 wt. % or between about 0 wt. % and about 1 wt. %, such as, e.g., ˜0.1 wt. %-˜2.5 wt. %, ˜0.2 wt. %-˜2 wt. %, or, e.g., ˜0.7 wt. %-˜1.7 wt. %, ˜0.8 wt. %-˜1.6 wt. %, ˜0.9-˜1.6 wt. %, or, e.g., ˜1 wt. %-˜1.5 wt. %.

In aspects, composition(s) comprise a solvent component in an amount of between about 10 wt. % and about 25 wt. %, such as, e.g., ˜10 wt. %-˜24 wt. %, ˜10 wt. %-˜23 wt. %, ˜10 wt. %-˜22 wt. %, ˜10 wt. %-˜21 wt. %, ˜10 wt. %-˜20 wt. %, ˜10 wt. %-˜19 wt. %, ˜10 wt. %-˜18 wt. %, ˜10 wt. %-˜17 wt. %, ˜10 wt. %-˜16 wt. %, or, e.g., ˜10 wt. %-˜15 wt. %.

In aspects, composition(s) comprise a solvent component in an amount of between about 10.5 wt. % and about 25 wt. %, such as, e.g., ˜11 wt. %-˜25 wt. %, ˜11.5 wt. %-˜25 wt. %, ˜12 wt. %-˜25 wt. %, ˜12.5 wt. %-˜25 wt. %, ˜13 wt. %-˜25 wt. %, ˜13.5 wt. %-˜25 wt. %, ˜14 wt. %-˜25 wt. %, ˜14.5 wt. %-˜25 wt. %, or, e.g., ˜15 wt. %-˜25 wt. %.

In aspects, composition(s) comprise a solvent component in an amount of between about 10.5 wt. % and about 24 wt. %, such as, e.g., ˜11 wt. %-˜23 wt. %, ˜11.5 wt. %-˜22 wt. %, ˜12 wt. %-˜21 wt. %, ˜12.5 wt. %-˜20 wt. %, ˜13 wt. %-˜19 wt. %, ˜13.5 wt. %-˜18 wt. %, ˜14 wt. %-˜17 wt. %, or, e.g., ˜14 wt. %-˜16 wt. %. In aspects, composition(s) can comprise ethanol in an amount of between about 10 wt. % and about 25 wt. %, such as, e.g., about 12 wt. % and about 18 wt. %, about 13 wt. % and about 17 wt. %, about 14 wt. % and about 16 wt. %, or, e.g., about 14.5 wt. % and about 15.5 wt. %, such as ethanol in an amount of about 15 wt. %.

According to certain aspects, composition(s) can comprise a solvent component in an amount representing at least about 5 wt. % of the composition, such as, e.g., at least about 5.5 wt. %, ≥˜6 wt. %, ≥˜6.5 wt. %, ≥˜7 wt. %, ≥˜7.5 wt. %, ≥˜8 wt. %, ≥˜8.5 wt. %, ≥˜9 wt. %, ≥˜9.5 wt. %, ≥˜10 wt. %, ≥˜10.5 wt. %, ≥˜11 wt. %, ≥˜11.5 wt. %, ≥˜12 wt. %, ≥˜12.5 wt. %, ≥˜13 wt. %, ≥˜13.5 wt. %, ≥˜14 wt. %, ≥˜14.5 wt. %, ≥˜15 wt. %, ≥˜15.5 wt. %, ≥˜16 wt. %, ≥˜16.5 wt. %, ≥˜17 wt. %, ≥˜17.5 wt. %, ≥˜18 wt. %, ≥˜18.5 wt. %, ≥˜19 wt. %, ≥˜19.5 wt. %, or, e.g., ≥˜20 wt. %, such as, e.g., in an amount of at least ˜6 wt. %, at least ˜11.25 wt. %, or, e.g., at least about 15 wt. % of the composition. In aspects, composition(s) comprise a C1-C6 alcohol compound/agent, e.g., a C1-C6 alcohol compound/agent characterizable as an alkyl alcohol, an aliphatic alcohol, or both, such as, e.g., a lower branched or linear alkyl (C1-C4) alcohol, such as ethanol, in an amount of at ≥˜4%, ≥˜5%, ≥˜6%, ≥˜7%, ≥˜8%, ≥˜9%, ≥˜10%, ≥˜11%, ≥˜12%, ≥˜13%, ≥˜14%, or, e.g., ≥˜15%.

In aspects, the amount of a C1-C6 alcohol compound/agent, e.g., a C1-C6 alcohol compound/agent characterizable as an alkyl alcohol, an aliphatic alcohol, or both, such as, e.g., a lower branched or linear alkyl (C1-C4) alcohol, such as ethanol, present in a composition is no more than about 20 wt. %, such as, e.g., ≤˜19 wt. %, ≤˜18 wt. %, ≤˜17 wt. %, ≤˜16 wt. %, ≤˜15 wt. %, ≤˜14 wt. %, ≤˜13 wt. %, ≤˜12 wt. %, ≤˜11 wt. %, or, e.g., ≤˜10 wt. %.

In aspects, the amount of a C1-C6 alcohol compound/agent, e.g., a C1-C6 alcohol compound/agent characterizable as an alkyl alcohol, an aliphatic alcohol, or both, such as, e.g., a lower branched or linear alkyl (C1-C4) alcohol, such as ethanol, present in a composition is no less than about 10 wt. %, such as, e.g., ≥˜11 wt. %, ≥˜12 wt. %, ≥˜13 wt. %, ≥˜14 wt. %, ≥˜15 wt. %, ≥˜16 wt. %, ≥˜17 wt. %, or, e.g., ≥˜18 wt. %.

According to certain specific aspects, composition(s) comprise a C1-C6 alcohol compound/agent characterizable as an alkyl alcohol, an aliphatic alcohol, or both, such as, e.g., a lower branched or linear alkyl (C1-C4) alcohol, such as ethanol, in an amount representing between about 13 wt. % and about 17 wt. % of a composition, such as, e.g., ˜13.5 wt. %-˜16.5 wt. % of a composition, ˜14 wt. %-˜16 wt. % of a composition, ˜14.5 wt. %-˜15.5 wt. % of a composition, or, e.g., about 15 wt. % of a composition. According to certain specific aspects, composition(s) comprise a C1-C6 alcohol compound/agent characterizable as an alkyl alcohol, an aliphatic alcohol, or both, such as, e.g., a lower branched or linear alkyl (C1-C4) alcohol, such as ethanol, in an amount representing between about 0.1 wt. % and about 2 wt. % of a composition, such as, e.g., ˜0.5 wt. %-˜1.5 wt. % of a composition, ˜0.7 wt. %-˜1.3 wt. % of a composition, ˜0.8 wt. %-˜1.2 wt. % of a composition, or, e.g., about 1 wt. % of a composition.

In certain aspects, composition(s) can comprise, e.g., glycerol, wherein glycerol is present in an amount of no more than about 2 wt. % of a composition, such as, e.g., ≤˜1.9 wt. %, ≤˜1.8 wt. %, ≤˜1.7 wt. %, ≤˜1.6 wt. %, ≤˜1.5 wt. %, ≤˜1.4 wt. %, ≤˜1.3 wt. %, ≤˜1.2 wt. %, ≤˜1.1 wt. %, ≤˜1 wt. %, ≤˜0.9 wt. %, ≤˜0.8 wt. %, ˜0.7 wt. %, ˜0.75 wt. %, or, e.g., ≤˜0.6 wt. %, ≤˜0.55 wt. %, ≤˜0.5 wt. %, ≤˜0.45 wt. %, ≤˜0.4 wt. %, ≤˜0.35 wt. %, ≤˜0.3 wt. %, ≤˜0.25 wt. %, ≤˜0.2 wt. %, ≤˜0.15 wt. %, ≤˜0.1 wt. %, ≤˜0.05 wt. %, or, e.g., ≤˜0.01 wt. % of a composition.

In aspects, composition(s) can comprise, e.g., glycerol, wherein glycerol is present in an amount of between about 0.7 wt. % and about 1.7 wt. %, such as, e.g., ˜0.8 wt. %-˜1.6 wt. %, ˜0.9 wt. %-˜1.6 wt. %, or, e.g., ˜1 wt. %-˜1.5 wt. %.

In aspects, composition(s) can comprise, e.g., glycerol, in an amount of between about 0.1 wt. % and about 1 wt. %, such as, e.g., ˜0.2 wt. %-˜1 wt. % or ˜0.4 wt. %-˜1 wt. %, such as, e.g., ˜0.1 wt. %-˜0.8 wt. % or ˜0.1 wt. %-˜0.6 wt. %, as in, e.g., between about 0.2 wt. % and about 0.8 wt. %, e.g., ˜0.4 wt. %-˜0.6 wt. %, such as, for example, ˜0.5 wt. %.

In certain aspects, composition(s) can comprise, e.g., glycerol, wherein glycerol is present in an amount of no less than about 1 wt. %, such as, e.g., ≥˜1.5 wt. %, ≥˜2 wt. %, ≥˜2.5 wt. %, ≥˜3 wt. %, ≥˜3.5 wt. %, ≥˜4 wt. %, ≥˜4.5 wt. %, ≥˜5 wt. %, ≥˜5.5 wt. %, ≤˜6 wt. %, ≥˜6.5 wt. %, ≥˜7 wt. %, ≥˜7.5 wt. %, ≥˜8 wt. %, ≥˜8.5 wt. %, ≥˜9 wt. %, ≥˜9.5 wt. %, or, e.g., ≥˜10 wt. %.

In certain aspects, where it is appropriate, or, e.g., recognizable by those in the art, to characterize a particular solvent as having one or more functional activities which is/are characterizable as an activity other than that of a solvent, e.g., characterizable as a suspending agent, a viscosity enhancing agent, or both, amount(s) disclosed herein as representing such activity, e.g., the amount of a suspension compound/agent or component or the amount of a viscosity-enhancement compound/agent or component can be applied to any one or more solvent compound(s)/agent(s)/constituent(s) described herein or can be applied to the solvent component described here.

### Solvent Means

In this and other component, compound/agent/constituent, or method aspect(s) of the invention, the invention also can be characterized as comprising a “means” for solubilizing component(s)/agent(s) of a composition or e.g., detectably or significantly increasing the compatibility between an API component and the propellant component. In such a respect, any known equivalents of such named solvent component(s), compound(s), or constituent(s) can also be, e.g., are, incorporated into composition(s) or method(s) of the invention. As with other sections similarly described herein, any of the component(s), compound(s), agent(s), constituent(s), or, e.g., method(s) of the invention can be, where suitable, described as means (e.g., the above-described solvent component(s), compound(s), or constituent(s) can be described as solvent means or means for solubilizing component(s)/agent(s), detectably or significantly increasing the compatibility between an API component and a propellant component, or both.)

### Viscosity Enhancing Agent(s)

In aspects, composition(s) herein can comprise a viscosity enhancement (viscosity enhancing) component comprising one or more viscosity enhancement (viscosity enhancer) compound(s)/agent(s). In aspects a viscosity enhancer is a composition agent/constituent which detectably or significantly increases the viscosity of composition(s), detectably or significantly increases the durability of a suspension nature of composition(s) of the invention (when composition(s) are provided as suspension(s)), or both as compared to a similar or the same composition(s) without such compound(s) (“viscosity enhancing agent(s)” or “viscosity enhancer(s)”.)

In aspects, a constituent of a viscosity enhancing component can comprise, e.g., one or more compound(s) recognized in the art as being capable of providing one or more activity(ies) aside from that of a viscosity enhancer. In aspects, viscosity enhancer compound(s) may further be characterizable as a suspension agent (e.g., providing for the detectable or significant suspension of one or more API(s)), a solvent (e.g., providing detectable or significant dissolution of one or more active pharmaceutical ingredients), or both, etc. In aspects, a viscosity enhancing compound can further be characterizable as such a compound and, uncontradicted, in aspects, can encompass such compounds and be present in such amounts described herein.

In aspects, composition(s) can comprise a viscosity enhancing component comprising any pharmaceutically acceptable and compositionally compatible viscosity enhancing compound(s)/agent(s), such as, for example, glycerol, polyethylene glycol (e.g., polyethylene glycol compounds of varying molecular weights), etc. In aspects, solvent(s), carrier(s), etc., such as, e.g., ethanol, water, etc. or combinations thereof present in varying amounts can detectably or significantly impact composition viscosity. In aspects, if such compound(s)/constituent(s) are present, composition(s) can comprise such constituent(s) in amount(s) described here or elsewhere herein.

### Viscosity Enhancing Agent(s) Amount

In certain aspects, composition(s) herein lack any component or compound/agent which is characterizable as a viscosity enhancer. In aspects, composition(s) herein are characterizable as viscosity-enhancer-free.

Here and in similar sections herein, amounts presented can, in aspects, represent the amount of a single viscosity enhancing compound/agent, the amount of a viscosity enhancing component (e.g., representing the total amount of viscosity enhancing compound(s) in a composition, or both. In aspects, a viscosity enhancing component of a composition can comprise two or more compounds each present in a composition in amount(s) described here.

In aspects, composition(s) herein comprise an effective amount of a pharmaceutically acceptable and compositionally compatible viscosity component. In aspects, composition(s) herein comprise a viscosity enhancing component wherein the viscosity enhancing component is present in an amount which represents between about 0 wt. % and about 2.5 wt. % of a composition, such as, e.g., ˜0 wt. %-˜2.4 wt. %, ˜0 wt. %-˜2.3 wt. %, ˜0 wt. %-˜2.2 wt. %, ˜0 wt. %-˜2.1 wt. %, ˜0 wt. %-˜2 wt. %, ˜0 wt. %-˜1.9 wt. %, ˜0 wt. %-˜1.8 wt. %, ˜0 wt. %-˜1.7 wt. %, ˜0 wt. %-˜1.6 wt. %, or, e.g., ˜0 wt. %-˜1.5 wt. % of a composition.

In aspects, composition(s) herein comprise a viscosity enhancing component wherein the viscosity enhancing component is present in an amount which represents between about 0.1 wt. % and about 2.5 wt. % of a composition, such as, e.g., ˜0.2 wt. %-˜2.5 wt. % of a composition, or ˜0.3 wt. %-˜2.5 wt. %, ˜0.4 wt. %-˜2.5 wt. %, ˜0.5 wt. %-˜2.5 wt. %, ˜0.6 wt. %-˜2.5 wt. %, ˜0.7 wt. %-˜2.5 wt. %, ˜0.8 wt. %-˜2.5 wt. %, ˜0.9 wt. %-˜2.5 wt. %, or, e.g., ˜1 wt. %-˜2.5 wt. % of a composition.

In aspects, composition(s) herein comprise a viscosity enhancing component wherein the viscosity enhancing component is present in an amount representing between about 0.2 wt. % and about 2.4 wt. % of a composition, such as, e.g., ˜0.3 wt. %-˜2.3 wt. %, ˜0.4 wt. %-˜2.2 wt. %, ˜0.5 wt. %-˜2.1 wt. %, ˜0.6 wt. %-˜2 wt. %, ˜0.7 wt. %-˜1.9 wt. %, ˜0.8 wt. %-˜1.8 wt. %, ˜0.9 wt. %-˜1.7 wt. %, ˜0.9 wt. %-˜1.6 wt. %, or, e.g., ˜1 wt. %-˜1.5 wt. % of a composition.

In certain aspects, composition(s) herein comprise a viscosity enhancing component comprising glycerol, wherein glycerol is present in an amount of, e.g., between about 0 wt. % and about 2.5 wt. %, such as, e.g., ˜0 wt. %-˜1 wt. %, ˜0.1 wt. %-˜2.5 wt. %, ˜0.2 wt. %-˜2 wt. %, ˜0.7 wt. %-˜1.7 wt. %, ˜0.8 wt. %-˜1.6 wt. %, or, e.g., ˜1 wt. %-˜1.5 wt. %, such as, for example, ˜0 wt. %-˜0.9 wt. %, ˜0 wt. %-˜0.8 wt. %, ˜0 wt. %-˜0.7 wt. %, ˜0 wt. %-˜0.6 wt. %, ˜0 wt. %-˜0.5 wt. %, ˜0 wt. %-˜0.4 wt. %, ˜0 wt. %-˜0.3 wt. %, ˜0 wt. %-˜0.2 wt. %, or, e.g., ˜0 wt. %-˜0.1 wt. %.

### Viscosity Enhancement Means

In this and other component, compound/agent/constituent, or method aspect(s) of the invention, the invention also can be characterized as comprising a “means” for increasing viscosity of composition(s). In such a respect, any known equivalents of such named viscosity-enhancing component(s), compound(s), or constituent(s) can also be, e.g., are, incorporated into composition(s) or method(s) of the invention. As with other sections similarly described herein, any of the component(s), compound(s), agent(s), constituent(s), or, e.g., method(s) of the invention can be, where suitable, described as means (e.g., the above-described viscosity-enhancing component(s), compound(s), or constituent(s)s can be described as viscosity-enhancing means, viscosity means, or means for increasing the viscosity of composition(s), increasing the durability of the suspension nature of composition(s) of the invention, or both.)

### Suspension Agent(s)

In aspects, composition(s) herein can comprise a suspension component comprising one or more suspension compound(s)/agent(s). In aspects, a suspension agent is a composition agent/constituent which detectably or significantly contributes to the establishment of a suspension (e.g., detectably or significantly increasing the maintenance of one or more composition component(s), e.g., active pharmaceutical ingredient(s), in suspension.)

In aspects, a constituent of a suspension component can comprise, e.g., one or more compound(s) recognized in the art as being capable of providing one or more activity(ies) aside from that of a suspension agent. In aspects, a suspension compound(s) may further be characterizable as, e.g., a dispersing agent, or, e.g., a solvent, and, in aspects, uncontradicted, can encompass dispersing agent(s), or, e.g., solvent(s) described herein, and, e.g., can be present in such disclosed amounts as described herein. In certain exemplary aspects, a suspension agent, such as, e.g., one or more polyethylene glycol (PEG) compound(s), glycerin (glycol), or both, can detectably or significantly increase the solubility of API(s), detectably or significantly increase composition viscosity, or both. In certain aspects, suspension agent(s), e.g., PEG(s), glycerin (glycol), etc. can detectably or significantly modulate particle size, e.g., aerosolized droplet size. In certain aspects, any such agent can be present alone or in combination with one or more other such agent(s), such as, e.g., PEG compound(s) can be present alone or in combination with glycerin (glycol), and, e.g., glycerin (glycol) can be present alone or in combination with PEG compound(s).

In aspects, composition(s) can comprise a suspension component comprising any pharmaceutically acceptable and compositionally compatible suspension compound(s)/agent(s). According to certain aspects, a suspension component of composition(s) provided herein can comprise, e.g., one or more PEGs, such as, e.g., PEG 400, PEG 600, PEG 1000, or, e.g., PVP K25, PVP K30, PVP VA64, lysine, glycine, leucine, phospholipid, cyclodextrin, one or more cremophor compound(s), etc. In aspects, composition(s) can comprise a mixture of any two or more compound(s)/agent(s) suspension component constituent(s) described in this paragraph or elsewhere herein.

According to certain aspects, a suspension component constituent is selected from PEG 400, PEG 600, PEG 1000, PVP K25, PVP K30, PVP VA64, lysine, glycine, leucine, phospholipid, cyclodextrin, a cremophor compound, or a combination thereof. In aspects, composition(s) comprise PEG600, PEG1000, glycerol, or any combination thereof.

### Suspension Agent Amount

In certain aspects, composition(s) herein lack any component or compound/agent which is characterizable as a suspension agent. In aspects, composition(s) herein are characterizable as suspension-agent free. In aspects, composition(s) here are provided as solutions. In aspects, composition(s) herein are not provided as suspension(s).

Here and in similar sections herein, amounts presented can, in aspects, represent the amount of a single suspension compound/agent, the amount of a suspension component (e.g., representing the total amount of suspension compound(s) in a composition, or both. In aspects, a suspension component of a composition can comprise two or more compounds each present in a composition in amount(s) described here.

In aspects, composition(s) herein comprise an effective amount of a pharmaceutically acceptable and compositionally compatible suspension component. In aspects, composition(s) provided herein comprise a suspension agent component in an amount representing between about 0.001 wt. % and about 10 wt. % of a composition, such as, e.g., ˜0.001 wt. %-˜9.5 wt. %, ˜0.001 wt. %-˜9 wt. %, ˜0.001 wt. %-˜8.5 wt. %, ˜0.001 wt. %-˜8 wt. %, ˜0.001 wt. %-˜7.5 wt. %, ˜0.001 wt. %-˜7 wt. %, ˜0.001 wt. %-˜6.5 wt. %, ˜0.001 wt. %-˜6 wt. %, ˜0.001 wt. %-˜5.5 wt. %, ˜0.001 wt. %-˜5 wt. %, ˜0.001 wt. %-˜4.5 wt. %, ˜0.001 wt. %-˜4 wt. %, ˜0.001 wt. %-˜3.5 wt. %, ˜0.001 wt. %-˜3 wt. %, ˜0.001 wt. %-˜2.5 wt. %, or, e.g., ˜0.001 wt. %-˜2 wt. % of a composition. In certain aspects, composition(s) can comprise a suspension agent component in an amount of ˜0.05 wt. %-˜0.2 wt. % or, e.g., ˜0.1 wt. %-˜0.2 wt. % or ˜0.1 wt. %-0.15 wt. % of the composition.

In aspects, composition(s) herein comprise a suspension agent component in an amount representing between about 0.005 wt. % and about 10 wt. % of a composition, such as, e.g., ˜0.01 wt. %-˜10 wt. %, ˜0.015 wt. %-˜10 wt. %, ˜0.02 wt. %-˜10 wt. %, ˜0.025 wt. %-˜10 wt. %, ˜0.03 wt. %-˜10 wt. %, ˜0.035 wt. %-˜10 wt. %, ˜0.04 wt. %-˜10 wt. %, ˜0.045 wt. %-˜10 wt. %, ˜0.05 wt. %-˜10 wt. %, ˜0.055 wt. %-˜10 wt. %, ˜0.06 wt. %-˜10 wt. %, ˜0.065 wt. %-˜10 wt. %, ˜0.07 wt. %-˜10 wt. %, ˜0.075 wt. %-˜10 wt. %, ˜0.08 wt. %-˜10 wt. %, ˜0.085 wt. %-˜10 wt. %, ˜0.09 wt. %-˜10 wt. %, ˜0.095 wt. %-˜10 wt. %, or, e.g., ˜0.1 wt. %-˜10 wt. % of a composition.

In aspects, composition(s) herein comprise a suspension agent component in an amount representing between about 0.005 wt. % and about 9.5 wt. % of a composition, such as, e.g., ˜0.01 wt. %-˜9 wt. %, ˜0.02 wt. %-˜8.5 wt. %, ˜0.03 wt. %-˜8 wt. %, ˜0.04 wt. %-˜7.5 wt. %, ˜0.05 wt. %-˜7 wt. %, ˜0.06 wt. %-˜6.5 wt. %, ˜0.07 wt. %-˜6 wt. %, ˜0.08 wt. %-˜5.5 wt. %, ˜0.09 wt. %-˜5 wt. %, or, e.g., ˜0.09 wt. %-˜4.5 wt. %, ˜0.09 wt. %-˜4 wt. %, ˜0.09 wt. %-˜3.5 wt. %, ˜0.09 wt. %-˜3 wt. %, or, e.g., ˜0.1 wt. %-˜2.5 wt. % of a composition.

According to certain aspects, composition(s) comprise a suspension agent component in an amount of between about 0.1 wt. % and about 2.5 wt. %, such as, e.g., ˜0.1 wt. %-˜2.4 wt. %, ˜0.1 wt. %-˜2.3 wt. %, ˜0.1 wt. %-˜2.2 wt. %, ˜0.1 wt. %-˜2.1 wt. %, ˜0.1 wt. %-˜2 wt. %, ˜0.1 wt. %-˜1.9 wt. %, ˜0.1 wt. %-˜1.8 wt. %, ˜0.1 wt. %-˜1.7 wt. %, ˜0.1 wt. %-˜1.6 wt. %, or, e.g., ˜0.1 wt. %-˜1.5 wt. % of a composition.

In aspects, composition(s) comprise a suspension agent component in an amount of ˜0.2 wt. %-˜2.5 wt. %, ˜0.3 wt. %-˜2.5 wt. %, ˜0.4 wt. %-˜2.5 wt. %, ˜0.5 wt. %-˜2.5 wt. %, ˜0.6 wt. %-˜2.5 wt. %, ˜0.7 wt. %-˜2.5 wt. %, ˜0.8 wt. %-˜2.5 wt. %, ˜0.9 wt. %-˜2.5 wt. %, or, e.g., ˜1 wt. %-˜2.5 wt. % of a composition.

In aspects, composition(s) comprise a suspension agent component in an amount of ˜0.2 wt. %-˜2.4 wt. %, ˜0.4 wt. %-˜2.2 wt. %, ˜0.6 wt. %-˜2 wt. %, ˜0.8 wt. %-˜1.8 wt. %, or, e.g., ˜1 wt. %-˜1.6 wt. % of a composition.

In aspects, composition(s) can comprise a suspension component wherein the suspension component comprises glycerol, and glycerol is present in a composition in an amount of between about 0.1 wt. % and about 2.5 wt. % of the composition. In certain aspects, composition(s) comprise a suspension component comprising one or more of PEG600, PEG1000, and glycerol, wherein (a) any one of PEG600, PEG1000, or glycerol, or (b) a combination of any or all thereof, is present in composition(s) in an amount of between about 0.000001 wt. % and about 1 wt. %, such as, e.g., ˜0.00001 wt. %-˜1 wt. %, ˜0.0001 wt. %-˜1 wt. %, ˜0.001 wt. %-˜1 wt. %, ˜0.01 wt. %-˜1 wt. %, ˜0.1 wt. %-˜1 wt. %, or, e.g., 0.5 wt. %-˜1 wt. % of the composition.

In aspects, composition(s) do not comprise a PEG compound. In aspects, one or more PEG compound(s) are present in composition(s) in an amount of, e.g., about 0.1 wt. %, ˜0.15 wt. %, ˜0.2 wt. %, ˜0.25 wt. %, ˜0.3 wt. %, ˜0.35 wt. %, ˜0.4 wt. %, ˜0.45 wt. %, or, e.g., ˜0.5 wt. % of the composition.

In certain aspects, composition(s) comprise a suspension component comprising, e.g., glycerol, wherein glycerol is present in an amount of no more than about 2 wt. % of a composition, such as, e.g., ≤˜1.9 wt. %, ≤˜1.8 wt. %, ≤˜1.7 wt. %, ≤˜1.6 wt. %, ≤˜1.5 wt. %, ≤˜1.4 wt. %, ≤˜1.3 wt. %, ≤˜1.2 wt. %, ≤˜1.1 wt. %, ≤˜1 wt. %, ≤˜0.9 wt. %, ≤˜0.8 wt. %, ≤˜0.7 wt. %, ≤˜0.75 wt. %, or, e.g., ≤˜0.6 wt. % of a composition.

In aspects, composition(s) can comprise a suspension component comprising, e.g., glycerol, wherein glycerol is present in an amount of between about 0.7 wt. % and about 1.7 wt. %, such as, e.g., ˜0.8 wt. %-˜1.6 wt. %, ˜0.9 wt. %-˜1.6 wt. %, or, e.g., ˜1 wt. %-˜1.5 wt. % of the composition.

In aspects, composition(s) can comprise a suspension component comprising PVP k25, wherein the PVP k25 is present in an amount of between about 0.00001 wt. % and about 0.001 wt. %, such as, e.g., ˜0.00005 wt. %-˜0.001 wt. %, e.g., ˜0.0001 wt. % of the composition.

In certain aspects, composition(s) can comprise a suspension component comprising, e.g., glycerol, wherein glycerol is present in an amount of no less than about 1 wt. %, such as, e.g., ≥˜1.5 wt. %, ≥˜2 wt. %, ≥˜2.5 wt. %, ≥˜3 wt. %, ≥˜3.5 wt. %, ≥˜4 wt. %, ≥˜4.5 wt. %, ≥˜5 wt. %, ≥˜5.5 wt. %, ≥˜6 wt. %, ≥˜6.5 wt. %, ≥˜7 wt. %, ≥˜7.5 wt. %, ≥˜8 wt. %, ≥˜8.5 wt. %, ≥˜9 wt. %, ≥˜9.5 wt. %, or, e.g., ≥˜10 wt. % of the composition.

### Suspension Means

In this and other component, compound/agent/constituent, or method aspect(s) of the invention, the invention also can be characterized as comprising a “means” for suspending one or more composition components, e.g., one or more compound(s), e.g., an API component or one or more API component constituents. In such a respect, any known equivalents of such named suspension component(s), compound(s), or constituent(s) can also be, e.g., are, incorporated into composition(s) or method(s) of the invention. As with other sections similarly described herein, any of the component(s), compound(s), agent(s), constituent(s), or, e.g., method(s) of the invention can be, where suitable, described as means (e.g., the above-described viscosity-enhancing component(s), compound(s), or constituent(s) can be described as suspension means or means for suspending one or more composition components, e.g., one or more compound(s), e.g., an API component or one or more API component constituents.)

### Bulking Agent(s)

In aspects, composition(s) herein can comprise a bulking component comprising one or more bulking compound(s)/agent(s). In aspects, a bulking compound/agent is any compound/agent which detectably or significantly forms an API-excipient matrix that detectably or significantly minimizes the ability of one or more API(s) to migrate out of the formulation, resulting in, e.g., improved dosing reproducibility.

In aspects, a constituent of a bulking component can comprise, e.g., one or more compound(s) recognized in the art as being capable of providing one or more activity(ies) aside from that of a bulking agent. In aspects, bulking agent(s)/compound(s) may further be characterizable as, e.g., a viscosity enhancing agent, solvent, etc. In aspects, a bulking compound/agent can further be characterizable as such a compound and, uncontradicted, in aspects, can encompass such compounds and be present in such amounts as described herein.

In aspects, composition(s) can comprise a bulking component comprising any pharmaceutically acceptable and compositionally compatible bulking compound(s)/agent(s). According to certain aspects, a bulking component of composition(s) provided herein can comprise, e.g., saccharides such as, but not limited to, monosaccharides, disaccharides, oligosaccharides, and polysaccharides for example lactose, maltose, glucose, fructose, galactose arabinose, dextrose, ribose, sucrose, sorbitol, mannitol, xylose, trehalose, raffinose, melezitose, glycerol, erythritol, xylitol, maltitol, lactitol, and D & L series of rare sugars, magnesium stearate, etc. In aspects, composition(s) can comprise a mixture of any two or more compound(s)/agent(s)/bulking component constituent(s) described in this paragraph or elsewhere herein.

According to certain aspects, a bulking component constituent is selected from lactose, magnesium stearate, and mannitol, or a combination of any or all thereof.

### Bulking Agent Amount

According to certain aspects, composition(s) herein lack any component or compound/agent which is characterizable as a bulking component/agent/compound. In aspects, composition(s) are characterizable as bulking-component/agent/compound free.

Here and in similar sections herein, amounts presented can, in aspects, represent the amount of a single bulking compound/agent, the amount of a bulking component (e.g., representing the total amount of bulking (or, e.g., co-solvent) compound(s)/agent(s) in a composition, or both. In aspects, a bulking component of a composition can comprise two or more compounds each present in a composition in amount(s) described here.

In aspects, composition(s) herein comprise an effective amount of a pharmaceutically acceptable and compositionally compatible bulking component. According to certain aspects, composition(s) comprise a bulking component wherein the bulking component is present in an amount representing between about 0.0001 wt. % and about 20 wt. % of the composition, such as, e.g., ˜0.0001 wt. %-˜19 wt. %, ˜0.0001 wt. %-˜18 wt. %, ˜0.0001 wt. %-˜17 wt. %, ˜0.0001 wt. %-˜16 wt. %, ˜0.0001 wt. %-˜15 wt. %, ˜0.0001 wt. %-˜14 wt. %, ˜0.0001 wt. %-˜13 wt. %, ˜0.0001 wt. %-˜12 wt. %, ˜0.0001 wt. %-˜11 wt. %, or, e.g., ˜0.0001 wt. %-˜10 wt. % of a composition.

In aspects, composition(s) comprise a bulking component wherein the bulking component is present in an amount representing between about 0.00012 wt. %-˜20 wt. % of a composition, such as, e.g., ˜0.00013 wt. %-˜20 wt. %, ˜0.00014 wt. %-˜20 wt. %, ˜0.00015 wt. %-˜20 wt. %, ˜0.00016 wt. %-˜20 wt. %, ˜0.00017 wt. %-˜20 wt. %, ˜0.00018 wt. %-˜20 wt. %, ˜0.00019 wt. %-˜20 wt. %, or, e.g., ˜0.001 wt. %-˜20 wt. % of a composition.

In aspects, composition(s) comprise a bulking component wherein the bulking component is present in an amount representing ˜0.00012 wt. %-˜19 wt. %, ˜0.00013 wt. %-˜18 wt. %, ˜0.00014 wt. %-˜17 wt. %, ˜0.00015 wt. %-˜16 wt. %, ˜0.00016 wt. %-˜15 wt. %, ˜0.00017 wt. %-˜14 wt. %, ˜0.00018 wt. %-˜13 wt. %, ˜0.00019 wt. %-˜12 wt. %, ˜0.001 wt. %-˜11 wt. %, or, e.g., ˜0.001 wt. %-˜10 wt. % of the composition.

In aspects, composition(s) comprise a bulking component wherein the bulking component is present in an amount representing between about 5 wt. % and about 600 wt. % of the drug, such as, e.g., ˜5 wt. %-˜590 wt. % of the drug, ˜5 wt. %-˜580 wt. % of the drug, ˜5 wt. %-˜570 wt. % of the drug, ˜5 wt. %-˜560 wt. % of the drug, ˜5 wt. %-˜550 wt. % of the drug, ˜5 wt. %-˜540 wt. % of the drug, ˜5 wt. %-˜530 wt. % of the drug, ˜5 wt. %-˜520 wt. % of the drug, ˜5 wt. %-˜510 wt. % of the drug, or, e.g., ˜5 wt. %-˜500 wt. % of the drug.

In aspects, composition(s) comprise a bulking component wherein the bulking component is present in an amount representing between about 6 wt. % and about 600 wt. % of the drug, such as, e.g., ˜7 wt. %-˜600 wt. %, ˜8 wt. %-˜600 wt. %, ˜9 wt. %-˜600 wt. %, or, e.g., ˜10 wt. %-˜600 wt. % of the drug.

In aspects, composition(s) comprise a bulking component wherein the bulking component is present in an amount representing between about 6 wt. %-˜590 wt. % of the drug, such as, e.g., ˜6.5 wt. %-˜580 wt. %, ˜7 wt. %-˜560 wt. %, ˜7.5 wt. %-˜540 wt. %, ˜8 wt. %-˜520 wt. %, ˜8.5 wt. %-˜500 wt. %, ˜9 wt. %-˜480 wt. % or, e.g., ˜10 wt. %-˜460 wt. % of the drug, such as, e.g., ˜10 wt. %-˜500 wt. % of the drug.

### Bulking Means

In this and other component, compound/agent/constituent, or method aspect(s) of the invention, the invention also can be characterized as comprising a “means” for bulking a composition, e.g., a means of detectably or significantly form an API-excipient matrix that detectably or significantly minimizes the ability of one or more API(s) to migrate out of the formulation, resulting in, e.g., improved dosing reproducibility. In such a respect, any known equivalents of such named bulking component(s), compound(s), or constituent(s) can also be, e.g., are, incorporated into composition(s) or method(s) of the invention. As with other sections similarly described herein, any of the component(s), compound(s), agent(s), constituent(s), or, e.g., method(s) of the invention can be, where suitable, described as means (e.g., the above-described bulk-enhancing component(s), compound(s), or constituent(s) can be described as bulk-enhancing means or bulking means or means for bulking a composition or detectably or significantly form an API-excipient matrix that detectably or significantly minimizes the ability of one or more API(s) to migrate out of the formulation, resulting in, e.g., improving dosing reproducibility.)

### Stabilizer(s)

In aspects, composition(s) herein can comprise a stabilizer component comprising one or more stabilizer compound(s)/agent(s). In aspects, a stabilizer compound/agent is any compound/agent which detectably or significantly increases the stability of the formulation, such as, e.g., detectably or significantly increases the shelf life of composition(s) compared to similar or the same composition(s) lacking such compound(s)/agent(s) or, e.g., detectably or significantly reduces, prevents, or otherwise delays the formation of one or more API-related impurity(ies). In aspects, a stabilizing compound does not detectably or significantly reduce surface tension. In aspects, a stabilizing compound can be a buffer acid. In aspects, stabilizer(s) detectably or significantly increase the chemical stability of API(s), such as, e.g., detectably or significantly prevent, reduce, or otherwise slow detectable or significant hydrolysis of APIs(s). In aspects, a stabilizer compound can be distinct from a surfactant compound in that while a stabilizer compound may detectably or significantly improve chemical stability of API(s), a surfactant compound may detectable or significantly improve physical stability, e.g., by detectably or significantly reducing particle cohesion, detectably or significantly reducing sticking to one or more components of a container, detectably or significantly increasing the interaction of API(s) with propellant(s), or a combination thereof. In aspects, stabilizing compound(s) are pharmaceutically acceptable and compositionally compatible compound(s), in certain aspects, e.g., small molecule compound(s), e.g., in certain further aspects small molecule acids, capable of detectably or significantly preventing or delaying any change in the homogeneity of a composition resulting in (a) the amount of light capable of at least substantially freely passing through the composition to be reduced or (b) the composition no longer being characterizable as a solution when formulated as a solution. In aspects, stabilizer(s) are acid(s). In aspects, stabilizer(s) are base(s). In aspects, stabilizer(s) provide pH modulation effect (e.g., are capable of modifying composition pH when present in an effective amount).

In aspects, a constituent of a stabilizer component can comprise, e.g., one or more compound(s) recognized in the art as being capable of providing one or more activity(ies) aside from that of a stabilizer. In aspects, stabilizer compound(s) may further be characterizable as, e.g., a surfactant, an antioxidant, an organic acid, a chelating agent, etc. In aspects, a stabilizer compound can further be characterizable as such a compound and, uncontradicted, in aspects, can encompass such compounds and be present in such amounts as described herein.

In certain aspects, a stabilizer is a pharmaceutically acceptable and compositionally compatible small compound stabilizing agent(s). In aspects, the small compound stabilizing agent(s) have a molecular weight of less than about 250 g/mol, such as, e.g., ≤˜245 g/mol, ≤˜240 g/mol, ≤˜235 g/mol, ≤˜230 g/mol, ≤˜225 g/mol, ≤˜220 g/mol, ≤˜215 g/mol, ≤˜210 g/mol, ≤˜205 g/mol, ≤˜200 g/mol, ≤˜195 g/mol, ≤˜190 g/mol, ≤˜185 g/mol, ≤˜180 g/mol, or, e.g., ≤˜175 g/mol.

In certain aspects, small compound stabilizing agent(s) present in composition(s) herein comprise no more than about 10 carbon atoms, such as, e.g., ≤˜9 carbons, ≤˜8 carbons, ≤˜7 carbons, ≤˜6 carbons, ≤˜5 carbons, ≤˜4 carbons, ≤˜3 carbons, ≤˜2 carbons, ≤˜1 carbon, or, e.g., in aspects, do not comprise a carbon atom.

In aspects, the small compound stabilizing agent(s) provide(s) detectable or significant chelating activity. In aspects, a small compound stabilizing agent(s) is capable of maintaining API(s) in a dissolved state such that the composition is characterizable as a solution for a detectably or significantly longer period of time than the same or at least substantially the same composition(s), e.g., comprising the same API(s) in the same or at least substantially the same amount(s), not comprising such small compound stabilizing agent(s). In aspects, a small compound stabilizing agent(s) is capable of detectably or significantly preventing or delaying any change in the homogeneity of the composition resulting in the amount of light capable of passing through the composition to be reduced. In aspects, a small compound stabilizing agent(s) is capable of detectably or significantly preventing or delaying any change in the homogeneity of the composition resulting in the composition no longer being characterizable as a solution. In aspects, a small compound stabilizing agent(s) is capable of detectably or significantly preventing or delaying any change in the homogeneity of the composition resulting in the amount of light capable of passing through the composition to be reduced such that the composition no longer being characterizable as a solution.

According to certain aspects, composition(s) herein comprise a small compound stabilizing agent as described above, wherein the small compound stabilizing agent comprises or is an organic acid compound. In aspects, composition(s) comprise at least two small compound stabilizing agents as described above. In aspects, the small compound stabilizing agents are organic acid compounds.

In aspects, composition(s) can comprise a stabilizer component comprising any pharmaceutically acceptable and compositionally compatible stabilizer compound(s)/agent(s). According to certain aspects, a stabilizer component of composition(s)) provided herein can comprise, e.g., one or more antioxidants such as sacrificial antioxidants, an organic acid, such as, e.g., ascorbic acid, fumaric acid, or, e.g., citric acid, phosphoric acid, HCl, calcium chloride, fumaric acid, acetic acid, malic acid, maleic acid, lactic acid, or, e.g., EDTA, etc. or salts of any or all thereof. In aspects, composition(s) can comprise a mixture of any two or more compound(s)/agent(s)/stabilizer component constituent(s) described in this paragraph or elsewhere herein.

In certain aspects, a stabilizer component comprises a stabilizer selected from citric acid, ascorbic acid, phosphoric acid, hydrochloric acid, calcium chloride, fumaric acid, acetic acid, malic acid, maleic acid, lactic acid, or EDTA, or salt(s) thereof or a combination of any or all thereof. According to certain aspects, a stabilizer component comprises an organic acid, such as, e.g., citric acid or a salt thereof. In aspects, composition(s) comprise citric acid, maleic acid, or both or salts of either or both thereof.

According to aspects, a stabilizer component does not comprise compound(s) characterizable as surfactant(s). In aspects, a stabilizer component does not comprise compound(s) capable of demonstrating detectable or significant surfactant activity(ies). In aspects, a stabilizer component can comprise compound(s)/agent(s) having co-surfactant property(ies); however, such compound(s) are typically or necessarily unsuitable to provide surfactant activity alone (e.g., without a demonstrable surfactant compound being present.)

### Stabilizer Amount

According to certain aspects, composition(s) herein lack any component or compound/agent which is characterizable as a stabilizer. In aspects, composition(s) are characterizable as stabilizer free. In certain aspects, composition(s) comprise no stabilizer characterizable as an organic acid.

Here and in similar sections herein, amounts presented can, in aspects, represent the amount of a single stabilizer compound/agent, the amount of a stabilizer component (e.g., representing the total amount of stabilizer compound(s) in a composition, or both. In aspects, a stabilizer component of a composition can comprise, e.g., two or more compounds each present in a composition in amount(s) described here.

In aspects, composition(s) herein comprise an effective amount of a pharmaceutically acceptable and compositionally compatible stabilizer component. In aspects, a sufficient amount of stabilizing agent(s)/compound(s), or, e.g., a stabilizing component, is present to maintain a stable composition for a period of at least about 1 week, such as, e.g., at least about month, ≥˜2 months, ≥˜3 months, ≥˜4 months, ≥˜6 months, ≥˜8 months, ≥˜10 months, ≥˜12 months, ≥˜16 months, ≥˜20 months, ≥˜24 months, ≥˜30 months, or, e.g., ≥˜36 months when stored at room temperature or under standard storage conditions.

In one specific example, composition(s) can comprise a stabilizing compound, such as, e.g., citric acid, malic acid, or, e.g., maleic acid. In aspects, the amount of such an acid can detectably decrease over the course of a storage period. In aspects, composition(s) can comprise an amount of a stabilizing compound such as, e.g., citric acid, malic acid, or, e.g., maleic acid, e.g., citric acid, present in an initial (immediately post-manufacturing) amount representing about 0.04 wt. % of the composition, wherein there is a sufficient amount of stabilizing agent, e.g., citric acid, present in the composition to stabilize the tiotropium compound(s) also present in the composition without detectable or significant formation of one or more impurity(ies) which are observed at higher (e.g., increased) concentrations of such acid (e.g., citric acid). In aspects, such composition(s) may comprise two small compound stabilizing agents, such as, e.g., citric acid and maleic acid.

In aspects, composition(s) comprise a stabilizer component, wherein the stabilizer component is present in composition(s) in an amount representing between about 0.00001 wt. % and about 0.5 wt. %, such as, e.g., ˜0.00001 wt. %-˜0.45 wt. %, ˜0.00001 wt. %-˜0.4 wt. %, ˜0.00001 wt. %-˜0.35 wt. %, ˜0.00001 wt. %-˜0.3 wt. %, ˜0.00001 wt. %-˜0.25 wt. %, ˜0.00001 wt. %-˜0.2 wt. %, ˜0.00001 wt. %-˜0.15 wt. %, or, e.g., ˜0.00001 wt. %-˜0.1 wt. % of the composition.

In aspects, composition(s) comprise a stabilizer component, wherein the stabilizer component is present in composition(s) in an amount representing between ˜0.00005 wt. %-˜0.5 wt. %, ˜0.0001 wt. %-˜0.5 wt. %, ˜0.0002 wt. %-˜0.5 wt. %, ˜0.0003 wt. %-˜0.5 wt. %, ˜0.0004 wt. %-˜0.5 wt. %, ˜0.0005 wt. %-˜0.5 wt. %, ˜0.0006 wt. %-˜0.5 wt. %, ˜0.0007 wt. %-˜0.5 wt. %, ˜0.0008 wt. %-˜0.5 wt. %, ˜0.0009 wt. %-˜0.5 wt. %, or, e.g., ˜0.001 wt. %-˜0.5 wt. % of the composition.

In aspects, composition(s) comprise a stabilizer component, wherein the stabilizer component is present in composition(s) in an amount representing ˜0.00005 wt. %-0.45 wt. %, ˜0.00006 wt. %-0.4 wt. %, ˜0.00007 wt. %-0.35 wt. %, ˜0.00008 wt. %-0.3 wt. %, ˜0.00009 wt. %-0.25 wt. %, ˜0.00009 wt. %-0.2 wt. %, ˜0.0005 wt. %-0.15 wt. %, or, e.g., ˜0.0001 wt. %-0.1 wt. %, such as, ˜0.005 wt. %-˜0.1 wt. % or, e.g., ˜0.001 wt. %-˜0.1 wt. % of the composition, such as, for example, about 0.04 wt. %, about 0.002 wt. %, or, e.g., between about 0.03 wt. % and about 0.05 wt. %, such as, e.g., between about 0.04 wt. % and about 0.05 wt. %.

In aspects, a pharmaceutically acceptable and compositionally compatible small compound stabilizing agent (e.g., a stabilizing compound having a molecular weight of less than about 200 g/mol, providing detectable or significant chelating activity, or both, wherein the small compound stabilizing agent is capable of detectably or significantly preventing or delaying any change in the homogeneity of the composition resulting in (a) the amount of light capable of passing through the composition to be reduced or (b) a composition no longer being characterizable as a solution when the composition is provided as a solution) is present in a composition provided herein in an amount of at least about 0.01 wt. %, such as, e.g., ≥˜0.015 wt. %, ≥˜0.02 wt. %, ≥˜0.025 wt. %, ≥˜0.03 wt. %, ≥˜0.035 wt. %, ≥˜0.04 wt. %, ≥˜0.045 wt. %, ≥˜0.05 wt. %, ≥˜0.055 wt. %, ≥˜0.06 wt. %, ≥˜0.065 wt. %, ≥˜0.07 wt. %, ≥˜0.075 wt. %, ≥˜0.08 wt. %, ≥˜0.085 wt. %, ≥˜0.09 wt. %, ≥˜0.095 wt. %, or, e.g., ≥˜0.1 wt. % of the composition.

In aspects, a stabilizing component can comprise, e.g., citric acid, malic acid, maleic acid, or, e.g., a combination of any two or more thereof. In aspects, when citric acid, malic acid, maleic acid, or a combination of two or more thereof is present in composition(s) provided by the invention, citric acid, malic acid, maleic, or a combination of two or more thereof can be present in an amount representing no less than about 0.01 wt. %, such as, e.g., ≥0.015 wt. %, ≥0.02 wt. %, ≥0.025 wt. %, ≥0.03 wt. %, ≥0.035 wt. %, ≥0.04 wt. %, ≥0.045 wt. %, ≥0.05 wt. % of the composition.

In aspects, a stabilizing component can comprise, e.g., citric acid, malic acid, maleic acid, or, e.g., a combination of any two or more thereof. In aspects, when citric acid, malic acid, maleic acid, or a combination of two or more thereof is present in composition(s) provided by the invention, citric acid, malic acid, maleic, or a combination of two or more thereof can be present in an amount representing no greater than about 0.15 wt. %, e.g., ≤˜0.1 wt. %, ≤˜0.09 wt. %, ≤˜0.08 wt. %, ≤˜0.07 wt. %, ≤˜0.06 wt. %, ˜0.05 wt. %, ≤˜0.04 wt. %, ≤˜0.03 wt. %, ≤˜0.02 wt. %, or, e.g., ≤˜0.01 wt. % of the composition.

In aspects, a stabilizing component can comprise, e.g., citric acid, malic acid, maleic acid, or, e.g., a combination of any two or more thereof. In aspects, when citric acid, malic acid, maleic acid, or a combination of two or more thereof is present in composition(s) provided by the invention, citric acid, malic acid, maleic, or a combination of two or more thereof can be present in an amount representing between about 0.01 wt. % and about 0.15 wt. %, such as, e.g., ˜0.02 wt. %-˜0.1 wt. %, or, e.g., ˜0.03 wt. %-˜0.05 wt. % of the composition.

Herein, when the salt of an acid stabilizing agent, e.g., a salt of citric acid, is present in composition(s) herein, weight percent values in this section and elsewhere herein should be understood to be based on the weight of free citric acid (i.e., without considering the weight of the cation). Applicable conversion factors as would be understood by those in the art can be applied as needed. In certain aspects, acid(s) in free form, such as, e.g., citric acid alone, is used.

### Stabilizing Means

In this and other component, compound/agent/constituent, or method aspect(s) of the invention, the invention also can be characterized as comprising a “means” for stabilizing composition(s). In such a respect, any known equivalents of such named stabilization component(s), compound(s), or constituent(s) can also be, e.g., are, incorporated into composition(s) or method(s) of the invention. As with other sections similarly described herein, any of the component(s), compound(s), agent(s), constituent(s), or, e.g., method(s) of the invention can be, where suitable, described as means (e.g., the above-described stabilization component(s), compound(s), or constituent(s) can be described as stabilizing means or means for increasing the stability of composition(s).)

### Organic Acid(s)

In aspects, composition(s) herein can comprise an organic acid component comprising on or more organic acid compound(s)/agent(s) or salt(s) thereof. In aspects, an organic acid compound can be, e.g., an organic acid or pharmaceutically acceptable salt thereof. In aspects, a particular organic acid may provide one or more functional activities, such as, e.g., stabilizing activity, surfactant activity, etc.

In aspects, a constituent of an organic acid component can comprise, e.g., one or more compound(s) recognized in the art as being capable of providing one or more activity(ies). In aspects, organic acid compound(s) may be characterizable as, e.g., a stabilizing agent. In aspects, organic acid compound(s) may be characterizable as a surfactant. In aspects, organic acid compound(s) can further be characterizable as such compound(s) and, uncontradicted, in aspects, can encompass such compounds and be present in such amounts as described herein.

In aspects, composition(s) can comprise an organic acid component comprising any pharmaceutically acceptable and compositionally compatible organic acid compound(s)/agent(s). According to certain aspects, an organic acid component of composition(s) provided herein can comprise, e.g., ascorbic acid, phosphoric acid, fumaric acid, citric acid, malic acid, maleic acid, acetic acid, lactic acid, ethylenediaminetetraacetic acid (EDTA), etc. or e.g., salts of any or all thereof. In aspects, composition(s) can comprise a mixture of any two or more compound(s)/agent(s)/organic acid component constituent(s) described in this paragraph or elsewhere herein. In aspects, composition(s) comprise citric acid or maleic acid or salt(s) thereof, such as citric acid or maleic acid.

### Organic Acid Amount

According to certain aspects, composition(s) herein lack any component or compound/agent which is characterizable as an organic acid. In aspects, composition(s) are characterizable as organic acid free.

Here and in similar sections herein, amounts presented can, in aspects, represent the amount of a single organic acid compound/agent, the amount of an organic acid component, or both. In aspects, an organic acid component of a composition can comprise two or more compounds each present in a composition in amount(s) described here.

In aspects, composition(s) herein comprise an effective amount of a pharmaceutically acceptable and compositionally compatible organic acid component. In certain aspects, composition(s) provided herein comprise one or more organic acid(s) in an amount of between about 0.01 wt. % and about 0.1 wt. %, such as, e.g., ˜0.0125 wt. %-˜0.1 wt. %, ˜0.015 wt. %-˜0.1 wt. %, ˜0.0175 wt. %-˜0.1 wt. %, ˜0.02 wt. %-˜0.1 wt. %, ˜0.0225 wt. %-˜0.1 wt. %, ˜0.025 wt. %-˜0.1 wt. %, ˜0.0275 wt. %-˜0.1 wt. %, ˜0.03 wt. %-˜0.1 wt. %, ˜0.0325 wt. %-˜0.1 wt. %, ˜0.035 wt. %-˜0.1 wt. %, ˜0.0375 wt. %-˜0.1 wt. %, ˜0.04 wt. %-˜0.1 wt. %, ˜0.0425 wt. %-˜0.1 wt. %, ˜0.045 wt. %-˜0.1 wt. %, ˜0.0475 wt. %-˜0.1 wt. %, or, e.g., ˜0.05 wt. %-˜0.1 wt. % of the composition, such as, e.g., ˜0.03 wt. %-˜0.05 wt. %, e.g., ˜0.035 wt. %-˜0.045 wt. %, e.g., ˜0.04 wt. % or ˜0.042 wt. % of the composition.

In aspects, composition(s) provided herein comprise one or more organic acid(s) in an amount of between about 0.01 wt. % and about 0.95 wt. %, such as, e.g., ˜0.01 wt. %-˜0.9 wt. %, ˜0.01 wt. %-˜0.85 wt. %, ˜0.01 wt. %-˜0.8 wt. %, ˜0.01 wt. %-˜0.75 wt. %, ˜0.01 wt. %-˜0.7 wt. %, ˜0.01 wt. %-˜0.65 wt. %, ˜0.01 wt. %-˜0.6 wt. %, ˜0.01 wt. %-˜0.55 wt. %, or, e.g., ˜0.01 wt. %-˜0.5 wt. % of the composition.

In aspects, composition(s) provided herein comprise one or more organic acid(s) in an amount of between about 0.02 wt. % and about 0.9 wt. %, such as, e.g., ˜0.025 wt. %-˜0.8 wt. %, ˜0.03 wt. %-˜0.7 wt. %, ˜0.035 wt. %-˜0.6 wt. %, or, e.g., ˜0.04 wt. %-˜0.5 wt. %, such as, e.g., ˜0.045 wt. %-˜0.5 wt. % or, e.g., ˜0.05 wt. %-˜0.5 wt. % of the composition.

In certain aspects, composition(s) herein comprise, e.g., citric acid, malic acid, or, e.g., maleic acid in an amount of, e.g., between about 0.0125 wt. % and about 0.15 wt. %, such as, e.g., about 0.05 wt. %.

According to certain aspects, wherein one or more organic acid(s), e.g., citric acid, malic acid, or, e.g., maleic acid, is present in composition(s), the one or more organic acid(s) such as citric acid, malic acid, or maleic acid is present in an amount of no less than about 0.04 wt. %, such as, e.g., ≥˜0.05 wt. %, ≥˜0.06 wt. %, ≥˜0.07 wt. %, ≥˜0.08 wt. %, ≥˜0.09 wt. %, ≥˜0.1 wt. %, ≥˜0.11 wt. %, ≥˜0.12 wt. %, ≥˜0.13 wt. %, ≥˜0.14 wt. %, ≥˜0.15 wt. %, ≥˜0.16 wt. %, ≥˜0.17 wt. %, ≥˜0.18 wt. %, ≥˜0.19 wt. %, or, e.g., ≥˜0.2 wt. % of the composition.

In certain aspects, composition(s) can comprise a surfactant characterizable as an organic acid, e.g., oleic acid. In aspects, composition(s) can comprise an organic acid such as oleic acid in an amount of, e.g., between about 0.0002 wt. % and about 0.45 wt. %, such as, e.g., ˜0.0003 wt. % and about 0.4 wt. %, ˜0.0004 wt. % and about 0.35 wt. %, ˜0.0005 wt. %-˜0.3 wt. %, ˜0.0006 wt. %-˜0.25 wt. %, ˜0.0007 wt. %-˜0.2 wt. %, ˜0.0008 wt. %-˜0.15 wt. %, ˜0.0009 wt. %-˜0.1 wt. %, or, e.g., ˜0.001 wt. %-˜0.1 wt. % of the composition.

### Dispersing Agent(s)

In aspects, composition(s) herein can comprise a dispersing agent component (dispersant component) comprising one or more dispersant compound(s)/agent(s) (dispersing agents). In aspects, a dispersing compound/agent is any compound/agent which detectably or significantly increases the dispersion of particles, detectably or significantly increases (or, e.g., improves upon) the separation of particles, detectably or significantly improves the separation of particles, detectably or significantly prevents the settling or clumping of particles, or any combination of any or all thereof.

In aspects, a constituent of a dispersing agent component can comprise, e.g., one or more compound(s) recognized in the art as being capable of providing one or more activity(ies) aside from that of a dispersing agent. In aspects, dispersing compound(s)/agent(s) (dispersant(s)) may further be characterizable as, e.g., a surfactant, or, e.g., a suspension agent. In aspects, a dispersing compound/agent can further be characterizable as such a compound and, uncontradicted, in aspects, can encompass such compounds and be present in such amounts as described herein.

In aspects, composition(s) can comprise a dispersing agent component comprising any pharmaceutically acceptable and compositionally compatible dispersing compound(s)/agent(s). According to certain aspects, a dispersant component of composition(s) provided herein can comprise, e.g., sorbitan trioleate, oleyl alcohol, oleic acid, lecithin, etc. In aspects, composition(s) can comprise a mixture of any two or more compound(s)/agent(s)/surfactant component constituent(s) described in this paragraph or elsewhere herein.

### Dispersing Agent Amount

According to certain aspects, composition(s) herein lack any component or compound/agent which is characterizable as a dispersant or dispersing compound/agent. In aspects, composition(s) are characterizable as dispersing agent free.

Here and in similar sections herein, amounts presented can, in aspects, represent the amount of a single dispersing compound/agent, the amount of a dispersing component (e.g., representing the total amount of dispersing compound(s) in a composition, or both. In aspects, a dispersing component of a composition can comprise two or more compounds each present in a composition in amount(s) described here.

According to certain aspects, composition(s) comprise a dispersing component wherein the dispersing component is present in the composition in an amount representing between about 0.0001 wt. % and about 0.5 wt. %, such as, e.g., ˜0.0001 wt. %-˜0.45 wt. %, ˜0.0001 wt. %-˜0.4 wt. %, ˜0.0001 wt. %-˜0.35 wt. %, ˜0.0001 wt. %-˜0.3 wt. %, ˜0.0001 wt. %-˜0.25 wt. %, ˜0.0001 wt. %-˜0.2 wt. %, ˜0.0001 wt. %-˜0.15 wt. %, or, e.g., ˜0.0001 wt. %-˜0.1 wt. % of the composition.

In certain aspects, composition(s) comprise a dispersing component wherein the dispersing component is present in the composition in an amount representing between about 0.0002 wt. %-˜0.5 wt. %, such as, e.g., ˜0.0003 wt. %-˜0.5 wt. %, ˜0.0004 wt. %-˜0.5 wt. %, ˜0.0005 wt. %-˜0.5 wt. %, ˜0.0006 wt. %-˜0.5 wt. %, ˜0.0007 wt. %-˜0.5 wt. %, ˜0.0008 wt. %-˜0.5 wt. %, ˜0.0009 wt. %-˜0.5 wt. %, or, e.g., ˜0.001 wt. %-˜0.5 wt. % of the composition.

In aspects, composition(s) comprise a dispersing component wherein the dispersing component is present in the composition in an amount representing ˜0.0002 wt. %-˜0.45 wt. %, ˜0.0003 wt. %-˜0.4 wt. %, ˜0.0004 wt. %-˜0.4 wt. %, ˜0.0005 wt. %-˜0.35 wt. %, ˜0.0006 wt. %-˜0.3 wt. %, ˜0.0007 wt. %-˜0.25 wt. %, ˜0.0008 wt. %-˜0.2 wt. %, ˜0.0009 wt. %-˜0.15 wt. %, or, e.g., ˜0.001 wt. %-˜0.1 wt. % of the composition, as in, for example, ˜0.002 wt. %-˜0.01 wt. %, ˜0.003 wt. %-˜0.006 wt. %, ˜0.004 wt. %-˜0.006 wt. %, or, e.g., ˜0.005 wt. % of the composition.

### Dispersion Means

In this and other component, compound/agent/constituent, or method aspect(s) of the invention, the invention also can be characterized as comprising a “means” for dispersion of particles, e.g., a means for improving the separation of particles, a means of detectably or significantly improving the separation of particles, a means of detectably or significantly preventing the settling or clumping of particles, or any combination of any or all thereof. In such a respect, any known equivalents of such named dispersion component(s), compound(s), or constituent(s) can also be, e.g., are, incorporated into composition(s) or method(s) of the invention. As with other sections similarly described herein, any of the component(s), compound(s), agent(s), constituent(s), or, e.g., method(s) of the invention can be, where suitable, described as means (e.g., the above-described dispersion component(s), compound(s), or constituent(s) can be described as dispersion means or means for dispersion of particles, e.g., a means for improving the separation of particles, a means of detectably or significantly improving the separation of particles, a means of detectably or significantly preventing the settling or clumping of particles, or any combination of any or all thereof.)

### Antioxidant(s)

In aspects, composition(s) herein can comprise an antioxidant component comprising one or more antioxidant compound(s)/agent(s). In aspects, an antioxidant compound/agent is a composition agent/constituent which detectably or significantly detectably or significantly reduces, delays, or prevents oxidation of one or more component(s), e.g., compound(s), of a composition.

In aspects, a constituent of an antioxidant component can comprise, e.g., one or more compound(s) recognized in the art as being capable of providing one or more activity(ies) aside from that of an antioxidant or is, for example, alternately characterizable as a specific type of compound, such as, e.g., an organic acid. In aspects, uncontradicted, an antioxidant can encompass certain organic acid(s) described herein.

In aspects, composition(s) can comprise an antioxidant component comprising any pharmaceutically acceptable and compositionally compatible antioxidant compound(s)/agent(s). According to certain aspects, an antioxidant component of composition(s) provided herein can comprise, e.g., ascorbic acid, alpha-tocopherol, BHT (butylhydroxytoluene), BHA (butylhydroxyanisole), etc. In aspects, composition(s) can comprise a mixture of any two or more compound(s)/agent(s) antioxidant component constituent(s) described in this paragraph or elsewhere herein.

### Antioxidant Amount

In certain aspects, composition(s) herein lack any component or compound/agent which is characterizable as an antioxidant. In aspects, composition(s) herein are characterizable as antioxidant free.

Here and in similar sections herein, amounts presented can, in aspects, represent the amount of a single antioxidant compound/agent, the amount of an antioxidant component (e.g., representing the total amount of antioxidant compound(s) in a composition, or both. In aspects, an antioxidant component of a composition can comprise two or more compounds each present in a composition in amount(s) described here.

In aspects, composition(s) herein comprise an effective amount of a pharmaceutically acceptable and compositionally compatible antioxidant component. According to certain aspects, composition(s) comprise an antioxidant component wherein the antioxidant component is present in the composition in an amount representing between about 0.00001 wt. % and about 0.5 wt. %, such as, e.g., ˜0.00001 wt. %-˜0.45 wt. %, ˜0.00001 wt. %-˜0.4 wt. %, ˜0.00001 wt. %-˜0.35 wt. %, ˜0.00001 wt. %-˜0.3 wt. %, ˜0.00001 wt. %-˜0.25 wt. %, ˜0.00001 wt. %-˜0.2 wt. %, ˜0.00001 wt. %-˜0.15 wt. %, or, e.g., ˜0.00001 wt. %-˜0.1 wt. % of the composition.

In certain aspects, composition(s) comprise an antioxidant component wherein the antioxidant component is present in the composition in an amount representing between about 0.00002 wt. %-˜0.5 wt. %, such as, e.g., ˜0.00004 wt. %-˜0.5 wt. %, ˜0.00006 wt. %-˜0.5 wt. %, ˜0.00008 wt. %-˜0.5 wt. %, ˜0.0001 wt. %-˜0.5 wt. %, ˜0.0002 wt. %-˜0.5 wt. %, ˜0.0004 wt. %-˜0.5 wt. %, ˜0.0006 wt. %-˜0.5 wt. %, ˜0.0008 wt. %-˜0.5 wt. % or, e.g., ˜0.001 wt. %-˜0.5 wt. % of the composition.

In aspects, composition(s) comprise an antioxidant component wherein the antioxidant component is present in the composition in an amount representing ˜0.00002 wt. %-˜0.45 wt. %, ˜0.00004 wt. %-˜0.4 wt. %, ˜0.00006 wt. %-˜0.4 wt. %, ˜0.00008 wt. %-˜0.35 wt. %, ˜0.0001 wt. %-˜0.3 wt. %, ˜0.0002 wt. %-˜0.25 wt. %, ˜0.0004 wt. %-˜0.2 wt. %, ˜0.0006 wt. %-˜0.15 wt. %, ˜0.0008 wt. %-˜0.15 wt. %, or, e.g., ˜0.001 wt. %-˜0.1 wt. % of the composition.

### Antioxidant Means

In this and other component, compound/agent/constituent, or method aspect(s) of the invention, the invention also can be characterized as comprising a “means” for detectably or significantly reducing, delaying, or preventing oxidation of one or more component(s), e.g., compound(s) of a composition. In such a respect, any known equivalents of such named antioxidant component(s), compound(s), or constituent(s) can also be, e.g., are, incorporated into composition(s) or method(s) of the invention. As with other sections similarly described herein, any of the component(s), compound(s), agent(s), constituent(s), or, e.g., method(s) of the invention can be, where suitable, described as means (e.g., the above-described antioxidant component(s), compound(s), or constituent(s) can be described as antioxidant means or means for detectably or significantly reducing, delaying, or preventing oxidation of one or more component(s), e.g., compound(s), of composition(s).)

### Mineral Acid(s)

In aspects, composition(s) herein can comprise a mineral acid component comprising one or more mineral acid compound(s)/agent(s). In aspects, a mineral acid may be present to, e.g., detectably or significantly adjust composition pH or, e.g., to perform one or more functional activity(ies).

In aspects, a constituent of a mineral acid component can comprise, e.g., one or more compound(s) recognized in the art as being capable of providing one or more activity(ies) described herein, such as, e.g., pH adjusting activity or, e.g., stabilizing activity. In aspects, a mineral acid can encompass such function(s) and be present in such amounts as described herein.

In aspects, presence of a mineral acid, e.g., in a composition provided as a solution or, e.g., a solution-suspension hybrid, detectably or significantly increases the stability of one or more API(s), e.g., tiotropium. In aspects, a detectable or significant stabilization by the mineral acid is provided via pH modulation capability. In certain aspects, a mineral acid is absent. In aspects, a mineral acid is absent in composition(s) provided as a suspension.

In aspects, composition(s) can comprise a mineral acid component comprising any pharmaceutically acceptable and compositionally compatible mineral acid(s). According to certain aspects, a suspension component of composition(s) provided herein can comprise, e.g., boric acid, chromic acid, hydrochloric acid, hydrofluoric acid, nitric acid, perchloric acid, phosphoric acid, sulfuric acid, etc.

### Mineral Acid Amount

In certain aspects, composition(s) herein lack any component or compound/agent which is characterizable as a mineral acid. In aspects, composition(s) herein are characterizable as mineral acid free.

Here and in similar sections herein, amounts presented can, in aspects, represent the amount of a single mineral acid compound/agent, the amount of a mineral acid component (e.g., representing the total amount of mineral acid compound(s) in a composition, or both. In aspects, a mineral acid component of a composition can comprise two or more compounds each present in a composition in amount(s) described here.

In aspects, composition(s) herein comprise an effective amount of a pharmaceutically acceptable and compositionally compatible mineral acid component. According to certain aspects, composition(s) comprise a mineral acid component wherein the mineral acid component is present in an amount representing between about 0.00001 wt. % and about 3 wt. %, such as, e.g., ˜0.00001 wt. %-˜2 wt. %, ˜0.00001 wt. %-˜1 wt. %, ˜0.00001 wt. %-˜0.9 wt. %, ˜0.00001 wt. %-˜0.85 wt. %, ˜0.00001 wt. %-˜0.8 wt. %, ˜0.00001 wt. %-˜0.75 wt. %, ˜0.00001 wt. %-˜0.7 wt. %, ˜0.00001 wt. %-˜0.65 wt. %, ˜0.00001 wt. %-˜0.6 wt. %, ˜0.00001 wt. %-˜0.55 wt. %, or, e.g., ˜0.00001 wt. %-˜0.5 wt. %, ˜0.00001 wt. %-˜0.45 wt. %, ˜0.00001 wt. %-˜0.4 wt. %, ˜0.00001 wt. %-˜0.35 wt. %, ˜0.00001 wt. %-˜0.3 wt. %, ˜0.00001 wt. %-˜0.25 wt. %, ˜0.00001 wt. %-˜0.2 wt. %, or, e.g., ˜0.00001 wt. %-˜0.15 wt. %, or ˜0.00001 wt. %-˜0.1 wt. % of the composition.

In aspects, composition(s) comprise a mineral acid component wherein the mineral acid component is present in an amount representing between about 0.0005 wt. % and about 5 wt. %, ˜4 wt. %, ˜3 wt. % or ˜2 wt. %, such as, e.g., ˜0.0001 wt. %-˜1 wt. %, ˜0.0002 wt. %-˜1 wt. %, ˜0.0003 wt. %-˜1 wt. %, ˜0.0004 wt. %-˜1 wt. %, ˜0.0005 wt. %-˜1 wt. %, ˜0.0006 wt. %-˜1 wt. %, ˜0.0007 wt. %-˜1 wt. %, ˜0.0008 wt. %-˜1 wt. %, ˜0.0009 wt. %-˜1 wt. %, or, e.g., ˜0.001 wt. %-˜1 wt. % of the composition.

In certain aspects, composition(s) comprise a mineral acid component wherein the mineral acid component is present in an amount representing between about 0.00001 wt. % and about 4.5 wt. %, ˜0.00005 wt. %-˜4 wt. %, ˜0.00001 wt. %-˜3.5 wt. %, ˜0.0005 wt. %-˜3 wt. %, ˜0.0001 wt. %-˜2.5 wt. %, ˜0.005 wt. %-˜2 wt. %, or, e.g., ˜0.001 wt. %-˜1.5 wt. %, or, e.g., ˜0.001 wt. %-˜1 wt. %, ˜0.001 wt. %-˜0.9 wt. %, ˜0.001 wt. %-˜0.8 wt. %, ˜0.001 wt. %-˜0.7 wt. %, ˜0.001 wt. %-˜0.6 wt. %, ˜0.001 wt. %-˜0.5 wt. %, ˜0.001 wt. %-˜0.4 wt. %, ˜0.001 wt. %-˜0.3 wt. %, ˜0.001 wt. %-˜0.2 wt. %, or, e.g., ˜0.001 wt. %-˜0.1 wt. %.

In aspects, composition(s) comprise, e.g., hydrochloric acid, or, e.g., phosphoric acid, in such amounts disclosed in this section. According to certain embodiments, composition(s) comprise hydrochloric acid in an amount of about 0.01 wt. %, ˜0.0125 wt. %, ˜0.015 wt. %, ˜0.016 wt. %, 0.0165 wt. %, ˜0.017 wt. %, ˜0.0175 wt. %, ˜0.018 wt. %, ˜0.0185 wt. %, ˜0.019 wt. %, ˜0.0195 wt. %, ˜0.02 wt. %, ˜0.0225 wt. %, ˜0.025 wt. %, ˜0.0275 wt. %, ˜0.03 wt. %, ˜0.0325 wt. %, ˜0.035 wt. %, ˜0.0375 wt. %, ˜0.04 wt. %, ˜0.0425 wt. %, ˜0.045 wt. %, ˜0.0475 wt. %, or, e.g., ˜0.05 wt. % of the composition.

In aspects, such amounts herein can be used to describe amounts of a mineral acid, such as, e.g., hydrochloric acid, as provided to composition(s) as a 1M solution.

### Absorbent(s)

In aspects, composition(s) herein can comprise an absorbent component comprising one or more absorbent compound(s)/agent(s). In aspects, an absorbent compound/agent is a composition agent/constituent which provides detectable or significant desiccant activity (e.g., is capable of detectably or significantly absorbing a detectable or significant amount of water).

In aspects, a constituent of an absorbent component can comprise, e.g., one or more compound(s) recognized in the art as being capable of providing one or more activity(ies). In aspects where such an absorbent component compound can be characterizable as, providing e.g., a function described elsewhere herein, uncontradicted, such absorbent(s) can encompass such compound(s) described herein, and, e.g., can be present in such disclosed amounts as described herein.

In aspects, composition(s) can comprise an absorption component comprising any pharmaceutically acceptable and compositionally compatible absorbent compound(s)/agent(s). According to certain aspects, a suspension component of composition(s) provided herein can comprise, e.g., sodium cromoglycate.

### Absorbent Amount

In certain aspects, composition(s) herein lack any component or compound/agent which is characterizable as an absorbent. In aspects, composition(s) herein are characterizable as absorbent free.

Here and in similar sections herein, amounts presented can, in aspects, represent the amount of a single absorbent compound/agent, the amount of an absorbent component (e.g., representing the total amount of absorbent compound(s) in a composition, or both. In aspects, an absorbent component of a composition can comprise two or more compounds each present in a composition in amount(s) described here.

In aspects, composition(s) herein comprise an effective amount of a pharmaceutically acceptable and compositionally compatible absorbent component. In aspects, composition(s) herein comprise an absorbent component, wherein the absorbent component is present in an amount of between about 0.0001 wt. % and about 0.2 wt. %, such as, e.g., ˜0.0001 wt. %-˜0.18 wt. %, ˜0.0001 wt. %-˜0.16 wt. %, ˜0.0001 wt. %-˜0.14 wt. %, ˜0.0001 wt. %-˜0.12 wt. %, or, e.g., ˜0.0001 wt. %-˜0.1 wt. % of the composition.

In aspects, composition(s) herein comprise an absorbent component, wherein the absorbent component is present in an amount of between about 0.0002 wt. % and ˜0.2 wt. %, such as, e.g., ˜0.0004 wt. %-˜0.2 wt. %, ˜0.0006 wt. %-˜0.2 wt. %, ˜0.0008 wt. %-˜0.2 wt. %, or, e.g., ˜0.001 wt. %-˜0.2 wt. % of the composition.

In aspects, composition(s) herein comprise an absorbent component, wherein the absorbent component is present in an amount of between about 0.002 wt. % and about 0.18 wt. %, such as, e.g., ˜0.004 wt. %-˜0.16 wt. %, ˜0.006 wt. %-˜0.14 wt. %, ˜0.008 wt. %-˜0.12 wt. %, or, e.g., about 0.1 wt. % of the composition. In aspects, compositions comprise an absorbent component, e.g., sodium cromoglycate, in an amount of between about 0.001 wt. % and about 0.005 wt. %, such as, ˜0.001 wt. %-˜0.004 wt. %, ˜0.001 wt. %-˜0.003 wt. %, or ˜0.0015 wt. %-˜0.0025 wt. %, e.g., ˜0.002 wt. % of the composition.

### Absorbent Means

In this and other component, compound, agent, or, e.g., constituent aspect(s) of the invention, the invention also can be characterized as comprising a “means” for providing absorbent/desiccant activity. In such a respect, any known equivalents of such named functionality can also be, e.g., are, incorporated into composition(s) or method(s) of the invention. As with other sections similarly described herein, any of the component(s), compound(s), agent(s), or, e.g., constituent(s) of the invention can be, where suitable, described as means (e.g., the above-described absorbent mechanisms can be described as absorbent means or means for providing absorbent/desiccant activity).

### pH Adjusting Agent(s)

In aspects, composition(s) herein can comprise a pH adjusting component comprising one or more pH adjusting compound(s)/agent(s). In aspects, a pH adjusting agent is a composition agent/constituent which detectably or significantly contributes to the establishment of a particular pH of a composition (e.g., is added to adjust the pH of composition(s) to a given target pH.)

In aspects, a constituent of a suspension component can comprise, e.g., one or more compound(s) recognized in the art as being capable of providing one or more activity(ies) aside from that of or is characterizable in a manner other than that of, a pH adjusting agent. In aspects, pH adjusting compound(s) may further be characterizable as, e.g., a mineral acid or, e.g., an organic acid, etc. In aspects, uncontradicted, such compound(s) can encompass such function(s) described herein, and, e.g., can be present in such disclosed amounts as described herein.

In aspects, composition(s) can comprise a pH adjusting component comprising any pharmaceutically acceptable and compositionally compatible pH adjusting compound(s)/agent(s). According to certain aspects, a pH adjusting component of composition(s) provided herein can comprise, e.g., one or more organic acids, one or more inorganic acids, or, e.g., both organic acid(s) and inorganic acid(s). In aspects, exemplary compound(s) include but are not limited to, e.g., citric acid, maleic acid, ascorbic acid, hydrochloric acid, sulfuric acid, nitric acid, or phosphoric acid, etc. In aspects, composition(s) can comprise a mixture of any two or more compound(s)/agent(s) suspension component constituent(s) described in this paragraph or elsewhere herein.

According to certain aspects, a pH adjusting agent component constituent is selected from one or more organic or inorganic acids which are non-mineral acid, such as, e.g., citric acid, maleic acid, or, e.g., ascorbic acid. In aspects, a pH adjusting agent component constituent is a mineral acid, such as, e.g., HCl. In certain aspects, composition(s) comprise both a mineral acid and at least one other acid, e.g., an organic acid, a stabilizing acid, a surfactant, or both. In some aspects, the specific acids used and, e.g., their combination amounts, detectably or significantly modify one or more properties of the composition(s), such as, e.g., solubilization of API, stability of API, aerosolization capability/performance, stability, etc.

### pH Adjusting Agent Amount

According to certain aspects, composition(s) herein lack any component or compound/agent which is characterizable as a pH adjusting agent. In aspects, composition(s) herein are characterizable as pH-adjusting agent free.

Here and in similar sections herein, amounts presented can, in aspects, represent the amount of a single pH adjusting compound/agent, the amount of a pH adjusting component (e.g., representing the total amount of pH adjusting compound(s) in a composition, or both. In aspects, a pH adjusting component of a composition can comprise two or more compounds each present in a composition in amount(s) described here.

In aspects, composition(s) herein comprise an effective amount of a pharmaceutically acceptable and compositionally compatible pH adjusting component. In aspects, composition(s) comprise a pH adjusting component wherein the pH adjusting component is present in an amount representing about 0.000001 wt. %-1 wt. % of the composition, such as, e.g., ˜0.000001 wt. %-˜0.8 wt. %, ˜0.000001 wt. %-˜0.6 wt. %, ˜0.000001 wt. %-˜0.4 wt. %, ˜0.000001 wt. %-˜0.2 wt. %, ˜0.000001 wt. %-˜0.1 wt. %, or, e.g., ˜0.000001 wt. %-˜0.08 wt. %, ˜0.000001 wt. %-˜0.06 wt. %, ˜0.000001 wt. %-˜0.04 wt. %, ˜0.000001 wt. %-˜0.02 wt. %, or, e.g., ˜0.000001 wt. %-˜0.01 wt. % of the composition.

In aspects, composition(s) comprise a pH adjusting component wherein the pH adjusting component is present in an amount representing about 0.000005 wt. %-˜1 wt. % of the composition, e.g., ˜0.00001 wt. %-˜1 wt. %, ˜0.0005 wt. %-˜1 wt. %, ˜0.001 wt. %-˜1 wt. %, or, e.g., ˜0.05 wt. %-˜1 wt. % or, e.g., ˜0.1 wt. %-˜1 wt. % of the composition.

In aspects, composition(s) comprise a pH adjusting component wherein the pH adjusting component is present in an amount representing about 0.000005 wt. %-˜0.8 wt. %, ˜0.00001 wt. %-˜0.6 wt. %, ˜0.0005 wt. %-˜0.4 wt. %, ˜0.001 wt. %-˜0.2 wt. %, ˜0.005 wt. %-˜0.1 wt. %, ˜0.006 wt. %-˜0.09 wt. %, ˜0.007 wt. %-˜0.08 wt. %, ˜0.008 wt. %-˜0.07 wt. %, ˜0.009 wt. %-˜0.06 wt. %, or, e.g., ˜0.01 wt. %-˜0.05 wt. % of the composition.

### pH Adjusting Means

In this and other component, compound/agent/constituent, or method aspect(s) of the invention, the invention also can be characterized as comprising a “means” for adjusting the pH of composition(s). In such a respect, any known equivalents of such named pH-adjusting component(s), compound(s), or constituent(s) can also be, e.g., are, incorporated into composition(s) or method(s) of the invention. As with other sections similarly described herein, any of the component(s), compound(s), agent(s), constituent(s), or, e.g., method(s) of the invention can be, where suitable, described as means (e.g., the above-described pH-adjusting component(s), compound(s), or constituent(s) can be described as pH-adjusting means or means for adjusting (e.g., changing) the pH of composition(s).)

### Water

In aspects, composition(s) herein can comprise water. In aspects, water is present as a solvent. In aspects, water may demonstrate one or more activity(ies) aside from that of a solvent. In aspects, in such cases, uncontradicted, such functionality(ies) as described herein may be encompassed, and water may be present in such disclosed amounts as described herein.

### Water Amount

In aspects, composition(s) can comprise water in a minimal amount, so as to minimize any interference water may contribute to compositional instability or, e.g., any interference water may cause to delivery device operation (such, as, e.g., by causing particulate sticking to one or more device component(s), etc.)

In aspects, composition(s) herein comprise an effective amount, e.g., a compositionally compatible, amount of water. In certain aspects, composition(s) herein are water free. In other aspects, composition(s) can comprise, e.g., between about 0.0001 wt. % and about 2 wt. % water, such as, e.g., ˜0.0001 wt. %-˜1.8 wt. %, ˜0.0001 wt. %-˜1.6 wt. %, ˜0.0001 wt. %-˜1.4 wt. %, ˜0.0001 wt. %-˜1.2 wt. %, ˜0.0001 wt. %-˜1 wt. %, ˜0.0001 wt. %-˜0.08 wt. %, ˜0.0001 wt. %-˜0.06 wt. %, ˜0.0001 wt. %-˜0.04 wt. %, ˜0.0001 wt. %-˜0.02 wt. %, or, e.g., ˜0.0001 wt. %-˜0.01 wt. % water.

In aspects, composition(s) herein comprise water in an amount of between about 0.0002 wt. % and about 2 wt. %, such as, e.g., ˜0.0004 wt. %-˜2 wt. %, ˜0.0006 wt. %-˜2 wt. %, ˜0.0008 wt. %-˜2 wt. %, ˜0.001 wt. %-˜2 wt. %, ˜0.002 wt. %-˜2 wt. %, ˜0.004 wt. %-˜2 wt. %, ˜0.006 wt. %-˜2 wt. %, ˜0.008 wt. %-˜2 wt. %, or, e.g., ˜0.01 wt. %-˜2 wt. %, ˜0.02 wt. %-˜2 wt. %, ˜0.04 wt. %-˜2 wt. %, ˜0.06 wt. %-˜2 wt. %, ˜0.08 wt. %-˜2 wt. %, ˜0.1 wt. %-˜2 wt. %, or, e.g., ˜0.15 wt. %-˜2 wt. % or ˜0.2 wt. %-˜2 wt. % of the composition. In certain aspects, composition(s) comprise water in an amount representing greater than about 0.05 wt. % of composition(s) but less than about 1 wt. % of composition(s).

In aspects, composition(s) herein comprise water in an amount of between about 0.002 wt. % and about 1.8 wt. %, such as, e.g., 0.004 wt. %-˜1.6 wt. %, 0.006 wt. %-˜1.4 wt. %, 0.008 wt. %-˜1.2 wt. %, 0.01 wt. %-˜1 wt. %, 0.01 wt. %-˜0.8 wt. %, 0.01 wt. %-˜0.6 wt. %, 0.01 wt. %-˜0.4 wt. %, 0.01 wt. %-˜0.2 wt. %, or, e.g., 0.01 wt. %-˜0.1 wt. %, such as, e.g., 0.1 wt. %-˜0.9 wt. %, 0.2 wt. %-˜0.8 wt. %, 0.3 wt. %-˜0.7 wt. %, 0.4 wt. %-˜0.6 wt. %, or, e.g., ˜0.5 wt. %, or, e.g., ˜0.02 wt. %, ˜0.03 wt. %, ˜0.04 wt. %, ˜0.05 wt. %, ˜0.06 wt. %, ˜0.07 wt. %, ˜0.08 wt. %, or, e.g., ˜0.09 wt. % of the composition.

According to certain aspects, composition(s) herein comprise water in an amount greater than about 0.05 wt. %, such as, e.g., ≥˜0.06 wt. %, ≥˜0.07 wt. %, ≥˜0.08 wt. %, ≥˜0.09 wt. %, or ≥˜0.1 wt. %, such as, e.g., ≥˜0.2 wt. %, ≥˜0.3 wt. %, ≥˜0.4 wt. %, ≥˜0.5 wt. %, ≥˜0.6 wt. %, ≥˜0.7 wt. %, ≥˜0.8 wt. %, ≥˜0.9 wt. %, or, e.g., ≥˜1 wt. %, as in, for example, ≥˜1.1 wt. %, ≥˜1.2 wt. %, ≥˜1.3 wt. %, ≥˜1.4 wt. %, ≥˜1.5 wt. %, ≥˜1.6 wt. %, ≥˜1.7 wt. %, ≥˜1.8 wt. %, ≥˜1.9 wt. %, or, e.g., ≥˜2 wt. %.

According to certain aspects, composition(s) herein comprise water in an amount of less than about 1 wt. %, such as, e.g., ≤˜0.9 wt. %, ≤˜0.8 wt. %, ≤˜0.7 wt. %, ≤˜0.6 wt. %, ≤˜0.5 wt. %, ≤˜0.4 wt. %, ≤˜0.3 wt. %, ≤˜0.2 wt. %, or, e.g., ≤˜0.1 wt. %, such as, e.g., ≤˜0.09 wt. %, ≤˜0.08 wt. %, ≤˜0.07 wt. %, ≤˜0.06 wt. %, or, e.g., ≤˜0.05 wt. %.

### Other Excipient(s)

In aspects, composition(s) herein can comprise one or more excipient(s) not otherwise described or disclosed elsewhere herein.

In aspects, composition(s) comprise one or more preservatives, such as, e.g., benzalkonium chloride, EDTA, benzoic acid, benzoates (e.g., sodium benzoate), etc. In aspects, composition(s) lack any one or more such compounds. In aspects, composition(s) comprise one or more chelating agents such as, an EDTA compound such as, e.g., edit acid, sodium EDTA, or sodium edetate (disodium EDTA dihydrate). In aspects, composition(s) lack any one or more such compounds.

In aspects, composition(s) can comprise one or more compound(s)/agent(s) which demonstrate one or more functionality(ies) described elsewhere herein but which also perform one or more functionality(ies) not otherwise described here. In aspects, for example, composition(s) herein can comprise one or more excipient compound(s) which detectably or significantly aids in providing an average aerodynamic diameter of aerosol particles which is less than about 10 μm, e.g., ≤˜9 μm, ≤˜8 μm, ≤˜7 μm, or ≤˜6 μm, such as, e.g., ≤˜5 μm, ≤˜4 μm, ≤˜3 μm, ≤˜2 μm, or, e.g., ≤˜1 μm. In aspects, composition(s)(s) herein can comprise one or more excipient compound(s) which detectably or significantly increases the percentage of particles which is less than about 10 μm, e.g., ≤˜9 μm, ≤˜8 μm, ≤˜7 μm, or ≤˜6 μm, such as, e.g., ≤˜5 μm, ≤˜4 μm, ≤˜3 μm, ≤˜2 μm, or, e.g., ≤˜1 μm. In aspects, composition(s) herein can comprise one or more excipient compound(s) which detectably or significantly aids in maintaining a median mass aerodynamic particle diameter (MMAD) of between about, e.g., ˜3 μm-˜3.7 μm, such as, e.g., ˜3.1 μm-˜3.6 μm, or, e.g., ˜3.2 μm-˜3.5 μm. In aspects, composition(s) herein can comprise one or more excipient compound(s) which detectably or significantly aids in establishing a fine particle fraction (FPF) less than about 5 μm of between about 30%-about 70%, such as, e.g., ˜32%-˜68%, ˜34%-˜66%, ˜36%-˜64%, ˜38%-˜62%, or, e.g., ˜40%-˜60%, such as, e.g., ˜42%-˜58%, ˜44%-˜56%, or, e.g., ˜46%-˜54%.

In other exemplary aspects, compositions can comprise, e.g., one or more taste modifying agents, compound(s) capable of, when present in effective amount(s), detectably or significantly modifying the taste of composition(s), including, e.g., detectably or significantly “masking” or “hiding” a taste which could be identified by a recipient of composition(s) as unpleasant or undesirable. In aspects, compositions can also or alternatively comprise, e.g., one or more odor modifying agents, compound(s) capable of, when present in effective amount(s), detectably or significantly modifying the odor (smell) of composition(s), including, e.g., detectably or significantly “masking” or “hiding” an odor/smell which could be identified by a recipient of composition(s) as unpleasant. In aspects, a taste modifying agent, odor modifying agent, or both can be, e.g., any inhalable compound capable of detectably or significantly modifying the taste, odor, or both of compositions when present in effective amount(s), such as, e.g., menthol, saccharin, or, e.g., any suitably inhalation-safe compound.

### Other Excipient Amount

In certain aspects, composition(s) herein explicitly lack any compound other than a compound specifically identified in this disclosure. In aspects, composition(s) herein lack a constituent which is not characterizable as having a functionality specifically identified in this disclosure. In aspects, composition(s) herein lack any one or more excipient(s) having any one or more of the functionality(ies) described in this “other excipient” section.

In aspects, composition(s) herein comprise an effective amount of one or more pharmaceutically acceptable and compositionally compatible other excipient component(s). In aspects, composition(s) herein can comprise one or more other excipient compound(s)/agent(s) in any pharmaceutically acceptable, compositionally compatible, and efficacious amount. For example, one or more preservative(s), if present, can be present in an amount representing between about 0.00001 and about 0.2 wt. %, such as, e.g., between about 0.0001 wt. % and about 0.2 wt. %, as in, e.g., between about 0.001 wt. % and about 0.2 wt. % or, e.g., between about 0.01 wt. % and about 0.2 wt. % of a composition. In another example, one or more chelating agent(s), if present, can be present in an amount representing between about 0.00001 and about 0.2 wt. %, such as, e.g., between about 0.0001 wt. % and about 0.2 wt. %, as in, e.g., between about 0.001 wt. % and about 0.2 wt. % or, e.g., between about 0.01 wt. % and about 0.2 wt. % of a composition. In aspects, compositions comprise less than about 1 wt. % of an odor or taste modifying agent, such as, e.g., ≤˜0.9 wt. %, ≤˜0.8 wt. %, ≤˜0.7 wt. %, ≤˜0.6 wt. %, ≤˜0.5 wt. %, ≤˜0.4 wt. %, ≤˜0.3 wt. %, ≤˜0.2 wt. %, or, e.g., ≤˜0.1 wt. % of odor, taste, or both modifying agent(s), such as, e.g., menthol, saccharin, or both.

### Ratios

According to aspects, any component(s) or compound(s)/agent(s) described herein can be present in composition(s) in therapeutically effective amount(s), compositionally compatible amount(s), or both. In aspects, any single component or compound/agent provided herein can be present in a relationship with, such as, e.g., in a ratio with, any one or more other single component or compound/agent. In aspects, any combination of component(s) or compound(s)/agent(s) provided herein can be present in a ratio with any other combination of component(s) or compound(s)/agent(s). In aspects, ratio(s) between such component(s) or compound(s)/agent(s) or combinations thereof can be established using any provided amounts for each disclosed herein, including, e.g., values within ranges of such amounts disclosed herein. To exemplify this disclosure, the following table is provided. Table 1 below, e.g., illustrating a ratio array, demonstrates the types of ratios which the reader should understand to be encompassed by the disclosure herein.

The reader should understand that the ratio array illustrated in Table 1 is exemplary and does not necessarily disclose all possible ratios encompassed by this disclosure. For example, groups of such provided components can be, e.g., present in relationship to, e.g., as a ratio with, other one or more, e.g., groups, of provided components.

TABLE 1
Exemplary Ratio Array.

| AP | BC | LC | AC | TI | BA | FO | MC | SC | IC |
|---|---|---|---|---|---|---|---|---|---|
| BC | — | LC:BC | AC:BC | TI:BC | BA:BC | FO:BC | MC:BC | SC:BC | IC:BC |
| LC | BC:LC | — | AC:LC | TI:LC | BA:LC | FO:LC | MC:LC | SC:LC | IC:LC |
| AC | BC:AC | LC:AC | — | TI:AC | BA:AC | FO:AC | MC:AC | SC:AC | IC:AC |
| TI | BC:TI | LC:TI | AC:TI | — | BA:TI | FO:TI | MC:TI | SC:TI | IC:TI |
| BA | BC:BA | LC:BA | AC:BA | TI:BA | — | FO:BA | MC:BA | SC:BA | IC:BA |
| FO | BC:FO | LC:FO | AC:FO | TI:FO | BA:FO | — | MC:FO | SC:FO | IC:FO |
| MC | BC:MC | LC:MC | AC:MC | TI:MC | BA:MC | FO:MC | — | SC:MC | IC:MC |
| SC | BC:SC | LC:SC | AC:SC | TI:SC | BA:SC | FO:SC | MC:SC | — | IC:SC |
| IC | BC:IC | LC:IC | AC:IC | TI:IC | BA:IC | FO:IC | MC:IC | SC:IC | — |
| PC | BCPC | LC:PC | AC:PC | TI:PC | BA:PC | FO:PC | MC:PC | SC:PC | IC:PC |
| HC | BC:HC | LC:HC | AC:HC | TI:HC | BA:HC | FO:HC | MC:HC | SC:HC | IC:HC |
| FC | BC:FC | LC:FC | AC:FC | TI:FC | BA:FC | FO:FC | MC:FC | SC:FC | IC:FC |
| VC | BC:VC | LC:VC | AC:VC | TI:VC | BA:VC | FO:VC | MC:VC | SC:VC | IC:VC |
| NC | BC:NC | LC:NC | AC:NC | TI:NC | BA:NC | FO:NC | MC:NC | SC:NC | IC:NC |
| KC | BC:KC | LC:KC | AC:KC | TI:KC | BA:KC | FO:KC | MC:KC | SC:KC | IC:KC |
| ZC | BC:ZC | LC:ZC | AC:ZC | TI:ZC | BA:ZC | FO:ZC | MC:ZC | SC:ZC | IC:ZC |
| OA | BC:OA | LC:OA | AC:OA | TI:OA | BA:OA | FO:OA | MC:OA | SC:OA | IC:OA |
| AB | BC:AB | LC:AB | AC:AB | TI:AB | BA:AB | FO:AB | MC:AB | SC:AB | IC:AB |
| AP | PC | HC | FC | VC | NC | KC | ZC | OA | AB |
| BC | PC:BC | HC:BC | FC:BC | VC:BC | NC:BC | KC:BC | ZC:BC | OA:BC | AB:BC |
| LC | PC:LC | HC:LC | FC:LC | VC:LC | NC:LC | KC:LC | ZC:LC | OA:LC | AB:LC |
| AC | PC:AC | HC:AC | FC:AC | VC:AC | NC:AC | KC:AC | ZC:AC | OA:AC | AB:AC |
| TI | PC:TI | HC:TI | FC:TI | VC:TI | NC:TI | KC:TI | ZC:TI | OA:TI | AB:TI |
| BA | PC:BA | HC:BA | FC:BA | VC:BA | NC:BA | KC:BA | ZC:BA | OA:BA | AB:BA |
| FO | PC:FO | HC:FO | FC:FO | VC:FO | NC:FO | KC:FO | ZC:FO | OA:FO | AB:FO |
| MC | PC:MC | HC:MC | FC:MC | VC:MC | NC:MC | KC:MC | ZC:MC | OA:MC | AB:MC |
| SC | PC:SC | HC:SC | FC:SC | VC:SC | NC:SC | KC:SC | ZC:SC | OA:SC | AB:SC |
| IC | PC:IC | HC:IC | FC:IC | VC:IC | NC:IC | KC:IC | ZC:IC | OA:IC | AB:IC |
| PC | — | HC:PC | FC:PC | VC:PC | NC:PC | KC:PC | ZC:PC | OA:PC | AB:PC |
| HC | PC:HC | — | FC:HC | VC:HC | NC:HC | KC:HC | ZC:HC | OA:HC | AB:HC |
| FC | PC:FC | HC:FC | — | VC:FC | NC:FC | KC:FC | ZC:FC | OA:FC | AB:FC |
| VC | PC:VC | HC:VC | FC:VC | — | NC:VC | KC:VC | ZC:VC | OA:VC | AB:VC |
| NC | PC:NC | HC:NC | FC:NC | VC:NC | — | KC:NC | ZC:NC | OA:NC | AB:NC |
| KC | PC:KC | HC:KC | FC:KC | VC:KC | NC:KC | — | ZC:KC | OA:KC | AB:KC |
| ZC | PC:ZC | HC:ZC | FC:ZC | VC:ZC | NC:ZC | KC:ZC | — | OA:ZC | AB:ZC |
| OA | PC:OA | HC:OA | FC:OA | VC:OA | NC:OA | KC:OA | ZC:OA | — | AB:OA |
| AB | PC:AB | HC:AB | FC:AB | VC:AB | NC:AB | KC:AB | ZC:AB | OA:AB | — |

Abbreviations:
AB—Absorbent component
AC—Anticholinergic component
AP—Active Pharmaceutical Ingredient (API)
BA—Beta-adrenergic component
BC—Bronchodilating component
BC—Bulking component
FC—Surfactant component
FO—Formoterol compound
HC—Hydrofluoroolefin component
IC—Inhaled corticosteroid component
LC—Long-acting bronchodilating component
MC—Anti-inflammatory component component
NC—Suspension component
OA—Organic acid component
PC—Propellant component
SC—Steroid component
TI—Tiotropium compound
VC—Solvent component
ZC—Stabilizing component

In aspects, composition(s) (as described herein) comprise a stabilizing agent characterizable as a small compound stabilizing agent (pharmaceutically acceptable and compositionally compatible small compound stabilizing agent(s)). In aspects, such a stabilizing agent has a molecular weight of less than about 200 g/mol; provides detectable or significant chelating activity; or both. In aspects, a small compound stabilizing agent is capable of detectably or significantly preventing or delaying any change in the homogeneity of the composition resulting in (a) the amount of light capable of passing through the composition to be reduced or (b) the composition no longer being characterizable as a solution when originally prepared as a solution formulation. In aspects, composition(s) further comprise at least one C1-C6 alcohol characterizable as an alkyl alcohol, an aliphatic alcohol, or both. In aspects, the ratio of a pharmaceutically acceptable and compositionally compatible small compound stabilizing agent to at least one C1-C6 alcohol characterizable as an alkyl alcohol, an aliphatic alcohol, or both is not greater than about 1:300, such as, e.g., ≤˜1:310, ≤˜1:320, ≤˜1:330, ≤˜1:340, ≤˜1:350, ≤˜1:360, ≤˜1:370, ≤˜1:380, ≤˜1:390, ≤˜1:400, ≤˜1:410, ≤˜1:420, ≤˜1:430, ≤˜1:440, ≤˜1:450, or, e.g., ≤˜1:460. In certain aspects, a small compound stabilizing agent is, e.g., citric acid, malic acid, or, e.g., maleic acid. In aspects, a C1-C6 alcohol is ethanol.

According to other aspects, compositions comprise a ratio of pharmaceutically acceptable and compositionally compatible small compound stabilizing agent to at least one C1-C6 alcohol characterizable as an alkyl alcohol, aliphatic alcohol, or both which is between about 1:1 and about 1:25,000, such as, e.g., ˜1:10-˜1:25,000, ˜1:100-˜1:25,000, ˜1:1000-˜1:25,000, or ˜1:10,000-˜1:25,000, such as, e.g., ˜1:1-˜1:15,000, ˜1:1-˜1:9,000, ˜1:1-˜1:5,000, ˜1:1-˜1,1000, ˜1:1-˜1:500, ˜1:1-˜1:100, ˜1:1-˜1:50, or, e.g., ˜1:1-˜1:25, for example about 1:14 to about 1:66.

In aspects, composition(s) comprise a propellant component wherein the propellant component is at least about 96%, at least about 97%, or, e.g., at least about 98% composed of a single (e.g., the same) propellant compound establishing the percent of the propellant component represented by a dominant propellant, and wherein the ratio of the percent of the propellant component represented by the dominant propellant to the pharmaceutically acceptable and compositionally compatible organic acid is at least about 320:1, ≥˜321:1, ≥˜322:1, ≥˜323:1, ≥˜324:1, ≥˜325:1, ≥˜326:1, ≥˜327:1, ≥˜328:1, ≥˜329:1, or, e.g., ≥˜330:1, such as, e.g., ≥˜331:1, ≥˜332:1, or, e.g., ≥˜ or about 333:1.

In aspects, composition(s) comprise one or more pharmaceutically acceptable and compositionally compatible small compound stabilizing agent(s) and a non-anticholinergic compound fraction of an active pharmaceutical ingredient component, wherein the ratio of the one or more pharmaceutically acceptable and compositionally compatible small compound stabilizing agent(s) to the non-anticholinergic compound fraction of the pharmaceutically active ingredient component is between about 1:370 and about 20:1, such as, e.g., ˜1:300-˜20:1, ˜1:200-˜20:1, ˜1:100-˜20:1, ˜1:50-˜20:1, ˜1:25-˜20:1, ˜1:5-˜20:1, ˜1:5-˜10:1, ˜1:5-˜5:1, or, e.g., ˜1:5-˜1:1, such as, for example, ˜1:1, ˜1:1.5, ˜1:2, ˜1:2.5, ˜1:3, ˜1:3.5, ˜1:4, ˜1:4.5, or, e.g., ˜1:5.

In certain aspects, composition(s) comprise a pharmaceutically acceptable and compositionally compatible small compound stabilizing agent and, e.g., a surfactant component comprising one or more surfactant compound(s). In aspects, within such composition(s), the ratio of the pharmaceutically acceptable and compositionally compatible small compound stabilizing agent to the surfactant compound(s) is not more than, e.g., is not greater than 100:1. In aspects, within such composition(s), the ratio of the pharmaceutically acceptable and compositionally compatible small compound stabilizing agent to the surfactant compound(s) is not less than 1:100. In aspects, within such composition(s), the ratio of the pharmaceutically acceptable and compositionally compatible small compound stabilizing agent to the surfactant compound(s) is not greater than 100:1 and is not less than 1:100.

### Optionally Absent/Excluded Components

According to aspects, composition(s) do not comprise a detectible or significant amount of any one or more of the following compound(s)/agent(s): a surfactant other than that selected from oleic acid, Span 85, Tween 80, cetylpyridinium chloride, lecithin, or palmitic acid. In aspects, composition(s) do not comprise, e.g., surfactant(s) such as, e.g., magnesium stearate, ascorbyl palmitate, isopropyl myristate, sorbitan trioleate, isopropyl myristate, tyloxapol, polysorbate 20, polysorbate 40, vitamin E-TPGS, or a macrogol hydroxystearate such as, e.g., macrogol-15-hydroxystearate or acetylated monoglycerides such as, e.g., Myvacet 9-45, and Myvacet 9-08. In aspects, composition(s) do not comprise polyoxyethylene ether(s), ethyloleate, glyceryl trioleate, glyceryl monolaurate, glyceryl monooleate, glyceryl monostearate, glyceryl monoricinoleate, cetylalcohol, sterylalcohol, cetyl pyridinium chloride, block polymer(s), natural oil(s), sorbitan fatty acid ester(s) such as, e.g., sorbitan trioleate, polyethoxylated sorbitan fatty acid esters (for example polyethoxylated sorbitan trioleate), sorbimarogol oleate, synthetic amphotensides (tritons) or ethylene oxide ethers of octylphenolformaldehyde condensation products, polyethoxylated fat(s), polyethoxylated oleotriglycerides and polyethoxylated fatty alcohol(s). In aspects, composition(s) do not comprise corn oil. In certain embodiments, composition(s) do not comprise polyvinylpyrrolidone. In some embodiments, composition(s) do not comprise polyethylene glycol(s). In some aspects, composition(s) do not comprise polyvinylpyrrolidone or polyethylene glycol(s). In aspects, composition(s) do not comprise lactose. In aspects, composition(s) do not comprise PEG 1000. In aspects, composition(s) do not comprise PVP K 25. In aspects, composition(s) are not provided as dry powder composition(s). In aspects, composition(s) do not comprise one or more carrier(s) known in the art as suitable carriers for inhaled dry powder formulations, such as, e.g., saccharides, e.g., monosaccharides, disaccharides, polysaccharides, or sugar alcohols such as, e.g., arabinose, glucose, fructose, ribose, mannose, sucrose, trehalose, lactose (e.g., lactose monohydrate), maltose, starch(s), dextran or mannitol. In certain composition(s) herein, particularly when, e.g., provided as a solution, composition(s) do not comprise oleic acid. In aspects, composition(s) do not comprise a bronchodilating compound other than a tiotropium compound, a formoterol compound, or both.

In aspects, composition(s) do not comprise an API component comprising an API which is provided in complexed form. In aspects, composition(s) do not comprise a complexing agent.

According to certain aspects, composition(s) do not comprise a propellant component comprising any propellant compound other than HFA-134a, HFA-227ea, HFA-152a, HFO-1234ze, or HFO-1234yl. In aspects, composition(s) do not comprise a propellant component comprising any propellant compound other than HFA-134a, HFA-152a, HFO-12347, or HFO-1234yl. In aspects, composition(s) do not comprise a propellant component comprising any propellant compound other than HFA-134a, HFO-1234z, or HFO-1234yl. In aspects, composition(s) do not comprise HFA-227ea. In certain aspects, composition(s) do not comprise HFA-152a.

### Compositions/Delivery Characteristics

### Aerosolized Suspension(s), Solutions(s), and Hybrid Suspension-Solution(s)

According to certain aspects, pharmaceutical composition(s) of the present invention are provided as aerosol composition(s) (e.g., aerosolizable composition(s), e.g., composition(s) which are aerosolized upon the actuation of a delivery device). In aspects, composition(s) are compatible with or designed for administration via a delivery device, such as, e.g., an inhaler, e.g., a metered dose inhaler (MDI), such as, e.g., a pMDI.

According to certain aspects, composition(s) provided herein are suspensions, such as, e.g., aerosolized suspensions. In aspects, composition(s) provided herein are solutions, e.g., aerosolized solutions. In aspects, composition(s) provided herein are suspension-solution hybrid composition(s), such as, e.g., aerosolized suspension-solution hybrids.

In aspects, a solution is characterized as maintaining API(s) (e.g., an active pharmaceutical ingredient component) in a sufficiently homogeneous mixture so as to allow light to at least substantially uniformly pass through the composition (as opposed to, e.g., comprising a detectable or significant amount of API maintained in suspension.)

In certain aspects, composition(s) herein are provided as solution-suspension hybrid composition(s). In aspects, composition(s) comprise one or more API(s) which is difficult to solubilize relative to one or more API(s) also present in the composition(s). In aspects, for example, composition(s) comprise an inhalable corticosteroid, such as, e.g., a fluticasone compound, e.g., fluticasone furoate, wherein the fluticasone compound has a low solubility in propellant(s) present in the composition, e.g., a low solubility in HFA-based propellant(s). In aspects, such low-solubility API(s), e.g., a fluticasone compound, maintains a low solubility even in the present of a co-solvent, such as, e.g., ethanol, etc. In certain aspects, composition(s) herein are provided wherein an at least first compound, e.g., a tiotropium compound, is solubilized while an at least second compound, e.g., a fluticasone compound, will be suspended.

In aspects, composition(s) herein demonstrate detectably or significantly reduced Oswalt ripening, e.g., detectably or significantly reduced increase in inhomogeneous structure over time, detectably or significantly reduced formation of small crystals, detectably or significantly reduced formation of sol particles, or both, which, e.g., in aspects, may dissolve and redeposit onto larger crystals or sol particles compared to similar composition(s) comprising the same or at least substantially the same APIs in the same or at least substantially the same amounts, and, e.g., delivered by inhalation.

Specific exemplary composition(s) are provided here, however description of such exemplary composition(s) should not be interpreted as limiting.

In aspects, composition(s) herein comprise a pharmaceutical composition comprising tiotropium or a pharmaceutically acceptable salt or solvate thereof and a propellant, e.g., HFO-1234ze, HFA-134a, HFA-152a or mixture thereof, optionally with one or more pharmaceutically acceptable excipients. Further, in aspects, such pharmaceutical composition(s) can comprise tiotropium and one or more additional actives. In aspects, the active ingredient can be selected from the group comprising beta-adrenergic agonists and/or inhaled corticosteroids. In aspects, a beta-adrenergic agonist can be, e.g., formoterol or a pharmaceutically acceptable salt or solvate thereof. In aspects, an inhaled corticosteroid can be, e.g., beclomethasone, ciclesonide, or, e.g., fluticasone. In aspects, excipient(s) can include, e.g., one or more of an organic acid or a salt thereof, glycerol, ethanol, and water.

In certain specific aspects, the invention provides composition(s) comprising tiotropium or a pharmaceutically acceptable salt or solvate thereof; formoterol or a pharmaceutically acceptable salt or solvate thereof; organic acid or a salt thereof; glycerol; ethanol; water and a propellant selected from the group of HFO-1234ze, HFA-134a, HFA-152a or mixture thereof; wherein the composition is provided as a solution.

In certain specific aspects, the invention provides composition(s) comprising tiotropium or a pharmaceutically acceptable salt or solvate thereof in an amount of between about 0.005 wt. % and about 0.01 wt. %; ethanol in an amount of between about 12 wt. % and about 18 wt. %, water in an amount of between about 0.02 wt. % and about 0.10 wt. %, propellant(s) selected from the group of HFO-1234ze, HFA-134a, HFA-152a or mixture thereof, citric acid in an amount of about 0.05 wt. %, hydrochloric acid in an amount of between about 0.01 wt. % and about 0.05 wt. %, and, e.g., PEG-600, PEG-1000, glycerol or a combination thereof in an amount of between about 0.0 and about 1.0 wt. %. In aspects, such a composition is provided as a solution.

In certain aspects, the invention provides a composition comprising tiotropium or a pharmaceutically acceptable salt or solvate thereof in an amount of between about 0.005 wt. % and about 0.01 wt. %; formoterol or a pharmaceutically acceptable salt or solvate thereof in an amount of about 0.008 wt. %; ethanol in an amount of between about 12 wt. % to 18 wt. %, water in an amount of between about 0.02 wt. % and about 0.10 wt. %, a propellant component comprising one or more propellants selected from one or more of HFO-1234ze, HFA-134a, and HFA-152a, e.g., including mixture(s) thereof, maleic acid in an amount of about 0.05 wt. %, hydrochloric acid in an amount of between about 0.01 wt. % and about 0.05 wt. %, and PEG-600, PEG-1000, and glycerol in an amount of between about 0.0 and about 1.0 wt. %.

In other aspects, the invention provides a composition comprising tiotropium or a pharmaceutically acceptable salt or solvate thereof in an amount of between about 0.005 wt. % and about 0.01 wt. %; formoterol or a pharmaceutically acceptable salt or solvate thereof in an amount of between about 0.008 wt. %; an inhaled corticosteroid such as, e.g., beclomethasone, ciclesonide, or a fluticasone compound (e.g., fluticasone furoate), or a pharmaceutically acceptable salt(s) or solvate(s) thereof, in an amount of between about 0.18 wt. % and about 0.36 wt. %; ethanol in an amount of between about 12 wt. % and about 18 wt. %, water in an amount of between about 0.02 wt. % and about 0.10 wt. %, a propellant component comprising one or more propellants selected from HFO-1234ze, HFA-134a, and HFA-152a, maleic acid in an amount of about 0.05 wt. %, hydrochloric acid in an amount of between about 0.01 wt. % and about 0.05 wt. %, PEG-600, PEG-1000, and glycerol in an amount of between about 0.0 and about 1.0 wt. %.

In further aspects, the invention provides an composition for delivery by inhalation comprising tiotropium or a pharmaceutically acceptable salt or solvate thereof; one or more organic acid(s) or salt(s) thereof; glycerol; ethanol; water and a propellant component; wherein the formulation is provided as a solution, and wherein the propellant comprises or consists at least mostly of, at least generally of, at least essentially of, essentially of, at least substantially of, or consists of HFO-1234ze. In aspects, the composition further comprises formoterol or a pharmaceutically acceptable salt or solvate there.

In certain further aspects, the invention provides a composition comprising tiotropium or a pharmaceutically acceptable salt or solvate thereof; formoterol or a pharmaceutically acceptable salt or solvate thereof; one or more inhaled corticosteroids such as, e.g., beclomethasone or a pharmaceutically acceptable salt or solvate thereof or ciclesonide or a pharmaceutically acceptable salt or solvate thereof; one or more organic acid(s) salt(s) thereof; glycerol; ethanol; water and a propellant component; and wherein the propellant component comprises, is comprised at least mostly of, at least generally of, at least essentially of, essentially of, at least substantially of, or consists of HFO-1234ze

In certain aspects, the invention provides a composition for delivery by inhalation, wherein the composition comprises tiotropium or a pharmaceutically acceptable salt or solvate thereof; formoterol or a pharmaceutically acceptable salt or solvate thereof; a propellant component, and one or more pharmaceutically acceptable excipients; wherein the formulation is provided as a suspension, and wherein the propellant component comprises, is comprised at least mostly of, at least generally of, at least essentially of, essentially of, at least substantially of, or consists of HFO-1234ze.

### Particle/Droplet Size

In aspects, composition(s) provided herein comprise particles, such as, e.g., particles of one or more API compound(s). In aspects, the ex-actuator (post-actuation of a delivery device) particle size is any particle size having been established safe and suitable for administration to a recipient, as, e.g., recognized in the art or by a recognized regulatory authority such as the United States Food and Drug Administration (US FDA).

According to certain aspects, composition(s) of the invention provided as a solution have a detectably or significantly smaller average aerosolized droplet size than the average particle size of an at least substantially similar composition provided as a suspension.

In certain aspects, the ex-actuator average particle size (e.g., average maximum diameter in any one direction) is, e.g., between about 1 μm and about 5 μm, such as, e.g., ˜1 μm-˜4 μm, ˜1 μm-˜3 μm, or ˜1 μm-˜2 μm, such as, e.g., ˜2 μm-˜5 μm, ˜3 μm-˜5 μm, or, e.g., ˜4 μm-˜5 μm, as in, e.g., ˜2 μm-˜4 μm, or, e.g., ˜2.5 μm-˜3.5 μm.

In aspects, particle(s) of API(s) comprise a mass of their fine particle dose of between about 0.2 and about 5 μg, such as, e.g., ˜0.5 Mg-˜5 μg, ˜0.7 μg-˜5 μg, ˜0.9 μg-˜5 μg, ˜1.1 μg-˜5 μg, ˜1.3 μg-˜5 μg, ˜1.5 μg-˜5 μg, ˜1.7 μg-˜5 μg, ˜1.9 μg-˜5 μg, ˜2.1 μg-˜5 μg, ˜2.3 μg-˜5 μg, or ˜2.5 μg-˜5 μg.

In aspects, particle(s) of API(s) comprise a mass of their fine particle dose of between about 0.2 μg and about 4.7 μg, such as, e.g., ˜0.2 μg-˜4.5 μg, ˜0.2 μg-˜4.3 μg, ˜0.2 μg-˜4.1 μg, ˜0.2 μg-˜3.9 μg, ˜0.2 μg-˜3.7 μg, ˜0.2 μg-˜3.5 μg, ˜0.2 μg-˜3.3 μg, ˜0.2 μg-˜3.1 μg, ˜0.2 μg-˜2.9 μg, ˜0.2 μg-˜2.7 μg, or, e.g., ˜0.2 μg-˜2.5 μg,

In aspects, particle(s) of API(s) comprise a mass of their fine particle dose of between about 0.3 μg and about 4.5 μg, such as, e.g., ˜0.4 μg-˜4 μg, or ˜0.5 μg-˜3 μg, e.g., ˜0.5 μg-˜3 μg.

### Deposition

According to certain aspects, composition(s) herein comprise a fine particle fraction of tiotropium compound(s), beta-adrenergic compound(s), corticosteroid compound(s), or any combination of any or all thereof, after storage at about 50° C. (+/−2º C) and about 75% (+/−2%) relative humidity, which is at least about 40% of the weight of the emitted dose of the API, respectively, such as, e.g., ≥˜42%, ≥˜44%, ≥˜46%, ≥˜48%, ≥˜50%, ≥˜52%, ≥˜54%, ≥˜56%, ≥˜58%, ≥˜60%, ≥˜62%, ≥˜64%, ≥˜66%, ≥˜68%, or, e.g., ≥˜70% or more of the emitted dose of each respective API. In aspects, such characteristic(s) result in a detectably or significantly increased amount of API(s) being deposited deeper into the lung compared to similar product(s) marketed to treat asthma, COPD, or both.

In aspects, composition(s) herein, can be subjected to particle size distribution study(ies) replicating the deposition of particles in a human lung upon actuation of a delivery device via the mouth. In aspects such study(ies) can comprise use of a Cascade Impactor. In aspects, a Cascade Impactor can comprise at least 7 stages, wherein, e.g., stage 0 represents the throat (particle sizes about 9 μm-about 10 μm), stage 1 (about 5.8 μm-about 9 μm), stage 2 (about 4.7 μm-about 5.8 μm), stage 3 (about 3.3 μm-about 4.7 μm), stage 4 (about 2.1 μm-about 3.3 μm), stage 5 (about 1.1 μm-about 2.1 μm), stage 6 (about 0.65 μm-about 1.1 μm), stage 7 (about 0.43 μm-about 0.65 μm), and a final filter (≤0.43 μm). In aspects, when composition(s) are subjected to particle deposition testing using such a model, composition(s) demonstrate that after a period of at least about 1 month of storage under normal storage condition(s) (as described elsewhere herein), such as, e.g., ≥˜2 months, ≥˜3 months, ≥˜6 months, ≥˜9 months, ≥˜12 months, ≥˜24 months, or, e.g., ≥˜36 months, the percent of the initial labeled amount of at least one API of the composition, such as most API(s), general all API(s), or all API(s), reaching at least the third stage of the Cascade Impactor, is at least about 9.3%, such as, e.g., ≥˜9.4%, ≥˜9.6%, ≥˜9.8%, ≥˜10%, ≥˜10.2%, ≥˜10.4%, ≥˜10.6%, ≥˜10.8%, ≥˜11%, ≥˜11.2%, ≥˜11.4%, ≥˜11.6%, ≥˜11.8%, ≥˜12%, ≥˜12.2%, ≥˜12.4%, ≥˜12.6%, ≥˜12.8%, ≥˜13%, ≥˜13.2%, ≥˜13.4%, ≥˜13.6%, ≥˜13.8%, or, ≥˜14%.

In certain aspects, after a period of at least about 1 month of storage under normal storage condition(s) (as described elsewhere herein), such as, e.g., ≥˜2 months, ≥˜3 months, ≥˜6 months, ≥˜9 months, ≥˜12 months, ≥˜24 months, or, e.g., ≥˜36 months, the percent of the initial labeled amount of at least one API of the composition, such as most API(s), general all API(s), or all API(s), reaching at least the third stage of the Cascade Impactor, is no less than about 10%, such as, e.g., no less than about 11%, no less than about 12%, no less than about 13%, no less than about 14%, no less than about 15%, no less than about 16%, no less than about 17%, no less than about 18%, no less than about 19%, or, e.g., no less than about 20%.

### Dissolved Oxygen

In aspects, composition(s) are characterizable by the amount of dissolved air, dissolved oxygen, dissolved nitrogen, or any one or more thereof present therein. In aspects, composition(s) comprise an amount of dissolved gas, e.g., oxygen, nitrogen, or both, which is suitable for maintaining a stable composition, e.g., suitable for providing compositions having one or more of the stability characteristics described herein. In aspects, composition(s) comprise an amount of dissolved oxygen which is suitable for maintaining a stable composition, e.g., suitable for providing compositions having one or more of the stability characteristics described herein. In aspects, at least generally all, at last substantially all, at least essentially all, or all dissolved oxygen is removed from composition(s) during their manufacture. In aspects, manufacturing process(es) can comprise a step which removes at least generally all, at least substantially all, at least essentially all, or all dissolved oxygen prior to final sealing of the composition in its final packaging. In aspects, a manufacturing process can comprise, e.g., use of a vacuum crimp procedure to remove at least generally all, at least substantially all, at least essentially all, or all dissolved oxygen prior to final sealing of the composition in its final packaging. In aspects, removal of at least generally all, at least substantially all, at least essentially all, or all dissolved oxygen prior to final sealing of the composition in its final packaging detectably or significantly improves one or more characteristics of product stability compared to compositions where less than such an amount of dissolved oxygen is removed or, e.g., the manufacturing process comprises no step for removing dissolved oxygen.

In aspects, one or more characteristics of composition performance is associated with the amount of dissolved air, e.g., both nitrogen and oxygen, in composition(s). In aspects, oxygen, nitrogen, or both will dissolve in composition(s) under detectably or significantly elevated pressure. In aspects, such dissolution further increases the pressure of the composition and can detectably or significantly modify the aerosolization of the composition(s). In aspects, such dissolution can detectably or significantly modify the aerodynamic performance of the composition(s). In aspects, composition(s) comprise an amount of dissolved air, dissolved oxygen, dissolved nitrogen, or both which is detectably or significantly different from that of compositions described in, e.g., one or more reference product(s) (reference product(s) being described elsewhere herein.) In aspects, composition(s) provided by the invention have a detectably or significantly improved aerodynamic performance compared to one or more reference product(s), as determined, e.g., by a study of drug delivery/deposition (e.g., amount of drug delivered to one or more location(s) within the respiratory tract,) which may be demonstrated by one or more appropriately conducted study(ies) or clinical trial(s).

### Mechanism of Delivery: MDI(s)

In aspects, composition(s) provided herein can be delivered by inhalation. In aspects, composition(s) can be delivered by any suitable means of inhalation, such as any means known or otherwise recognized in the art, such as, e.g., delivery devices such as inhalers. In aspects, composition(s) can be delivered by an inhaler such as, e.g., a metered dose inhaler (MDI), such as, e.g., a pressurized metered dose inhaler (pMDI) or, e.g., a breath actuated metered dose inhaler. In aspects, any type of inhaler device can be used. In most common embodiments, composition(s) are delivered by MDI.

The design and operation of MDIs is well described in the art. An MDI comprise(s) a pressurized container maintaining the composition, a nozzle, and a valve assembly capable of dispensing a controlled quantity of the composition through the nozzle when it is activated (e.g., upon actuation). The nozzle and valve assembly are typically located in a housing that is equipped with a mouthpiece.

When an MDI is used, in aspects, any type of MDI known in the art designed to deliver, on demand, a discrete and accurate amount of a drug to the respiratory tract of a patient using a liquefied propellant in which the drug is dissolved, suspended, or dispersed can be employed. In aspects, a delivery device, e.g., metered dose inhaler, is equipped with a metering valve. In aspects, a delivery device, e.g., metered dose inhaler, comprises a container suitable for maintaining composition(s) until the delivery device is actuated, e.g., an aerosol canister. In certain aspects, the invention herein provides a delivery device, e.g., inhaler, e.g., metered dose inhaler, comprising at least one container suitable for maintaining a composition until the delivery device is actuated, e.g., a canister, wherein the canister(s) comprise one or more composition(s) described herein.

In aspects, a container suitable for maintaining composition(s) until the delivery device is actuated can be any container known in the art, such as, e.g., a canister, e.g., a sealed canister. In aspects, a suitable container, e.g., sealed canister, is one capable of containing composition(s) described herein under pressure, e.g., under a presser which is detectably or significantly greater than ambient atmospheric pressure.

In aspects, a container, e.g., an aerosol canister is fitted with a valve. In aspects, a canister can have any suitable volume. In aspects, the full (e.g., “brimful”) capacity of the canister will depend on the volume of the formulation used to fill the canister. In aspects, a container, e.g., a canister, will have a sufficient volume to contain enough of a composition to deliver an appropriate/suitable/target number of doses. Such appropriate/suitable/target numbers of doses are described elsewhere herein.

In certain exemplary applications, the canister can have a volume of between about 1 mL and about 100 mL, such as, e.g., ˜2 mL-˜90 mL, ˜3-˜80 mL, ˜4-˜70 mL, or, e.g., ˜5 mL-˜60 mL or, e.g., about 5 mL to 50 mL, such as, e.g., ˜5 mL-˜45 mL, ˜5 mL-˜40 mL, ˜5 mL-˜35 mL, ˜5 mL-˜30 mL, ˜5 mL-˜25 mL, ˜5 mL-˜20 mL, or, ˜5 mL-˜15 mL.

In aspects, a canister can have a volume of between about 6 mL to about 50 mL, such as, e.g., ˜7 mL-˜40 mL, ˜8 mL-˜30 mL, or, e.g., ˜9 mL-˜20 mL.

In certain aspects, a canister can have a volume of between about 8 mL and about 20 mL, such as, e.g., about 8 mL and about 15 mL.

In aspects, a container, e.g., a canister, comprises a valve. In aspects, the valve is affixed, e.g., crimpled, onto the container (canister) by way of a cap or ferrule. In aspects, the cap or ferrule is made of aluminum or an aluminum alloy. In aspects, the cap or ferrule is a component of a valve assembly. In aspects, one or more seals can be located between the canister and the ferrule. In aspects, seal(s) can be one or more of O-ring seals, gasket seals, etc. In aspects, a valve is a metered dose valve. In aspects, a valve has a size within the range of about 10 μL-˜120 μL, such as, e.g., about 20 μL to 100 μL Specific exemplary valve size include, e.g., 25 μL, 50 μL, 60 μL, and, e.g., 63 μL valve sizes.

According to certain aspects, a metered dose inhaler suitable for use in the delivery of composition(s) herein can comprise a metering valve and a low orifice actuator. In aspects any sized orifice actuator can be used. In aspects, the orifice diameter is between about 0.2 mm and about 0.65 mm. In aspects, a 0.2 mm to 0.4 mm may be particularly useful for delivery of solution composition(s), such as the solution composition(s) discussed herein. In aspects, a suitable low orifice actuator ranges from between about 0.1 mm and about 0.8 mm, such as, e.g., ˜0.2 mm-˜0.7 mm, ˜0.3 mm-˜0.6 mm, or, e.g., ˜0.4 mm-˜0.5 mm.

In aspects, an actuator has a patient port, e.g., a mouthpiece. In aspects, the patient port, e.g., mouthpiece facilitates the delivery of a/the composition contained in the container (e.g., canister). In aspects, a patient port can be configured in a variety of ways depending on the intended destination of the formulation. For example, in aspects, a patient port designed for administration to the nasal cavities will generally have an upward slope to direct the formulation/composition to the nose. In aspects, an actuator is made out of a plastic material. In aspects, exemplary plastic materials can include, e.g., at least one of polyethylene and polypropylene. In aspects, an MDI suitable for use in the delivery of composition(s) described herein comprise an actuator with a nozzle. In aspects, in use, the aerosol spray can emerge from this nozzle before exiting the mouthpiece of the actuator. In aspects, the nozzle can be characterized by an orifice diameter and a jet length. In aspects, any suitable orifice diameter can be used as described above. In aspects, a suitable orifice jet length is between about 0.5 mm and about 1 mm. Specific examples include orifice diameters of, e.g., ˜0.25 mm, ˜0.3 mm, or ˜0.4 mm, any of which can have a jet length of e.g., about 0.8 mm.

A metered dose valve can be, in aspects, located at least partially within the container, e.g., canister, and can be, in aspects, at least partially in communication with the actuator. In aspects, metered dose valve(s) include a metering chamber that is at least partially defined by an inner valve body through which a valve stem passes. In aspects, a valve stem can be biased outwardly by a compression spring to be in a sliding, sealing engagement with an inner tank seal and outer diaphragm seal. In aspects, a valve can comprise a second valve body in the form of a bottle emptier. In aspects, an inner valve body, sometimes referred to as, e.g., the primary valve body, defines, in part, a metering chamber. In aspects, a second valve body, sometimes referred to as, e.g., the secondary valve body, defines, at least in part, a pre-metering region (sometimes referred to as a pre-metering chamber) in addition to serving as a bottle emptier. In aspects, outer wall(s) of the portion of the metered dose valve that are located within the canister, as well as the inner wall(s) of the canister, define a composition chamber for containing the pharmaceutical composition.

In aspects, in use, a pharmaceutical composition maintained within a container of an MDI can pass from the composition chamber into the metering chamber. In moving to the metering chamber, the formulation can, in aspects, pass into the above-described pre-metering chamber through a space, e.g., an annular space, between the secondary valve body (or a flange of the secondary valve body) and the primary valve body. In aspects, pressing the valve stem towards the interior of the container actuates the valve. In aspects, actuation of the valve allows the pharmaceutical composition to pass from the pre-metering chamber through a side hole in the valve stem and through an outlet in the valve stem, to an actuator nozzle, and finally through the patient port to the patient/device user. In aspects, when the valve stem is released, the pharmaceutical composition enters the valve, typically to the pre-metering chamber, through a space, e.g., an annular space, and then travels to the metering chamber.

### Standard Manufacturing (Uncoated)

According to certain aspects, devices, e.g., inhalation devices, e.g., inhalers, such as, e.g., metered dose inhalers, suitable for delivery of composition(s) described herein are made using typical, standard manufacturing method(s) and material(s). In aspects, suitable container(s) (e.g., canisters) for maintaining composition(s) described herein, one or more other component(s) of an MDI, or both, are made of plain aluminum or stainless steel. In certain aspects, components of a delivery device are not made of any specifically elected material intended to avoid undesirable interaction between the composition(s) and the device(s). In aspects, components of a delivery device are not coated with any specifically elected coating intended to avoid undesirable interaction between the composition(s) and the devices(s).

In one particular embodiment, an advantage of composition(s) provided herein is that composition(s) can be delivered using standard MDI device(s), comprising typical, standard materials, not requiring coating by any intentionally selected coating, such that devices can be manufactured using standard manufacturing techniques and the cost of design and manufacturing of such specialized devices is avoided.

### Special Manufacturing

According to certain aspects, as noted above, special manufacturing techniques/processes, including, e.g., the use of specialized materials in the design of one or more delivery device components, the use of specialized coatings for one or more delivery device components, or any combination thereof, is not required.

In aspects, one or more component(s) of a device is not coated with a polymer such as, e.g., fluorocarbon copolymers such as FEP-PES (fluorinated ethylene propylene and polyethersulphone), PFA-PES (perfluoroalkoxyalkane and polyethersulphone), epoxy and ethylene or combinations thereof. In aspects, one or more component(s) of a device is not anodized. In aspects, one or more components of a device, e.g., one or more of the canister(s), valve(s), gasket(s) (when present), seal(s), O-ring(s), etc. are not coated with a material described in, e.g., U.S. Pat. Nos. 8,414,956, 8,815,325, and, e.g., U.S. Patent Application No. US20120097159. In aspects, composition(s) lack any of the components suitable for applying such coatings, such as, e.g., those described in, e.g., U.S. Patent Publication No. US20190054010 (or, similarly, US20210260310). In aspects, one or more component(s) of a device is not coated with a polyphenylsulphone compound, such as, e.g., a polyphenylsulphone compound having an average molecular weight of between about 10,000 daltons (D) and about 80,000 D, such as, e.g., between about 10,000 D and about 30,000 D. In aspects, one or more component(s) of a device is not coated with a coating comprising a polyethersulphone, fluoropolymer(s) such as PTFE, FEP, or PFA. In aspects, one or more component(s) of a device is not coated with a non-metal coating such as a non-metal coating described in, e.g., US20210260310.

In aspects, composition(s) do not comprise a component made of a butyl or halo-butyl rubber.

According to certain alternative aspects, one or more component(s) of a device can comprise one or more of the material(s) described in this section, can be coated with one or more of the material(s) described in this section, or both.

### Means of Composition Delivery

In this and other component, compound/agent/constituent, or method aspect(s) of the invention, the invention also can be characterized as comprising a “means” for delivering composition(s). In such a respect, any known equivalents of such named delivery mechanism(s) can also be, e.g., are, incorporated into composition(s) or method(s) of the invention. As with other sections similarly described herein, any of the component(s), compound(s), agent(s), constituent(s), or, e.g., method(s) of the invention can be, where suitable, described as means (e.g., the above-described delivery device can be described as delivery means or means for delivering composition(s)).

### Stability & Storage Conditions

According to certain aspects, composition(s) provided herein are stable under typical storage conditions for commercially relevant period(s) of time. In aspects, a typical, or standard, standard storage condition is, e.g., about room temperature, e.g., between about 20° C. and about 30° C., such as, e.g., about 25° C.+/−about 2° C.; between about 2° C. and about 8° C.; or any combination thereof. In aspects, composition(s) are stable at about room temperature for a commercially relevant period of time, e.g., such that they demonstrate a commercially relevant and pharmaceutically acceptable shelf-life.

According to certain aspects, tiotropium-containing composition(s) described herein are sufficiently physically and chemically stable so as to facilitate their delivery via an MDI. In aspects, one or more excipient(s) in the amount(s) described herein, and combination(s) thereof, provide for composition(s) demonstrating such commercially relevant stability. In aspects, Tiotropium-containing composition(s) further comprising one or more API(s) (therapeutic agent(s)) demonstrate such commercially relevant stability.

In aspects, composition(s) provided by the invention comprise less than about 0.3 wt. % total impurities, such as, e.g., ≤˜0.25 wt. %, ˜0.2 wt. %, ≤˜0.15 wt. %. ≤˜0.1 wt. %. ≤˜0.05 wt. %. or, e.g., ≤˜0.01 wt. %. total impurities, from the degradation of any one, more, or all API(s) (individually or, in aspects, in combination) based on the total weight of such one, more, or all API(s) in the composition and any associated impurity(ies), after storage at about 40° C. and about 75% relative humidity for a period of at least about 1 month, such as, e.g., ≥˜2 months, ≥˜3 months, ≥˜6 months, ≥˜12 months, ≥˜15 months, ≥˜18 months, ≥˜21 months, ≥˜24 months, ≥˜27 months, ≥˜30 months, ≥˜33 months, or, e.g., ≥˜36 months.

In aspects, composition(s) provided by the invention comprise less than about 0.3 wt. % total impurities, such as, e.g., ≤˜0.25 wt. %, ≤˜0.2 wt. %, ≤˜0.15 wt. %. ≤˜0.1 wt. %. ≤˜0.05 wt. %. or, e.g., ≤˜0.01 wt. %. total impurities, from the degradation of any tiotropium compound based on the total weight of tiotropium compound and tiotropium impurities in the composition, after storage at about 40° C. and about 75% relative humidity for a period of at least about 1 month, such as, e.g., ≥˜2 months, ≥˜3 months, ≥˜6 months, ≥˜12 months, ≥˜15 months, ≥˜18 months, ≥˜21 months, ≥˜24 months, ≥˜27 months, ≥˜30 months, ≥˜33 months, or, e.g., ≥˜36 months.

In certain aspects, composition(s) provided by the invention comprise at least one tiotropium compound and at least one non-anticholinergic bronchodilator, such as, e.g., a beta-adrenergic agonist, such as, e.g., a formoterol compound, wherein the composition comprises less than about 0.3 wt. % total impurities, such as, e.g., ≤˜0.25 wt. %, ≤˜0.2 wt. %, ≤˜0.15 wt. %. ≤˜0.1 wt. %, ≤˜0.05 wt. %. or, e.g., ≤˜0.01 wt. %. total impurities, from the degradation of any tiotropium compound based on the total weight of tiotropium compound and tiotropium impurities in the composition, after storage at about 40° C. and about 75% relative humidity for a period of at least about 1 month, such as, e.g., ≥˜2 months, ≥˜3 months, ≥˜6 months, ≥˜12 months, ≥˜15 months, ≥˜18 months, ≥˜21 months, ≥˜24 months, ≥˜27 months, ≥˜30 months, ≥˜33 months, or, e.g., ≥˜36 months.

In certain aspects, composition(s) provided by the invention comprise at least one tiotropium compound, at least one non-anticholinergic bronchodilator, such as, e.g., a beta-adrenergic agonist, such as, e.g., a formoterol compound, and, at least one non-bronchodilating compound, e.g., an anti-inflammatory compound, e.g., a steroid compound, e.g., a corticosteroid compound, wherein the composition comprises less than about 0.3 wt. % total impurities, such as, e.g., ≤˜0.25 wt. %, ≤˜0.2 wt. %, ≤˜0.15 wt. %, ≤˜0.1 wt. %, ≤˜0.05 wt. %. or, e.g., ≤˜0.01 wt. %. total impurities, from the degradation of any tiotropium compound based on the total weight of tiotropium compound and tiotropium impurities in the composition, after storage at about 40° C. and about 75% relative humidity for a period of at least about 1 month, such as, e.g., ≥˜2 months, ≥˜3 months, ≥˜6 months, ≥˜12 months, ≥˜15 months, ≥˜18 months, ≥˜21 months, ≥˜24 months, ≥˜27 months, ≥˜30 months, ≥˜33 months, or, e.g., ≥˜36 months.

In aspects, composition(s) provided by the invention comprise less than about 0.3 wt. % total impurities, such as, e.g., ≤˜0.25 wt. %, ≤˜0.2 wt. %, ≤˜0.15 wt. %, ≤˜0.1 wt. %, ≤˜0.05 wt. %. or, e.g., ≤˜0.01 wt. %. total impurities, from the degradation of any non-anticholinergic compound, e.g., beta-adrenergic agonist compound, e.g., formoterol compound based on the total weight of such compound, e.g., formoterol compound and associated impurities, in the composition, after storage at about 40° C. and about 75% relative humidity for a period of at least about 1 month, such as, e.g., ≥˜2 months, ≥˜3 months, ≥˜6 months, ≥˜12 months, ≥˜15 months, ≥˜18 months, ≥˜21 months, ≥˜24 months, ≥˜27 months, ≥˜30 months, ≥˜33 months, or, e.g., ≥˜36 months.

In aspects, composition(s) provided by the invention comprise less than about 0.3 wt. % total impurities, such as, e.g., ≤˜0.25 wt. %, ˜0.2 wt. %, ˜0.15 wt. %, ≤˜0.1 wt. %. ≤˜0.05 wt. %. or, e.g., ≤˜0.01 wt. %. total impurities, from the degradation of any anti-inflammatory compound, e.g., steroid compound, e.g., corticosteroid compound, e.g., ciclesonide compound, beclomethasone compound, or, e.g., fluticasone compound (e.g., fluticasone furoate) based on the total weight of such compound and associated impurities after storage at about 40° C. and about 75% relative humidity for a period of at least about 1 month, such as, e.g., ≥˜2 months, ≥˜3 months, ≥˜6 months, ≥˜12 months, ≥˜15 months, ≥˜18 months, ≥˜21 months, ≥˜24 months, ≥˜27 months, ≥˜30 months, ≥˜33 months, or, e.g., ≥˜36 months.

In aspects, composition(s) provided by the invention comprise one or more API(s), wherein the composition(s) maintain at least about 95%, such as, e.g., ≥˜95.5%, ≥˜96%, ≥˜96.5%, ≥˜97%, ≥˜97.5%, ≥˜98%, ≥˜98.5%, ≥˜99%, ≥˜99.5%, or, e.g., ˜100% by weight of each of the at least one API(s) contained in the composition immediately following manufacture after storage at about 40° C. and about 75% relative humidity for a period of at least about 1 month, such as, e.g., ≥˜2 months, ≥˜3 months, ≥˜6 months, ≥˜12 months, ≥˜15 months, ≥˜18 months, ≥˜21 months, ≥˜24 months, ≥˜27 months, ≥˜30 months, ≥˜33 months, or, e.g., ≥˜36 months.

In aspects, composition(s) provided by the invention comprise at least one tiotropium compound and the composition(s) maintain at least about 95%, such as, e.g., ≥˜95.5%, ≥˜96%, ≥˜96.5%, ≥˜97%, ≥˜97.5%, ≥˜98%, ≥˜98.5%, ≥˜99%, ≥˜99.5%, or, e.g., ˜100% by weight of each of the at least one tiotropium compound(s) contained in the composition immediately following manufacture after storage at about 40° C. and about 75% relative humidity for a period of at least about 1 month, such as, e.g., ≥˜2 months, ≥˜3 months, ≥˜6 months, ≥˜12 months, ≥˜15 months, ≥˜18 months, ≥˜21 months, ≥˜24 months, ≥˜27 months, ≥˜30 months, ≥˜33 months, or, e.g., ≥˜36 months.

In aspects, composition(s) provided by the invention comprise at least one tiotropium compound and at least one nonanticholinesterase compound, e.g., at least one beta-adrenergic compound, e.g., at least one formoterol compound, and the composition(s) maintain at least about 95%, such as, e.g., ≥˜95.5%, ≥˜96%, ≥˜96.5%, ≥˜97%, ≥˜97.5%, ≥˜98%, ≥˜98.5%, ≥˜99%, ≥˜99.5%, or, e.g., ˜100% by weight of each of the at least one tiotropium compound(s) contained in the composition immediately following manufacture after storage at about 40° C. and about 75% relative humidity for a period of at least about 1 month, such as, e.g., ≥˜2 months, ≥˜3 months, ≥˜6 months, ≥˜12 months, ≥˜15 months, ≥˜18 months, ≥˜21 months, ≥˜24 months, ≥˜27 months, ≥˜30 months, ≥˜33 months, or, e.g., ≥˜36 months.

In aspects, composition(s) provided by the invention comprise at least one tiotropium compound and at least one nonanticholinesterase compound, e.g., at least one beta-adrenergic compound, e.g., at least one formoterol compound, and, further, at least one non-bronchodilating compound, e.g., at least one anti-inflammatory compound, e.g., steroid compound, e.g., corticosteroid compound, and the composition(s) maintain at least about 95%, such as, e.g., ≥˜95.5%, ≥˜96%, ≥˜96.5%, ≥˜97%, ≥˜97.5%, ≥˜98%, ≥˜98.5%, ≥˜99%, ≥˜99.5%, or, e.g., ˜100% by weight of each of the at least one tiotropium compound(s) contained in the composition immediately following manufacture after storage at about 40° C. and about 75% relative humidity for a period of at least about 1 month, such as, e.g., ≥˜2 months, ≥˜3 months, ≥˜6 months, ≥˜12 months, ≥˜15 months, ≥˜18 months, ≥˜21 months, ≥˜24 months, ≥˜27 months, ≥˜30 months, ≥˜33 months, or, e.g., ≥˜36 months.

In aspects, composition(s) provided by the invention comprise at least one nonanticholinesterase compound, e.g., at least one beta-adrenergic compound, e.g., at least one formoterol compound and the composition(s) maintain at least about 95%, such as, e.g., ≥˜95.5%, ≥˜96%, ≥˜96.5%, ≥˜97%, ≥˜97.5%, ≥˜98%, ≥˜98.5%, ≥˜99%, ≥˜99.5%, or, e.g., ˜100% by weight of each of the at least one nonanticholinesterase compound, e.g., at least one beta-adrenergic compound, e.g., at least one formoterol compound contained in the composition immediately following manufacture after storage at about 40° C. and about 75% relative humidity for a period of at least about 1 month, such as, e.g., ≥˜2 months, ≥˜3 months, ≥˜6 months, ≥˜12 months, ≥˜15 months, ≥˜18 months, ≥˜21 months, ≥˜24 months, ≥˜27 months, ≥˜30 months, ≥˜33 months, or, e.g., ≥˜36 months.

In aspects, composition(s) provided by the invention comprise at least one nonbronchodilating compound, e.g., at least one anti-inflammatory compound, such as at least one steroid compound, e.g., at least one corticosteroid compound, and the composition(s) maintain at least about 95%, such as, e.g., ≥˜95.5%, ≥˜96%, ≥˜96.5%, ≥˜97%, ≥˜97.5%, ≥˜98%, ≥˜98.5%, ≥˜99%, ≥˜99.5%, or, e.g., ˜100% by weight of each of the at least one nonbronchodilating compound, e.g., at least one anti-inflammatory compound, such as at least one steroid compound, e.g., at least one corticosteroid compound, such as, e.g., a ciclesonide compound, beclomethasone compound, or, e.g., fluticasone compound (e.g., fluticasone furoate), contained in the composition immediately following manufacture after storage at about 40° C. and about 75% relative humidity for a period of at least about 1 month, such as, e.g., ≥˜2 months, ≥˜3 months, ≥˜6 months, ≥˜12 months, ≥˜15 months, ≥˜18 months, ≥˜21 months, ≥˜24 months, ≥˜27 months, ≥˜30 months, ≥˜33 months, or, e.g., ≥˜36 months.

In aspects, composition(s) herein maintain at least about 95%, such as, e.g., ≥˜96%, ≥˜97%, ≥˜98%, ≥˜98.2%, ≥˜98.4%, ≥˜98.6%, ≥˜98.8%, ≥˜99%, ≥˜99.2%, ≥˜99.4%, ≥˜99.6%, or, e.g., ≥˜99.8% of the original pharmaceutical activity of the composition after storage at about 40° C. and about 75% relative humidity for a period of at least about 1 month, such as, e.g., ≥˜2 months, ≥˜3 months, ≥˜6 months, ≥˜12 months, ≥˜15 months, ≥˜18 months, ≥˜21 months, ≥˜24 months, ≥˜27 months, ≥˜30 months, ≥˜33 months, or, e.g., ≥˜36 months.

In aspects, composition(s) herein can be characterized by comparison to one or more reference product(s), such reference product(s) being described elsewhere herein. In aspects, composition(s) provided by the invention demonstrate a stability as characterized by one or more the stability measurements/characteristics described in this section, which is detectably or significantly better than, e.g., more stable than, a reference product.

### Dose & Administration

### Canister Volume

In aspects, composition(s) will be provided in an amount so as to facilitate the delivery of a suitable number of doses, e.g., such a number of doses as described herein, or which may be known in the art. In common aspects, composition(s) herein can be provided in an amount of between about 1 mL and about 100 mL. In aspects, composition(s) are provided in a container, e.g., a canister, having a sufficient volume to contain enough medicament (e.g., composition, API(s), etc.) for delivering an appropriate number of doses.

In aspects, composition(s) herein are provided in a container comprising between about 1-about 100 mL, such as, e.g., ˜1 mL-˜90 mL, ˜1 mL-˜80 mL, ˜1 mL-˜70 mL, ˜1 mL-˜60 mL, or, e.g., ˜1 mL-˜50 mL, such as, e.g., ˜5 mL-˜100 mL, ˜10 mL-˜100 mL, ˜15 mL-˜100 mL, ˜20 mL-˜100 mL, ˜25 mL-˜100 mL, ˜30 mL-˜100 mL, ˜35 mL-˜100 mL, ˜40 mL-˜100 mL, ˜45 mL-˜100 mL, or, e.g., ˜50 mL-˜100 mL.

In aspects, composition(s) herein are provided in a container, e.g., canister, comprising ˜1 mL-˜80 mL, ˜2 mL-˜70 mL, ˜3 mL-˜60 mL, ˜4 mL-˜50 mL, or, e.g., ˜5 mL-˜50 mL, e.g., ˜6 mL-˜50 mL, ˜7 mL-˜50 mL, ˜8 mL-˜50 mL, ˜9 mL-˜50 mL, ˜10 mL-˜50 mL, such as, for example ˜5 mL-˜40 mL, ˜5 mL-˜30 mL, or ˜5 mL-˜20 mL, or, e.g., ˜8 mL-˜20 mL, or ˜8 mL-˜15 mL.

### Doses Available/Canister

In aspects, as is described elsewhere herein, composition(s) provided by the invention are provided by a delivery device, e.g., an inhaler, e.g., a metered dose inhaler. Typical inhalers, such as metered dose inhalers, are designed to deliver a specified number of doses of a pharmaceutical composition. In aspects, a dose is deliverable by a single actuation of the device, e.g., inhaler. In alternative aspects, a dose is deliverable by a plurality of actuations of the device (inhaler), such as, e.g., two, three, four, or more actuations. In aspects, composition(s) are provided in a sufficient amount so as to facilitate the delivery of a plurality of individual doses. In aspects, composition(s) are provided in an amount suitable for the administration of, e.g., about 5 to about 150 doses, e.g., ˜5-˜140 doses, ˜5-˜130 doses, ˜5-˜120 doses, ˜5-˜110 doses, ˜5-˜100 doses, ˜5-˜90 doses, ˜5-˜80 doses, ˜5-˜70 doses, ˜5-˜60 doses, or ˜5-˜50 doses.

In aspects, composition(s) are provided in an amount suitable for the administration of, e.g., ˜10-˜150 doses, ˜20-˜150 doses, ˜30-˜150 doses, ˜40-˜150 doses, ˜50-˜150 doses, ˜60-˜150 doses, ˜70-˜150 doses, ˜80-˜150 doses, ˜90-˜150 doses, or, e.g., ˜100-˜150 doses.

In aspects, composition(s) are provided in an amount suitable for the administration of, e.g., ˜6-˜140 doses, ˜7-˜130 doses, ˜8-˜120 doses, ˜9-˜110 doses, ˜10-˜100 doses, ˜12-˜90 doses, ˜14-˜80 doses, ˜16-˜70 doses, ˜18-˜60 doses, ˜20-˜50 doses, or, e.g., about 10 to about 120 doses or, e.g., about 20 to about 40 doses. In one certain aspect, a metered dose inhaler is provided to deliver about 120 doses of composition, whereby each dose is delivered in two actuations. Such a delivery system can be employed with any of the composition(s) or delivery device(s), e.g., inhaler type(s) described herein.

### API/Canister

Amounts of API have been provided here in both weight percent (percent by weight, wt. %) of a composition and mg/mL of a composition. Thus, the reader should understand that the amount of API/container, when composition(s) are provided in a container for the delivery of a plurality of doses thereof, will depend on the volume of the composition provided in the container. The amount of API within the container/canister can be calculated given the possible concentrations of API(s) in the composition, disclosed here and or using other known therapeutically effective concentrations of such API(s) known or disclosed in the art. The amount of API per container/canister, therefore, should be considered as encompassed by/included in this disclosure.

### Bronchodilator/Canister

Amounts of bronchodilating component (e.g., bronchodilating compound(s)/agent(s)) has/have been provided here in both weight percent (percent by weight, wt. %) of a composition and mg/mL of a composition. Thus, the reader should understand that the amount of a bronchodilating component/container, when composition(s) are provided in a container for the delivery of a plurality of doses thereof, will depend on the volume of the composition provided in the container. The amount of bronchodilating component within the container/canister can be calculated given the possible concentrations of bronchodilating component in the composition, disclosed here and or using other known therapeutically effective concentrations of such bronchodilator(s) known or disclosed in the art. The amount of bronchodilating component per container/canister, therefore, should be considered as encompassed by/included in this disclosure.

### Anticholinergic/Canister

Amounts of anticholinergic component (e.g., anticholinergic compound(s)/agent(s)) has/have been provided here in both weight percent (percent by weight, wt. %) of a composition and mg/mL of a composition. Thus, the reader should understand that the amount of an anticholinergic component/container, when composition(s) are provided in a container for the delivery of a plurality of doses thereof, will depend on the volume of the composition provided in the container. The amount of an anticholinergic component within the container/canister can be calculated given the possible concentrations of anticholinergic component in the composition, disclosed here and or using other known therapeutically effective concentrations of such anticholinergic compound(s)/agents known or disclosed in the art. The amount of anticholinergic component per container/canister, therefore, should be considered as encompassed by/included in this disclosure.

### Non-Anticholinergic/Canister

Amounts of a non-anticholinergic component (e.g., non-anticholinergic compound(s)/agent(s)) has/have been provided here in both weight percent (percent by weight, wt. %) of a composition and mg/mL of a composition. Thus, the reader should understand that the amount of a non-anticholinergic component/container, when composition(s) are provided in a container for the delivery of a plurality of doses thereof, will depend on the volume of the composition provided in the container. The amount of a non-anticholinergic component within the container/canister can be calculated given the possible concentrations of non-anticholinergic component in the composition, disclosed here and or using other known therapeutically effective concentrations of such non-anticholinergic compound(s)/agent(s) known or disclosed in the art. The amount of non-anticholinergic component per container/canister, therefore, should be considered as encompassed by/included in this disclosure.

### Beta-Adrenergic Agonist/Canister

Amounts of a beta-adrenergic agonist component (e.g., beta-adrenergic agonist compound(s)/agent(s)) has/have been provided here in both weight percent (percent by weight, wt. %) of a composition and mg/mL of a composition. Thus, the reader should understand that the amount of a beta-adrenergic agonist component/container, when composition(s) are provided in a container for the delivery of a plurality of doses thereof, will depend on the volume of the composition provided in the container. The amount of a beta-adrenergic agonist component within the container/canister can be calculated given the possible concentrations of beta-adrenergic agonist component in the composition, disclosed here and or using other known therapeutically effective concentrations of such beta-adrenergic agonist compound(s)/agent(s) known or disclosed in the art. The amount of beta-adrenergic agonist component per container/canister, therefore, should be considered as encompassed by/included in this disclosure.

### Non-Bronchodilator/Canister

Amounts of a non-bronchodilating component (e.g., non-bronchodilating compound(s)/agent(s), such as, e.g., an anti-inflammatory component) either is given here or can be determined here in both weight percent (percent by weight, wt. %) of a composition and mg/mL of a composition. Thus, the reader should understand that the amount of a non-bronchodilating component/container, when composition(s) are provided in a container for the delivery of a plurality of doses thereof, will depend on the volume of the composition provided in the container. The amount of a non-bronchodilating component within the container/canister can be calculated given the possible concentrations of non-bronchodilating component in the composition, disclosed here and or using other known therapeutically effective concentrations of such non-bronchodilating compound(s)/agent(s) known or disclosed in the art. The amount of non-bronchodilating component per container/canister, therefore, should be considered as encompassed by/included in this disclosure.

### Anti-Inflammatory/Canister

Amounts of an anti-inflammatory component are given here in both weight percent (percent by weight, wt. %) of a composition and mg/mL of a composition. Thus, the reader should understand that the amount of an anti-inflammatory component/container, when composition(s) are provided in a container for the delivery of a plurality of doses thereof, will depend on the volume of the composition provided in the container. The amount of an anti-inflammatory component within the container/canister can be calculated given the possible concentrations of an anti-inflammatory component in the composition, disclosed here and or using other known therapeutically effective concentrations of such anti-inflammatory compound(s)/agent(s) known or disclosed in the art. The amount of anti-inflammatory component per container/canister, therefore, should be considered as encompassed by/included in this disclosure.

### Corticosteroid/Canister

Amounts of a corticosteroid component are given here in both weight percent (percent by weight, wt. %) of a composition and mg/mL of a composition. Thus, the reader should understand that the amount of a corticosteroid component/container, when composition(s) are provided in a container for the delivery of a plurality of doses thereof, will depend on the volume of the composition provided in the container. The amount of a corticosteroid component within the container/canister can be calculated given the possible concentrations of a corticosteroid component in the composition, disclosed here and or using other known therapeutically effective concentrations of such corticosteroid compound(s)/agent(s) known or disclosed in the art. The amount of corticosteroid component per container/canister, therefore, should be considered as encompassed by/included in this disclosure.

### API/Dose

In aspects, the amount of API component, or, similarly, e.g., any sub-component of an API component, such as, e.g., a bronchodilator component or constituent(s) thereof, anticholinergic component or constituent(s) thereof, non-anticholinergic component or constituent(s) thereof, beta-adrenergic component or constituent(s) thereof, non-bronchodilator component or constituent(s) thereof, anti-inflammatory component or constituent(s) thereof, or corticosteroid component or constituent(s) thereof, delivered per dose, is dependent upon one or more valve(s) present in the delivery device used to deliver composition(s), e.g., by inhalation. In aspects, delivery device(s) can comprise, e.g., any suitably sized valve capable of delivery any suitable volume of composition per dose, such as, e.g., a valve capable of delivering a dose of between about 25 μL and about 100 μL (or, stated alternatively, delivery device(s) can comprise a 25 μL-100 μL valve, e.g., a 50 μL valve). Herein, in exemplary aspects, an amount of API per dose (API/dose) may be provided without an associated exemplary valve size. In certain exemplary aspects, an exemplary amount of API/dose may be provided along with an exemplary valve size. Readers can modify the exemplary amounts utilizing percent composition amount(s) of API(s) provided herein, according to valve size(s) present in delivery device(s), and such calculations and resulting API/dose amounts should be considered disclosed herein.

In certain aspects, the API comprises a single bronchodilator. In aspects, the API comprises at least two bronchodilators. In aspects, the API comprises at least one bronchodilator and at least one non-bronchodilator. In aspects, the API comprises a tiotropium compound, a formoterol compound, an anti-inflammatory compound (e.g., a steroid anti-inflammatory compound, e.g., a corticosteroid compound, such as, e.g., a ciclesonide compound, a beclomethasone compound, or a fluticasone compound), or, e.g., combination(s) of any or all thereof.

In aspects, composition(s) comprise between about 1 μg and about 250 μg of API(s) (API component) per dose, such as, e.g., ˜1 μg-˜240 μg, ˜1 μg-˜220 μg, ˜1 μg-˜200 μg, ˜1 μg-˜180 μg, ˜1 μg-˜160 μg, ˜1 μg-˜140 μg, ˜1 μg-˜120 μg, ˜1 μg-˜100 μg, ˜1 μg-˜80 μg, ˜1 μg-˜60 μg, ˜1 μg-˜40 μg, ˜1 μg-˜20 μg, or, e.g., ˜1 μg-˜10 μg of API component per dose.

In aspects, composition(s) comprise between about 2 μg and about 250 μg of API component per dose, such as, e.g., ˜4 μg-˜250 μg, ˜6 μg-˜250 μg, ˜8 μg-˜250 μg, ˜10 μg-˜250 μg, ˜12 μg-˜250 μg, ˜14 μg-˜250 μg, ˜16 μg-˜250 μg, ˜18 μg-˜250 μg, ˜20 μg-˜250 μg, ˜30 μg-˜250 μg, ˜40 μg-˜250 μg, ˜50 μg-˜250 μg, ˜60 μg-˜250 μg, ˜80 μg-˜250 μg, ˜100 μg-˜250 μg, ˜120 μg-˜250 μg, ˜140 μg-˜250 μg, ˜160 μg-˜250 μg, ˜180 μg-˜250 μg, or ˜200 μg-˜250 μg of API component per dose.

In aspects, composition(s) comprise between about 2 μg and about 225 μg of API component per dose, such as, e.g., ˜5 μg-˜200 μg, ˜10 μg-˜180 μg, ˜20 μg-˜170 μg, ˜30 μg-˜160 μg, ˜40 μg-˜150 μg, ˜50 μg-˜140 μg, ˜60 μg-˜130 μg, ˜70 μg-˜120 μg, ˜80 μg-˜110 μg, ˜90 μg-˜110 μg, or, e.g., ˜100 μg-˜110 μg of API component per dose.

In aspects, composition(s) may be provided by an inhalation device comprising a valve size of between about 25 μL and about 100 μL, such as, e.g., about 25 μL. In such aspects, compositions may comprise, e.g., between about 1 μg and about 60 μg of API component per dose, such as, e.g., ˜2 μg, ˜4 μg, ˜6 μg, ˜8 μg, ˜10 μg, ˜12 μg, ˜14 μg, ˜16 μg, ˜18 μg, ˜20 μg, ˜22 μg, ˜24 μg, ˜26 μg, ˜28 μg, ˜30 μg, ˜32 μg, ˜34 μg, ˜36 μg, ˜38 μg, ˜40 μg, ˜42 μg, ˜44 μg, ˜46 μg, ˜48 μg, ˜50 μg, ˜52 μg, ˜54 μg, ˜56 μg, ˜58 μg, or, e.g., ˜60 μg API component per dose, or an amount of API component provided within a range established using any two such doses.

In aspects, composition(s) may be provided by an inhalation device comprising a valve size of between about 25 μL and about 100 μL, such as, e.g., about 50 μL. In such aspects, compositions may comprise, e.g., between about 2 μg and about 120 μg of API component per dose, such as, e.g., ˜4 μg, ˜8 μg, ˜12 μg, ˜16 μg, ˜20 μg, ˜24 μg, ˜28 μg, ˜32 μg, ˜36 μg, ˜40 μg, ˜44 μg, ˜48 μg, ˜52 μg, ˜56 μg, ˜60 μg, ˜64 μg, ˜68 μg, ˜72 μg, ˜76 μg, ˜80 μg, ˜84 μg, ˜88 μg, ˜92 μg, ˜96 μg, ˜100 μg, ˜104 μg, ˜108 μg, ˜112 μg, ˜116 μg, or, e.g., ˜120 μg API component per dose, or an amount of API component provided within a range established using any two such doses.

In aspects, composition(s) may be provided by an inhalation device comprising a valve size of between about 25 μL and about 100 μL, such as, e.g., about 75 μL. In such aspects, compositions may comprise, e.g., between about 4 μg and about 180 μg of API component per dose, such as, e.g., ˜6 μg, ˜12 μg, ˜18 μg, ˜24 μg, ˜30 μg, ˜36 μg, ˜42 μg, ˜48 μg, ˜54 μg, ˜60 μg, ˜66 μg, ˜72 μg, ˜78 μg, ˜84 μg, ˜90 μg, ˜96 μg, ˜102 μg, ˜108 μg, ˜114 μg, ˜120 μg, ˜126 μg, ˜132 μg, ˜138 μg, ˜144 μg, ˜150 μg, ˜156 μg, ˜162 μg, ˜168 μg, ˜174 μg, or, e.g., ˜180 μg API per dose, or an amount of API component provided within a range established using any two such doses.

In aspects, composition(s) may be provided by an inhalation device comprising a valve size of between about 25 μL and about 100 μL, such as, e.g., about 100 μL. In such aspects, compositions may comprise, e.g., between about 4 μg and about 240 μg of API component per dose, such as, e.g., ˜8 μg, ˜16 μg, ˜24 μg, ˜32 μg, ˜40 μg, ˜48 μg, ˜56 μg, ˜64 μg, ˜72 μg, ˜80 μg, ˜88 μg, ˜96 μg, ˜104 μg, ˜112 μg, ˜120 μg, ˜128 μg, ˜136 μg, ˜144 μg, ˜152 μg, ˜160 μg, ˜168 μg, ˜176 μg, ˜184 μg, ˜192 μg, ˜200 μg, ˜208 μg, ˜216 μg, ˜224 μg, ˜232 μg, or, e.g., ˜240 μg API per dose, or an amount of API component provided within a range established using any two such doses.

In aspects, compositions comprise at least about 2 μg of an API component per dose, such as, e.g., ≥˜4 μg, ≤˜6 μg, ≥˜8 μg, ≥˜12 μg, ≥˜16 μg, ≥˜20 μg, ≥˜25 μg, ≥˜50 μg, ≥˜75 μg, ≥˜100 μg, ≥˜150 μg, ≥˜200 μg, or ≥˜210 μg of an API component per dose.

In aspects, compositions comprise no more than (e.g., comprise less than) about 220 μg of an API component per dose, such as, e.g., ≤˜216 μg, ≤˜210 μg, ≤˜200 μg, ≤˜175 μg, ≤˜150 μg, ≤˜125 μg, ≤˜100 μg, ≤˜75 μg, ≤˜50 μg, ≤˜25 μg, ≤˜20 μg, ≤˜18 μg, ≤˜16 μg, ≤˜14 μg, ≤˜12 μg, ≤˜10 μg, ≤˜8 μg, ≤˜6 μg, ≤˜4 μg, or, e.g., ≤˜2 μg of an API component per dose.

### Bronchodilator/Dose

In certain aspects, composition(s) comprise a single bronchodilator compound. In aspects, a composition(s) comprise at least two bronchodilator compounds. In aspects, composition(s) comprise at least one anticholinergic compound. In aspects, composition(s) comprise at least one beta-adrenergic compound. In aspects, composition(s) comprise at least one anticholinergic compound and at least one beta-adrenergic compound. In aspects, composition(s) comprise a tiotropium compound, a formoterol compound, or both a tiotropium compound and a formoterol compound.

In aspects, composition(s) comprise between about 1 μg and about 20 μg of bronchodilator compound(s) per dose, such as, e.g., ˜1 μg-˜20 μg, ˜1 μg-˜19 μg, ˜1 μg-˜18 μg, ˜1 μg-˜17 μg, ˜1 μg-˜16 μg, ˜1 μg-˜15 μg, ˜1 μg-˜14 μg, ˜1 μg-˜13 μg, ˜1 μg-˜12 μg, ˜1 μg-˜11 μg, ˜1 μg-˜10 μg, ˜1 μg-˜9 μg, ˜1 μg-˜8 μg, ˜1 μg-˜7 μg, ˜1 μg-˜6 μg, ˜1 μg-˜5 μg, or, e.g., ˜1 μg-˜4 μg bronchodilator compound(s) per dose.

In aspects, composition(s) comprise between about 2 μg and about 20 μg of bronchodilator compound(s) per dose, such as, e.g., ˜1 μg-˜20 μg, ˜2 μg-˜20 μg, ˜3 μg-˜20 μg, ˜4 μg-˜20 μg, ˜5 μg-˜20 μg, ˜6 μg-˜20 μg, ˜7 μg-˜20 μg, ˜8 μg-˜20 μg, ˜9 μg-˜20 μg, ˜10 μg-˜20 μg, ˜11 μg-˜20 μg, ˜12 μg-˜20 μg, ˜13 μg-˜20 μg, ˜14 μg-˜20 μg, ˜15 μg-˜20 μg, or, e.g., ˜16 μg-˜20 μg bronchodilator compound(s) per dose.

In aspects, composition(s) comprise between about 2 μg and about 19 μg of bronchodilator compound(s) per dose, such as, e.g., ˜3 μg-˜18 μg, ˜4 μg-˜17 μg, ˜5 μg-˜16 μg, ˜6 μg-˜15 μg, ˜7 μg-˜14 μg, or, e.g., ˜7 μg-˜13 μg, ˜7 μg-˜12 μg, ˜7 μg-˜11 μg, ˜7 μg-˜10 μg, or, e.g., ˜7 μg-˜9 μg bronchodilator compound(s) per dose.

In aspects, composition(s) may be provided by an inhalation device comprising a valve size of between about 25 μL and about 100 μL, such as, e.g., about 25 μL. In such aspects, compositions may comprise, e.g., between about 1 μg and about 10 μg of a bronchodilator component per dose, such as, e.g., ˜1 μg, ˜2 μg, ˜3 μg, ˜4 μg, ˜5 μg, ˜6 μg, ˜7 μg, ˜8 μg, ˜9 μg, or ˜10 μg bronchodilator component per dose, or an amount of a bronchodilator component provided within a range established using any two such doses.

In aspects, composition(s) may be provided by an inhalation device comprising a valve size of between about 25 μL and about 100 μL, such as, e.g., about 50 μL. In such aspects, compositions may comprise, e.g., between about 2 μg and about 20 μg of a bronchodilator component per dose, such as, e.g., ˜2 μg, ˜4 μg, ˜6 μg, ˜8 μg, ˜10 μg, ˜12 μg, ˜14 μg, ˜16 μg, ˜18 μg, or, e.g., ˜20 μg bronchodilator component per dose, or an amount of a bronchodilator component provided within a range established using any two such doses.

In aspects, composition(s) may be provided by an inhalation device comprising a valve size of between about 25 μL and about 100 μL, such as, e.g., about 75 μL. In such aspects, compositions may comprise, e.g., between about 4 μg and about 30 μg of a bronchodilator component per dose, such as, e.g., ˜4 μg, ˜6 μg, ˜8 μg, ˜10 μg, ˜12 μg, ˜14 μg, ˜16 μg, ˜18 μg, ˜20 μg, ˜22 μg, ˜24 μg, ˜26 μg, ˜28 μg, or, e.g., ˜30 μg of a bronchodilator component per dose, or an amount of a bronchodilator component provided within a range established using any two such doses.

In aspects, composition(s) may be provided by an inhalation device comprising a valve size of between about 25 μL and about 100 μL, such as, e.g., about 100 μL. In such aspects, compositions may comprise, e.g., between about 6 μg and about 50 μg of a bronchodilator component per dose, such as, e.g., ˜6 μg, ˜8 μg, ˜10 μg, ˜12 μg, ˜14 μg, ˜16 μg, ˜18 μg, ˜20 μg, ˜22 μg, ˜24 μg, ˜26 μg, ˜28 μg, ˜30 μg, ˜32 μg, ˜34 μg, ˜36 μg, ˜38 μg, ˜40 μg, ˜42 μg, ˜44 μg, ˜46 μg, ˜48 μg, or, e.g., ˜50 μg of a bronchodilator component per dose, or an amount of a bronchodilator component provided within a range established using any two such doses.

In aspects, compositions comprise at least about 2 μg of a bronchodilator component per dose, such as, e.g., ≥˜4 μg, ≥˜5 μg, ≥˜6 μg, ≥˜7 μg, ≥˜8 μg, ≥˜9 μg, ≥˜10 μg, ≥˜11 μg, ≥˜12 μg, ≥˜13 μg, ≥˜14 μg, ≥˜15 μg, or ≥˜16 μg of a bronchodilator component per dose.

In aspects, compositions comprise no more than (e.g., comprise less than) about 20 μg of a bronchodilator component per dose, such as, e.g., ≤˜18 μg, ≤˜17 μg, ≤˜16 μg, ≤˜15 μg, ≤˜14 μg, ≤˜13 μg, ≤˜12 μg, ≤˜11 μg, ≤˜10 μg, ≤˜9 μg, ≤˜8 μg, ≤˜7 μg, ≤˜6 μg, ≤˜5 μg, ≤˜4 μg, ≤˜3 μg, or, e.g., ≤˜2 of a μg bronchodilator component per dose.

### Anticholinergic/Dose

In aspects, composition(s) comprise a single anticholinergic compound. In aspects, composition(s) comprise at least two anticholinergic agent(s). In aspects, composition(s) comprise a tiotropium compound.

In aspects, composition(s) comprise an anticholinergic component in an amount of between about 1 μg and about 20 μg per dose, such as, e.g., ˜1 μg-˜19 μg, ˜1 μg-˜18 μg, ˜1 μg-˜17 μg, ˜1 μg-˜16 μg, ˜1 μg-˜15 μg, ˜1 μg-˜14 μg, ˜1 μg-˜13 μg, ˜1 μg-˜12 μg, ˜1 μg-˜11 μg, ˜1 μg-˜10 μg, ˜1 μg-˜9 μg, ˜1 μg-˜8 μg, ˜1 μg-˜7 μg, ˜1 μg-˜6 μg, ˜1 μg-˜5 μg, ˜1 μg-˜4 μg, ˜1 μg-˜3 μg, or ˜1 μg-˜2 μg anticholinergic component per dose.

In aspects, composition(s) comprise an anticholinergic component in an amount of between about 2 μg and about 20 μg per dose, such as, e.g., ˜3 μg-˜20 μg, ˜4 μg-˜20 μg, ˜5 μg-˜20 μg, ˜6 μg-˜20 μg, ˜7 μg-˜20 μg, ˜8 μg-˜20 μg, ˜9 μg-˜20 μg, ˜10 μg-˜20 μg, ˜11 μg-˜20 μg, ˜12 μg-˜20 μg, ˜13 μg-˜20 μg, ˜14 μg-˜20 μg, ˜15 μg-˜20 μg, ˜16 μg-˜20 μg, ˜17 μg-˜20 μg, ˜18 μg-˜20 μg, or, e.g., ˜19 μg-˜20 μg anticholinergic component per dose.

In aspects, composition(s) comprise an anticholinergic component in an amount of between about 1.5 μg and about 18 μg per dose, such as, e.g., ˜2 μg-˜16 μg, ˜2.5 μg-˜14 μg, ˜3 μg-˜12 μg, ˜3 μg-˜10 μg, or, e.g., ˜3 μg-˜8 μg, ˜3 μg-˜7 μg, ˜3 μg-˜6 μg, or, e.g., ˜3 μg-˜5 μg anticholinergic component per dose.

In aspects, composition(s) may be provided by an inhalation device comprising a valve size of between about 25 μL and about 100 μL, such as, e.g., about 25 μL. In such aspects, compositions may comprise, e.g., between about 1 μg and about 5 μg of an anticholinergic component per dose, such as, e.g., ˜1 μg, ˜2 μg, ˜3 μg, ˜4 μg, or ˜5 μg of an anticholinergic component per dose, or an amount of an anticholinergic component provided within a range established using any two such doses.

In aspects, composition(s) may be provided by an inhalation device comprising a valve size of between about 25 μL and about 100 μL, such as, e.g., about 50 μL. In such aspects, compositions may comprise, e.g., between about 2 μg and about 8 μg of an anticholinergic component per dose, such as, e.g., ˜2 μg, ˜3 μg, ˜4 μg, ˜5 μg, ˜6 μg, ˜7 μg, or, e.g., ˜8 μg of an anticholinergic component per dose, or an amount of anticholinergic component provided within a range established using any two such doses.

In aspects, composition(s) may be provided by an inhalation device comprising a valve size of between about 25 μL and about 100 μL, such as, e.g., about 75 μL. In such aspects, compositions may comprise, e.g., between about 2 μg and about 10 μg of an anticholinergic component per dose, such as, e.g., ˜2 μg, ˜3 μg, ˜4 μg, ˜5 μg, ˜6 μg, ˜7 μg, ˜8 μg, ˜9 μg, or, e.g., ˜10 μg of an anticholinergic component per dose, or an amount of anticholinergic component provided within a range established using any two such doses.

In aspects, composition(s) may be provided by an inhalation device comprising a valve size of between about 25 μL and about 100 μL, such as, e.g., about 100 μL. In such aspects, compositions may comprise, e.g., between about 4 μg and about 20 μg of anticholinergic component per dose, such as, e.g., ˜4 μg, ˜5 μg, ˜6 μg, ˜7 μg, ˜8 μg, ˜9 μg, ˜10 μg, ˜11 μg, ˜12 μg, ˜13 μg, ˜14 μg, ˜15 μg, ˜16 μg, ˜17 μg, ˜18 μg, ˜19 μg, or, e.g., ˜20 μg of an anticholinergic component per dose, or an amount of anticholinergic component provided within a range established using any two such doses.

In aspects, the total amount of an anticholinergic component, e.g., an anticholinergic compound/agent, e.g., a tiotropium compound, such as, e.g., tiotropium bromide, that is delivered in a single actuation of a delivery device can be any suitable dose and may depend, e.g., on the nature of the condition being treated and patient population that the delivery device, e.g., inhaler, is designed to treat. In aspects, the total dose of an anticholinergic compound/agent, e.g., a tiotropium compound, such as, e.g., tiotropium bromide, delivered per dose, e.g., per actuation of an inhalation device, is not less than (e.g., is at least) about 1 μg, such as, e.g., ≥˜2 μg, ≥˜3 μg, ≥˜4 μg, ≥˜5 μg, ≥˜6 μg, ≥˜7 μg, ≥˜8 μg, ≥˜9 μg, ≥˜10 μg, ≥˜11 μg, ≥˜12 μg, ≥˜13 μg, ≥˜14 μg, ≥˜15 μg, ≥˜16 μg, ≥˜17 μg, ≥˜18 μg, ≥˜19 μg, or, e.g., ≤˜20 μg of anticholinergic component per dose.

In aspects, the total dose of an anticholinergic component/compound/agent, e.g., a tiotropium compound, such as, e.g., tiotropium bromide, delivered per dose, e.g., per actuation of an inhalation device, is no more than about 20 μg, such as, e.g., ≤˜19 μg, ≤˜18 μg, ≤˜17 μg, ≤˜16 μg, ≤˜15 μg, ≤˜14 μg, ≤˜13 μg, ≤˜12 μg, ≤˜11 μg, ˜10 μg, ≤˜9 μg, ≤˜8 μg, ≤˜7 μg, ≤˜6 μg, ≤˜5 μg, ≤˜4 μg, ≤˜3 μg, or, e.g., ≤˜2 μg of anticholinergic component/dose.

### Non-Anticholinergic/Dose

In aspects, composition(s) comprise at least one non-anticholinergic compound. In aspects, composition(s) comprise at least two non-anticholinergic compounds. In aspects, composition(s) comprise at least one non-anticholinergic bronchodilating compound and at least one non-anticholinergic, non-bronchodilating compound. In aspects, composition(s) comprise at least one beta-adrenergic compound and at least one non-bronchodilating compound. In aspects, composition(s) comprise a formoterol compound and at least one anti-inflammatory compound, such as, e.g., at least one steroid compound, e.g., a corticosteroid compound, e.g., a beclomethasone, ciclesonide, or fluticasone compound.

In aspects, composition(s) comprise between about 1 μg and about 250 μg of a non-anticholinergic component per dose, such as, e.g., ˜1 μg-˜240 μg, ˜1 μg-˜220 μg, ˜1 μg-˜200 μg, ˜1 μg-˜180 μg, ˜1 μg-˜160 μg, ˜1 μg-˜140 μg, ˜1 μg-˜120 μg, ˜1 μg-˜100 μg, ˜1 μg-˜80 μg, ˜1 μg-˜60 μg, ˜1 μg-˜40 μg, ˜1 μg-˜20 μg, or, e.g., ˜1 μg-˜10 μg of a non-anticholinergic component per dose.

In aspects, composition(s) comprise between about 2 μg and about 250 μg of a non-anticholinergic component per dose, such as, e.g., ˜4 μg-˜250 μg, ˜6 μg-˜250 μg, ˜8 μg-˜250 μg, ˜10 μg-˜250 μg, ˜12 μg-˜250 μg, ˜14 μg-˜250 μg, ˜16 μg-˜250 μg, ˜18 μg-˜250 μg, ˜20 μg-˜250 μg, ˜30 μg-˜250 μg, ˜40 μg-˜250 μg, ˜50 μg-˜250 μg, ˜60 μg-˜250 μg, ˜80 μg-˜250 μg, ˜100 μg-˜250 μg, ˜120 μg-˜250 μg, ˜140 μg-˜250 μg, ˜150 μg-˜250 μg, ˜160 μg-˜250 μg, ˜180 μg-˜250 μg, or ˜200 μg-˜250 μg of a non-anticholinergic component per dose.

In aspects, composition(s) comprise between about 2 μg and about 225 μg of a non-anticholinergic component per dose, such as, e.g., ˜5 μg-˜200 μg, ˜10 μg-˜180 μg, ˜20 μg-˜170 μg, ˜30 μg-˜160 μg, ˜40 μg-˜150 μg, ˜50 μg-˜140 μg, ˜60 μg-˜130 μg, ˜70 μg-˜120 μg, ˜80 μg-˜110 μg, ˜90 μg-˜110 μg, or, e.g., ˜100 μg-˜110 μg of a non-anticholinergic component per dose.

In aspects, composition(s) may be provided by an inhalation device comprising a valve size of between about 25 μL and about 100 μL, such as, e.g., about 25 μL. In such aspects, compositions may comprise, e.g., between about 1 μg and about 75 μg of a non-anticholinergic component per dose, such as, e.g., ˜2 μg, ˜4 μg, ˜6 μg, ˜8 μg, ˜10 μg, ˜12 μg, ˜14 μg, ˜16 μg, ˜18 μg, ˜20 μg, ˜22 μg, ˜24 μg, ˜26 μg, ˜28 μg, ˜30 μg, ˜32 μg, ˜34 μg, ˜36 μg, ˜38 μg, ˜40 μg, ˜42 μg, ˜44 μg, ˜46 μg, ˜48 μg, ˜50 μg, ˜52 μg, ˜54 μg, ˜56 μg, ˜58 μg, ˜60 μg, ˜62 μg, ˜64 μg, ˜68 μg, ˜70 μg, ˜72 μg, ˜74 μg, or, e.g., ˜75 μg of a non-anticholinergic component per dose, or an amount of a non-anticholinergic component provided within a range established using any two such doses.

In aspects, composition(s) may be provided by an inhalation device comprising a valve size of between about 25 μL and about 100 μL, such as, e.g., about 50 μL. In such aspects, compositions may comprise, e.g., between about 2 μg and about 120 μg of a non-anticholinergic component per dose, such as, e.g., ˜2 μg, ˜4 μg, ˜6 μg, ˜8 μg, ˜10 μg, ˜20 μg, ˜30 μg, ˜40 μg, ˜50 μg, ˜60 μg, ˜70 μg, ˜80 μg, ˜90 μg, ˜100 μg, ˜110 μg, or, e.g., ˜120 μg of a non-anticholinergic component per dose, or an amount of a non-anticholinergic component provided within a range established using any two such doses.

In aspects, composition(s) may be provided by an inhalation device comprising a valve size of between about 25 μL and about 100 μL, such as, e.g., about 75 μL. In such aspects, compositions may comprise, e.g., between about 4 μg and about 180 μg of API/dose, such as, e.g., ˜4 μg, ˜6 μg, ˜8 μg, ˜10 μg, ˜20 μg, ˜30 μg, ˜40 μg, ˜50 μg, ˜60 μg, ˜70 μg, ˜80 μg, ˜90 μg, ˜100 μg, ˜110 μg, ˜120 μg, ˜130 μg, ˜140 μg, ˜150 μg, ˜160 μg, ˜170 μg, or, e.g., ˜180 μg of a non-anticholinergic component per dose, or an amount of a non-anticholinergic component provided within a range established using any two such doses.

In aspects, composition(s) may be provided by an inhalation device comprising a valve size of between about 25 μL and about 100 μL, such as, e.g., about 100 μL. In such aspects, compositions may comprise, e.g., between about 6 μg and about 240 μg of API/dose, such as, e.g., ˜6 μg, ˜8 μg, ˜10 μg, ˜20 μg, ˜40 μg, ˜60 μg, ˜80 μg, ˜100 μg, ˜120 μg, ˜140 μg, ˜160 μg, ˜180 μg, ˜200 μg, ˜220 μg, or, e.g., ˜240 μg of a non-anticholinergic component per dose, or an amount of a non-anticholinergic component provided within a range established using any two such doses.

In aspects, the total amount of a non-anticholinergic component, e.g., non-anticholinergic compound(s)/agent(s), e.g., a formoterol compound, such as, e.g., formoterol fumarate, and optionally anti-inflammatory agent(s), such as steroid(s), e.g., corticosteroid(s), such as, e.g., ciclesonide compound, beclomethasone compound, or fluticasone compound, that is delivered in a single actuation of a delivery device can be any suitable dose and may depend, e.g., on the nature of the condition being treated and patient population that the delivery device, e.g., inhaler, is designed to treat. In aspects, the total dose of a non-anticholinergic component delivered per dose, e.g., per actuation of an inhalation device, is not less than (e.g., is at least) about 2 μg, such as, e.g., ≥˜2 μg, ≥˜4 μg, ≤˜6 μg, ≤˜8 μg, ≤˜10 μg, ≥˜20 μg, ≤˜40 μg, ≥˜50 μg, ≥˜60 μg, ≥˜80 μg, ≥˜100 μg, ≥˜120 μg, ≥˜140 μg, ≥˜160 μg, ≥˜180 μg, ≥˜200 μg, ≥˜220 μg, or, e.g., ≥˜240 μg of a non-anticholinergic component per dose.

In aspects, the In aspects, the total dose of a non-anticholinergic component delivered per dose, e.g., per actuation of an inhalation device, is no more than about 250 μg, such as, e.g., ≤˜240 μg, ≤˜220 μg, ≤˜200 μg, ≤˜180 μg, ≤˜160 μg, ≤˜140 μg, ≤˜120 μg, ≤˜100 μg, ≤˜80 μg, ≤˜60 μg, ≤˜50 μg, ≤˜40 μg, ≤˜20 μg, ≤˜10 μg, ≤˜8 μg, ≤˜6 μg, ≤˜4 μg, or, e.g., ≤˜2 μg of a non-anticholinergic component per dose.

### Beta-Adrenergic/Dose

In aspects, composition(s) comprise a single beta-adrenergic compound. In aspects, composition(s) comprise at least two beta-adrenergic agent(s). In aspects, composition(s) comprise a formoterol compound.

In aspects, composition(s) comprise a beta-adrenergic component in an amount of between about 1 μg and about 20 μg per dose, such as, e.g., ˜1 μg-˜19 μg, ˜1 μg-˜18 μg, ˜1 μg-˜17 μg, ˜1 μg-˜16 μg, ˜1 μg-˜15 μg, ˜1 μg-˜14 μg, ˜1 μg-˜13 μg, ˜1 μg-˜12 μg, ˜1 μg-˜11 μg, ˜1 μg-˜10 μg, ˜1 μg-˜9 μg, ˜1 μg-˜8 μg, ˜1 μg-˜7 μg, ˜1 μg-˜6 μg, ˜1 μg-˜5 μg, ˜1 μg-˜4 μg, ˜1 μg-˜3 μg, or ˜1 μg-˜2 μg of a beta-adrenergic component per dose.

In aspects, composition(s) comprise a beta-adrenergic component in an amount of between about 2 μg and about 20 μg per dose, such as, e.g., ˜3 μg-˜20 μg, ˜4 μg-˜20 μg, ˜5 μg-˜20 μg, ˜6 μg-˜20 μg, ˜7 μg-˜20 μg, ˜8 μg-˜20 μg, ˜9 μg-˜20 μg, ˜10 μg-˜20 μg, ˜11 μg-˜20 μg, ˜12 μg-˜20 μg, ˜13 μg-˜20 μg, ˜14 μg-˜20 μg, ˜15 μg-˜20 μg, ˜16 μg-˜20 μg, ˜17 μg-˜20 μg, ˜18 μg-˜20 μg, or, e.g., ˜19 μg-˜20 μg of a beta-adrenergic component per dose.

In aspects, composition(s) comprise a beta-adrenergic component in an amount of between about 1.5 μg and about 18 μg per dose, such as, e.g., ˜2 μg-˜16 μg, ˜2.5 μg-˜14 μg, ˜3 μg-˜12 μg, ˜3 μg-˜10 μg, or, e.g., ˜3 μg-˜8 μg, ˜3 μg-˜7 μg, ˜3 μg-˜6 μg, or, e.g., ˜3 μg-˜5 μg of a beta-adrenergic component per dose.

In aspects, composition(s) may be provided by an inhalation device comprising a valve size of between about 25 μL and about 100 μL, such as, e.g., about 25 μL. In such aspects, compositions may comprise, e.g., between about 1 μg and about 5 μg of beta-adrenergic component per dose, such as, e.g., ˜1 μg, ˜2 μg, ˜3 μg, ˜4 μg, or ˜5 μg of beta-adrenergic component per dose, or an amount of beta-adrenergic component provided within a range established using any two such doses.

In aspects, composition(s) may be provided by an inhalation device comprising a valve size of between about 25 μL and about 100 μL, such as, e.g., about 50 μL. In such aspects, compositions may comprise, e.g., between about 2 μg and about 8 μg of beta-adrenergic component per dose, such as, e.g., ˜2 μg, ˜3 μg, ˜4 μg, ˜5 μg, ˜6 μg, ˜7 μg, or, e.g., ˜8 μg of beta-adrenergic component per dose, or an amount of a beta-adrenergic component provided within a range established using any two such doses.

In aspects, composition(s) may be provided by an inhalation device comprising a valve size of between about 25 μL and about 100 μL, such as, e.g., about 75 μL. In such aspects, compositions may comprise, e.g., between about 2 μg and about 10 μg of beta-adrenergic component per dose, such as, e.g., ˜2 μg, ˜3 μg, ˜4 μg, ˜5 μg, ˜6 μg, ˜7 μg, ˜8 μg, ˜9 μg, or, e.g., ˜10 μg of beta-adrenergic component per dose, or an amount of a beta-adrenergic component provided within a range established using any two such doses.

In aspects, composition(s) may be provided by an inhalation device comprising a valve size of between about 25 μL and about 100 μL, such as, e.g., about 100 μL. In such aspects, compositions may comprise, e.g., between about 4 μg and about 20 μg of a beta-adrenergic component per dose, such as, e.g., ˜4 μg, ˜5 μg, ˜6 μg, ˜7 μg, ˜8 μg, ˜9 μg, ˜10 μg, ˜11 μg, ˜12 μg, ˜13 μg, ˜14 μg, ˜15 μg, ˜16 μg, ˜17 μg, ˜18 μg, ˜19 μg, or, e.g., ˜20 μg of a beta-adrenergic component per dose, or an amount of a beta-adrenergic component provided within a range established using any two such doses.

In aspects, the total amount of a beta-adrenergic component, e.g., a beta-adrenergic compound/agent, e.g., a formoterol compound, such as, e.g., formoterol fumarate, that is delivered in a single actuation of a delivery device can be any suitable dose and may depend, e.g., on the nature of the condition being treated and patient population that the delivery device, e.g., inhaler, is designed to treat. In aspects, the total dose of a beta-adrenergic component, e.g., a formoterol compound, such as, e.g., formoterol fumarate, delivered per dose, e.g., per actuation of an inhalation device, is not less than (e.g., is at least) about 1 μg, such as, e.g., ≥˜2 μg, ≥˜3 μg, ≥˜4 μg, ≥˜5 μg, ≥˜6 μg, ≥˜7 μg, ≤˜8 μg, ≥˜9 μg, ≥˜10 μg, ≥˜11 μg, ≥˜12 μg, ≥˜13 μg, ≥˜14 μg, ≥˜15 μg, ≥˜16 μg, ≥˜17 μg, ≥˜18 μg, ≥˜19 μg, or, e.g., ≥˜20 μg of a beta-adrenergic component per dose.

In aspects, the total dose of a beta-adrenergic component/compound/agent, e.g., a formoterol compound, such as, e.g., formoterol fumarate, delivered per dose, e.g., per actuation of an inhalation device, is no more than about 20 μg, such as, e.g., ≤˜19 μg, ≤˜18 μg, ≤˜17 μg, ≤˜16 μg, ≤˜15 μg, ≤˜14 μg, ≤˜13 μg, ≤˜12 μg, ≤˜11 μg, ≤˜10 μg, ≤˜9 μg, ≤˜8 μg, ≤˜7 μg, ≤˜6 μg, ≤˜5 μg, ≤˜4 μg, ≤˜3 μg, or, e.g., ≤˜2 μg of a beta-adrenergic component per dose.

### Non-Bronchodilator/Dose

In aspects, composition(s) comprise at least one non-bronchodilating compound. In aspects, composition(s) comprise at least one anti-inflammatory compound. In aspects, composition(s) comprise at least one steroid compound. In aspects, composition(s) comprise at least one corticosteroid compound. In aspects, composition(s) comprise at least one of a ciclesonide compound, beclomethasone compound, or fluticasone compound.

In aspects, composition(s) can comprise between about 25 μg and about 250 μg of a non-bronchodilator component per dose, such as, e.g., ˜25 μg-˜225 μg, ˜25 μg-˜200 μg, ˜25 μg-˜175 μg, ˜25 μg-˜150 μg, ˜25 μg-˜125 μg, ˜25 μg-˜100 μg, ˜25 μg-˜75 μg, or, e.g., ˜25 μg-˜50 μg of a non-bronchodilator component per dose.

In aspects, composition(s) can comprise between about 30 μg and about 250 μg of a non-bronchodilator component per dose, such as, e.g., ˜35 μg-˜250 μg, ˜40 μg-˜250 μg, ˜45 μg-˜250 μg, ˜50 μg-˜250 μg, ˜75 μg-˜250 μg, ˜100 μg-˜250 μg, ˜125 μg-˜250 μg, ˜150 μg-˜250 μg, ˜175 μg-˜250 μg, ˜200 μg-˜250 μg, or, e.g., ˜225 μg-˜250 μg of a non-bronchodilator component per dose.

In aspects, composition(s) can comprise between about ˜35 μg-˜225 μg, ˜40 μg-˜200 μg, ˜45 μg-˜175 μg, ˜50 μg-˜150 μg, ˜75 μg-˜125 μg, or, e.g., ˜90 μg-˜110 μg of a non-bronchodilator component per dose.

In aspects, composition(s) may be provided by an inhalation device comprising a valve size of between about 25 μL and about 100 μL, such as, e.g., about 25 μL. In such aspects, compositions may comprise, e.g., between about 10 μg and about 100 μg of a non-bronchodilator component per dose, such as, e.g., ˜10 μg, ˜20 μg, ˜30 μg, ˜40 μg, ˜50 μg, ˜60 μg, ˜70 μg, ˜80 μg, ˜90 μg, or ˜100 μg of a non-bronchodilator component per dose, or an amount of a non-bronchodilator component provided within a range established using any two such doses.

In aspects, composition(s) may be provided by an inhalation device comprising a valve size of between about 25 μL and about 100 μL, such as, e.g., about 50 μL. In such aspects, compositions may comprise, e.g., between about 50 μg and about 150 μg of a non-bronchodilator component per dose, such as, e.g., ˜50 μg, ˜60 μg, ˜70 μg, ˜80 μg, ˜90 μg, ˜100 μg, ˜110 μg, ˜120 μg, ˜130 μg, ˜140 μg, or, e.g., ˜150 μg of a non-bronchodilator component per dose, or an amount of a non-bronchodilator component provided within a range established using any two such doses.

In aspects, composition(s) may be provided by an inhalation device comprising a valve size of between about 25 μL and about 100 μL, such as, e.g., about 75 μL. In such aspects, compositions may comprise, e.g., between about 100 μg and about 200 μg of a non-bronchodilator component per dose, such as, e.g., ˜100 μg, ˜110 μg, ˜120 μg, ˜130 μg, ˜140 μg, ˜150 μg, ˜160 μg, ˜170 μg, ˜180 μg, ˜190 μg, or, e.g., ˜200 μg of a non-bronchodilator component per dose, or an amount of a non-bronchodilator component provided within a range established using any two such doses.

In aspects, composition(s) may be provided by an inhalation device comprising a valve size of between about 25 μL and about 100 μL, such as, e.g., about 100 μL. In such aspects, compositions may comprise, e.g., between about 150 μg and about 250 μg of a non-bronchodilator component per dose, such as, e.g., ˜150 μg, ˜160 μg, ˜170 μg, ˜180 μg, ˜190 μg, ˜200 μg, ˜210 μg, ˜220 μg, ˜230 μg, ˜240 μg, or, e.g., ˜250 μg of a non-bronchodilator component per dose, or an amount of a non-bronchodilator component provided within a range established using any two such doses.

In aspects, the total amount of a non-bronchodilator component, e.g., an anti-inflammatory component/compound/agent, e.g., a steroid component/compound/agent, e.g., a corticosteroid component/compound/agent, such as, e.g., a ciclesonide compound, beclomethasone compound, or fluticasone compound, that is delivered in a single actuation of a delivery device can be any suitable dose and may depend, e.g., on the nature of the condition being treated and patient population that the delivery device, e.g., inhaler, is designed to treat. In aspects, the total dose of a non-bronchodilator component, e.g., an anti-inflammatory component/compound/agent, e.g., a steroid component/compound/agent, e.g., a corticosteroid component/compound/agent, such as, e.g., a ciclesonide compound, beclomethasone compound, or fluticasone compound, delivered per dose, e.g., per actuation of an inhalation device, is not less than (e.g., is at least) about 25 μg, such as, e.g., ≥˜30 μg, ≥˜35 μg, ≥˜40 μg, ≥˜45 μg, ≥˜50 μg, ≥˜55 μg, ≥˜60 μg, ≥˜65 μg, ≥˜70 μg, ≥˜75 μg, ≥˜100 μg, ≥˜125 μg, ≥˜150 μg, ≥˜175 μg, ≥˜200 μg, ≥˜225 μg, or, e.g., ≥˜250 μg of a non-bronchodilator component per dose.

In aspects, the total dose of a non-bronchodilator component, e.g., an anti-inflammatory component/compound/agent, e.g., a steroid component/compound/agent, e.g., a corticosteroid component/compound/agent, such as, e.g., a ciclesonide compound, beclomethasone compound, or fluticasone compound, delivered per dose, e.g., per actuation of an inhalation device, is no more than about 250 μg, such as, e.g., ≤˜240 μg, ≤˜220 μg, ≤˜200 μg, ≤˜180 μg, ≤˜160 μg, ≤˜150 μg, ≤˜140 μg, ≤˜130 μg, ≤˜120 μg, ≤˜110 μg, ≤˜100 μg, ≤˜90 μg, ≤˜80 μg, ≤˜70 μg, ≤˜60 μg, ≤˜55 μg, or, e.g., ≤˜50 μg of a non-bronchodilator component per dose.

### Anti-Inflammatory/Dose

In aspects, composition(s) comprise at least one anti-inflammatory compound. In aspects, composition(s) comprise at least one steroid compound. In aspects, composition(s) comprise at least one corticosteroid compound. In aspects, composition(s) comprise at least one of a ciclesonide compound, beclomethasone compound, or fluticasone compound.

In aspects, composition(s) can comprise between about 25 μg and about 250 μg of an anti-inflammatory component per dose, such as, e.g., ˜25 μg-˜225 μg, ˜25 μg-˜200 μg, ˜25 μg-˜175 μg, ˜25 μg-˜150 μg, ˜25 μg-˜125 μg, ˜25 μg-˜100 μg, ˜25 μg-˜75 μg, or, e.g., ˜25 μg-˜50 μg of an anti-inflammatory component per dose.

In aspects, composition(s) can comprise between about 30 μg and about 250 μg of an anti-inflammatory component per dose, such as, e.g., ˜35 μg-˜250 μg, ˜40 μg-˜250 μg, ˜45 μg-˜250 μg, ˜50 μg-˜250 μg, ˜75 μg-˜250 μg, ˜100 μg-˜250 μg, ˜125 μg-˜250 μg, ˜150 μg-˜250 μg, ˜175 μg-˜250 μg, ˜200 μg-˜250 μg, or, e.g., ˜225 μg-˜250 μg of an anti-inflammatory component per dose.

In aspects, composition(s) can comprise between about ˜35 μg-˜225 μg, ˜40 μg-˜200 μg, ˜45 μg-˜175 μg, ˜50 μg-˜150 μg, ˜75 μg-˜125 μg, or, e.g., ˜90 μg-˜110 μg of an anti-inflammatory component per dose.

In aspects, composition(s) may be provided by an inhalation device comprising a valve size of between about 25 μL and about 100 μL, such as, e.g., about 25 μL. In such aspects, compositions may comprise, e.g., between about 10 μg and about 100 μg of an anti-inflammatory component per dose, such as, e.g., ˜10 μg, ˜20 μg, ˜30 μg, ˜40 μg, ˜50 μg, ˜60 μg, ˜70 μg, ˜80 μg, ˜90 μg, or ˜100 μg of an anti-inflammatory component per dose, or an amount of an anti-inflammatory component provided within a range established using any two such doses.

In aspects, composition(s) may be provided by an inhalation device comprising a valve size of between about 25 μL and about 100 μL, such as, e.g., about 50 μL. In such aspects, compositions may comprise, e.g., between about 50 μg and about 150 μg of an anti-inflammatory component per dose, such as, e.g., ˜50 μg, ˜60 μg, ˜70 μg, ˜80 μg, ˜90 μg, ˜100 μg, ˜110 μg, ˜120 μg, ˜130 μg, ˜140 μg, or, e.g., ˜150 μg of an anti-inflammatory component per dose, or an amount of an anti-inflammatory component provided within a range established using any two such doses.

In aspects, composition(s) may be provided by an inhalation device comprising a valve size of between about 25 μL and about 100 μL, such as, e.g., about 75 μL. In such aspects, compositions may comprise, e.g., between about 100 μg and about 200 μg of an anti-inflammatory component per dose, such as, e.g., ˜100 μg, ˜110 μg, ˜120 μg, ˜130 μg, ˜140 μg, ˜150 μg, ˜160 μg, ˜170 μg, ˜180 μg, ˜190 μg, or, e.g., ˜200 μg of an anti-inflammatory component per dose, or an amount of an anti-inflammatory component provided within a range established using any two such doses.

In aspects, composition(s) may be provided by an inhalation device comprising a valve size of between about 25 μL and about 100 μL, such as, e.g., about 100 μL. In such aspects, compositions may comprise, e.g., between about 150 μg and about 250 μg of an anti-inflammatory component per dose, such as, e.g., ˜150 μg, ˜160 μg, ˜170 μg, ˜180 μg, ˜190 μg, ˜200 μg, ˜210 μg, ˜220 μg, ˜230 μg, ˜240 μg, or, e.g., ˜250 μg of an anti-inflammatory component per dose, or an amount of an anti-inflammatory component provided within a range established using any two such doses.

In aspects, the total amount of an anti-inflammatory component, e.g., a steroid component/compound/agent, e.g., a corticosteroid component/compound/agent, such as, e.g., a ciclesonide compound, beclomethasone compound, or fluticasone compound, that is delivered in a single actuation of a delivery device can be any suitable dose and may depend, e.g., on the nature of the condition being treated and patient population that the delivery device, e.g., inhaler, is designed to treat. In aspects, the total dose of an anti-inflammatory component, e.g., a steroid component/compound/agent, e.g., a corticosteroid component/compound/agent, such as, e.g., a ciclesonide compound, beclomethasone compound, or fluticasone compound, delivered per dose, e.g., per actuation of an inhalation device, is not less than (e.g., is at least) about 25 μg, such as, e.g., ≥˜30 μg, ≥˜35 μg, ≥˜40 μg, ≤˜45 μg, ≥˜50 μg, ≥˜55 μg, ≥˜60 μg, ≥˜65 μg, ≥˜70 μg, ≥˜75 μg, ≥˜100 μg, ≥˜125 μg, ≥˜150 μg, ≥˜175 μg, ≥˜200 μg, ≥˜225 μg, or, e.g., ≥˜250 μg of an anti-inflammatory component per dose.

In aspects, the total dose of an anti-inflammatory component, e.g., a steroid component/compound/agent, e.g., a corticosteroid component/compound/agent, such as, e.g., a ciclesonide compound, beclomethasone compound, or fluticasone compound, delivered per dose, e.g., per actuation of an inhalation device, is no more than about 250 μg, such as, e.g., ≤˜240 μg, ≤˜220 μg, ≤˜200 μg, ≤˜180 μg, ≤˜160 μg, ≤˜150 μg, ≤˜140 μg, ≤˜130 μg, ≤˜120 μg, ≤˜110 μg, ≤˜100 μg, ≤˜90 μg, ≤˜80 μg, ≤˜70 μg, ≤˜60 μg, ≤˜55 μg, or, e.g., ≤˜50 μg of an anti-inflammatory component per dose.

### Corticosteroid/Dose

In aspects, composition(s) comprise at least one corticosteroid compound. In aspects, composition(s) comprise at least one of a ciclesonide compound, beclomethasone compound, or fluticasone compound.

In aspects, composition(s) can comprise between about 25 μg and about 250 μg of a corticosteroid component per dose, such as, e.g., ˜25 μg-˜225 μg, ˜25 μg-˜200 μg, ˜25 μg-˜175 μg, ˜25 μg-˜150 μg, ˜25 μg-˜125 μg, ˜25 μg-˜100 μg, ˜25 μg-˜75 μg, or, e.g., ˜25 μg-˜50 μg of a corticosteroid component per dose.

In aspects, composition(s) can comprise between about 30 μg and about 250 μg of a corticosteroid component per dose, such as, e.g., ˜35 μg-˜250 μg, ˜40 μg-˜250 μg, ˜45 μg-˜250 μg, ˜50 μg-˜250 μg, ˜75 μg-˜250 μg, ˜100 μg-˜250 μg, ˜125 μg-˜250 μg, ˜150 μg-˜250 μg, ˜175 μg-˜250 μg, ˜200 μg-˜250 μg, or, e.g., ˜225 μg-˜250 μg of a corticosteroid component per dose.

In aspects, composition(s) can comprise between about ˜35 μg-˜225 μg, ˜40 μg-˜200 μg, ˜45 μg-˜175 μg, ˜50 μg-˜150 μg, ˜75 μg-˜125 μg, or, e.g., ˜90 μg-˜110 μg of a corticosteroid component per dose.

In aspects, composition(s) may be provided by an inhalation device comprising a valve size of between about 25 μL and about 100 μL, such as, e.g., about 25 μL. In such aspects, compositions may comprise, e.g., between about 10 μg and about 100 μg of a corticosteroid component per dose, such as, e.g., ˜10 μg, ˜20 μg, ˜30 μg, ˜40 μg, ˜50 μg, ˜60 μg, ˜70 μg, ˜80 μg, ˜90 μg, or ˜100 μg of a corticosteroid component per dose, or an amount of a corticosteroid component provided within a range established using any two such doses.

In aspects, composition(s) may be provided by an inhalation device comprising a valve size of between about 25 μL and about 100 μL, such as, e.g., about 50 μL. In such aspects, compositions may comprise, e.g., between about 50 μg and about 150 μg of a corticosteroid component per dose, such as, e.g., ˜50 μg, ˜60 μg, ˜70 μg, ˜80 μg, ˜90 μg, ˜100 μg, ˜110 μg, ˜120 μg, ˜130 μg, ˜140 μg, or, e.g., ˜150 μg of a corticosteroid component per dose, or an amount of a corticosteroid component provided within a range established using any two such doses.

In aspects, composition(s) may be provided by an inhalation device comprising a valve size of between about 25 μL and about 100 μL, such as, e.g., about 75 μL. In such aspects, compositions may comprise, e.g., between about 100 μg and about 200 μg of a corticosteroid component per dose, such as, e.g., ˜100 μg, ˜110 μg, ˜120 μg, ˜130 μg, ˜140 μg, ˜150 μg, ˜160 μg, ˜170 μg, ˜180 μg, ˜190 μg, or, e.g., ˜200 μg of a corticosteroid component per dose, or an amount of a corticosteroid component provided within a range established using any two such doses.

In aspects, composition(s) may be provided by an inhalation device comprising a valve size of between about 25 μL and about 100 μL, such as, e.g., about 100 μL. In such aspects, compositions may comprise, e.g., between about 150 μg and about 250 μg of a corticosteroid component per dose, such as, e.g., ˜150 μg, ˜160 μg, ˜170 μg, ˜180 μg, ˜190 μg, ˜200 μg, ˜210 μg, ˜220 μg, ˜230 μg, ˜240 μg, or, e.g., ˜250 μg of a corticosteroid component per dose, or an amount of a corticosteroid component provided within a range established using any two such doses.

In aspects, the total amount of a corticosteroid component, such as, e.g., a ciclesonide compound, beclomethasone compound, or fluticasone compound, that is delivered in a single actuation of a delivery device can be any suitable dose and may depend, e.g., on the nature of the condition being treated and patient population that the delivery device, e.g., inhaler, is designed to treat. In aspects, the total dose of a corticosteroid component, such as, e.g., a ciclesonide compound, beclomethasone compound, or fluticasone compound, delivered per dose, e.g., per actuation of an inhalation device, is not less than (e.g., is at least) about 25 μg, such as, e.g., ≥˜30 μg, ≥˜35 μg, ≥˜40 μg, ≥˜45 μg, ≥˜50 μg, ≥˜55 μg, ≥˜60 μg, ≤˜65 μg, ≥˜70 μg, ≥˜75 μg, ≥˜100 μg, ≥˜125 μg, ≥˜150 μg, ≥˜175 μg, ≥˜200 μg, ≥˜225 μg, or, e.g., ≥˜250 μg of a corticosteroid component per dose.

In aspects, the total dose of a corticosteroid component, such as, e.g., a ciclesonide compound, beclomethasone compound, or fluticasone compound, delivered per dose, e.g., per actuation of an inhalation device, is no more than about 250 μg, such as, e.g., ≤˜240 μg, ≤˜220 μg, ≤˜200 μg, ≤˜180 μg, ≤˜160 μg, ≤˜150 μg, ≤˜140 μg, ≤˜130 μg, ≤˜120 μg, ≤˜110 μg, ≤˜100 μg, ≤˜90 μg, ≤˜80 μg, ≤˜70 μg, ≤˜60 μg, 5-55 μg, or, e.g., ≤˜50 μg of a corticosteroid component per dose.

### Administration Schedule

In aspects, composition(s) provided by the invention can be administered according to an administration schedule sufficient to provide a therapeutically effective amount of any one or more API(s) present in the composition. In aspects, composition(s) provided herein can be administered, e.g., about 5 times, per day, about 4 times per day, about 3 times per day, about 2 times per day, or, e.g., about once per day. In certain aspects, composition(s) herein are not administered more than about 5 times per day, more than about 4 times per day, more than about 3 times per day, more than about 2 times per day, or, e.g., are not administered more than about once per day. In aspects, composition(s) are administered about 1 to about 3 times per day, such as about once or twice per day. In aspects, composition(s) are administered twice per day. In aspects, composition(s) are administered no more than once per day.

According to certain aspects, the invention provides a method of treating or preventing asthma, chronic obstructive pulmonary disease, similar or related respiratory disorders, or one or more symptoms related to one or more such conditions, wherein the method provides the administration of a therapeutically effective amount of composition(s) herein, e.g., sufficient to generate a detectable or significant therapeutic effect, according to a schedule wherein composition(s) are administered between about 1 time and about 5 times per day, such as, e.g., between about 1 time and about 4 times per day, between about 1 time and about 3 times per day, between about once and about twice per day, or, e.g., wherein composition(s) are administered once or twice per day. In certain aspects, the invention provides a method of treating or preventing asthma, chronic obstructive pulmonary disease, similar or related respiratory disorders, or one or more symptoms related to one or more such conditions, wherein the method provides the administration of a therapeutically effective amount of composition(s) herein no more than once per day.

### Kits

According to certain embodiments, composition(s) herein can be provided as component(s) of kit(s). In aspects, composition(s) can be provided, e.g., packaged in container(s), such as, e.g., canister(s), ready for use in delivery device(s), e.g., an inhaler device(s).

In aspects, composition(s) can be provided as a component of kit(s), wherein kit(s) comprise a composition described herein packaged in a container, e.g., a canister.

In aspects, composition(s) can be provided as a component of kit(s), wherein kit(s) comprise a composition described herein packaged in a container, e.g., a canister, and the kit further comprises a delivery device suited to receive the container, e.g., canister, such as, e.g., an inhaler device, such as a metered dose inhaler, e.g., a pressurized metered dose inhaler or a breath actuated inhaler.

In aspects, two or more packaged composition(s), e.g., two or more canisters each providing a composition described herein, can be provided in a kit.

In certain aspects, a kit can comprise, e.g., one or more composition(s) packaged in one or more respective containers, and, further, the kit can comprise one or more delivery device(s) suited to receive a container, e.g., canister, such as, e.g., an inhaler device, such as a metered dose inhaler, e.g., a pressurized metered dose inhaler or a breath actuated inhaler.

According to aspects, kit(s) described herein can comprise use of any one or more composition(s) disclosed herein. In aspects, kit(s) provided by the invention can be used in any one or more method(s) described herein.

### Methods of Use

In aspects, the invention provides method(s) of directly treating or prophylactically treating in a mammal, e.g., a human, chronic obstructive pulmonary disease (COPD), asthma, one or more similar or related respiratory disorders, such as, e.g., disorders related to the obstruction of airflow to or from the lung(s); any one or more symptoms related to any one or more of such conditions; or any combination of any or all thereof. In aspects, method(s) comprise the administration of a therapeutically effective amount of pharmaceutical composition(s) described herein.

In aspects, “similar or related respiratory disorders” or, e.g., “disorders related to the obstruction of airflow to or from the lung(s), used herein, can include, e.g., inflammatory airway diseases or conditions, obstructive airway diseases or conditions, or any combination of any or all thereof. In aspects, exemplary conditions include, e.g., acute lung injury (ALI), acute respiratory distress syndrome (ARDS), chronic obstructive pulmonary airways disease (COAD), chronic obstructive lung disease (COLD), chronic bronchitis and emphysema, bronchiectasis and exacerbation of airways hyperreactivity resulting from one or more separate drug therapy(ies) (such as, e.g., one or more separate inhalation therapy(ies)), and pneumoconiosis(es), such as, e.g., aluminosis, anthracosis, asbestosis, chalicosis, ptilosis, siderosis, silicosis, tabacosis, and, e.g., byssinosis.

In aspects, the invention provides method(s) of using one or more composition(s) described herein in the direct treatment or prophylactic treatment of a mammal, e.g., a human, suffering from or at risk of suffering from chronic obstructive pulmonary disease (COPD), asthma, one or more similar or related respiratory disorders, such as, e.g., disorders related to the obstruction of airflow to or from the lung(s); any one or more symptoms related to any one or more of such conditions; or any combination of any or all thereof. In aspects, method(s) of using composition(s) in such treatment comprise the administration of therapeutically effective amount(s) of pharmaceutical composition(s) described herein.

In aspects, the invention provides method(s) of detectably or significantly expanding, e.g., detectably or significantly dilating one or more airway(s), e.g., bronchioles, in a recipient, e.g., a mammal such as a human, suffering from a restriction of such one or more airways. In aspects, method(s) of detectably or significantly expanding, e.g., dilating, one or more airway(s) comprise(s) the administration of a therapeutically effective amount of pharmaceutical composition(s) described herein.

In aspects, the invention provides method(s) of detectably or significantly reducing inflammation associated with one or more airway(s) or systems related to respiration in a recipient, e.g., a mammal such as a human, suffering from inflammation of such airway(s) or related respiratory system(s). In aspects, method(s) of detectably or significantly reducing inflammation are method(s) of reducing inflammation associated with asthma, COPD, or one or more similar or related respiratory disorders, such as, e.g., disorders related to the obstruction of airflow to or from the lung(s). In aspects, method(s) of detectably or significantly reducing inflammation associated with one or more airway(s) or related respiratory system(s) comprise(s) the administration of a therapeutically effective amount of pharmaceutical composition(s) described herein.

According to certain specific embodiments, the invention provides a method for prophylaxis or treatment of asthma, chronic obstructive pulmonary disease or related respiratory disorders by administering therapeutically effective amount(s) of composition(s) comprising (a) a bronchodilating component, e.g., comprising a tiotropium compound, e.g., tiotropium or a pharmaceutically acceptable salt or solvate thereof; (b) a stabilizing component, e.g., comprising an acid, e.g., comprising an organic acid, such as, e.g., citric acid, maleic acid, or a salt of such an acid; (c) a solvent component comprising, e.g., three or more solvent compound(s) such as, e.g., glycerol, one or more C1-C6 alcohol compound(s)/agent(s) (such as, e.g., ethanol), or both, and, e.g., water; and (d) a propellant component comprising one or more propellant compound(s)/agent(s) selected from HFO-1234ze, HFA-134a, HFA-152a, HFA-227ea, or mixture thereof. In certain aspects, the bronchodilating component of such composition(s) can further comprise a beta-adrenergic agonist component comprising, e.g., a formoterol compound, e.g., formoterol fumarate, e.g., formoterol fumarate dihydrate.

According to another aspect of the present invention, the invention provides the use of a composition comprising (a) a bronchodilating component, e.g., comprising a tiotropium compound, e.g., tiotropium or a pharmaceutically acceptable salt or solvate thereof; (b) a stabilizing component, e.g., comprising an acid, e.g., comprising an organic acid, such as, e.g., citric acid, or a salt of such an acid; (c) a solvent component comprising, e.g., three or more solvent compound(s) such as, e.g., glycerol, one or more C1-C6 alcohol compound(s)/agent(s) (such as, e.g., ethanol), or both, and, e.g., water; and (d) a propellant component comprising one or more propellant compound(s)/agent(s) selected from HFO-1234ze, HFA-227ea, HFA-134a, HFA-152a, or mixture thereof for the prophylaxis or treatment of asthma, chronic obstructive pulmonary disease or related respiratory disorders. In certain aspects, the bronchodilating component of such composition(s) can further comprise a beta-adrenergic agonist component comprising, e.g., a formoterol compound, e.g., formoterol fumarate, e.g., formoterol fumarate dihydrate.

According to another aspect of the present invention, the invention provides the use of a composition comprising (a) a bronchodilating component, e.g., comprising a tiotropium compound, e.g., tiotropium or a pharmaceutically acceptable salt or solvate thereof; (b) a stabilizing component, e.g., comprising an acid, e.g., comprising an organic acid, such as, e.g., citric acid, or a salt of such an acid; (c) a solvent component comprising, e.g., three or more solvent compound(s) such as, e.g., glycerol, one or more C1-C6 alcohol compound(s)/agent(s) (such as, e.g., ethanol), or both, and, e.g., water; and (d) a propellant component comprising one or more propellant compound(s)/agent(s) selected from HFO-1234ze, HFA-134a, HFA-227ea, HFA-152a, or mixture thereof in the manufacture of a medicament for the prophylaxis or treatment of asthma, chronic obstructive pulmonary disease or related respiratory disorders. In certain aspects, the bronchodilating component of such composition(s) can further comprise a beta-adrenergic agonist component comprising, e.g., a formoterol compound, e.g., formoterol fumarate, e.g., formoterol fumarate dihydrate.

In another aspect, the invention provides a method for the prophylaxis or treatment of asthma, chronic obstructive pulmonary disease or related respiratory disorders comprising administering a therapeutically effective amount of a composition comprising (a) a bronchodilating component, comprising, e.g., an anticholinergic component, comprising, e.g., a tiotropium compound, e.g., tiotropium or a pharmaceutically acceptable salt or solvate thereof; (b) an anti-inflammatory component comprising, e.g., a steroid component, e.g., a corticosteroid component, e.g., a beclomethasone compound such as beclomethasone or a pharmaceutically acceptable salt or solvate thereof, a ciclesonide compound, or a fluticasone compound, e.g., fluticasone furoate; (c) a stabilizing component, e.g., comprising an acid, e.g., comprising an organic acid, such as, e.g., maleic acid, citric acid, or a salt of such an acid; a solvent component comprising, e.g., three or more solvent compound(s) such as, e.g., glycerol, one or more C1-C6 alcohol compound(s)/agent(s) (such as, e.g., ethanol), or both, and, e.g., water; and (d) a propellant component comprising one or more propellant compound(s)/agent(s) selected from HFO-1234ze, HFA-134a, HFA-152a, HFA-227ea, or mixture thereof. In certain aspects, the bronchodilating component of such composition(s) can further comprise a beta-adrenergic agonist component comprising, e.g., a formoterol compound, e.g., formoterol fumarate, e.g., formoterol fumarate dihydrate.

According to another aspect of the present invention, there is provided the use of a composition comprising (a) a bronchodilating component, comprising, e.g., an anticholinergic component, comprising, e.g., a tiotropium compound, e.g., tiotropium or a pharmaceutically acceptable salt or solvate thereof; (b) an anti-inflammatory component comprising, e.g., a steroid component, e.g., a corticosteroid component, e.g., a beclomethasone compound such as beclomethasone or a pharmaceutically acceptable salt or solvate thereof, a ciclesonide compound, or a fluticasone compound, e.g., fluticasone furoate; (c) a stabilizing component, e.g., comprising an acid, e.g., comprising an organic acid, such as, e.g., citric acid, maleic acid, or a salt of such an acid; a solvent component comprising, e.g., three or more solvent compound(s) such as, e.g., glycerol, one or more C1-C6 alcohol compound(s)/agent(s) (such as, e.g., ethanol), or both, and, e.g., water; and (d) a propellant component comprising one or more propellant compound(s)/agent(s) selected from HFO-1234ze, HFA-227ea, HFA-134a, HFA-152a, or mixture thereof for the prophylaxis or treatment of asthma, chronic obstructive pulmonary disease or related respiratory disorders. In certain aspects, the bronchodilating component of such composition(s) can further comprise a beta-adrenergic agonist component comprising, e.g., a formoterol compound, e.g., formoterol fumarate, e.g., formoterol fumarate dihydrate.

According to another aspect of the present invention, there is provided the use of a composition comprising (a) a bronchodilating component, comprising, e.g., an anticholinergic component, comprising, e.g., a tiotropium compound, e.g., tiotropium or a pharmaceutically acceptable salt or solvate thereof; (b) an anti-inflammatory component comprising, e.g., a steroid component, e.g., a corticosteroid component, e.g., a beclomethasone compound such as beclomethasone or a pharmaceutically acceptable salt or solvate thereof, a ciclesonide compound, or a fluticasone compound, e.g., fluticasone furoate; (c) a stabilizing component, e.g., comprising an acid, e.g., comprising an organic acid, such as, e.g., citric acid, maleic acid, or a salt of such an acid; a solvent component comprising, e.g., three or more solvent compound(s) such as, e.g., glycerol, one or more C1-C6 alcohol compound(s)/agent(s) (such as, e.g., ethanol), or both, and, e.g., water; and (d) a propellant component comprising one or more propellant compound(s)/agent(s) selected from HFO-1234ze, HFA-134a, HFA-227ea, HFA-152a, or mixture thereof in the manufacture of a medicament for the prophylaxis or treatment of asthma, chronic obstructive pulmonary disease or related respiratory disorders. In certain aspects, the bronchodilating component of such composition(s) can further comprise a beta-adrenergic agonist component comprising, e.g., a formoterol compound, e.g., formoterol fumarate, e.g., formoterol fumarate dihydrate.

According to certain aspects, the invention provides method(s) of administering one or more bronchodilating compound(s), such as, e.g., one or more anticholinergic compound(s), one or more non-anticholinergic compound(s), or both; one or more beta-adrenergic compound(s); or any combination thereof, the method comprising the administration of a therapeutically effective amount of a composition described herein. According to certain aspects, the invention provides method(s) of administering one or more non-bronchodilating compound(s), such as, e.g., one or more anti-inflammatory compound(s), such as, e.g., one or more steroid(s), e.g., one or more corticosteroid(s), the method comprising the administration of a therapeutically effective amount of a composition described herein. According to certain aspects, the invention provides method(s) of administering one or more inhaled corticosteroid compound(s), the method comprising the administration of a therapeutically effective amount of a composition described herein. In aspects, the invention provides method(s) of administering any one or more of the compound(s) described in this paragraph wherein the method comprises the administration of a therapeutically effective amount of a composition described herein, wherein the composition comprises one or more excipient(s) described herein, such as, e.g., one or more of a co-solvent, low volatility component, stabilizer, dispersing agent, pH adjusting agent, surface active agent, or mixtures thereof. In aspects, the invention provides method(s) of administering any one or more of the compound(s) described in this paragraph wherein the method comprises the administration of a therapeutically effective amount of a composition described herein, wherein the composition comprises a propellant component, wherein the propellant component comprises one or more of HFA-134a, HFA-152a, HFA-227ea, and HFO-1234ze, such as, e.g., HFA-134a, HFA-152a, and HFO-1234ze, such as, e.g., only HFO-1234ze. e.g., only HFO-1234ze.

In aspects, method(s) described in this section comprise the provision of, e.g., the delivery of, composition(s) to recipient(s) via a delivery device, e.g., an inhaler, device, e.g., a metered dose inhaler such as a pressurized metered dose inhaler, or, e.g., a breath actuated metered dose inhaler. In aspects, such an inhaler comprises a metering valve. In aspects, such an inhaler comprises a low orifice actuator. In aspects, the low orifice actuator has a diameter of between about 0.1 mm to about 0.7 mm, such as, e.g., about 0.2 mm to about 0.6 mm, such as, e.g., about 0.3 mm to about 0.5 mm or, e.g., about 0.4 mm to about 0.5 mm.

According to aspects, method(s) described herein can comprise use of any one or more composition(s) disclosed herein. In aspects, method(s) described herein can comprise use of any one or more kit(s) described herein.

In aspects, the invention provides the method(s) described herein, wherein the application of the method(s) results in a detectably or significantly greater efficiency of delivery of one or more API(s) of the composition compared to a reference product, wherein the reference product is one or more reference products described herein. In aspects, the invention provides the method(s) described herein, wherein the application of the method(s) results in a detectable or significant clinical improvement in one or more measures of chronic obstructive pulmonary disease, asthma, one or more similar or related disorders, such as, e.g., disorders related to the obstruction of airflow to or from the lung(s); any one or more symptoms related to any one or more of such condition(s); or any combination of any or all thereof. In aspects, the invention provides the method(s) described herein, wherein the application of the method(s) results in a detectably or significantly greater clinical improvement in one or more measure(s) of chronic obstructive pulmonary disease, asthma, one or more similar or related disorder(s), such as, e.g., disorder(s) related to the obstruction of airflow to or from the lung(s); any one or more symptoms related to any one or more of such condition(s); or any combination of any or all thereof, compared to a reference product, wherein the reference product is one or more reference product(s) as described herein. In aspects, efficacy, outcome(s), or measures of clinical improvement demonstrated by method(s) provided herein is/are determined by an appropriately conducted and appropriately powered clinical study, such as an appropriately conducted clinical study recognized by an accepted regulatory authority such as, e.g., the U.S. FDA. In aspects, an outcome or measure of efficacy is an outcome described herein, such as, e.g., a performance characteristic, such as, e.g., a detectable or significant improvement in delivery efficiency; a detectable or significant reduction in frequency, severity, or frequency and severity of symptomatic attack (e.g., of acute asthmatic or bronchoconstrictor attack); a detectable or significant improvement in lung function; a detectable or significant improvement in airway hyperreactivity; a detectable or significant reduction in the requirement for one or more other symptomatic therapy(ies), e.g., one or more therapy(ies) for or intended to restrict or abort symptomatic attack when it occurs; a detectable or significant reduction in morning dipping (treatment between about 4 AM and about 6 AM, or, e.g., other similar period of time generally most distant from any previous treatment/administration of composition(s)); or any combination thereof or similar or related measure of composition efficacy/performance or other measure(s) of efficacy described herein or otherwise known in the art.

### Efficacy

In aspects, efficacy of composition(s), stability of composition(s), or other characteristic(s) of compositions described herein can be described in terms of performance characteristic(s) in relation to a reference product. In aspects, a reference product as used herein, (e.g., as it relates to efficacy, stability, etc.) can be a composition comprising at least mostly the same, at least generally the same, at least essentially the same, essentially the same, at least substantially the same, or the same active pharmaceutical ingredient(s) delivered by inhalation. In aspects, a reference product can be a composition comprising at least mostly the same, at least generally the same, at least essentially the same, essentially the same, at least substantially the same, or the same active pharmaceutical ingredient(s) delivered by inhalation, present in at least mostly the same, at least generally the same, at least essentially the same, essentially the same, at least substantially the same, or the same amount(s). In aspects, a reference product can be a composition sharing one or more excipient(s). In aspects, a reference product can be a composition sharing one or more excipient(s) in the same amount(s). In aspects, a reference product can be a composition comprising (a) at least mostly the same, at least generally the same, at least essentially the same, essentially the same, at least substantially the same, or the same active pharmaceutical ingredient(s), (b) at least mostly the same, at least generally the same, at least essentially the same, essentially the same, at least substantially the same, or the same active pharmaceutical ingredient(s) present in at least mostly the same, at least generally the same, at least essentially the same, essentially the same, at least substantially the same, or the same amount(s); (c) one or more of the same excipient(s); (d) one or more of the same excipient(s) in at least mostly the same, at least generally the same, at least essentially the same, essentially the same, at least substantially the same, or the same amount(s); or (e) any combination thereof, administered by inhalation, but comprising at least one propellant component constituent which varies from that of the present composition(s). In aspects, a reference product can be a product comprising at least mostly the same, at least generally the same, at least essentially the same, essentially the same, at least substantially the same, or the same active pharmaceutical ingredient(s) in at least mostly the same, at least generally the same, at least essentially the same, essentially the same, at least substantially the same, or the same amount(s), delivered by the same method of administration, e.g., inhalation, utilizing the same type of delivery device, e.g., an inhaler, such as the same type of inhaler, e.g., a metered dose inhaler, but wherein the reference product is provided in a different compositional form, such as, e.g., the reference product is provided as a suspension when the present composition(s) is/are provided as a solution.

In aspects, a reference product can be a product demonstrating bioequivalence to a reference product as described here (such as, e.g., a reference product recognized by an accepted regulatory authority, such as, e.g., the United States Food and Drug Administration (U.S. FDA), wherein such bioequivalence is determined by an appropriately conducted clinical study recognized by an accepted regulatory authority such as, e.g., the U.S. FDA.

In certain specific aspects, a reference product can be a product comprising the same API(s) in at least substantially the same amount(s), such as, e.g., a tiotropium compound, delivered via the same delivery mechanism, e.g., via inhalation (such as by an MDI), but wherein the reference product is provided in a different form, such as, e.g., as a suspension, when the present composition is provided as a solution. In certain aspects, efficiency of delivery of composition(s) herein provided as a solution is detectably or significantly greater than a reference composition provided as a suspension. In a specific example, the delivery efficiency of active pharmaceutical ingredient(s), e.g., tiotropium in composition(s) of the invention provided as a solution is detectably or significantly improved (e.g., greater than) that of a similar, e.g., reference, composition wherein the API(s), e.g., tiotropium, is/are provided in suspension. Here, “efficiency of delivery” refers to the amount of API, e.g., tiotropium reaching location(s) of air passageway(s) suitable for the delivery of detectable or significant treatment of such airway or airway-related condition, such as, e.g., detectable or significant reduction in frequency, severity, or frequency and severity of symptomatic attack (e.g., of acute asthmatic or bronchoconstrictor attack); detectable or significant improvement in lung function; a detectable or significant improvement in airway hyperreactivity; a detectable or significant reduction in the requirement for one or more other symptomatic therapy(ies), e.g., one or more therapy(ies) for or intended to restrict or abort symptomatic attack when it occurs; a detectable or significant reduction in morning dipping (treatment between about 4 AM and about 6 AM, or, e.g., other similar period of time generally most distant from any previous treatment/administration of composition(s)); or any combination thereof or similar or related symptom or condition described herein.

In aspects, administration of a therapeutically effective amount of composition(s) herein provide(s) a detectable or significant reduction in frequency, severity, or frequency and severity of symptomatic attack, e.g., of acute asthmatic or bronchoconstrictor attack, as, e.g., reported by patient(s), physician(s), or, e.g., as determined by an appropriated conducted and powered clinical study. In aspects, administration of a therapeutically effective amount of composition(s) herein provide(s) a detectably or significant reduction in frequency, severity, or frequency and severity of symptomatic attack, e.g. of acute asthmatic or bronchoconstrictor attack compared to reference product, as determined by an appropriately conducted clinical study or as recognized by an authoritative regulatory body such as, e.g., the U.S. FDA.

In aspects, administration of a therapeutically effective amount of composition(s) herein provide(s) a detectable or significant improvement in lung function, such as, e.g., lung function as reported by patient(s), physician(s), as determined by any one or more assessments or measures of lung function known in the art, or, e.g., as determined by an appropriately conducted and powered clinical study. In aspects, administration of a therapeutically effective amount of composition(s) herein provide(s) a detectable or significant improvement in lung function compared to reference product, as determined by an appropriately conducted clinical study or as recognized by an authoritative regulatory body such as, e.g., the U.S. FDA.

In aspects, administration of a therapeutically effective amount of composition(s) herein provide(s) a detectable or significant improvement in airway hyperreactivity, such as, e.g., airway hyperreactivity as reported by patient(s), physician(s), as determined by any one or more assessments or measures of airway hyperreactivity known in the art, or, e.g., as determined by an appropriately conducted and powered clinical study. In aspects, administration of a therapeutically effective amount of composition(s) herein provide(s) a detectable or significant improvement in airway hyperreactivity compared to reference product, as determined by an appropriately conducted clinical study or as recognized by an authoritative regulatory body such as, e.g., the U.S. FDA.

In aspects, administration of a therapeutically effective amount of composition(s) herein provide(s) a detectable or significant reduction in the requirement for one or more other symptomatic therapy(ies), e.g., one or more therapy(ies) for or intended to restrict or abort symptomatic attack when it occurs, as reported by patient(s), physician(s), as determined by any one or more assessments or measures of the same known in the art, or, e.g., as determined by an appropriately conducted and powered clinical study. In aspects, administration of a therapeutically effective amount of composition(s) herein provide(s) a detectably or significantly reduced requirement for one or more other symptomatic therapy(ies), e.g., one or more therapy(ies) for or intended to restrict or abort symptomatic attack when it occurs, compared to reference product, as determined by an appropriately conducted clinical study or as recognized by an authoritative regulatory body such as, e.g., the U.S. FDA.

In aspects, administration of a therapeutically effective amount of composition(s) herein provide(s) a detectable or significant reduction in morning dipping (treatment between about 4 AM and about 6 AM, or, e.g., other similar period of time generally most distant from any previous treatment/administration of composition(s)) as reported by patient(s), physician(s), or, e.g., as determined by an appropriately conducted and powered clinical study. In aspects, administration of a therapeutically effective amount of composition(s) herein provide(s) a detectably or significantly reduced frequency of morning dipping (treatment between about 4 AM and about 6 AM, or, e.g., other similar period of time generally most distant from any previous treatment/administration of composition(s)) compared to reference product, as determined by an appropriately conducted clinical study or as recognized by an authoritative regulatory body such as, e.g., the U.S. FDA.

In aspects, administration of a therapeutically effective amount of composition(s) herein comprising a combination therapy, e.g., comprising a long-acting muscarinic antagonist (LAMA)+long-acting beta agonist (LABA) combination therapy; a LAMA+inhaled corticosteroid (ICS) combination therapy; a LAMA+LABA+ICS combination therapy; or, e.g., a LABA+ICS combination therapy is detectably or significantly more effective, e.g., as measured by any indicator of effectiveness known in the art or described herein, than the separate use of one or more bronchodilator(s), the separate use of one or more anti-inflammatory(ies), e.g., the separate use of one or more corticosteroid(s), or, e.g., the separate use of both one or more bronchodilator(s) and the separate use of one or more anti-inflammatory(ies), e.g., one or more corticosteroid(s).

In aspects, administration of a therapeutically effective amount of composition(s) herein comprising one or more tiotropium compound(s) provide(s) a detectable or significant improvement in dyspnea as reported by patient(s), physician(s), as determined by any one or more assessments or measures of the same known in the art, or, e.g., as determined by an appropriately conducted and powered clinical study. In aspects, administration of a therapeutically effective amount of composition(s) herein comprising one or more tiotropium compound(s) provide(s) a detectable or significant improvement in dyspnea compared to a reference composition comprising short-term bronchodilating agent(s) as reported by patient(s), physician(s), as determined by any one or more assessments or measures of the same known in the art, or, e.g., as determined by an appropriately conducted and powered clinical study. In aspects, administration of a therapeutically effective amount of composition(s) herein provide(s) a detectably or significantly improved dyspnea compared to a reference product, as determined by an appropriately conducted clinical study or as recognized by an authoritative regulatory body such as, e.g., the U.S. FDA.

In aspects, administration of a therapeutically effective amount of composition(s) herein comprising one or more tiotropium compound(s) provide(s) a detectable or significant reduction in the need for, e.g., in the frequency of requirement for, rescue therapy, as reported by patient(s), physician(s), or, e.g., as determined by an appropriately conducted and powered clinical study. In aspects, administration of a therapeutically effective amount of composition(s) herein comprising one or more tiotropium compound(s) provide(s) a detectable or significant reduction in the need for, e.g., in the required frequency of receiving, one or more rescue therapy(ies) as reported by patient(s), physician(s), or, e.g., as determined by an appropriately conducted and powered clinical study. In aspects, administration of a therapeutically effective amount of composition(s) herein provide(s) a detectably or significantly improved dyspnea compared to a reference product, as determined by an appropriately conducted clinical study or as recognized by an authoritative regulatory body such as, e.g., the U.S. FDA.

In aspects, composition(s) herein demonstrate (a) detectably or significantly improved chemical stability; (b) detectably or significantly improved aerosolization performance; (c) detectably or significantly improved drug delivery; (d) detectably or significantly greater formulation (e.g., physical) stability; (e) detectably or significantly reduced environmental impact, (e.g., reduced GWP, reduced ODP, or both); (f) suitable compatibility with MDI component(s) manufactured using standard technique(s) and material(s) without having to modify such technique(s) or material(s) to address compositional compatibility issues; or (g) any combination of any or all of (a)-(f). In aspects, composition(s) herein demonstrate (a) detectably or significantly improved chemical stability; (b) detectably or significantly improved aerosolization performance; (c) detectably or significantly improved drug delivery; (d) detectably or significantly greater formulation (e.g., physical) stability; (e) detectably or significantly reduced environmental impact, (e.g., reduced GWP, reduced ODP, or both); or (f) any combination of (a)-(e) compared to a reference product as determined by an appropriately conducted clinical study or as recognized by an authoritative regulatory body such as, e.g., the U.S. FDA.

### Methods of Manufacturing

In certain aspects, composition(s) herein can be manufactured using one or more manufacturing step(s), process(es), material(s), or combination(s) thereof. In aspects, an advantage of composition(s) herein can be, e.g., that the specific type(s) and amount(s) of excipients and, e.g., propellant(s), provide an increased ease, cost, or both of manufacturing compared to one or more reference composition(s), similar composition(s) in the art, or both.

According to certain aspects, the invention provides a process for the preparation of composition(s) described herein. In aspects, the invention provides composition(s) made by processes(s) described herein. In aspects, method(s) of treatment can comprise, e.g., method(s) comprising composition(s) made by the method(s) of production/manufacturing described herein.

In aspects, the invention provides a process for preparing composition(s) comprising (1) a bronchodilating component, the bronchodilating component comprising (a) one or more anticholinergic compound(s), e.g., a tiotropium compound; (b) one or more beta-adrenergic agonist compound(s), e.g., a formoterol compound; or both (a) and (b); (2) a non-bronchodilating component, the non-bronchodilating component comprising, e.g., an anti-inflammatory component, the anti-inflammatory component comprising, e.g., a steroid component, e.g., a corticosteroid component, comprising, e.g., beclomethasone or a pharmaceutically acceptable salt or solvate thereof, a ciclesonide compound, or a fluticasone compound, e.g., fluticasone furoate, or a combination thereof; or (3) both (1) and (2), wherein the composition(s) demonstrate a detectably or significantly improved stability over a reference composition, such as, e.g., a reference composition described elsewhere herein, as determined by one or more measures of stability described elsewhere herein.

In aspects, a process for preparing the pharmaceutical composition(s) of the present invention comprises a step of admixing at least one bronchodilating compound, such as, e.g., at least one anticholinergic compound, e.g., at least one tiotropium compound, with a propellant comprising at least one propellant compound/agent. In aspects, such an admixing step comprises admixing one or more pharmaceutically acceptable excipients with the at least one bronchodilating compound (e.g., anticholinergic compound, e.g., tiotropium compound), with the one or more propellant compound(s)/agent(s), or both.

In aspects, composition(s) herein, e.g., composition(s) for delivery by inhalation by a delivery device, such as, e.g., an inhaler, e.g., an metered dose inhaler, comprise (1) an active pharmaceutical ingredient (API) component comprising (a) a bronchodilating component comprising, e.g. one or more bronchodilating compound(s), such as, e.g., tiotropium or a pharmaceutically acceptable salt or solvate thereof and formoterol or a pharmaceutically acceptable salt or solvate thereof; and optionally (b) a non-bronchodilating component, e.g., an anti-inflammatory component comprising, e.g., one or more steroid(s), e.g., one or more corticosteroid(s), such as, e.g., a beclomethasone compound such as beclomethasone or a pharmaceutically acceptable salt or solvate thereof, a ciclesonide compound, or a fluticasone compound, e.g., fluticasone furoate; (2) a non-volatile ingredient component comprising one or more excipient compounds; and (3) a propellant component comprising at least one propellant compound, wherein the composition is made by a process comprising (i) mixing the API component constituent(s) with the non-volatile ingredient component constituents; (ii) adding the mixture resulting from (i) to a container, e.g., a canister, suitable for use in a delivery system such as, e.g., a metered dose inhaler system; (iii) adding a valve to the canister of (ii); and (iv) pressure filling the propellant into the cannister through the valve. In certain aspects, no component(s) of the delivery system (e.g., canister, valve, etc. is/are coated).

In aspects, composition(s) herein can be manufactured according to following exemplary process. In aspects, uncontradicted, step(s) of the exemplified manufacturing process can be performed in any order or, e.g., simultaneously with other step(s).

In aspects, in the manufacture of composition(s) comprising two or more active pharmaceutical ingredients, at least a first API is combined with at least a second API in a first container. In aspects, this step is repeated until all APIs are combined.

In aspects, in the manufacture of composition(s) comprising a single API, the single API; or, in composition(s) comprising two or more APIs, the combination of APIs resulting from the above manufacturing step(s), are mixed with at least one other non-volatile composition component (e.g., composition excipient), such as, e.g., one, some, or all remaining non-volatile composition component(s) (excipient(s)).

In aspects, if not all remaining composition component(s) (excipient(s)) have been combined in the preceding step(s), at least one other non-volatile composition component (excipient), such as, e.g., one, some, or all remaining non-volatile composition component(s) (excipient(s)) are added to the container. In aspects, this step is repeated until all non-volatile composition components (excipients) have been added.

In aspects, the combined components of the preceding manufacturing process step(s) are mixed until uniformly suspended (as in, e.g., in the preparation of a suspension composition), or, e.g., until sufficiently mixed to facilitate addition of a propellant as is described below.

In certain aspects, upon attaining a sufficiently homogeneous mixture, the required quantity of the composition is added to a container, e.g., a canister suitable for use with a metered dose inhaler (MDI), or other final packaging unit (such exemplary amount(s) being described elsewhere herein.) In aspects, the addition can be accomplished by a cold filling method or a pressure filling method, each such method exemplified here.

In certain aspects, the manufacturing process comprises use of a metal container, the container is uncoated with any polymeric compound(s), the container is non-anodized, the MDI comprises no component, e.g., valve(s) or ring(s) made of a butyl or halo-butyl rubber, or any combination thereof. In certain alternative embodiments of manufacturing processes, the container is a non-metal container, the container is coated with one or more of polymeric compound(s), is an anodized container, the MDI comprises one or more components, e.g., valve(s) or ring(s) made of a butyl or halo-butyl rubber, or any combination thereof.

According to certain embodiments, the required amount of propellant is added to the composition resulting from the above step(s) and the resulting composition is mixed until the composition is completely dissolved (in, e.g., a composition provided as a solution) or until the composition is uniformly suspended (in, e.g., a composition provided as a suspension).

In aspects, pharmaceutical composition(s) can be placed into (e.g., filled into) a container, e.g., canister, by any method known in the art. In common aspects a cold filling process or a pressure filling process are used.

According to certain embodiments, the addition of the composition to a container, e.g., a canister, is completed by a cold filling process. In aspects, the cold filing process comprises (a) chilling the composition resulting from above step(s) to a suitable temperature, such as, e.g., between about −50° C.-about −60° C., e.g., about −50° C., about −51° C., about −52° C., about −53° C., about −54° C., about −55° C., about −56° ° C., about −57° C., about −58° C., about −59° C., or, e.g., about −60° C.; (b) adding the composition to the container, e.g., canister; (c) attaching a valve, e.g., a metered dose valve, to the container, e.g., canister, e.g., by crimping the valve, e.g., metered dose valve, to the container, e.g., canister; (d) allowing the container, e.g., canister, to warm to ambient temperature, resulting in an increase in pressure within the container, e.g., canister.

In alternative embodiments, the addition of the composition to a container, e.g., canister, is completed by a pressure filling process. In aspects, a pressure filling process comprises (a) adding the required amount of propellant to the composition resulting from above step(s); (b) attaching a valve, e.g., a metered dose valve, to a container, e.g., canister, e.g., by crimping the valve, e.g., metered dose valve, to the container, e.g., canister; (c) adding the composition resulting from (a) to the container, e.g., canister, through the valve, e.g., metered dose valve, by way of applied pressure.

In aspects, the addition of the composition to a container, e.g., canister, is completed by a pressure filling process comprising (a) adding all non-volatile composition component(s) from above step(s) to the container, e.g., canister; (b) attaching a valve, e.g., a metered dose valve, to the container, e.g., canister; (c) adding the volatile composition component(s), e.g., propellant(s) to the container, e.g., canister, through the valve, e.g., metered dose valve, by way of applied pressure.

According to certain aspects, the invention provides for composition(s) made by any one or more of the above-described process(es). In aspect, the invention provides for the use of such composition(s) made by such process(es) in one or more method(s) described herein. In aspects, composition(s) manufactured by such process(es) described here can be present in kit(s) provided by the invention.

According to one specific aspect, the invention provides composition(s) for delivery by inhalation via a metered dose inhaler, the composition(s) comprising (1) an active pharmaceutical ingredient (API) component comprising a bronchodilating component, comprising, e.g., an anticholinergic component, comprising, e.g., a tiotropium compound, e.g., tiotropium or a pharmaceutically acceptable salt or solvate thereof, the bronchodilating component further comprising a beta-adrenergic agonist component comprising, e.g., a formoterol compound, such as, e.g., formoterol or a pharmaceutically acceptable salt or solvate thereof; (2) a non-volatile ingredient component comprising one or more excipient component(s), or, e.g., one or more excipient compound(s)/agent(s); and (3) a propellant component comprising at least one propellant compound, wherein the composition is made by a process comprising (a) mixing the API component constituents with the non-volatile ingredient component constituents; (b) adding the mixture resulting from (a) to a container, e.g., a canister, suitable for use in a metered dose inhaler (MDI) delivery device; (c) adding a valve to the container/canister of (b); and (d) pressure filling the propellant into the cannister through the valve. In aspects, the API component of composition(s) described in this paragraph further comprises a non-bronchodilating component, e.g., an anti-inflammatory component, such as, e.g., a steroid component, e.g., an inhaled corticosteroid component comprising one or more inhaled corticosteroid compound(s)/agent(s) (including pharmaceutically acceptable salt(s) or solvate(s) thereof.)

According to certain exemplary aspects, composition(s) herein can be manufactured via the following step(s). In aspects, excipient(s) are weighed. In aspects, weighed excipient(s) are transferred to a first container, e.g., a vessel (referred to in this example as a “pre-mix vessel”). In aspects, performance-enhancing agent(s) are weighed. In aspects, weighed performance-enhancing agent(s) are added to the pre-mix vessel. In aspects, ethanol is transferred to the pre-mix vessel. In aspects wherein water is utilized, water is transferred to the premix vessel. In aspects, the contents of the pre-mix vessel are mixed until a clear solution is formed. In aspects, API(s) are added. In aspects wherein the composition being manufactured is to comprise fluticasone compound(s), all API(s) except fluticasone compound(s) are added to the pre-mix vessel. In aspects, contents of the pre-mix vessel are mixed until a clear solution forms. For manufacture of composition(s) comprising fluticasone compound(s), fluticasone compound(s), e.g., fluticasone furoate, is added to the pre-mix vessel. In aspects, the composition is homogenized. In aspects, the composition is homogenized for at least about 5 minutes, such as, e.g., ≥˜10 minutes, ≥˜15 minutes, ≥˜20 minutes, ≥˜25 minutes, ≥˜30 minutes, ≥˜35 minutes, ≥˜40 minutes, ≥˜45 minutes, ≥˜50 minutes, ≥˜55 minutes, ≥˜60 minutes, ≥˜65 minutes, ≥˜70 minutes, ≥˜75 minutes, ≥˜80 minutes, ≥˜85 minutes, or, e.g., ≥˜90 minutes. In aspects, the composition is homogenized using high shear mixing. In aspects, resulting compositions (with or without fluticasone compound(s)) are transferred from the pre-mix vessel to a second container, e.g., a second vessel, e.g., referred to here as a “main vessel.” In aspects, propellant(s) is/are added to the main vessel. In aspects, propellant and the composition are allowed to mix thoroughly. In aspects, final packaging containers, e.g., canisters, e.g., canisters crimped with valves, are filled. In aspects, compositions are filled in canisters that have been crimped with valves of about 20 to about 80 microliters, e.g., ˜20 μL-˜75 μL, ˜20 μL-˜70 μL, ˜20 μL-˜65 μL, ˜20 μL-˜60 μL, ˜20 μL-˜55 μL, or ˜20 μL-˜50 μL, such as, e.g., ˜25v-˜80 μL, ˜30 μL-˜80 μL, ˜35 μL-˜80 μL, ˜40 μL-˜80 μL, ˜45 μL-˜80 μL, or ˜50 μL-˜80 μL, e.g., ˜25 μL-˜75 μL microliters.

According to certain aspects, composition(s) in the form of a solution provided by the invention can be manufactured according to the following process. In aspects, the required amount(s) performance modulating agent(s), e.g., ethanol, glycerol, or both, are weighed/collected and added to a first container (e.g., a first vessel), e.g., a “pre-mix” vessel. In aspects, the required amount of water is added to the pre-mix vessel. In aspects, one or more acid(s) is added to the pre-mix vessel, e.g., one or more of citric acid, maleic acid, hydrochloric acid, etc. In aspects, the content of the pre-mix vessel is mixed until a clear solution forms.

In aspects between about 50% and about 80%, such as, e.g., ˜50%-˜75% or ˜50%-˜70%, such as, e.g., ˜60%-˜80% or ˜65%-˜80%, e.g., about 70%, of the required amount of propellant component is added to a second container (e.g., a second vessel), e.g., a “main” vessel. In aspects, the content of the pre-mix vessel is then added to the main vessel. In aspects, the required content of API(s) is added to the main vessel. In aspects, the main vessel is brought to a final target volume (or, e.g., final target weight), with propellant component.

In aspects, content of the main vessel is then mixed until a clear solution forms. In alternative aspects, for composition(s) comprising an API which does not completely solubilize, e.g., a hybrid solution-suspension composition, such as, e.g., composition(s) comprising a fluticasone compound, the content of the main vessel is homogenized (e.g., using high shear mixing) for a period of at least about 10 minutes, such as, e.g., ≥˜15 minutes, ≥˜20 minutes, ≥˜25 minutes, ≥˜30 minutes, ≥˜35 minutes, ≥˜40 minutes, ≥˜45 minutes, ≥˜50 minutes, ≥˜55 minutes, or, e.g., ≥˜60 minutes, such as ˜15 minutes-˜60 minutes or ˜20 minutes-˜60 minutes.

In aspects, final packaging, e.g., canisters, can be filled, e.g., pressure filed. In aspects, final packaging, e.g., canisters, are vacuum crimped. In aspects, vacuum crimping occurs prior to filling. In aspects, final packaging can be, e.g., canisters comprising a valve so as to facilitate administration by inhalation. In aspects, valves can be, e.g., 25 μL-100 μL valves, e.g., 25 μL-75 μL valves.

According to certain aspects, composition(s) in the form of a suspension provided by the invention can be manufactured according to the following process. In aspects, the required amount(s) of solid form excipient(s) (e.g., excipient(s) provided in solid form) is/are weighed/collected and added to a container (e.g., a vessel), e.g., a “main” vessel. In aspects, the required amount(s) of semi-solid form excipient(s) (e.g., excipient(s) provided in semi-solid form) is/are weight/collected and added to the main vessel. In aspects between about 70% and about 90%, such as, e.g., ˜70%-˜85% or ˜70%-˜80%, such as, e.g., ˜75%-˜90% or ˜80%-˜90%, e.g., about 80%, of the required amount of propellant component is added to the main vessel. In aspects, if ethanol is utilized in the formulation, ethanol is then added to the main vessel. In aspects, the required amount of API(s) is weighed/collected and added to the main vessel. In aspects, the main vessel is brought up to a final target volume (or, e.g., a final target weight) with propellant component.

In aspects, the content of the main vessel is then mixed for at least about 10 minutes, such as, e.g., ≥˜12 minutes, ≥˜14 minutes, ≥˜16 minutes, ≥˜18 minutes, or, e.g., ≥˜20 minutes.

In aspects, the content of the main vessel is homogenized for a period of at least about 10 minutes, such as, e.g., ≥˜15 minutes, ≥˜20 minutes, ≥˜25 minutes, ≥˜30 minutes, ≥˜35 minutes, ≥˜40 minutes, ≥˜45 minutes, ≥˜50 minutes, ≥˜55 minutes, or, e.g., ≥˜60 minutes, such as ˜15 minutes-˜60 minutes or ˜20 minutes-˜60 minutes. In aspects, any suitable homogenization process/equipment can be used. In certain aspects, an inline homogenizer is used. In aspects, an in-dwelling homogenizer is used. In aspects, a bottom load homogenizer is used. In aspects, a homogenizer characterizable as a high shear mixer is used.

In aspects, final packaging, e.g., canisters, can be filled, e.g., pressure filed. In aspects, final packaging, e.g., canisters, are vacuum crimped. In aspects, vacuum crimping occurs prior to filling. In aspects, final packaging can be, e.g., canisters comprising a valve so as to facilitate administration by inhalation. In aspects, valves can be, e.g., 25 μL-100 μL valves, e.g., 25 μL-75 μL valves.

### REPRESENTATIVE EXPERIMENTS/EMBODIMENTS (“EXAMPLES”)

The following detailed exemplary expository descriptions or experiments involving embodiments, applications, or related principles, of or otherwise related to the invention (“Examples”) are provided to assist readers in further understanding aspects of the invention or principles related to the invention or practice of aspects of the invention.

Any particular materials, methods, steps, and conditions employed/described in the following Examples, and any results thereof, are merely intended to further illustrate aspects of the invention. These Examples reflect exemplary embodiments of the invention, and the specific methods, findings, principles of such Examples, and the general implications thereof, can be combined with any other part of this disclosure. However, readers should understand that the invention is not limited by these Examples or any part thereof.

### Example 1

Table 2 below provides an exemplary composition provided by the invention.

TABLE 2
Exemplary Composition 1 (Solution).

| Ingredient | Quantity (wt. %) |
|---|---|
| Tiotropium | 0.005%-0.01% |
| Ethanol | 12%-18% |
| Propellant (HFA-134a; HFA-152a; HFO-1234ze) | q.s.* |
| Citric Acid | 0.05% |
| Glycerol | 0.0-1.0% |
| Water | 0.02%-0.10% |

*“q.s.” refers to the amount of the propellant which is required to make up the desired volume of the inhaler, excluding the volume of the other components stated.

### Example 2

Table 3 below provides an exemplary composition provided by the invention.

TABLE 3
Exemplary Composition 2 (Solution).

| Ingredient | Quantity (wt. %) |
|---|---|
| Tiotropium | 0.005%-0.01% |
| Formoterol | 0.008% |
| Ethanol | 12%-18% |
| Propellant (HFA-134a; HFA-152a; HFO-1234ze) | q.s.* |
| Maleic acid | 0.05% |
| Glycerol | 0.0-1.0% |
| Water | 0.02%-0.10% |

*“q.s.” refers to the amount of the propellant which is required to make up the desired volume of the inhaler, excluding the volume of the other components stated.

### Example 3

Table 4 below provides an exemplary composition provided by the invention.

TABLE 4
Exemplary Composition 3 (Solution).

| Ingredient | Quantity (wt. %) |
|---|---|
| Tiotropium | 0.005%-0.01% |
| Formoterol | 0.008% |
| Beclomethasone | 0.18%-0.36% |
| Ethanol | 12%-18% |
| Propellant (HFA-134a; HFA-152a; HFO-1234ze) | q.s.* |
| Maleic acid | 0.05% |
| Glycerol | 0.0-1.0% |
| Water | 0.02%-0.10% |

*“q.s.” refers to the amount of the propellant which is required to make up the desired volume of the inhaler, excluding the volume of the other components stated.

### Example 4

Table 5 below provides an exemplary composition provided by the invention.

TABLE 5
Exemplary Composition 4 (Solution).

| Ingredient | Quantity (wt. %) |
|---|---|
| Tiotropium | 0.005%-0.01% |
| Formoterol | 0.008% |
| Ciclesonide | 0.18%-0.36% |
| Ethanol | 12%-18% |
| Propellant (HFA-134a; HFA-152a; HFO-1234ze) | q.s.* |
| Maleic acid | 0.05% |
| Glycerol | 0.0-1.0% |
| Water | 0.02%-0.10% |

*“q.s.” refers to the amount of the propellant which is required to make up the desired volume of the inhaler, excluding the volume of the other components stated.

### Example 5

Table 6 below provides an exemplary composition provided by the invention.

TABLE 6
Exemplary Composition 5 (Solution-Suspension Hybrid).

| Ingredient | Quantity (wt. %) |
|---|---|
| Tiotropium | 0.005%-0.01% |
| Formoterol | 0.008% |
| Fluticasone furoate | 0.18%-0.36% |
| Ethanol | 12%-18% |
| Propellant (HFA-134a; HFA-152a; HFO-1234ze) | q.s.* |
| Maleic acid | 0.05% |
| Glycerol | 0.0-1.0% |
| Water | 0.02%-0.10% |

*“q.s.” refers to the amount of the propellant which is required to make up the desired volume of the inhaler, excluding the volume of the other components stated.

### Example 6

Table 7 below provides an exemplary composition provided by the invention.

TABLE 7
Exemplary Composition 6 (Suspension).

| Ingredient | Quantity (wt. %) |
|---|---|
| Tiotropium Bromide | 0.01%-0.1% |
| Formoterol Fumarate Dihydrate | 0.005%-0.01% |
| Surfactant (s) | 0.001%-0.1% |
| Solvent (s) | 0.1%-2.5% |
| Suspending Agent (s) | 0.001%-10% |
| Bulking Agent (s) | 0.001%-10% |
| Stabilizer (s) | 0.001%-0.1% |
| Absorbent (s) | 0.001%-0.1% |
| Propellant | q.s.* |

*“q.s.” refers to the amount of the propellant which is required to make up the desired volume of the inhaler, excluding the volume of the other components stated.

### Example 7

Table 8 below provides an exemplary composition provided by the invention.

TABLE 8
Exemplary Composition 7 (Suspension).

| Ingredient | Quantity (wt. %) |
|---|---|
| Tiotropium Bromide | 0.01%-0.1% |
| Formoterol Fumarate Dihydrate | 0.005%-0.01% |
| Oleic Acid | 0.001%-0.1% |
| Ethanol | 0.1%-2.5% |
| PEG 400 | 0.001%-10% |
| Magnesium Stearate | 0.001%-10% |
| Citric Acid | 0.001%-0.1% |
| Sodium Cromoglycate | 0.001%-0.1% |
| Propellant | q.s.* |

*“q.s.” refers to the amount of the propellant which is required to make up the desired volume of the inhaler, excluding the volume of the other components stated.

### Example 8

Table 9 below provides an exemplary composition provided by the invention.

TABLE 9
Exemplary Composition 8 (Suspension).

| Ingredient | Quantity (wt. %) |
|---|---|
| Tiotropium Bromide | 0.01%-0.1% |
| Formoterol Fumarate Dihydrate | 0.005%-0.01% |
| Oleic Acid | 0.001%-0.1% |
| Ethanol | 0.1%-2.5% |
| PEG 400 | 0.001%-10% |
| Magnesium Stearate | 0.001%-10% |
| Maleic acid | 0.001%-0.1% |
| Sodium Cromoglycate | 0.001%-0.1% |
| Propellant | q.s.* |

*“q.s.” refers to the amount of the propellant which is required to make up the desired volume of the inhaler, excluding the volume of the other components stated.

### Example 9

Table 10 below provides an exemplary composition provided by the invention.

TABLE 10
Exemplary Composition 9 (Suspension).

| Ingredient | Quantity (wt. %) |
|---|---|
| Tiotropium Bromide | 0.01%-0.1% |
| Formoterol Fumarate Dihydrate | 0.005%-0.01% |
| Oleic Acid | 0.001%-0.1% |
| Ethanol | 0.1%-2.5% |
| PEG 400 | 0.001%-10% |
| Lactose | 0.001%-10% |
| Malic acid | 0.001%-0.1% |
| Sodium Cromoglycate | 0.001%-0.1% |
| Propellant | q.s.* |

*“q.s.” refers to the amount of the propellant which is required to make up the desired volume of the inhaler, excluding the volume of the other components stated.

### Example 10

Table 11 below provides additional exemplary compositions, e.g., solution compositions and a hybrid solution-suspension composition provided by the invention. In Table 11, the following shortened column names are utilized: “API(s)” (active pharmaceutical ingredient(s)); “Cosolv(s)” (cosolvent(s)); “Propell(s)” (propellant(s)); “Excip(s)” (excipient(s)); “Perform. Mod(s)” (performance modulator(s)); and “Form” (product form).

TABLE 11
Exemplary Compositions (Solutions/Solution-Suspension Hybrid).

| | | | | Perform. | | |
|---|---|---|---|---|---|---|
| API(s) | Cosolv(s) | | Excip(s) | Mod(s) | | |
| (wt. %) | (wt. %) | Propell(s) | (wt. %) | (wt. %) | Form | Storage |
| Tiotropium | Ethanol | HFA-134a | Citric | PEG-600 | Solution | RT or 2- |
| 0.005-0.01%* | 12-18% | HFA-152a | Acid | PEG-1000 | | 8° C. |
| | Water | HFO-1234ze | 0.05% | Glycerol | | |
| | 0.02- | | and 1M | (Combined | | |
| | 0.10% | | HCl 0.01- | 0.0- 1.0%) | | |
| | | | 0.05% | | | |
| Tiotropium | Ethanol | HFA-134a | Maleic | PEG-600 | Solution | RT or 2- |
| 0.005-0.01% | 12-18% | HFA-152a | Acid | PEG-1000 | | 8° C. |
| Formoterol | Water | HFO-1234ze | 0.05% | Glycerol | | |
| 0.008% | 0.02- | | | (Combined | | |
| | 0.10% | | | 0.0- 1.0%) | | |
| Tiotropium | Ethanol | HFA-134a | Maleic | PEG-600 | Solution | RT or 2- |
| 0.005-0.01% | 12-18% | HFA-152a | Acid | PEG-1000 | | 8° C. |
| Formoterol | Water | HFO-1234ze | 0.05% | Glycerol | | |
| 0.008% | 0.02- | | | (Combined | | |
| Beclomethasone | 0.10% | | | 0.0- 1.0%) | | |
| 0.18%-0.36 | | | | | | |
| Tiotropium | Ethanol | HFA-134a | Maleic | PEG-600 | Solution | RT or 2- |
| 0.005-0.01% | 12-18% | HFA-152a | Acid | PEG-1000 | | 8° C. |
| Formoterol | Water | HFO-1234ze | 0.05% | Glycerol | | |
| 0.008% | 0.02- | | | (Combined | | |
| Ciclesonide | 0.10% | | | 0.0-1.0%) | | |
| 0.18-0.36% | | | | | | |
| Tiotropium | Ethanol | HFA-134a | Maleic | PEG-600 | Solution - | RT or 2- |
| 0.005-0.01% | 12-18% | HFA-152a | Acid | PEG-1000 | Suspension | 8° C. |
| Formoterol | | HFO-1234ze | 0.05% | Glycerol | Hybrid | |
| 0.008% | | | | (Combined | | |
| Fluticasone | | | | 0.0-1.0%) | | |
| Furoate | | | | | | |
| 0.18-0.36% | | | | | | |

### Example 11

A brief description of exemplary manufacturing process(es) is/are provided here. Such manufacturing process(es) can be used to manufacture the exemplary compositions provided in Examples 1-10 above. Manufacturing steps provided in this Example can, uncontradicted, be performed in any order.
Step 1: In compositions comprising two or more active pharmaceutical ingredients (API), at least a first API is combined with at least a second API.
Step 2: Step 1 is repeated until all APIs have been combined.
Step 3: In compositions comprising a single API, the single API; or, in compositions comprising two or more APIs, the combination of APIs resulting from the above manufacturing step(s), are mixed with at least one other non-volatile composition component (e.g., composition excipient), such as, e.g., one, some, or all remaining non-volatile composition component(s) (excipient(s)).
Step 4: If not all remaining composition component(s) (excipient(s)) have been combined in step 3, at least one other non-volatile composition component (excipient), such as, e.g., one, some, or all remaining non-volatile composition component(s) (excipient(s)) are added. This step is repeated until all non-volatile composition components (excipients) have been added.
Step 5: The combined components of manufacturing process step 4 (or step 3 as appropriate) are mixed until uniformly suspended (as in, e.g., in the preparation of a suspension composition), or, e.g., until sufficiently mixed to facilitate addition of a propellant as is described in step 6.a.i, below.
Step 6: The required quantity of the composition is added to a container, e.g., a canister suitable for use with a metered dose inhaler (MDI), or other final packaging unit. Such addition can be accomplished by a cold filling method (described in steps 6.a and 6.b) or a pressure filling method (described in steps 7 or alternatively step 8). In certain manufacturing processes, the container is a metal container, the container is uncoated with any polymeric compound(s), the container is non-anodized, the MDI comprises no component, e.g., valve(s) or ring(s) made of a butyl or halo-butyl rubber, or any combination thereof. In certain alternative embodiments of manufacturing processes, the container is a non-metal container, the container is coated with one or more of polymeric compound(s), is an anodized container, the MDI comprises one or more components, e.g., valve(s) or ring(s) made of a butyl or halo-butyl rubber, or any combination thereof.
Step 6.a: The required amount of propellant is added to the composition resulting from step 5 and the resulting composition is mixed until the composition is completely dissolved (in, e.g., a composition provided as a solution) or until the composition is uniformly suspended (in, e.g., a composition provided as a suspension).
Step 6.b: The addition of the composition to a container, e.g., a canister, is completed by a cold filling process comprising (a) chilling the composition resulting from step 6.a.i. to a suitable temperature, such as, e.g., between about −50° C.-about −60° C.; (b) adding the composition to the container, e.g., canister; (c) attaching a valve, e.g., a metered dose valve, to the container, e.g., canister, e.g., by crimping the valve, e.g., metered dose valve, to the container, e.g., canister; (d) allowing the container, e.g., canister, to warm to ambient temperature, resulting in an increase in pressure within the container, e.g., canister.
Step 7: The addition of the composition to a container, e.g., canister, is completed by a pressure filling process comprising (a) adding the required amount of propellant to the composition resulting from step 5; (b) attaching a valve, e.g., a metered dose valve, to a container, e.g., canister, e.g., by crimping the valve, e.g., metered dose valve, to the container, e.g., canister; (c) adding the composition resulting from (a) to the container, e.g., canister, through the valve, e.g., metered dose valve, by way of applied pressure.
Step 8. The addition of the composition to a container, e.g., canister, is completed by a pressure filling process comprising (a) adding all non-volatile composition component(s) from step 5 to the container, e.g., canister; (b) attaching a valve, e.g., a metered dose valve, to the container, e.g., canister; (c) adding the volatile composition component(s), e.g., propellant(s) to the container, e.g., canister, through the valve, e.g., metered dose valve, by way of applied pressure.

### Example 12

Table 12 below provides 16 additional exemplary compositions (E1-E16), e.g., solution or solution-suspension hybrid compositions, provided by the invention. An exemplary process for manufacturing the exemplified compositions provided here was followed to generate the provided compositions. Table 12 provides the amount of each respective API per dispensation when the composition is dispensed from device(s) comprising a 50 μL valve.

TABLE 12
Exemplary Compositions (Solutions/Solution-Suspension Hybrid).

| | (wt. %) | | | | | | | |
|---|---|---|---|---|---|---|---|---|
| API/excipient | E1 | E2 | E3 | E4 | E5 | E6 | E7 | E8 |
| Tiotropium Bromide | 0.008 | 0.008 | 0.008 | 0.008 | 0.008 | 0.008 | 0.008 | 0.008 |
| Formoterol | 0.008 | 0.008 | 0.008 | 0.008 | 0.008 | 0.008 | 0.008 | 0.008 |
| Fumarate Dihydrate | | | | | | | | |
| Beclomethasone | — | — | — | — | — | — | — | — |
| Dipropionate | | | | | | | | |
| Ciclesonide | — | — | — | — | — | — | — | — |
| Fluticasone Furoate | — | — | — | — | — | — | — | — |
| Citric Acid | — | — | 0.04 | 0.04 | — | — | 0.04 | 0.04 |
| Hydrochloric Acid | — | — | — | — | 0.016 | 0.016 | 0.016 | 0.016 |
| (1.0 M Solution) | | | | | | | | |
| Maleic Acid | 0.002 | 0.002 | 0.002 | 0.002 | — | — | — | — |
| Ethanol | 15 | 15 | 15 | 15 | 15 | 15 | 15 | 15 |
| Water | — | 0.5 | — | 0.5 | — | 0.5 | — | 0.5 |
| Glycerol | — | — | — | — | — | — | — | — |
| PEG-1000 | — | — | — | — | — | — | — | — |
| Propellant (one of | Q.S. | Q.S. | Q.S. | Q.S. | Q.S. | Q.S. | Q.S. | Q.S. |
| HFA-134a, HFA- | | | | | | | | |
| 152a or HFO-1234ze) | | | | | | | | |
| Amount of API per | 4/4 | 4/4 | 4/4 | 4/4 | 4/4 | 4/4 | 4/4 | 4/4 |
| dispensation (ug/ | | | | | | | | |
| actuation), in | | | | | | | | |
| sequence of above | | | | | | | | |
| APIs (50 uL valve) | | | | | | | | |

(wt. %)

| API/excipient | E9 | E10 | E11 | E12 | E13 | E14 | E15 | E16 |
|---|---|---|---|---|---|---|---|---|
| Tiotropium | 0.008 | 0.008 | 0.008 | 0.008 | 0.008 | 0.008 | 0.008 | 0.008 |
| Bromide | | | | | | | | |
| Formoterol | 0.008 | 0.008 | 0.008 | 0.008 | 0.008 | 0.008 | 0.008 | 0.008 |
| Fumarate Dihydrate | | | | | | | | |
| Beclomethasone | — | — | 0.2 | 0.2 | — | — | — | — |
| Dipropionate | | | | | | | | |
| Ciclesonide | — | | — | — | — | — | 0.2 | 0.2 |
| Fluticasone Furoate | — | — | | — | 0.2 | 0.2 | — | — |
| Citric Acid | 0.04 | 0.04 | 0.04 | 0.04 | 0.04 | 0.04 | 0.04 | 0.04 |
| Hydrochloric Acid | — | — | — | — | — | — | — | — |
| (1.0 M Solution) | | | | | | | | |
| Maleic Acid | 0.002 | 0.002 | 0.002 | 0.002 | 0.002 | 0.002 | 0.002 | 0.002 |
| Ethanol | 15 | 15 | 15 | 15 | 10 | 10 | 10 | 10 |
| Water | — | 0.5 | — | 0.5 | — | 0.5 | — | 0.5 |
| Glycerol | 0.5 | 0.5 | 0.5 | 0.5 | 0.5 | 0.5 | 0.5 | 0.5 |
| PEG-1000 | 0.1 | 0.1 | 0.1 | 0.1 | 0.1 | 0.1 | 0.1 | 0.1 |
| Propellant (one of | Q.S. | Q.S. | Q.S. | Q.S. | Q.S. | Q.S. | Q.S. | Q.S. |
| HFA-134a, HFA- | | | | | | | | |
| 152a or HFO- | | | | | | | | |
| 1234ze) | | | | | | | | |
| Amount of API per | 4/4 | 4/4 | 4/4/100 | 4/4/100 | 4/4/100 | 4/4/100 | 4/4/100 | 4/4/100 |
| dispensation (μg/ | | | | | | | | |
| actuation), in | | | | | | | | |
| sequence of above | | | | | | | | |
| APIs (50 μL valve) | | | | | | | | |

The following exemplary manufacturing process was used to generate each of the exemplary compositions provided in Table 12.

The performance modulating agent (glycerol/ethanol, as applicable) was weighed and added to a first, pre-mix, vessel.

Ethanol was collected and transferred to the pre-mix vessel.

Water was collected (where applicable) and transferred to the premix vessel.

Acidic compounds (citric acid, hydrochloric acid, maleic acid, where applicable) were weighted and transferred to the pre-mix vessel.

The content of the pre-mix vessel was mixed well until a clear solution formed.

About 70% of the propellant was added to a second, main, vessel.

The solution in the pre-mix vessel was transferred from the pre-mix vessel to the main vessel.

The API(s) were weighed/collected and added to the main vessel.

The main vessel was topped up with propellant to bring the composition to a target volume (target weight).

The content of the main vessel was mixed until a clear solution formed.

For composition(s)/formulation(s) comprising a fluticasone compound, the composition was homogenized using a high shear mix for 20-60 minutes.

Canisters which had been vacuum crimped with valves of 25-75 mcl were pressure filled.

### Example 13

Table 13 below provides 8 additional exemplary compositions (E1-E8), e.g., suspension compositions, provided by the invention. An exemplary process for manufacturing the exemplified compositions provided here was followed to generate the provided compositions. Table 13 provides the amount of each respective API per dispensation when the composition is dispensed from device(s) comprising a 50 μL valve.

TABLE 13
Exemplary Compositions (Suspensions).

| | (wt. %) | | | | | | | |
|---|---|---|---|---|---|---|---|---|
| API/excipient | E1 | E2 | E3 | E4 | E5 | E6 | E7 | E8 |
| Tiotropium Bromide | 0.008 | 0.008 | 0.008 | 0.008 | 0.008 | 0.008 | 0.008 | 0.008 |
| Formoterol | 0.008 | 0.008 | 0.008 | 0.008 | 0.008 | 0.008 | 0.008 | 0.008 |
| Fumarate Dihydrate | | | | | | | | |
| Fluticasone Furoate | — | — | — | — | 0.2 | 0.2 | 0.2 | 0.2 |
| Ethanol | 1 | — | — | 1 | 1 | — | — | 1 |
| PEG-1000 | — | 0.1 | 0.1 | — | — | 0.1 | 0.1 | — |
| Oleic Acid | 0.005 | — | — | 0.005 | 0.005 | — | — | 0.005 |
| PVP k25 | — | 0.0001 | 0.0001 | — | — | 0.0001 | 0.0001 | — |
| Sodium | — | — | 0.002 | 0.002 | — | — | 0.002 | 0.002 |
| Cromoglycate | | | | | | | | |
| Propellant (one of | Q.S | Q.S | Q.S | Q.S | Q.S | Q.S | Q.S | Q.S |
| HFA-134a, HFA- | | | | | | | | |
| 152a or HFO- | | | | | | | | |
| 1234ze) | | | | | | | | |
| Amount of API per | 4/4 | 4/4 | 4/4 | 4/4 | 4/4/100 | 4/4/100 | 4/4/100 | 4/4/100 |
| dispensation (μg/ | | | | | | | | |
| actuation), in | | | | | | | | |
| sequence of above | | | | | | | | |
| APIs (50 μL valve) | | | | | | | | |

The following exemplary manufacturing process was used to generate each of the exemplary compositions provided in Table 13.

All solid-form excipients (excipients provided in solid form) were weighed and transferred into a main vessel.

All semi-solid form excipients (excipients provided in semi-solid form) were weighted and transferred into the main vessel.

Approximately 80% of the required amount of propellant was added to the main vessel.

Ethanol (as applicable) was collected and transferred into the main vessel.

The applicable amount(s) of API(s) were added to the main vessel.

The main vessel was topped off with propellant to bring the composition to a target volume (target weight).

The content of the vessel was mixed for at least 20 min.

The content of the vessel was homogenized (high shear mixing) for 20-60 minutes using an inline homogenizer, an in-dwelling homogenizer, or a bottom load homogenizer/high shear mixer.

Final packaging (canisters) that had been vacuum crimped with valves of 25 μL-75 μL were filled using pressure filling.

What is claimed is:

1. A pharmaceutically acceptable composition in the form of a solution comprising (a) an active pharmaceutical ingredient component wherein each constituent of the active pharmaceutical ingredient component is at least substantially dissolved such that the solution is sufficiently homogeneous so as to allow light to at least substantially uniformly pass through the composition, wherein the active pharmaceutical ingredient component comprises an effective amount of at least one pharmaceutically acceptable tiotropium compound and an effective amount of at least one pharmaceutically acceptable formoterol compound; (b) a solvent component comprising an effective amount of at least one C1-C6 alcohol characterizable as an alkyl alcohol, an aliphatic alcohol, or both; (c) a pharmaceutically acceptable and compositionally compatible small compound stabilizing agent having a molecular weight of less than about 200 g/mol, providing detectable or significant chelating activity, or both, wherein the small compound stabilizing agent is capable of detectably or significantly preventing or delaying any change in the homogeneity of the composition resulting in (i) the amount of light capable of passing through the composition to be reduced or (ii) the composition no longer being characterizable as a solution; and (d) a propellant component comprising one or more pharmaceutically acceptable propellant compounds.

2. The composition of claim 1, wherein the active pharmaceutical ingredient component further comprises an effective amount of at least one pharmaceutically acceptable inhalable corticosteroid compound.

3. The pharmaceutically acceptable composition of claim 2, wherein at least one of the at least one pharmaceutically acceptable inhalable corticosteroid compounds is beclomethasone, ciclesonide, or fluticasone furoate.

4. The pharmaceutically acceptable composition of claim 1, wherein (1) the C1-C6 alcohol characterizable as an alkyl alcohol, aliphatic alcohol, or both, and is present in a concentration of at least about 6 wt. %, (2) the ratio of the pharmaceutically acceptable and compositionally compatible small compound stabilizing agent to the at least one C1-C6 alcohol characterizable as an alkyl alcohol, an aliphatic alcohol, or both, is not greater than about 1:300, or (3) the C1-C6 alcohol characterizable as an alkyl alcohol, aliphatic alcohol, or both, and is present in a concentration of at least about 6 wt. % and the ratio of the pharmaceutically acceptable and compositionally compatible small compound stabilizing agent to the at least one C1-C6 alcohol characterizable as an alkyl alcohol, an aliphatic alcohol, or both, is not greater than about 1:300.

5. The composition of claim 1, wherein at least about 96% of the propellant component is composed of a dominant propellant compound and the ratio of the concentration of the dominant propellant compound to the concentration of the pharmaceutically acceptable and compositionally compatible organic acid compound is at least about 320:1.

6. The pharmaceutically acceptable composition of claim 5, wherein the propellant component does not comprise HFA-227.

7. The pharmaceutically acceptable composition of claim 6, wherein the pharmaceutically acceptable composition is free of any compound that exhibits significant surfactant activity.

8. The pharmaceutically acceptable composition of claim 1, wherein (a) the pharmaceutically acceptable and compositionally compatible small compound stabilizing agent comprises an organic acid compound; (b) the composition further comprises a sugar alcohol; or (c) the pharmaceutically acceptable and compositionally compatible small compound stabilizing agent comprises an organic acid and the composition further comprises a sugar alcohol.

9. A method of promoting airflow in a mammal comprising administering an effective amount of the composition of claim 1 an effective number of times over a therapeutically effective period, wherein performing the method causes a detectable dilation of an airway in the mammal.

10. A pharmaceutically acceptable composition comprising (1) an active pharmaceutical ingredient component comprising at least one pharmaceutically acceptable tiotropium compound and an effective amount of at least one pharmaceutically acceptable formoterol compound where at least a significant portion of one or more constituents of the active pharmaceutical ingredient component remain undissolved such that light cannot pass uniformly through the composition and the composition is characterizable as a suspension or a hybrid solution-suspension, (2) one or more pharmaceutically acceptable and compositionally compatible small compound stabilizing agent(s) having a molecular weight of less than about 200 g/mol, providing detectable or significant chelating activity, or both, wherein the small compound stabilizing agent is capable of detectably or significantly preventing or delaying any change in the homogeneity of the composition and the pharmaceutically acceptable and compositionally compatible small compound stabilizing agent(s) is present in an amount of at least about 0.04 wt. %; (3) a surfactant compound; and (3) a propellant component comprising one or more pharmaceutically acceptable propellant compounds, wherein the propellant component does not comprise HFA-227.

11. The composition of claim 10, wherein the tiotropium compound is tiotropium bromide, and the tiotropium bromide is present in a concentration of about 0.005-about 0.01 wt. %.

12. The composition of claim 11, wherein the formoterol compound is a pharmaceutically acceptable salt of formoterol.

13. The composition of claim 12, wherein the formoterol compound is formoterol fumarate dihydrate, and the formoterol fumarate dihydrate is present in a concentration of about 0.005-about 0.01 wt. %.

14. The composition of claim 13, wherein the active pharmaceutical ingredient component further comprises an effective amount of at least one pharmaceutically acceptable inhalable corticosteroid compound.

15. The composition of claim 14, wherein at least one of the at least one pharmaceutically acceptable inhalable corticosteroid compounds is beclomethasone, ciclesonide, or fluticasone furoate.

16. The composition of claim 13, wherein the composition further comprises (1) a glycerol in a concentration of about 0.00001 wt. %-about 1 wt. %, (2) a C1-C6 alcohol present in a concentration of about 0.1 wt. %-about 18 wt. %, or (3) both a glycerol in a concentration of about 0.00001 wt. %-about 1 wt. % and a C1-C6 alcohol present in a concentration of about 0.1 wt. %-about 18 wt. %.

17. A method of promoting airflow in a mammal, comprising administering an effective amount of the composition of claim 10 to the mammal an effective number of times over a therapeutically effective period, wherein performing the method causes a detectable dilation of an airway in the mammal.

18. A pharmaceutically acceptable composition comprising (a) an active pharmaceutical ingredient component wherein each constituent of the active pharmaceutical ingredient component is at least substantially dissolved such that the solution is sufficiently homogeneous so as to allow light to at least substantially uniformly pass through the composition, wherein the active pharmaceutical ingredient component comprises about 0.006 wt. %-about 0.01 wt. % tiotropium bromide and about 0.006 wt. %-about 0.01 wt. % formoterol fumarate dihydrate; (b) 0.01%-0.1% malic acid, citric acid, or maleic acid; and (c) a propellant component that is composed of HFO-1234ze, HFA-134a, HFA-152a, or a mixture of some or all thereof.

19. The composition of claim 18, wherein the composition comprises about 0.008 wt. % tiotropium bromide and about 0.008 wt. % formoterol fumarate dihydrate.

20. A method of promoting airflow in a mammal, comprising administering an effective amount of the composition of claim 18 to the mammal an effective number of times over a therapeutically effective period, wherein performing the method causes a detectable dilation of an airway in the mammal.
